SEQUENCE LISTING (417 sequences)

SEQ ID NO: 1 - protein, 254 aa, Synthetic construct
  REGION 1..254: note = mature
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 2 - protein, 208 aa, Synthetic construct [sequence omitted]

SEQ ID NO: 3 - DNA, 321 bp, Synthetic construct
  misc_feature 1..321: note = CD80_165
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacgtgaatatatggcccgagtacaagaaccggaccatcagtgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 4 - DNA, 321 bp, Synthetic construct
  misc_feature 1..321: note = CD80_166
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacgtgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 5 - DNA, 321 bp, Synthetic construct
  misc_feature 1..321: note = CD80_167
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactattatgtctggggacgtgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgac

SEQ ID NO: 6 - DNA, 321 bp, Synthetic construct
  misc_feature 1..321: note = CD80_168
  source 1..321: mol_type = other DNA
gttatccacgtgaccaaggaagtgaatggagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcacttctaacctctccattgtgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 7 - DNA, 321 bp, Synthetic construct
  misc_feature 1..321: note = CD80_169
  source 1..321: mol_type = other DNA
gttatccacgtgaccaaggaagtgaaaggagtggcaacgctgtcctgtggtcacaatgtttctgttgaagagctggcacaaactcgcatctactggcaaaaggggaagaaaatggtgctgactatgatgtctggggaggttaatatatggcccgagtacaagaaccggaccatctttgatatcactaataacctctccattatgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 8 - DNA, 321 bp, Synthetic construct
  misc_feature 1..321: note = CD80_170
  source 1..321: mol_type = other DNA
gttatccacgtgaccaaggaagtgaatggagtggcaacgctgtcctgtggtcacaatgtttctgttgaagagctggcacaaactcgcatctactggcaaaaggggaagaaaatggtgctgactatgatgtctggggaggttaatatatggcccgagtacaagaaccggaccatctttgatatcactaataacctctccattatgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 9 - DNA, 321 bp, Synthetic construct
  misc_feature 1..321: note = CD80_171
  source 1..321: mol_type = other DNA
gttatccacgtgaccaagtctgtgaaagaagtggcaacgctgtcctgtggtatcaatttgtctgttgaagagctgaagcaaactcgcatctactggcaaaaggagaagaaaatggtgctgactatgatgtctggggacctgaatatatggcccgagtacaagaaccggaccatttttgatatcactaataacctctccattgtgatcctgaatctgcgcccatctgacgagggcacatacgagtgtgttgttctgaagtatgaaaaagacgctttcaagcgggaacacctggctgaagtgacgttatcagtcaaagctgac

SEQ ID NO: 10 - DNA, 321 bp, Synthetic construct
  misc_feature 1..321: note = CD80_172
  source 1..321: mol_type = other DNA
gttatccacgtgaccaaggaagtgagatcttatgcaacgctgtcctgtggtcacaatgtttctgttgaagagctggcacaaactcgcatctactggcaaaaggagaagaaaatggtgctgactctgatgtctggggacatgaatatatggcccgagtacaagaaccggaccatcatggatatcactaataacctctccattatgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 11 - DNA, 321 bp, Synthetic construct
  misc_feature 1..321: note = CD80_173
  source 1..321: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 12 - DNA, 321 bp, Synthetic construct
  misc_feature 1..321: note = CD80_174
  source 1..321: mol_type = other DNA
gttatccacgtgaccaaggaagtgaaacgggtggcaacgctgtcctgtggtcacaatgtttctgttgaagagctggcacaaactcgcatctactggcaaaaggagaagaaaatggtgctgactatgatgtctggggacaggaatatatggcccgagtacaagaaccggaccatctttgatatcactattaacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 13 - DNA, 321 bp, Synthetic construct
  misc_feature 1..321: note = CD80_175
  source 1..321: mol_type = other DNA
gttatccacgtgaccaaggaagtgaaatcatttgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatctctaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 14 - DNA, 321 bp, Synthetic construct
  misc_feature 1..321: note = CD80_176
  source 1..321: mol_type = other DNA
gttatccacgtgaccaaggaagtgaaacgggtggcaacgctgtcctgtggttataatgtttctgttgaagagctgggacaaactcgcatctactggcaaaaggataagaaaatggtgctgactatgatgtctggggacatgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 15 - DNA, 321 bp, Synthetic construct
  misc_feature 1..321: note = CD80_177
  source 1..321: mol_type = other DNA
gttatccacgtgaccaaggaagtgaaagggtgggcaacgctgtcctgtggtcacaatgtttcttctgaagagctggcacaacctcgcatctactggcaaaaggagaaaaaaatggtgctgactatgatgtctggggacatgaatatatggcccgagtacaagaaccggaccatctttgatatcactaataacctctccattgtgatcctgggtctgcgcccatctgacgagggcacatacaagtgtgttgttctgaagagggaaaaagacgctttcaagcgggaacacctggctgaagtgacgttatcagtcaaagctgac

SEQ ID NO: 16 - DNA, 321 bp, Synthetic construct
  misc_feature 1..321: note = CD80_178
  source 1..321: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttgagaagattgaaaaagacgctttcaagcgggaacacctggctgaagtgacgttatcagtcaaagctgac

SEQ ID NO: 17 - DNA, 321 bp, Synthetic construct
  misc_feature 1..321: note = CD80_179
  source 1..321: mol_type = other DNA
gttatccacgtgaccaaggaagtgaaagaagtggcaacgctgtcctgtggtttcaatgtttctgttgaagagctggcacaaactcgcatctactggcaaaaggagaagaaaatggtgctgactgggatgtctggggacatgaatatatggcccgagtacaagaaccggaccatctatgatatcactaataacctctccattaatatcctggctctgcgcccatctgacgagggcacatacgagtgtgttgttctgaagtatgaaaaagacgctttcaagcgggaacacctggctgaagtgacgttatcagtcaaagctgac

SEQ ID NO: 18 - DNA, 321 bp, Synthetic construct
  misc_feature 1..321: note = CD80_180
  source 1..321: mol_type = other DNA
gttatccacgtgaccaaggaagtgaaagggtgggcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 19 - DNA, 321 bp, Synthetic construct
  misc_feature 1..321: note = CD80_181
  source 1..321: mol_type = other DNA
gttatccacgtgaccaaggaagtgagtcgatatgcaacgctgtcctgtggtcacaatgtttctgttgaagagctggcacaaactcgcatctactggcaaaaggagaagaaaatggtgctgactatgatgtctggggacctgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgac

SEQ ID NO: 20 - DNA, 321 bp, Synthetic construct
  misc_feature 1..321: note = CD80_182
  source 1..321: mol_type = other DNA
gttatccacgtgaccaaggaagtgaaagggtgggcaacgctgtcctgtggtcacaatgtttctgttgaagagctggcacaaactcgcatctactggcaaaaggggaagaaaatggtgctgactatgatgtctggggacctgaatatatggcccgagtacaagaaccggaccatctttgatatcactaataacctctccattgtgatcctgggtctgcgcccatctgacgagggcacatacgagtgtgttgttctgaagagggaaaaagacgctttcaagcgggaacacctggctgaagtgacgttatcagtcaaagctgac

SEQ ID NO: 21 - DNA, 321 bp, Synthetic construct
  misc_feature 1..321: note = CD80_183
  source 1..321: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctgggtctgcgcccatctgacgagggcacatacgagtgtgttgttctgaagagggaaaaagacgctttcaagcgggaacacctggctgaagtgacgttatcagtcaaagctgac

SEQ ID NO: 22 - DNA, 321 bp, Synthetic construct
  misc_feature 1..321: note = CD80_184
  source 1..321: mol_type = other DNA
gttatccacgtgaccaaggaagtgaaagaatatgcaacgctgtcctgtggtcacaatgtttctgttgaagagctgcagcaaactcgcatctactggcaaaaggagaagaaaatggtgctgactatgatgtctggggacctgaatatatggcccgagtacaagaaccggaccatctttgatatcactaataacctctccattgtgatcctgggtctgcgcccatctgacgagggcacatacgagtgtgttgttctgaagagggaaaaagacgctttcaagcgggaacacctggctgaagtgacgttatcagtcaaagctgac

SEQ ID NO: 23 - DNA, 321 bp, Synthetic construct
  misc_feature 1..321: note = CD80_185
  source 1..321: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 24 - DNA, 321 bp, Synthetic construct
  misc_feature 1..321: note = CD80_186
  source 1..321: mol_type = other DNA
gttatccacgtgaccaagaaagtgaaagaatgggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactcataacctctccattgtgatcctgggtctgcgcccatctgacgagggcacatacgagtgtgttgttctgaagaaggaaaaagacgctttcaagcgggaacacctggctgaagtgacgttatcagtcaaagctgac

SEQ ID NO: 25 - DNA, 321 bp, Synthetic construct
  misc_feature 1..321: note = CD80_187
  source 1..321: mol_type = other DNA
gttatccacgtgaccaaggaagtgaaagaatatgcaacgctgtcctgtggttataatgtttctgttgaagagctggcacaaactcgcatctactggcaaaaggggaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttcagaagtatgaaaaagacgctttcaagcgggaacacctggctgaagtgacgttatcagtcaaagctgac

SEQ ID NO: 26 - DNA, 321 bp, Synthetic construct
  misc_feature 1..321: note = CD80_188
  source 1..321: mol_type = other DNA
gttatccacgtgaccaaggaagtgaaagaagtggcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 27 - DNA, 321 bp, Synthetic construct
  misc_feature 1..321: note = CD80_189
  source 1..321: mol_type = other DNA
gttatccacgtgaccaaggaagtgaaagggtgggcaacgctgtcctgtggtcacaatgtttctgttgaagagctggcacaaactcgcatctactggcaaaaggagaagaaaatggtgctgactatgatgtctggggacgtgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 28 - DNA, 321 bp, Synthetic construct
  misc_feature 1..321: note = CD80_190
  source 1..321: mol_type = other DNA
gttatccacgtgaccaaggaagtgaaagaagtggcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 29 - DNA, 321 bp, Synthetic construct
  misc_feature 1..321: note = CD80_191
  source 1..321: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 30 - DNA, 321 bp, Synthetic construct
  misc_feature 1..321: note = CD80_192
  source 1..321: mol_type = other DNA
gttatccacgtgaccaagaatgtgaaagaagtggcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgcgtttatcagtcaaagctgac

SEQ ID NO: 31 - DNA, 321 bp, Synthetic construct
  misc_feature 1..321: note = CD80_193
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgacttggatgtctggggacatgaatatatggcccgagtacaagaaccggaccatctttgatatcgctaataacctctccattgtgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 32 - DNA, 321 bp, Synthetic construct
  misc_feature 1..321: note = CD80_194
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgacttggatgtctggggacatgaatatatggcccgagtacaagaaccggaccatctttgatatcgctaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 33 - DNA, 321 bp, Synthetic construct
  misc_feature 1..321: note = CD80_195
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgacttggatgtctggggacatgaatatatggcccgagtacaagaaccggaccatctatgatatcactaataacctctccattaatatcctggctctgcgcccatctgacgagggcacatacgagtgtgttgttctgaagtatgaaaaagacgctttcaagcgggaacacctggctgaagtgacgttatcagtcaaagctgac

SEQ ID NO: 34 - DNA, 321 bp, Synthetic construct
  misc_feature 1..321: note = CD80_196
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgacttggatgtctggggacatgaatatatggcccaagtacaagaaccggaccatctttgatatcgctaataacctctccattgtgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 35 - DNA, 321 bp, Synthetic construct
  misc_feature 1..321: note = CD80_197
  source 1..321: mol_type = other DNA
gttatccacgtgaccaaggaagtgaaagaatatgcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagcaggaaaaagacgctttcaagcgggaacacctggctgaagtgaggttatcagtcaaagctgac

SEQ ID NO: 36 - DNA, 321 bp, Synthetic construct
  misc_feature 1..321: note = CD80_198
  source 1..321: mol_type = other DNA
gttatccacgtgaccaaggaagtgaaagaagtggcaacgctgtcgtgtggttacaatgtttctgttgaagagctggaacaaactagcatctactggcaaaaggataagaaaatggtgctgactatgatgtctggggacttgaatatatggcccgagtacaagaaccggaccatctttgatatcactaataacctctccattatgatcctgggtctgcgcccatctgacgggggcacatacgagtgtgttgttctgaagtatgaaaaaggcgctttcaagcgggaacacctggctgaagtgacgttatcagtcaaagctgac

SEQ ID NO: 37 - DNA, 321 bp, Synthetic construct
  misc_feature 1..321: note = CD80_199
  source 1..321: mol_type = other DNA
gttatccacgtgaccaaggaagtgaaaggggtggcaacgctgtcctgtggttacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 38 - DNA, 321 bp, Synthetic construct
  misc_feature 1..321: note = CD80_200
  source 1..321: mol_type = other DNA
gttatccacgtgaccaaggaagtgaatagagtggcaacgctgtcctgtggtgtgaatgtttctgttgaagagctggcacaatatcgcatctactggcaaaaggagaagaaaatggtgctgactatgatgtctggggacatgaatatatggcccgagtacaagaaccggaccatctttgatatcactaataacctctccattgtgatcctggggctgcgcccatctgacgagggcacatacgagtgtgttgttctgaagtatgaaaaagacgctttcaagcgggaacacctggctgaagtgacgttatcagtcaaagctgac

SEQ ID NO: 39 - DNA, 321 bp, Synthetic construct
  misc_feature 1..321: note = CD80_201
  source 1..321: mol_type = other DNA
gttatccacgtgaccaaggaagtgaaaggggtggcaacgctgtcctgtggttacaatgtttctgttgaagagctggcacaatatcgcatctactggcaaaaggagaagaaaatggtgctgactatgatgtctggggactggaatatatggcccgagtacaagaaccggaccatctttgatatctctaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 40 - DNA, 321 bp, Synthetic construct
  misc_feature 1..321: note = CD80_202
  source 1..321: mol_type = other DNA
gttatccacgtgaccaagcatgtgaaagaagtggcaacgctgtcctgtggtctcaatatttctgttgaagagctggcacaatatcgcatctactggcaaaaggagaagaaaatggtgctgactatgatgtctgggtccatgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgac

SEQ ID NO: 41 - DNA, 321 bp, Synthetic construct
  misc_feature 1..321: note = CD80_203
  source 1..321: mol_type = other DNA
gttatccacgtgaccaaggaagtgaatgcatgggcaacgctgtcctgtggtttcaatgtttctgttgaagagctggcacaacatcgcatctactggcaaaaggagaagaaaatggtgctgactatgatgtctggggacttgaatatatggcccgagtacaagaaccggaccatctttgatatcgagaataacctctccattgtgatcctggctctggacccatctgacgagggcacatacgagtgtgttgttctgaagtatgaaaaagacgctttcaagcgggaacacctggctgaagtgacgttatcagtcaaagctgac

SEQ ID NO: 42 - DNA, 321 bp, Synthetic construct
  misc_feature 1..321: note = CD80_204
  source 1..321: mol_type = other DNA
gttatccacgtgaccaaggaagtgaaagaagtggcaacgctgtcctgtggtgttaatatttctgtggaagagctggcacaatatcgcatctactggcaaaaggggaagaaaatggtgctgactatgatgtctggggacgtgaatatatggcccgagtacaagaaccggaccatctttgatatcactaataacctctccattgtgatcctggctctggagccatctgacgagggcacatacgagtgtgttgttctgaagtatgaaaaagacgctttcaagcgggaacacctggctgaagtgacgttatcagtcaaagctgac

SEQ ID NO: 43 - DNA, 321 bp, Synthetic construct
  misc_feature 1..321: note = CD80_205
  source 1..321: mol_type = other DNA
gttatccacgtgaccaaggaagtgaaaggtgtggcaacgctgtcctgtggtcacaatgtttctgttgaagagctggcacaaactcgcatctactggcaaaaggagaagaaaatggtgctgactatgatgtctgggaagatgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 44 - DNA, 321 bp, Synthetic construct
  misc_feature 1..321: note = CD80_206
  source 1..321: mol_type = other DNA
gttatccacgtgaccaaggaagtgaaaggagtggcaacgctgtcctgtggtaccaatgtttctgttgaagagctggcaacaactcgcatctactggcaaaaggagaagaaaatggtgctgactatgatgtctggggagttgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 45 - DNA, 321 bp, Synthetic construct
  misc_feature 1..321: note = CD80_207
  source 1..321: mol_type = other DNA
gttatccacgtgaccaaggaagtgaaagaagtggcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatctttgatatcactaataacctctccattactatcctggctctgcgcccatctgacgagggcacatacgagtgtgttgttctgaagtatgaaaaagacgctttcaagcgggaacacctggctgaagtgaagttatcagtcaaagctgac

SEQ ID NO: 46 - DNA, 321 bp, Synthetic construct
  misc_feature 1..321: note = CD80_208
  source 1..321: mol_type = other DNA
gttatccacgtgaccaaggaagtgaaagaatatgcaacgctgtcctgtggtcacaatgtttctgttgaagagctggcacaaactcgcatctactggcaaaaggggaagaaaatggtgctgactggtatgtctggggacatgaatatatggcccgagtacaagaaccggaccatcagtgatatcactaataacctctccattgtgatcctggctctgcgcccatctgacgagggcacatacgagtgtgttgttttgaagtatgaaaaagacgctttcaagcgggaacacctggctgaagtgacgttatcagtcaaagctgac

SEQ ID NO: 47 - DNA, 321 bp, Synthetic construct
  misc_feature 1..321: note = CD80_209
  source 1..321: mol_type = other DNA
gttatccacgtgaccaaggaagtgaaagaatatgcaacgctgtcctgtggtcacaatgtttctgttgaagagctggcacaacgtcgcatctactggcaaaaggggaagaaaatggtgctgactatgatgtctggggacttgaatatatggcccgagtacaagaaccggaccatcagtgatatcactaataacctctccattgtgatcctggctctgcgcccatctgacgagggcacatacgagtgtgttgttttgaagtatgaaaaagacgctttcaagcgggaacacctggctgaagtgacgttatcagtcaaagctgac

SEQ ID NO: 48 - DNA, 321 bp, Synthetic construct
  misc_feature 1..321: note = CD80_210
  source 1..321: mol_type = other DNA
gttatccacgtgaccaagaatgtgaaagaagtggcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatcagtgatatcactaataacctctccattgtgatcctggctctgcgcccatctgacgagggcacatacgagtgtgttgttttgaagtatgaaaaagacgctttcaagcgggaacacctggctgaagtgacgttatcagtcaaagctgac

SEQ ID NO: 49 - DNA, 321 bp, Synthetic construct
  misc_feature 1..321: note = CD80_211
  source 1..321: mol_type = other DNA
gttatccacgtgaccaaggaagtgaaagaatatgcaacgctgtcctgtggtcacaatgtttctgttgaagagctggcacaacgtcgcatctactggcaaaaggggaagaaaatggtgctgactatgatgtctggggacttgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgac

SEQ ID NO: 50 - DNA, 321 bp, Synthetic construct
  misc_feature 1..321: note = CD80_212
  source 1..321: mol_type = other DNA
gttatccacgtgaccaaggaagtgcgagcagtggcaacgctgtcctgtggtcacaatgtttctgttgaagagctggcacaaactcgcatctactggcaaaagggtaagaaaatggtgctgactatgatgtctggggacatgaatatatggcccgagtacaagaaccggaccatctttgatatcactaataacctctccattactatcctggctctgcgcccatctgacgagggcacatacgagtgtgttgttctgaagtatgaaaaagacgctttcaagcgggaacacctggctgaagtgaagttatcagtcaaagctgac

SEQ ID NO: 51 - DNA, 321 bp, Synthetic construct
  misc_feature 1..321: note = CD80_213
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattacgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 52 - DNA, 321 bp, Synthetic construct
  misc_feature 1..321: note = CD80_214
  source 1..321: mol_type = other DNA
gttatccacgtgaccaaggaagtgaaaggagtggcaacgctgtcctgtggtcacaatgtttctgttgaagagctggcacaaactcgcatctactggcaaaaggggaagaaaatggtgctgactatgatgtctggggaggttaatatatggcccgagtacaagaaccggaccatctttgatatcactaataacctctccattatgatcctggctctgcgcccatctgacgagggcacatacgagtgtgttgttctgaagtatgaaaaagacgctttcaagcgggaacacctggctgaagtgaagttatcagtcaaagctgac

SEQ ID NO: 53 - DNA, 321 bp, Synthetic construct
  misc_feature 1..321: note = CD80_215
  source 1..321: mol_type = other DNA
gttatccacgtgaccaaggaagtgcgagcagtggcaacgctgtcctgtggtcacaatgtttctgttgaagagctggcacaaactcgcatctactggcaaaagggtaagaaaatggtgctgactatgatgtctggggacatgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 54 - DNA, 321 bp, Synthetic construct
  misc_feature 1..321: note = CD80_216
  source 1..321: mol_type = other DNA
gttatccacgtgaccaaggaagtgaaagaatgggcaacgctgtcctgtggtcacaatgtttctgttgaagagctggcacaatatcgcatctactggcaaaaggagaagaaaatggtgctgactatgatgtctggggttatgaatatatggcccgagtacaagaaccggaccatctttgatatcactaataacctctccattgtgatcctggctctggagccatctgacgagggcacatacgagtgtgttgttctgaagtatgaaaaagacgctttaaagcgggaacacctggctgaagtgacgttatcagtcaaagctgac

SEQ ID NO: 55 - DNA, 321 bp, Synthetic construct
  misc_feature 1..321: note = CD80_217
  source 1..321: mol_type = other DNA
gttatccacgtgaccaaggaagtgaatggagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagaaggaaaaagacgctttcaagcgggaacacctggctgaagtgcagttatcagtcaaagctgac

SEQ ID NO: 56 - DNA, 321 bp, Synthetic construct
  misc_feature 1..321: note = CD80_218
  source 1..321: mol_type = other DNA
gttatccacgtgaccaaggaagtgaaagaatgggcaacgctgtcctgtggtcacaatgtttctgttgaagagctggcacaatatcgcatctactggcaaaaggagaagaaaatggtgctgactatgatgtctggggttatgaatatatggcccgagtacaagaaccggaccatctttgatatcactaataacctctccattactatcctggctctgaccccatctgacgagggcacatacgagtgtgttgttctgaagaatgaaaaagacgctttcaagcgggaacacctggctgaagtgacgttatcagtcaaagctgac

SEQ ID NO: 57 - DNA, 321 bp, Synthetic construct
  misc_feature 1..321: note = CD80_219
  source 1..321: mol_type = other DNA
gttatccacgtgaccaaggaagtgaaatcatatgcaacgctgtcctgtggtcacaatgtttctgttgaagagctggcacaaactcgcatctactggcaaaaggagaagaaaatggtgctgactcggatgtctggggacatgaatatatggcccgagtacaagaaccggaccatctttgatatcactaataacctctccattgtgatcctggtgctgcgcccatctgacgagggcacatacgagtgtgttgttctgaagtatgaaaaagacgctttcaagcgggaacacctggctgaagtgacgttatcagtcaaagctgac

SEQ ID NO: 58 - DNA, 321 bp, Synthetic construct
  misc_feature 1..321: note = CD80_220
  source 1..321: mol_type = other DNA
gttatccacgtgaccaaggaagtgaaagaagtggcaacgctgtcctgtggttttaatgtttctgttgaagagctggcacaagttcgcatctactggcaaaaggagaagaaaatggtgctgactatgatgtctggggaccttaatatatggcccgagtacaagaaccggaccatctttgatatcactaataacctctccatttcgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 59 - DNA, 321 bp, Synthetic construct
  misc_feature 1..321: note = CD80_221
  source 1..321: mol_type = other DNA
gttatccacgtgaccaaggaagtgaaaggggtggcaacgctgtcctgtggtcacaatgtttctgttgaagagctgtcacaatatcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 60 - DNA, 321 bp, Synthetic construct
  misc_feature 1..321: note = CD80_222
  source 1..321: mol_type = other DNA
gttatccacgtgaccaaggaagtgaaagaagtggcaacgctgtcctgtggtcacaatgtttctgttgaagagctggcacaaactcgcatctactggcaaaaggataagaaaatggtgctgactatgatgtctgggcagatgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 61 - DNA, 321 bp, Synthetic construct
  misc_feature 1..321: note = CD80_223
  source 1..321: mol_type = other DNA
gttatccacgtgaccaaggaagtgaaatcggtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattatgatcctggctctgcgcccatctgacgagggcacatacgagtgtgttgttctgaagcctgaaaaagacgctttcaagcgggaacacctggctgaagtgacgttatcagtcaaagctgac

SEQ ID NO: 62 - DNA, 321 bp, Synthetic construct
  misc_feature 1..321: note = CD80_224
  source 1..321: mol_type = other DNA
gttatccacgtgaccaaggaagtgaatgaatgggcaacgctgtcctgtggtcacaatgtttctgttgaagagctggcacaaactcgcatctactggcaaaaggagaagaaaatggtgctgactatgatgtctggggacttgaatatatggcccgagtacaagaaccggaccatctttgatatcactaataacctctccattactatcctggctctgaccccatctgacgagggcacatacgagtgtgttgttctgaagaatgaaaaagacgctttcaagcgggaacacctggctgaagtgacgttatcagtcaaagctgac

SEQ ID NO: 63 - DNA, 321 bp, Synthetic construct
  misc_feature 1..321: note = CD80_225
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacttgaatatatggcccgagtacaagaaccggaccatctttgatatcactaataacctctccattactatcctggctctgcgcccatctgacgagggcacatacgagtgtgttgttctgaagtatgaaaaagacgctttcaagcgggaacacctggctgaagtgacgttatcagtcaaagctgac

SEQ ID NO: 64 - DNA, 321 bp, Synthetic construct
  misc_feature 1..321: note = CD80_226
  source 1..321: mol_type = other DNA
gttatccacgtgaccaaggaagtgagagaagtggcaacgctgtcctgtggtcacaatgtttctgttgaagagctgacgcaatatcgcatctactggcaaaaggcgaagaaaatggtgctgactatgatgtctggggacttgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgac

SEQ ID NO: 65 - DNA, 321 bp, Synthetic construct
  misc_feature 1..321: note = CD80_227
  source 1..321: mol_type = other DNA
gttatccacgtgaccaaggaagtgaaagaatgggcaacgctgtcctgtggtcacaatgtttctgttgaagagctggcacaacatcgcatctactggcaaaaggagaagaaaatggtgctgactatgatgtctgggcagatgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgac

SEQ ID NO: 66 - DNA, 321 bp, Synthetic construct
  misc_feature 1..321: note = CD80_228
  source 1..321: mol_type = other DNA
gttatccacgtgaccaagcaagtgaaagaatatgcaacgctgtcctgtggtcacaatgtttctgttgaagagctggcacaaactcacatctactggcaaaaggagaagaaaatggtgctgactatgatgtctggggacttgaatatatggcccgagtacaagaaccggaccatctttgatatcactaataacctctccattactatcctggctctgcgcccatctgacgagggcacatacgagtgtgttgttctgaagtatgaaaaagacgctttcaagcgggaacacctggctgaagtgacgttatcagtcaaagctgac

SEQ ID NO: 67 - DNA, 321 bp, Synthetic construct
  misc_feature 1..321: note = CD80_229
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacgtgaatatatggcccgagtacaagaaccggaccatctttgatatcactaataacctctccattgtgatcctgggtctgcgcccatctgacgagggcacatacgagtgtgttgttgagaagtatgaaaaagacgctttcaagcgggaacacctggctgaagtgacgttatcagtcaaagctgac

SEQ ID NO: 68 - DNA, 321 bp, Synthetic construct
  misc_feature 1..321: note = CD80_230
  source 1..321: mol_type = other DNA
gttatccacgtgaccaaggaagtgaaaggagtggcaacgctgtcctgtggtcacaatgtttctgttgaagagctggcatttactcgcatctactggcaaaaggagaagaaaatggtgctgactatgatgtctgggaacatgaatatatggcccgagtacaagaaccggaccatctttgatatcactaataacctctccattgtgatcctgggtctgcgcccatctgacgagggcacatacgagtgtgttgttttgaagtatgaaaaaggtgctttcaagcgggaacacctggctgaagtgacgttatcagtcaaagctgac

SEQ ID NO: 69 - DNA, 321 bp, Synthetic construct
  misc_feature 1..321: note = CD80_231
  source 1..321: mol_type = other DNA
gttatccacgtgaccaaggaagtgaaagaatgggcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgacatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 70 - DNA, 321 bp, Synthetic construct
  misc_feature 1..321: note = CD80_232
  source 1..321: mol_type = other DNA
gttatccacgtgaccaaggaagtgaaagaatttgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgacatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 71 - DNA, 321 bp, Synthetic construct
  misc_feature 1..321: note = CD80_233
  source 1..321: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgacatcactaataacctctccattatgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 72 - DNA, 321 bp, Synthetic construct
  misc_feature 1..321: note = CD80_234
  source 1..321: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgacatcactaataacctctccattctgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 73 - DNA, 321 bp, Synthetic construct
  misc_feature 1..321: note = CD80_235
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacctgaatatatggcccgagtacaagaaccggaccatctttgacatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgac

SEQ ID NO: 74 - DNA, 321 bp, Synthetic construct
  misc_feature 1..321: note = CD80_236
  source 1..321: mol_type = other DNA
gttatccacgtgaccaaggaagtgaaagaatatgcaacgctgtcctgtggtcacaatgtttctgttgaagagctggcacaataccgcatctactggcaaaaggagaagaaaatggtgctgactatgatgtctggggacctgaatatatggcccgagtacaagaaccggaccatctttgacatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgac

SEQ ID NO: 75 - DNA, 321 bp, Synthetic construct
  misc_feature 1..321: note = CD80_237
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacctgaatatatggcccgagtacaagaaccggaccatctttgacatcactaataacctctccattatgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 76 - DNA, 321 bp, Synthetic construct
  misc_feature 1..321: note = CD80_238
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggaggtgaatatatggcccgagtacaagaaccggaccatctttgacatcactaataacctctccattatgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 77 - DNA, 321 bp, Synthetic construct
  misc_feature 1..321: note = CD80_239
  source 1..321: mol_type = other DNA
gttatccacgtgaccaaggaagtgaaagaatatgcaacgctgtcctgtggtcacaatgtttctgttgaagagctggcacaataccgcatctactggcaaaaggagaagaaaatggtgctgactatgatgtctggggaggtgaatatatggcccgagtacaagaaccggaccatctttgacatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 78 - DNA, 321 bp, Synthetic construct
  misc_feature 1..321: note = CD80_240
  source 1..321: mol_type = other DNA
gttatccacgtgaccaaggaagtgaaagaatatgcaacgctgtcctgtggtcacaatgtttctgttgaagagctggcacaataccgcatctactggcaaaaggagaagaaaatggtgctgactatgatgtctggggaggtgaatatatggcccgagtacaagaaccggaccatctttgacatcactaataacctctccattatgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 79 - DNA, 321 bp, Synthetic construct
  misc_feature 1..321: note = CD80_241
  source 1..321: mol_type = other DNA
gttatccacgtgaccaaggaagtgaaatcctttgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgacatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 80 - DNA, 321 bp, Synthetic construct
  misc_feature 1..321: note = CD80_242
  source 1..321: mol_type = other DNA
gttatccacgtgaccaaggaagtgaaagaatttgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgacatctccaataacctctccattgtgatcctggctctgcgcccatctgacgagggcacatacgagtgtgttgttctgaagtatgaaaaagacgctttcaagcgggaacacctggctgaagtgacgttatcagtcaaagctgac

SEQ ID NO: 81 - DNA, 321 bp, Synthetic construct
  misc_feature 1..321: note = CD80_243
  source 1..321: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacctgaatatatggcccgagtacaagaaccggaccatctttgacatcactaataacctctccattgtgatcctgggtctgcgcccatctgacgagggcacatacgagtgtgttgttctgaagagagaaaaagacgctttcaagcgggaacacctggctgaagtgacgttatcagtcaaagctgac

SEQ ID NO: 82 - DNA, 321 bp, Synthetic construct
  misc_feature 1..321: note = CD80_244
  source 1..321: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgacatcactaataacctctccattgtgatcctggctctgcgcccatctgacgagggcacatacgagtgtgttgttctgaagagagaaaaagacgctttcaagcgggaacacctggctgaagtgacgttatcagtcaaagctgac

SEQ ID NO: 83 - DNA, 1026 bp, Synthetic construct
  misc_feature 1..1026: note = CD80_165-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacgtgaatatatggcccgagtacaagaaccggaccatcagtgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 84 - DNA, 1026 bp, Synthetic construct
  misc_feature 1..1026: note = CD80_166-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacgtgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 85 - DNA, 1026 bp, Synthetic construct
  misc_feature 1..1026: note = CD80_167-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactattatgtctggggacgtgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 86 - DNA, 1026 bp, Synthetic construct
  misc_feature 1..1026: note = CD80_168-Fc
  source 1..1026: mol_type = other DNA
gttatccacgtgaccaaggaagtgaatggagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcacttctaacctctccattgtgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 87 - DNA, 1026 bp, Synthetic construct
  misc_feature 1..1026: note = CD80_169-Fc
  source 1..1026: mol_type = other DNA
gttatccacgtgaccaaggaagtgaaaggagtggcaacgctgtcctgtggtcacaatgtttctgttgaagagctggcacaaactcgcatctactggcaaaaggggaagaaaatggtgctgactatgatgtctggggaggttaatatatggcccgagtacaagaaccggaccatctttgatatcactaataacctctccattatgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 88 - DNA, 1026 bp, Synthetic construct
  misc_feature 1..1026: note = CD80_170-Fc
  source 1..1026: mol_type = other DNA
gttatccacgtgaccaaggaagtgaatggagtggcaacgctgtcctgtggtcacaatgtttctgttgaagagctggcacaaactcgcatctactggcaaaaggggaagaaaatggtgctgactatgatgtctggggaggttaatatatggcccgagtacaagaaccggaccatctttgatatcactaataacctctccattatgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 89 - DNA, 1026 bp, Synthetic construct
  misc_feature 1..1026: note = CD80_171-Fc
  source 1..1026: mol_type = other DNA
gttatccacgtgaccaagtctgtgaaagaagtggcaacgctgtcctgtggtatcaatttgtctgttgaagagctgaagcaaactcgcatctactggcaaaaggagaagaaaatggtgctgactatgatgtctggggacctgaatatatggcccgagtacaagaaccggaccatttttgatatcactaataacctctccattgtgatcctgaatctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 90 - DNA, 1026 bp, Synthetic construct
  misc_feature 1..1026: note = CD80_172-Fc
  source 1..1026: mol_type = other DNA
gttatccacgtgaccaaggaagtgagatcttatgcaacgctgtcctgtggtcacaatgtttctgttgaagagctggcacaaactcgcatctactggcaaaaggagaagaaaatggtgctgactctgatgtctggggacatgaatatatggcccgagtacaagaaccggaccatcatggatatcactaataacctctccattatgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 91 - DNA, 1026 bp, Synthetic construct
  misc_feature 1..1026: note = CD80_173-Fc
  source 1..1026: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 92 - DNA, 1026 bp, Synthetic construct
  misc_feature 1..1026: note = CD80_174-Fc
  source 1..1026: mol_type = other DNA
gttatccacgtgaccaaggaagtgaaacgggtggcaacgctgtcctgtggtcacaatgtttctgttgaagagctggcacaaactcgcatctactggcaaaaggagaagaaaatggtgctgactatgatgtctggggacaggaatatatggcccgagtacaagaaccggaccatctttgatatcactattaacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 93 - DNA, 1026 bp, Synthetic construct
  misc_feature 1..1026: note = CD80_175-Fc
  source 1..1026: mol_type = other DNA
gttatccacgtgaccaaggaagtgaaatcatttgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatctctaataacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 94 - DNA, 1026 bp, Synthetic construct
  misc_feature 1..1026: note = CD80_176-Fc
  source 1..1026: mol_type = other DNA
gttatccacgtgaccaaggaagtgaaacgggtggcaacgctgtcctgtggttataatgtttctgttgaagagctgggacaaactcgcatctactggcaaaaggataagaaaatggtgctgactatgatgtctggggacatgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 95 - DNA, 1026 bp, Synthetic construct
  misc_feature 1..1026: note = CD80_177-Fc
  source 1..1026: mol_type = other DNA
gttatccacgtgaccaaggaagtgaaagggtgggcaacgctgtcctgtggtcacaatgtttcttctgaagagctggcacaacctcgcatctactggcaaaaggagaaaaaaatggtgctgactatgatgtctggggacatgaatatatggcccgagtacaagaaccggaccatctttgatatcactaataacctctccattgtgatcctgggtctgcgcccatctgacgagggcacatacaagtgtgttgttctgaagagggaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 96 - DNA, 1026 bp, Synthetic construct
  misc_feature 1..1026: note = CD80_178-Fc
  source 1..1026: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttgagaagattgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 97 - DNA, 1026 bp, Synthetic construct
  misc_feature 1..1026: note = CD80_179-Fc
  source 1..1026: mol_type = other DNA
gttatccacgtgaccaaggaagtgaaagaagtggcaacgctgtcctgtggtttcaatgtttctgttgaagagctggcacaaactcgcatctactggcaaaaggagaagaaaatggtgctgactgggatgtctggggacatgaatatatggcccgagtacaagaaccggaccatctatgatatcactaataacctctccattaatatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 98 - DNA, 1026 bp, Synthetic construct
  misc_feature 1..1026: note = CD80_180-Fc
  source 1..1026: mol_type = other DNA
gttatccacgtgaccaaggaagtgaaagggtgggcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 99 - DNA, 1026 bp, Synthetic construct
  misc_feature 1..1026: note = CD80_181-Fc
  source 1..1026: mol_type = other DNA
gttatccacgtgaccaaggaagtgagtcgatatgcaacgctgtcctgtggtcacaatgtttctgttgaagagctggcacaaactcgcatctactggcaaaaggagaagaaaatggtgctgactatgatgtctggggacctgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 100 - DNA, 1026 bp, Synthetic construct
  misc_feature 1..1026: note = CD80_182-Fc
  source 1..1026: mol_type = other DNA
gttatccacgtgaccaaggaagtgaaagggtgggcaacgctgtcctgtggtcacaatgtttctgttgaagagctggcacaaactcgcatctactggcaaaaggggaagaaaatggtgctgactatgatgtctggggacctgaatatatggcccgagtacaagaaccggaccatctttgatatcactaataacctctccattgtgatcctgggtctgcgcccatctgacgagggcacatacgagtgtgttgttctgaagagggaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 101 - DNA, 1026 bp, Synthetic construct
  misc_feature 1..1026: note = CD80_183-Fc
  source 1..1026: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctgggtctgcgcccatctgacgagggcacatacgagtgtgttgttctgaagagggaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 102 - DNA, 1026 bp, Synthetic construct
  misc_feature 1..1026: note = CD80_184-Fc
  source 1..1026: mol_type = other DNA
gttatccacgtgaccaaggaagtgaaagaatatgcaacgctgtcctgtggtcacaatgtttctgttgaagagctgcagcaaactcgcatctactggcaaaaggagaagaaaatggtgctgactatgatgtctggggacctgaatatatggcccgagtacaagaaccggaccatctttgatatcactaataacctctccattgtgatcctgggtctgcgcccatctgacgagggcacatacgagtgtgttgttctgaagagggaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 103 - DNA, 1026 bp, Synthetic construct
  misc_feature 1..1026: note = CD80_185-Fc
  source 1..1026: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 104 - DNA, 1026 bp, Synthetic construct
  misc_feature 1..1026: note = CD80_186-Fc
  source 1..1026: mol_type = other DNA
gttatccacgtgaccaagaaagtgaaagaatgggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactcataacctctccattgtgatcctgggtctgcgcccatctgacgagggcacatacgagtgtgttgttctgaagaaggaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 105 - DNA, 1026 bp, Synthetic construct
  misc_feature 1..1026: note = CD80_187-Fc
  source 1..1026: mol_type = other DNA
gttatccacgtgaccaaggaagtgaaagaatatgcaacgctgtcctgtggttataatgtttctgttgaagagctggcacaaactcgcatctactggcaaaaggggaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttcagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 106 - DNA, 1026 bp, Synthetic construct
  misc_feature 1..1026: note = CD80_188-Fc
  source 1..1026: mol_type = other DNA
gttatccacgtgaccaaggaagtgaaagaagtggcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 107 - DNA, 1026 bp, Synthetic construct
  misc_feature 1..1026: note = CD80_189-Fc
  source 1..1026: mol_type = other DNA
gttatccacgtgaccaaggaagtgaaagggtgggcaacgctgtcctgtggtcacaatgtttctgttgaagagctggcacaaactcgcatctactggcaaaaggagaagaaaatggtgctgactatgatgtctggggacgtgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 108 - DNA, 1026 bp, Synthetic construct
  misc_feature 1..1026: note = CD80_190-Fc
  source 1..1026: mol_type = other DNA
gttatccacgtgaccaaggaagtgaaagaagtggcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 109 - DNA, 1026 bp, Synthetic construct
  misc_feature 1..1026: note = CD80_191-Fc
  source 1..1026: mol_type = other DNA
gttatccacgtgaccaaggaagtgaaagaatatgcaacgctgtcctgtggtcacaatgtttctgttgaagagctggcacaatatcgcatctactggcaaaaggagaagaaaatggtgctgactatgatgtctggggacctgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 110 - DNA, 1026 bp, Synthetic construct
  misc_feature 1..1026: note = CD80_192-Fc
  source 1..1026: mol_type = other DNA
gttatccacgtgaccaagaatgtgaaagaagtggcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgcgtttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 111 - DNA, 1026 bp, Synthetic construct
  misc_feature 1..1026: note = CD80_193-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgacttggatgtctggggacatgaatatatggcccgagtacaagaaccggaccatctttgatatcgctaataacctctccattgtgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 112 - DNA, 1026 bp, Synthetic construct
  misc_feature 1..1026: note = CD80_194-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgacttggatgtctggggacatgaatatatggcccgagtacaagaaccggaccatctttgatatcgctaataacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 113 - DNA, 1026 bp, Synthetic construct
  misc_feature 1..1026: note = CD80_195-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgacttggatgtctggggacatgaatatatggcccgagtacaagaaccggaccatctatgatatcactaataacctctccattaatatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 114 - DNA, 1026 bp, Synthetic construct
  misc_feature 1..1026: note = CD80_196-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgacttggatgtctggggacatgaatatatggcccaagtacaagaaccggaccatctttgatatcgctaataacctctccattgtgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 115 - DNA, 1026 bp, Synthetic construct
  misc_feature 1..1026: note = CD80_197-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagcaggaaaaagacgctttcaagcgggaacacctggctgaagtgaggttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 116 - DNA, 1026 bp, Synthetic construct
  misc_feature 1..1026: note = CD80_198-Fc
  source 1..1026: mol_type = other DNA
gttatccacgtgaccaaggaagtgaaagaagtggcaacgctgtcgtgtggttacaatgtttctgttgaagagctggaacaaactagcatctactggcaaaaggataagaaaatggtgctgactatgatgtctggggacttgaatatatggcccgagtacaagaaccggaccatctttgatatcactaataacctctccattatgatcctgggtctgcgcccatctgacgggggcacatacgagtgtgttgttctgaagtatgaaaaaggcgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 117 - DNA, 1026 bp, Synthetic construct
  misc_feature 1..1026: note = CD80_199-Fc
  source 1..1026: mol_type = other DNA
gttatccacgtgaccaaggaagtgaaaggggtggcaacgctgtcctgtggttacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 118 - DNA, 1026 bp, Synthetic construct
  misc_feature 1..1026: note = CD80_200-Fc
  source 1..1026: mol_type = other DNA
gttatccacgtgaccaaggaagtgaatagagtggcaacgctgtcctgtggtgtgaatgtttctgttgaagagctggcacaatatcgcatctactggcaaaaggagaagaaaatggtgctgactatgatgtctggggacatgaatatatggcccgagtacaagaaccggaccatctttgatatcactaataacctctccattgtgatcctggggctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 119 - DNA, 1026 bp, Synthetic construct
  misc_feature 1..1026: note = CD80_201-Fc
  source 1..1026: mol_type = other DNA
gttatccacgtgaccaaggaagtgaaaggggtggcaacgctgtcctgtggttacaatgtttctgttgaagagctggcacaatatcgcatctactggcaaaaggagaagaaaatggtgctgactatgatgtctggggactggaatatatggcccgagtacaagaaccggaccatctttgatatctctaataacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 120 - DNA, 1026 bp, Synthetic construct
  misc_feature 1..1026: note = CD80_202-Fc
  source 1..1026: mol_type = other DNA
gttatccacgtgaccaagcatgtgaaagaagtggcaacgctgtcctgtggtctcaatatttctgttgaagagctggcacaatatcgcatctactggcaaaaggagaagaaaatggtgctgactatgatgtctgggtccatgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 121 - DNA, 1026 bp, Synthetic construct
  misc_feature 1..1026: note = CD80_203-Fc
  source 1..1026: mol_type = other DNA
gttatccacgtgaccaaggaagtgaatgcatgggcaacgctgtcctgtggtttcaatgtttctgttgaagagctggcacaacatcgcatctactggcaaaaggagaagaaaatggtgctgactatgatgtctggggacttgaatatatggcccgagtacaagaaccggaccatctttgatatcgagaataacctctccattgtgatcctggctctggacccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 122 - DNA, 1026 bp, Synthetic construct
  misc_feature 1..1026: note = CD80_204-Fc
  source 1..1026: mol_type = other DNA
gttatccacgtgaccaaggaagtgaaagaagtggcaacgctgtcctgtggtgttaatatttctgtggaagagctggcacaatatcgcatctactggcaaaaggggaagaaaatggtgctgactatgatgtctggggacgtgaatatatggcccgagtacaagaaccggaccatctttgatatcactaataacctctccattgtgatcctggctctggagccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 123 - DNA, 1026 bp, Synthetic construct
  misc_feature 1..1026: note = CD80_205-Fc
  source 1..1026: mol_type = other DNA
gttatccacgtgaccaaggaagtgaaaggtgtggcaacgctgtcctgtggtcacaatgtttctgttgaagagctggcacaaactcgcatctactggcaaaaggagaagaaaatggtgctgactatgatgtctgggaagatgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 124 - DNA, 1026 bp, Synthetic construct
  misc_feature 1..1026: note = CD80_206-Fc
  source 1..1026: mol_type = other DNA
gttatccacgtgaccaaggaagtgaaaggagtggcaacgctgtcctgtggtaccaatgtttctgttgaagagctggcaacaactcgcatctactggcaaaaggagaagaaaatggtgctgactatgatgtctggggagttgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 125 - DNA, 1026 bp, Synthetic construct
  misc_feature 1..1026: note = CD80_207-Fc
  source 1..1026: mol_type = other DNA
gttatccacgtgaccaaggaagtgaaagaagtggcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatctttgatatcactaataacctctccattactatcctggctctgcgcccatctgacgagggcacatacgagtgtgttgttctgaagtatgaaaaagacgctttcaagcgggaacacctggctgaagtgaagttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 126 - DNA, 1026 bp, Synthetic construct
  misc_feature 1..1026: note = CD80_208-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggggaagaaaatggtgctgactggtatgtctggggacatgaatatatggcccgagtacaagaaccggaccatcagtgatatcactaataacctctccattgtgatcctggctctgcgcccatctgacgagggcacatacgagtgtgttgttttgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 127 - DNA, 1026 bp, Synthetic construct
  misc_feature 1..1026: note = CD80_209-Fc
  source 1..1026: mol_type = other DNA
gttatccacgtgaccaaggaagtgaaagaatatgcaacgctgtcctgtggtcacaatgtttctgttgaagagctggcacaacgtcgcatctactggcaaaaggggaagaaaatggtgctgactatgatgtctggggacttgaatatatggcccgagtacaagaaccggaccatcagtgatatcactaataacctctccattgtgatcctggctctgcgcccatctgacgagggcacatacgagtgtgttgttttgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 128 - DNA, 1026 bp, Synthetic construct
  misc_feature 1..1026: note = CD80_210-Fc
  source 1..1026: mol_type = other DNA
gttatccacgtgaccaagaatgtgaaagaagtggcaacgctgtcctgtggtcacaatgtttctgttgaagagctggcacaaactcgcatctactggcaaaaggataagaaaatggtgctgactatgatgtctggggacatgaatatatggcccgagtacaagaaccggaccatcagtgatatcactaataacctctccattgtgatcctggctctgcgcccatctgacgagggcacatacgagtgtgttgttttgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 129 - DNA, 1026 bp, Synthetic construct
  misc_feature 1..1026: note = CD80_211-Fc
  source 1..1026: mol_type = other DNA
gttatccacgtgaccaaggaagtgaaagaatatgcaacgctgtcctgtggtcacaatgtttctgttgaagagctggcacaacgtcgcatctactggcaaaaggggaagaaaatggtgctgactatgatgtctggggacttgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 130 - DNA, 1026 bp, Synthetic construct
  misc_feature 1..1026: note = CD80_212-Fc
  source 1..1026: mol_type = other DNA
gttatccacgtgaccaaggaagtgcgagcagtggcaacgctgtcctgtggtcacaatgtttctgttgaagagctggcacaaactcgcatctactggcaaaagggtaagaaaatggtgctgactatgatgtctggggacatgaatatatggcccgagtacaagaaccggaccatctttgatatcactaataacctctccattactatcctggctctgcgcccatctgacgagggcacatacgagtgtgttgttctgaagtatgaaaaagacgctttcaagcgggaacacctggctgaagtgaagttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 131 - DNA, 1026 bp, Synthetic construct
  misc_feature 1..1026: note = CD80_213-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattacgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 132 - DNA, 1026 bp, Synthetic construct
  misc_feature 1..1026: note = CD80_214-Fc
  source 1..1026: mol_type = other DNA
gttatccacgtgaccaaggaagtgaaaggagtggcaacgctgtcctgtggtcacaatgtttctgttgaagagctggcacaaactcgcatctactggcaaaaggggaagaaaatggtgctgactatgatgtctggggaggttaatatatggcccgagtacaagaaccggaccatctttgatatcactaataacctctccattatgatcctggctctgcgcccatctgacgagggcacatacgagtgtgttgttctgaagtatgaaaaagacgctttcaagcgggaacacctggctgaagtgaagttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 133 - DNA, 1026 bp, Synthetic construct
  misc_feature 1..1026: note = CD80_215-Fc
  source 1..1026: mol_type = other DNA
gttatccacgtgaccaaggaagtgcgagcagtggcaacgctgtcctgtggtcacaatgtttctgttgaagagctggcacaaactcgcatctactggcaaaagggtaagaaaatggtgctgactatgatgtctggggacatgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 134 - DNA, 1026 bp, Synthetic construct
  misc_feature 1..1026: note = CD80_216-Fc
  source 1..1026: mol_type = other DNA
gttatccacgtgaccaaggaagtgaaagaatgggcaacgctgtcctgtggtcacaatgtttctgttgaagagctggcacaatatcgcatctactggcaaaaggagaagaaaatggtgctgactatgatgtctggggttatgaatatatggcccgagtacaagaaccggaccatctttgatatcactaataacctctccattgtgatcctggctctggagccatctgacgagggcacatacgagtgtgttgttctgaagtatgaaaaagacgctttaaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 135 - DNA, 1026 bp, Synthetic construct
  misc_feature 1..1026: note = CD80_217-Fc
  source 1..1026: mol_type = other DNA
gttatccacgtgaccaaggaagtgaatggagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagaaggaaaaagacgctttcaagcgggaacacctggctgaagtgcagttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 136 - DNA, 1026 bp, Synthetic construct
  misc_feature 1..1026: note = CD80_218-Fc
  source 1..1026: mol_type = other DNA
gttatccacgtgaccaaggaagtgaaagaatgggcaacgctgtcctgtggtcacaatgtttctgttgaagagctggcacaatatcgcatctactggcaaaaggagaagaaaatggtgctgactatgatgtctggggttatgaatatatggcccgagtacaagaaccggaccatctttgatatcactaataacctctccattactatcctggctctgaccccatctgacgagggcacatacgagtgtgttgttctgaagaatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 137 - DNA, 1026 bp, Synthetic construct
  misc_feature 1..1026: note = CD80_219-Fc
  source 1..1026: mol_type = other DNA
gttatccacgtgaccaaggaagtgaaatcatatgcaacgctgtcctgtggtcacaatgtttctgttgaagagctggcacaaactcgcatctactggcaaaaggagaagaaaatggtgctgactcggatgtctggggacatgaatatatggcccgagtacaagaaccggaccatctttgatatcactaataacctctccattgtgatcctggtgctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 138 - DNA, 1026 bp, Synthetic construct
  misc_feature 1..1026: note = CD80_220-Fc
  source 1..1026: mol_type = other DNA
gttatccacgtgaccaaggaagtgaaagaagtggcaacgctgtcctgtggttttaatgtttctgttgaagagctggcacaagttcgcatctactggcaaaaggagaagaaaatggtgctgactatgatgtctggggaccttaatatatggcccgagtacaagaaccggaccatctttgatatcactaataacctctccatttcgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 139 - DNA, 1026 bp, Synthetic construct
  misc_feature 1..1026: note = CD80_221-Fc
  source 1..1026: mol_type = other DNA
gttatccacgtgaccaaggaagtgaaaggggtggcaacgctgtcctgtggtcacaatgtttctgttgaagagctgtcacaatatcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 140 - DNA, 1026 bp, Synthetic construct
  misc_feature 1..1026: note = CD80_222-Fc
  source 1..1026: mol_type = other DNA
gttatccacgtgaccaaggaagtgaaagaagtggcaacgctgtcctgtggtcacaatgtttctgttgaagagctggcacaaactcgcatctactggcaaaaggataagaaaatggtgctgactatgatgtctgggcagatgaatatatggcccgagtacaagaaccggaccatctttgatatcactaataacctctccattgtgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 141 - DNA, 1026 bp, Synthetic construct
  misc_feature 1..1026: note = CD80_223-Fc
  source 1..1026: mol_type = other DNA
gttatccacgtgaccaaggaagtgaaatcggtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattatgatcctggctctgcgcccatctgacgagggcacatacgagtgtgttgttctgaagcctgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 142 - DNA, 1026 bp, Synthetic construct
  misc_feature 1..1026: note = CD80_224-Fc
  source 1..1026: mol_type = other DNA
gttatccacgtgaccaaggaagtgaatgaatgggcaacgctgtcctgtggtcacaatgtttctgttgaagagctggcacaaactcgcatctactggcaaaaggagaagaaaatggtgctgactatgatgtctggggacttgaatatatggcccgagtacaagaaccggaccatctttgatatcactaataacctctccattactatcctggctctgaccccatctgacgagggcacatacgagtgtgttgttctgaagaatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 143 - DNA, 1026 bp, Synthetic construct
  misc_feature 1..1026: note = CD80_225-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacttgaatatatggcccgagtacaagaaccggaccatctttgatatcactaataacctctccattactatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 144 - DNA, 1026 bp, Synthetic construct
  misc_feature 1..1026: note = CD80_226-Fc
  source 1..1026: mol_type = other DNA
gttatccacgtgaccaaggaagtgagagaagtggcaacgctgtcctgtggtcacaatgtttctgttgaagagctgacgcaatatcgcatctactggcaaaaggcgaagaaaatggtgctgactatgatgtctggggacttgaatatatggcccgagtacaagaaccggaccatctttgatatcactaataacctctccattgtgatcctgggtctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 145 - DNA, 1026 bp, Synthetic construct
  misc_feature 1..1026: note = CD80_227-Fc
  source 1..1026: mol_type = other DNA
gttatccacgtgaccaaggaagtgaaagaatgggcaacgctgtcctgtggtcacaatgtttctgttgaagagctggcacaacatcgcatctactggcaaaaggagaagaaaatggtgctgactatgatgtctgggcagatgaatatatggcccgagtacaagaaccggaccatctttgatatcactaataacctctccattttgatcctggctctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 146 - DNA, 1026 bp, Synthetic construct
  misc_feature 1..1026: note = CD80_228-Fc
  source 1..1026: mol_type = other DNA
gttatccacgtgaccaagcaagtgaaagaatatgcaacgctgtcctgtggtcacaatgtttctgttgaagagctggcacaaactcacatctactggcaaaaggagaagaaaatggtgctgactatgatgtctggggacttgaatatatggcccgagtacaagaaccggaccatctttgatatcactaataacctctccattactatcctggctctgcgcccatctgacgagggcacatacgagtgtgttgttctgaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 147 - DNA, 1026 bp, Synthetic construct
  misc_feature 1..1026: note = CD80_229-Fc
  source 1..1026: mol_type = other DNA
gttatccacgtgaccaaggaagtgaaagaatatgcaacgctgtcctgtggtcacaatgtttctgttgaagagctggcacaaactcgcatctactggcaaaaggagaagaaaatggtgctgactatgatgtctggggacgtgaatatatggcccgagtacaagaaccggaccatctttgatatcactaataacctctccattgtgatcctgggtctgcgcccatctgacgagggcacatacgagtgtgttgttgagaagtatgaaaaagacgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 148 - DNA, 1026 bp, Synthetic construct
  misc_feature 1..1026: note = CD80_230-Fc
  source 1..1026: mol_type = other DNA
gttatccacgtgaccaaggaagtgaaaggagtggcaacgctgtcctgtggtcacaatgtttctgttgaagagctggcatttactcgcatctactggcaaaaggagaagaaaatggtgctgactatgatgtctgggaacatgaatatatggcccgagtacaagaaccggaccatctttgatatcactaataacctctccattgtgatcctgggtctgcgcccatctgacgagggcacatacgagtgtgttgttttgaagtatgaaaaaggtgctttcaagcgggaacacctggctgaagtgacgttatcagtcaaagctgacggatccggtggaggagggtcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 149 - DNA, 321 bp, Synthetic construct; identical to SEQ ID NO: 69
  misc_feature 1..321: note = CD80_231-Fc
  source 1..321: mol_type = other DNA

SEQ ID NO: 150 - DNA, 321 bp, Synthetic construct; identical to SEQ ID NO: 70
  misc_feature 1..321: note = CD80_232-Fc
  source 1..321: mol_type = other DNA

SEQ ID NO: 151 - DNA, 321 bp, Synthetic construct; identical to SEQ ID NO: 71
  misc_feature 1..321: note = CD80_233-Fc
  source 1..321: mol_type = other DNA

SEQ ID NO: 152 - DNA, 321 bp, Synthetic construct; identical to SEQ ID NO: 72
  misc_feature 1..321: note = CD80_234-Fc
  source 1..321: mol_type = other DNA

SEQ ID NO: 153 - DNA, 321 bp, Synthetic construct; identical to SEQ ID NO: 73
  misc_feature 1..321: note = CD80_235-Fc
  source 1..321: mol_type = other DNA

SEQ ID NO: 154 - DNA, 321 bp, Synthetic construct; identical to SEQ ID NO: 74
  misc_feature 1..321: note = CD80_236-Fc
  source 1..321: mol_type = other DNA

SEQ ID NO: 155 - DNA, 321 bp, Synthetic construct; identical to SEQ ID NO: 75
  misc_feature 1..321: note = CD80_237-Fc
  source 1..321: mol_type = other DNA

SEQ ID NO: 156 - DNA, 321 bp, Synthetic construct; identical to SEQ ID NO: 76
  misc_feature 1..321: note = CD80_238-Fc
  source 1..321: mol_type = other DNA

SEQ ID NO: 157 - DNA, 321 bp, Synthetic construct; identical to SEQ ID NO: 77
  misc_feature 1..321: note = CD80_239-Fc
  source 1..321: mol_type = other DNA

SEQ ID NO: 158 - DNA, 321 bp, Synthetic construct; identical to SEQ ID NO: 78
  misc_feature 1..321: note = CD80_240-Fc
  source 1..321: mol_type = other DNA

SEQ ID NO: 159 - DNA, 321 bp, Synthetic construct; identical to SEQ ID NO: 79
  misc_feature 1..321: note = CD80_241-Fc
  source 1..321: mol_type = other DNA

SEQ ID NO: 160 - DNA, 321 bp, Synthetic construct; identical to SEQ ID NO: 80
  misc_feature 1..321: note = CD80_242-Fc
  source 1..321: mol_type = other DNA

SEQ ID NO: 161 - DNA, 321 bp, Synthetic construct; identical to SEQ ID NO: 81
  misc_feature 1..321: note = CD80_243-Fc
  source 1..321: mol_type = other DNA

SEQ ID NO: 162 - DNA, 321 bp, Synthetic construct; identical to SEQ ID NO: 82
  misc_feature 1..321: note = CD80_244-Fc
  source 1..321: mol_type = other DNA

SEQ ID NO: 163 - protein, 101 aa, Synthetic construct
  REGION 1..101: note = CD80 WT
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVT

SEQ ID NO: 164 - protein, 107 aa, Synthetic construct
  REGION 1..107: note = WT IgV
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAD

SEQ ID NO: 165 - protein, 107 aa, Synthetic construct
  REGION 1..107: note = CD80_165
VIHVTKEVKEYATLSCGHNVSVEELAQTRIYWQKEKKMVLTMMSGDVNIWPEYKNRTISDITNNLSIVILALRPSDEGTYECVVEKYEKDAFKREHLAEVTLSVKAD

SEQ ID NO: 166 - protein, 107 aa, Synthetic construct
  REGION 1..107: note = CD80_166
VIHVTKEVKEYATLSCGHNVSVEELAQTRIYWQKEKKMVLTMMSGDVNIWPEYKNRTIFDITNNLSIVILALRPSDEGTYECVVEKYEKDAFKREHLAEVTLSVKAD

SEQ ID NO: 167 - protein, 107 aa, Synthetic construct
  REGION 1..107: note = CD80_167
VIHVTKEVKEYATLSCGHNVSVEELAQTRIYWQKEKKMVLTIMSGDVNIWPEYKNRTIFDITNNLSIVILGLRPSDEGTYECVVLKYEKDAFKREHLAEVTLSVKAD

SEQ ID NO: 168 - protein, 107 aa, Synthetic construct
  REGION 1..107: note = CD80_168
VIHVTKEVNGVATLSCGHNVSVEELAQTRIYWQKEKKMVLTMMSGDMNIWPEYKNRTIFDITSNLSIVILALRPSDEGTYECVVEKYEKDAFKREHLAEVTLSVKAD

SEQ ID NO: 169 - protein, 107 aa, Synthetic construct
  REGION 1..107: note = CD80_169
VIHVTKEVKGVATLSCGHNVSVEELAQTRIYWQKGKKMVLTMMSGEVNIWPEYKNRTIFDITNNLSIMILALRPSDEGTYECVVLKYEKDAFKREHLAEVTLSVKAD

SEQ ID NO: 170 - protein, 107 aa, Synthetic construct
  REGION 1..107: note = CD80_170
VIHVTKEVNGVATLSCGHNVSVEELAQTRIYWQKGKKMVLTMMSGEVNIWPEYKNRTIFDITNNLSIMILALRPSDEGTYECVVLKYEKDAFKREHLAEVTLSVKAD

SEQ ID NO: 171 - protein, 107 aa, Synthetic construct
  REGION 1..107: note = CD80_171
VIHVTKSVKEVATLSCGINLSVEELKQTRIYWQKEKKMVLTMMSGDLNIWPEYKNRTIFDITNNLSIVILNLRPSDEGTYECVVLKYEKDAFKREHLAEVTLSVKAD

SEQ ID NO: 172 - protein, 107 aa, Synthetic construct
  REGION 1..107: note = CD80_172
VIHVTKEVRSYATLSCGHNVSVEELAQTRIYWQKEKKMVLTLMSGDMNIWPEYKNRTIMDITNNLSIMILALRPSDEGTYECVVEKYEKDAFKREHLAEVTLSVKAD

SEQ ID NO: 173 - protein, 107 aa, Synthetic construct
  REGION 1..107: note = CD80_173
  VARIANT 85: note = X = Any Amino Acid
VIHVTKEVKEYATLSCGHNVSVEELAQYRIYWQKEKKMVLTMMSGDLNIWPEYKNRTIFDITNNLSIVILALRPSDEGTYECVVXKYEKDAFKREHLAEVTLSVKAD

SEQ ID NO: 174 - protein, 107 aa, Synthetic construct
  REGION 1..107: note = CD80_174
VIHVTKEVKRVATLSCGHNVSVEELAQTRIYWQKEKKMVLTMMSGDRNIWPEYKNRTIFDITINLSIVILALRPSDEGTYECVVLKYEKDAFKREHLAEVTLSVKAD

SEQ ID NO: 175 - protein, 107 aa, Synthetic construct
  REGION 1..107: note = CD80_175
VIHVTKEVKSFATLSCGHNVSVEELAQYRIYWQKEKKMVLTMMSGDLNIWPEYKNRTIFDISNNLSIVILALRPSDEGTYECVVLKYEKDAFKREHLAEVTLSVKAD

SEQ ID NO: 176 - protein, 107 aa, Synthetic construct
  REGION 1..107: note = CD80_176
VIHVTKEVKRVATLSCGYNVSVEELGQTRIYWQKDKKMVLTMMSGDMNIWPEYKNRTIFDITNNLSILILALRPSDEGTYECVVEKYEKDAFKREHLAEVTLSVKAD

SEQ ID NO: 177 - protein, 107 aa, Synthetic construct
  REGION 1..107: note = CD80_177
VIHVTKEVKGWATLSCGHNVSSEELAQPRIYWQKEKKMVLTMMSGDMNIWPEYKNRTIFDITNNLSIVILGLRPSDEGTYKCVVLKREKDAFKREHLAEVTLSVKAD

SEQ ID NO: 178 - protein, 107 aa, Synthetic construct
  REGION 1..107: note = CD80_178
VIHVTKEVKEYATLSCGHNVSVEELAQYRIYWQKEKKMVLTMMSGDMNIWPEYKNRTIFDITNNLSIVILALRPSDEGTYECVVEKIEKDAFKREHLAEVTLSVKAD

SEQ ID NO: 179 - protein, 107 aa, Synthetic construct
  REGION 1..107: note = CD80_179
VIHVTKEVKEVATLSCGFNVSVEELAQTRIYWQKEKKMVLTGMSGDMNIWPEYKNRTIYDITNNLSINILALRPSDEGTYECVVLKYEKDAFKREHLAEVTLSVKAD

SEQ ID NO: 180 - protein, 107 aa, Synthetic construct
  REGION 1..107: note = CD80_180
VIHVTKEVKGWATLSCGHNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 181 - protein, 107 aa, Synthetic construct
  REGION 1..107: note = CD80_181
VIHVTKEVSRYATLSCGHNVSVEELAQTRIYWQKEKKMVLTMMSGDLNIWPEYKNRTIFDITNNLSIVILGLRPSDEGTYECVVLKYEKDAFKREHLAEVTLSVKAD

SEQ ID NO: 182 - protein, 107 aa, Synthetic construct
  REGION 1..107: note = CD80_182
VIHVTKEVKGWATLSCGHNVSVEELAQTRIYWQKGKKMVLTMMSGDLNIWPEYKNRTIFDITNNLSIVILGLRPSDEGTYECVVLKREKDAFKREHLAEVTLSVKAD

SEQ ID NO: 183 - protein, 107 aa, Synthetic construct
  REGION 1..107: note = CD80_183
VIHVTKEVKEYATLSCGHNVSVEELAQYRIYWQKEKKMVLTMMSGDLNIWPEYKNRTIFDITNNLSIVILGLRPSDEGTYECVVLKREKDAFKREHLAEVTLSVKAD

SEQ ID NO: 184 - protein, 107 aa, Synthetic construct
  REGION 1..107: note = CD80_184
VIHVTKEVKEYATLSCGHNVSVEELQQTRIYWQKEKKMVLTMMSGDLNIWPEYKNRTIFDITNNLSIVILGLRPSDEGTYECVVLKREKDAFKREHLAEVTLSVKAD

SEQ ID NO: 185 - protein, 107 aa, Synthetic construct
  REGION 1..107: note = CD80_185
VIHVTKEVKEYATLSCGHNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 186 - protein, 107 aa, Synthetic construct
  REGION 1..107: note = CD80_186
VIHVTKKVKEWATLSCGHNVSVEELAQTRIYWQKEKKMVLTMMSGDMNIWPEYKNRTIFDITHNLSIVILGLRPSDEGTYECVVLKKEKDAFKREHLAEVTLSVKAD

SEQ ID NO: 187 - protein, 107 aa, Synthetic construct
  REGION 1..107: note = CD80_187
VIHVTKEVKEYATLSCGYNVSVEELAQTRIYWQKGKKMVLTMMSGDMNIWPEYKNRTIFDITNNLSIVILALRPSDEGTYECVVQKYEKDAFKREHLAEVTLSVKAD

SEQ ID NO: 188 - protein, 107 aa, Synthetic construct
  REGION 1..107: note = CD80_188
VIHVTKEVKEVATLSCGHNVSVEELAQTRIYWQKDKKMVLTMMSGDMNIWPEYKNRTIFDITNNLSILILALRPSDEGTYECVVEKYEKDAFKREHLAEVTLSVKAD

SEQ ID NO: 189 - protein, 107 aa, Synthetic construct
  REGION 1..107: note = CD80_189
VIHVTKEVKGWATLSCGHNVSVEELAQTRIYWQKEKKMVLTMMSGDVNIWPEYKNRTIFDITNNLSIVILALRPSDEGTYECVVEKYEKDAFKREHLAEVTLSVKAD

SEQ ID NO: 190 - protein, 107 aa, Synthetic construct
  REGION 1..107: note = CD80_190
VIHVTKEVKEVATLSCGHNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 191 - protein, 107 aa, Synthetic construct
  REGION 1..107: note = CD80_191
VIHVTKEVKEYATLSCGHNVSVEELAQYRIYWQKEKKMVLTMMSGDLNIWPEYKNRTIFDITNNLSILILALRPSDEGTYECVVEKYEKDAFKREHLAEVTLSVKAD

SEQ ID NO: 192 - protein, 107 aa, Synthetic construct
  REGION 1..107: note = CD80_192
VIHVTKNVKEVATLSCGHNVSVEELAQTRIYWQKDKKMVLTMMSGDMNIWPEYKNRTIFDITNNLSIVILALRPSDEGTYECVVLKYEKDAFKREHLAEVRLSVKAD

SEQ ID NO: 193 - protein, 107 aa, Synthetic construct
  REGION 1..107: note = CD80_193
VIHVTKEVKEYATLSCGHNVSVEELAQTRIYWQKEKKMVLTWMSGDMNIWPEYKNRTIFDIANNLSIVILALRPSDEGTYECVVEKYEKDAFKREHLAEVTLSVKAD

SEQ ID NO: 194 - protein, 107 aa, Synthetic construct
  REGION 1..107: note = CD80_194
VIHVTKEVKEYATLSCGHNVSVEELAQTRIYWQKEKKMVLTWMSGDMNIWPEYKNRTIFDIANNLSIVILALRPSDEGTYECVVLKYEKDAFKREHLAEVTLSVKAD

SEQ ID NO: 195 - protein, 107 aa, Synthetic construct
  REGION 1..107: note = CD80_195
VIHVTKEVKEYATLSCGHNVSVEELAQTRIYWQKEKKMVLTWMSGDMNIWPEYKNRTIYDITNNLSINILALRPSDEGTYECVVLKYEKDAFKREHLAEVTLSVKAD

SEQ ID NO: 196 - protein, 107 aa, Synthetic construct
  REGION 1..107: note = CD80_196
VIHVTKEVKEYATLSCGHNVSVEELAQTRIYWQKEKKMVLTWMSGDMNIWPKYKNRTIFDIANNLSIVILALRPSDEGTYECVVEKYEKDAFKREHLAEVTLSVKAD

SEQ ID NO: 197 - protein, 107 aa, Synthetic construct
  REGION 1..107: note = CD80_197
VIHVTKEVKEYATLSCGHNVSVEELAQTRIYWQKDKKMVLTMMSGDMNIWPEYKNRTIFDITNNLSIVILALRPSDEGTYECVVLKQEKDAFKREHLAEVRLSVKAD

SEQ ID NO: 198 - protein, 107 aa, Synthetic construct
  REGION 1..107: note = CD80_198
VIHVTKEVKEVATLSCGYNVSVEELEQTSIYWQKDKKMVLTMMSGDLNIWPEYKNRTIFDITNNLSIMILGLRPSDGGTYECVVLKYEKGAFKREHLAEVTLSVKAD

SEQ ID NO: 199 - protein, 107 aa, Synthetic construct
  REGION 1..107: note = CD80_199
VIHVTKEVKGVATLSCGYNVSVEELAQTRIYWQKEKKMVLTMMSGDMNIWPEYKNRTIFDITNNLSIVILALRPSDEGTYECVVLKYEKDAFKREHLAEVTLSVKAD

SEQ ID NO: 200 - protein, 107 aa, Synthetic construct
  REGION 1..107: note = CD80_200
VIHVTKEVNRVATLSCGVNVSVEELAQYRIYWQKEKKMVLTMMSGDMNIWPEYKNRTIFDITNNLSIVILGLRPSDEGTYECVVLKYEKDAFKREHLAEVTLSVKAD

SEQ ID NO: 201 - protein, 107 aa, Synthetic construct
  REGION 1..107: note = CD80_201
VIHVTKEVKGVATLSCGYNVSVEELAQYRIYWQKEKKMVLTMMSGDWNIWPEYKNRTIFDISNNLSIVILALRPSDEGTYECVVLKYEKDAFKREHLAEVTLSVKAD

SEQ ID NO: 202 - protein, 107 aa, Synthetic construct
  REGION 1..107: note = CD80_202
VIHVTKHVKEVATLSCGLNISVEELAQYRIYWQKEKKMVLTMMSGSMNIWPEYKNRTIFDITNNLSIVILGLRPSDEGTYECVVLKYEKDAFKREHLAEVTLSVKAD

SEQ ID NO: 203 - protein, 107 aa, Synthetic construct
  REGION 1..107: note = CD80_203
VIHVTKEVNAWATLSCGFNVSVEELAQHRIYWQKEKKMVLTMMSGDLNIWPEYKNRTIFDIENNLSIVILALDPSDEGTYECVVLKYEKDAFKREHLAEVTLSVKAD

SEQ ID NO: 204 - protein, 107 aa, Synthetic construct
  REGION 1..107: note = CD80_204
VIHVTKEVKEVATLSCGVNISVEELAQYRIYWQKGKKMVLTMMSGDVNIWPEYKNRTIFDITNNLSIVILALEPSDEGTYECVVLKYEKDAFKREHLAEVTLSVKAD

SEQ ID NO: 205 - protein, 107 aa, Synthetic construct
  REGION 1..107: note = CD80_205
VIHVTKEVKGVATLSCGHNVSVEELAQTRIYWQKEKKMVLTMMSGKMNIWPEYKNRTIFDITNNLSIVILALRPSDEGTYECVVEKYEKDAFKREHLAEVTLSVKAD

SEQ ID NO: 206 - protein, 107 aa, Synthetic construct
  REGION 1..107: note = CD80_206
VIHVTKEVKGVATLSCGTNVSVEELATTRIYWQKEKKMVLTMMSGELNIWPEYKNRTIFDITNNLSIVILALRPSDEGTYECVVLKYEKDAFKREHLAEVTLSVKAD

SEQ ID NO: 207 - protein, 107 aa, Synthetic construct
  REGION 1..107: note = CD80_207
VIHVTKEVKEVATLSCGHNVSVEELAQTRIYWQKDKKMVLTMMSGDMNIWPEYKNRTIFDITNNLSITILALRPSDEGTYECVVLKYEKDAFKREHLAEVKLSVKAD

SEQ ID NO: 208 - protein, 107 aa, Synthetic construct
  REGION 1..107: note = CD80_208
VIHVTKEVKEYATLSCGHNVSVEELAQTRIYWQKGKKMVLTGMSGDMNIWPEYKNRTISDITNNLSIVILALRPSDEGTYECVVLKYEKDAFKREHLAEVTLSVKAD

SEQ ID NO: 209 - protein, 107 aa, Synthetic construct
  REGION 1..107: note = CD80_209
VIHVTKEVKEYATLSCGHNVSVEELAQRRIYWQKGKKMVLTMMSGDLNIWPEYKNRTISDITNNLSIVILALRPSDEGTYECVVLKYEKDAFKREHLAEVTLSVKAD

SEQ ID NO: 210 - protein, 107 aa, Synthetic construct
  REGION 1..107: note = CD80_210
VIHVTKNVKEVATLSCGHNVSVEELAQTRIYWQKDKKMVLTMMSGDMNIWPEYKNRTISDITNNLSIVILALRPSDEGTYECVVLKYEKDAFKREHLAEVTLSVKAD

SEQ ID NO: 211 - protein, 107 aa, Synthetic construct
  REGION 1..107: note = CD80_211
VIHVTKEVKEYATLSCGHNVSVEELAQRRIYWQKGKKMVLTMMSGDLNIWPEYKNRTIFDITNNLSIVILGLRPSDEGTYECVVLKYEKDAFKREHLAEVTLSVKAD

SEQ ID NO: 212 - protein, 107 aa, Synthetic construct
  REGION 1..107: note = CD80_212
VIHVTKEVRAVATLSCGHNVSVEELAQTRIYWQKGKKMVLTMMSGDMNIWPEYKNRTIFDITNNLSITILALRPSDEGTYECVVLKYEKDAFKREHLAEVKLSVKAD

SEQ ID NO: 213 - protein, 107 aa, Synthetic construct
  REGION 1..107: note = CD80_213
VIHVTKEVKEYATLSCGHNVSVEELAQTRIYWQKEKKMVLTMMSGDMNIWPEYKNRTIFDITNNLSITILALRPSDEGTYECVVLKYEKDAFKREHLAEVTLSVKAD

SEQ ID NO: 214 - protein, 107 aa, Synthetic construct
  REGION 1..107: note = CD80_214
VIHVTKEVKGVATLSCGHNVSVEELAQTRIYWQKGKKMVLTMMSGEVNIWPEYKNRTIFDITNNLSIMILALRPSDEGTYECVVLKYEKDAFKREHLAEVKLSVKAD

SEQ ID NO: 215 - protein, 107 aa, Synthetic construct
  REGION 1..107: note = CD80_215
VIHVTKEVRAVATLSCGHNVSVEELAQTRIYWQKGKKMVLTMMSGDMNIWPEYKNRTIFDITNNLSILILALRPSDEGTYECVVEKYEKDAFKREHLAEVTLSVKAD

SEQ ID NO: 216 - protein, 107 aa, Synthetic construct
  REGION 1..107: note = CD80_216
VIHVTKEVKEWATLSCGHNVSVEELAQYRIYWQKEKKMVLTMMSGVMNIWPEYKNRTIFDITNNLSIVILALEPSDEGTYECVVLKYEKDALKREHLAEVTLSVKAD

SEQ ID NO: 217 - protein, 107 aa, Synthetic construct
  REGION 1..107: note = CD80_217
VIHVTKEVNGVATLSCGHNVSVEELAQTRIYWQKEKKMVLTMMSGDMNIWPEYKNRTIFDITNNLSIVILALRPSDEGTYECVVLKKEKDAFKREHLAEVQLSVKAD

SEQ ID NO: 218 - protein, 107 aa, Synthetic construct
  REGION 1..107: note = CD80_218
VIHVTKEVKEWATLSCGHNVSVEELAQYRIYWQKEKKMVLTMMSGVMNIWPEYKNRTIFDITNNLSITILALTPSDEGTYECVVLKNEKDAFKREHLAEVTLSVKAD

SEQ ID NO: 219 - protein, 107 aa, Synthetic construct
  REGION 1..107: note = CD80_219
VIHVTKEVKSYATLSCGHNVSVEELAQTRIYWQKEKKMVLTRMSGDMNIWPEYKNRTIFDITNNLSIVILVLRPSDEGTYECVVLKYEKDAFKREHLAEVTLSVKAD

SEQ ID NO: 220 - protein, 107 aa, Synthetic construct
  REGION 1..107: note = CD80_220
VIHVTKEVKEVATLSCGFNVSVEELAQVRIYWQKEKKMVLTMMSGDLNIWPEYKNRTIFDITNNLSISILALRPSDEGTYECVVLKYEKDAFKREHLAEVTLSVKAD

SEQ ID NO: 221 - protein, 107 aa, Synthetic construct
  REGION 1..107: note = CD80_221
VIHVTKEVKGVATLSCGHNVSVEELSQYRIYWQKEKKMVLTMMSGDMNIWPEYKNRTIFDITNNLSIVILALRPSDEGTYECVVLKYEKDAFKREHLAEVTLSVKAD

SEQ ID NO: 222 - protein, 107 aa, Synthetic construct
  REGION 1..107: note = CD80_222
VIHVTKEVKEVATLSCGHNVSVEELAQTRIYWQKDKKMVLTMMSGQMNIWPEYKNRTIFDITNNLSIVILALRPSDEGTYECVVEKYEKDAFKREHLAEVTLSVKAD

SEQ ID NO: 223 - protein, 107 aa, Synthetic construct
  REGION 1..107: note = CD80_223
VIHVTKEVKSVATLSCGHNVSVEELAQTRIYWQKEKKMVLTMMSGDMNIWPEYKNRTIFDITNNLSIMILALRPSDEGTYECVVLKPEKDAFKREHLAEVTLSVKAD

SEQ ID NO: 224 - protein, 107 aa, Synthetic construct
  REGION 1..107: note = CD80_224
VIHVTKEVNEWATLSCGHNVSVEELAQTRIYWQKEKKMVLTMMSGDLNIWPEYKNRTIFDITNNLSITILALTPSDEGTYECVVLKNEKDAFKREHLAEVTLSVKAD

SEQ ID NO: 225 - protein, 107 aa, Synthetic construct
  REGION 1..107: note = CD80_225
VIHVTKEVKEYATLSCGHNVSVEELAQTRIYWQKEKKMVLTMMSGDLNIWPEYKNRTIFDITNNLSITILALRPSDEGTYECVVLKYEKDAFKREHLAEVTLSVKAD

SEQ ID NO: 226 - protein, 107 aa, Synthetic construct
  REGION 1..107: note = CD80_226
VIHVTKEVREVATLSCGHNVSVEELTQYRIYWQKAKKMVLTMMSGDLNIWPEYKNRTIFDITNNLSIVILGLRPSDEGTYECVVLKYEKDAFKREHLAEVTLSVKAD

SEQ ID NO: 227 - protein, 107 aa, Synthetic construct
  REGION 1..107: note = CD80_227
VIHVTKEVKEWATLSCGHNVSVEELAQHRIYWQKEKKMVLTMMSGQMNIWPEYKNRTIFDITNNLSILILALRPSDEGTYECVVEKYEKDAFKREHLAEVTLSVKAD

SEQ ID NO: 228 - protein, 107 aa, Synthetic construct
  REGION 1..107: note = CD80_228
VIHVTKQVKEYATLSCGHNVSVEELAQTHIYWQKEKKMVLTMMSGDLNIWPEYKNRTIFDITNNLSITILALRPSDEGTYECVVLKYEKDAFKREHLAEVTLSVKAD

SEQ ID NO: 229 - protein, 107 aa, Synthetic construct
  REGION 1..107: note = CD80_229
VIHVTKEVKEYATLSCGHNVSVEELAQTRIYWQKEKKMVLTMMSGDVNIWPEYKNRTIFDITNNLSIVILGLRPSDEGTYECVVEKYEKDAFKREHLAEVTLSVKAD

SEQ ID NO: 230 - protein, 107 aa, Synthetic construct
  REGION 1..107: note = CD80_230
VIHVTKEVKGVATLSCGHNVSVEELAFTRIYWQKEKKMVLTMMSGNMNIWPEYKNRTIFDITNNLSIVILGLRPSDEGTYECVVLKYEKGAFKREHLAEVTLSVKAD

SEQ ID NO: 231 - protein, 107 aa, Synthetic construct
  REGION 1..107: note = CD80_231
VIHVTKEVKEWATLSCGHNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 232 - protein, 107 aa, Synthetic construct
  REGION 1..107: note = CD80_232
VIHVTKEVKEFATLSCGHNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 233 - protein, 107 aa, Synthetic construct
  REGION 1..107: note = CD80_233
VIHVTKEVKEYATLSCGHNVSVEELAQYRIYWQKEKKMVLTMMSGDLNIWPEYKNRTIFDITNNLSIMILALRPSDEGTYECVVLKYEKDAFKREHLAEVTLSVKAD

SEQ ID NO: 234 - protein, 107 aa, Synthetic construct
  REGION 1..107: note = CD80_234
VIHVTKEVKEYATLSCGHNVSVEELAQYRIYWQKEKKMVLTMMSGDLNIWPEYKNRTIFDITNNLSILILALRPSDEGTYECVVLKYEKDAFKREHLAEVTLSVKAD

SEQ ID NO: 235 - protein, 107 aa, Synthetic construct
  REGION 1..107: note = CD80_235
VIHVTKEVKEYATLSCGHNVSVEELAQTRIYWQKEKKMVLTMMSGDLNIWPEYKNRTIFDITNNLSIVILGLRPSDEGTYECVVLKYEKDAFKREHLAEVTLSVKAD

SEQ ID NO: 236 - protein, 107 aa, Synthetic construct
  REGION 1..107: note = CD80_236
VIHVTKEVKEYATLSCGHNVSVEELAQYRIYWQKEKKMVLTMMSGDLNIWPEYKNRTIFDITNNLSIVILGLRPSDEGTYECVVLKYEKDAFKREHLAEVTLSVKAD

SEQ ID NO: 237 - protein, 107 aa, Synthetic construct
  REGION 1..107: note = CD80_237
VIHVTKEVKEYATLSCGHNVSVEELAQTRIYWQKEKKMVLTMMSGDLNIWPEYKNRTIFDITNNLSIMILALRPSDEGTYECVVLKYEKDAFKREHLAEVTLSVKAD

SEQ ID NO: 238 - protein, 107 aa, Synthetic construct
  REGION 1..107: note = CD80_238
VIHVTKEVKEYATLSCGHNVSVEELAQTRIYWQKEKKMVLTMMSGEVNIWPEYKNRTIFDITNNLSIMILALRPSDEGTYECVVLKYEKDAFKREHLAEVTLSVKAD

SEQ ID NO: 239 - protein, 107 aa, Synthetic construct
  REGION 1..107: note = CD80_239
VIHVTKEVKEYATLSCGHNVSVEELAQYRIYWQKEKKMVLTMMSGEVNIWPEYKNRTIFDITNNLSIVILALRPSDEGTYECVVLKYEKDAFKREHLAEVTLSVKAD

SEQ ID NO: 240 - protein, 107 aa, Synthetic construct
  REGION 1..107: note = CD80_240
VIHVTKEVKEYATLSCGHNVSVEELAQYRIYWQKEKKMVLTMMSGEVNIWPEYKNRTIFDITNNLSIMILALRPSDEGTYECVVLKYEKDAFKREHLAEVTLSVKAD

SEQ ID NO: 241 - protein, 107 aa, Synthetic construct
  REGION 1..107: note = CD80_241
VIHVTKEVKSFATLSCGHNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 242 - protein, 107 aa, Synthetic construct
  REGION 1..107: note = CD80_242
VIHVTKEVKEFATLSCGHNVSVEELAQYRIYWQKEKKMVLTMMSGDLNIWPEYKNRTIFDISNNLSIVILALRPSDEGTYECVVLKYEKDAFKREHLAEVTLSVKAD

SEQ ID NO: 243 - protein, 107 aa, Synthetic construct
  REGION 1..107: note = CD80_243
VIHVTKEVKEYATLSCGHNVSVEELAQTRIYWQKEKKMVLTMMSGDLNIWPEYKNRTIFDITNNLSIVILGLRPSDEGTYECVVLKREKDAFKREHLAEVTLSVKAD

SEQ ID NO: 244 - protein, 107 aa, Synthetic construct
  REGION 1..107: note = CD80_244
VIHVTKEVKEYATLSCGHNVSVEELAQYRIYWQKEKKMVLTMMSGDLNIWPEYKNRTIFDITNNLSIVILALRPSDEGTYECVVLKREKDAFKREHLAEVTLSVKAD

SEQ ID NO: 245 - protein, 342 aa, Synthetic construct
  REGION 1..342: note = CD80_165-Fc
VIHVTKEVKEYATLSCGHNVSVEELAQTRIYWQKEKKMVLTMMSGDVNIWPEYKNRTISDITNNLSIVILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 246 - protein, 342 aa, Synthetic construct
  REGION 1..342: note = CD80_166-Fc
VIHVTKEVKEYATLSCGHNVSVEELAQTRIYWQKEKKMVLTMMSGDVNIWPEYKNRTIFDITNNLSIVILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 247 - protein, 342 aa, Synthetic construct
  REGION 1..342: note = CD80_167-Fc
VIHVTKEVKEYATLSCGHNVSVEELAQTRIYWQKEKKMVLTIMSGDVNIWPEYKNRTIFDITNNLSIVILGLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 248 - protein, 342 aa, Synthetic construct
  REGION 1..342: note = CD80_168-Fc
VIHVTKEVNGVATLSCGHNVSVEELAQTRIYWQKEKKMVLTMMSGDMNIWPEYKNRTIFDITSNLSIVILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 249 - protein, 342 aa, Synthetic construct
  REGION 1..342: note = CD80_169-Fc
VIHVTKEVKGVATLSCGHNVSVEELAQTRIYWQKGKKMVLTMMSGEVNIWPEYKNRTIFDITNNLSIMILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 250 - protein, 342 aa, Synthetic construct
  REGION 1..342: note = CD80_170-Fc
VIHVTKEVNGVATLSCGHNVSVEELAQTRIYWQKGKKMVLTMMSGEVNIWPEYKNRTIFDITNNLSIMILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 251 - protein, 342 aa, Synthetic construct
  REGION 1..342: note = CD80_171-Fc
VIHVTKSVKEVATLSCGINLSVEELKQTRIYWQKEKKMVLTMMSGDLNIWPEYKNRTIFDITNNLSIVILNLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 252 - protein, 342 aa, Synthetic construct
  REGION 1..342: note = CD80_172-Fc
VIHVTKEVRSYATLSCGHNVSVEELAQTRIYWQKEKKMVLTLMSGDMNIWPEYKNRTIMDITNNLSIMILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 253 - protein, 342 aa, Synthetic construct
  REGION 1..342: note = CD80_173-Fc
  VARIANT 85: note = X = Any Amino Acid
VIHVTKEVKEYATLSCGHNVSVEELAQYRIYWQKEKKMVLTMMSGDLNIWPEYKNRTIFDITNNLSIVILALRPSDEGTYECVVXKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 254 - protein, 342 aa, Synthetic construct
  REGION 1..342: note = CD80_174-Fc
VIHVTKEVKRVATLSCGHNVSVEELAQTRIYWQKEKKMVLTMMSGDRNIWPEYKNRTIFDITINLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 255 - protein, 342 aa, Synthetic construct
  REGION 1..342: note = CD80_175-Fc
VIHVTKEVKSFATLSCGHNVSVEELAQYRIYWQKEKKMVLTMMSGDLNIWPEYKNRTIFDISNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 256 - protein, 342 aa, Synthetic construct
  REGION 1..342: note = CD80_176-Fc
VIHVTKEVKRVATLSCGYNVSVEELGQTRIYWQKDKKMVLTMMSGDMNIWPEYKNRTIFDITNNLSILILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 257 - protein, 342 aa, Synthetic construct
  REGION 1..342: note = CD80_177-Fc
VIHVTKEVKGWATLSCGHNVSSEELAQPRIYWQKEKKMVLTMMSGDMNIWPEYKNRTIFDITNNLSIVILGLRPSDEGTYKCVVLKREKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 258 - protein, 342 aa, Synthetic construct
  REGION 1..342: note = CD80_178-Fc
VIHVTKEVKEYATLSCGHNVSVEELAQYRIYWQKEKKMVLTMMSGDMNIWPEYKNRTIFDITNNLSIVILALRPSDEGTYECVVEKIEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 259 - protein, 342 aa, Synthetic construct
  REGION 1..342: note = CD80_179-Fc
VIHVTKEVKEVATLSCGFNVSVEELAQTRIYWQKEKKMVLTGMSGDMNIWPEYKNRTIYDITNNLSINILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 260 - protein, 342 aa, Synthetic construct
  REGION 1..342: note = CD80_180-Fc
VIHVTKEVKGWATLSCGHNVSVEELAQYRIYWQKEKKMVLTMMSGDLNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 261 - protein, 342 aa, Synthetic construct
  REGION 1..342: note = CD80_181-Fc
VIHVTKEVSRYATLSCGHNVSVEELAQTRIYWQKEKKMVLTMMSGDLNIWPEYKNRTIFDITNNLSIVILGLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 262 - protein, 342 aa, Synthetic construct
  REGION 1..342: note = CD80_182-Fc
VIHVTKEVKGWATLSCGHNVSVEELAQTRIYWQKGKKMVLTMMSGDLNIWPEYKNRTIFDITNNLSIVILGLRPSDEGTYECVVLKREKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 263 - protein, 342 aa, Synthetic construct
  REGION 1..342: note = CD80_183-Fc
VIHVTKEVKEYATLSCGHNVSVEELAQYRIYWQKEKKMVLTMMSGDLNIWPEYKNRTIFDITNNLSIVILGLRPSDEGTYECVVLKREKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 264 - protein, 342 aa, Synthetic construct
  REGION 1..342: note = CD80_184-Fc
VIHVTKEVKEYATLSCGHNVSVEELQQTRIYWQKEKKMVLTMMSGDLNIWPEYKNRTIFDITNNLSIVILGLRPSDEGTYECVVLKREKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 265 - protein, 342 aa, Synthetic construct
  REGION 1..342: note = CD80_185-Fc
VIHVTKEVKEYATLSCGHNVSVEELAQYRIYWQKEKKMVLTMMSGDLNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 266 - protein, 342 aa, Synthetic construct
  REGION 1..342: note = CD80_186-Fc
VIHVTKKVKEWATLSCGHNVSVEELAQTRIYWQKEKKMVLTMMSGDMNIWPEYKNRTIFDITHNLSIVILGLRPSDEGTYECVVLKKEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 267 - protein, 342 aa, Synthetic construct
  REGION 1..342: note = CD80_187-Fc
VIHVTKEVKEYATLSCGYNVSVEELAQTRIYWQKGKKMVLTMMSGDMNIWPEYKNRTIFDITNNLSIVILALRPSDEGTYECVVQKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 268 - protein, 342 aa, Synthetic construct
  REGION 1..342: note = CD80_188-Fc
VIHVTKEVKEVATLSCGHNVSVEELAQTRIYWQKDKKMVLTMMSGDMNIWPEYKNRTIFDITNNLSILILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 269 - protein, 342 aa, Synthetic construct
  REGION 1..342: note = CD80_189-Fc
VIHVTKEVKGWATLSCGHNVSVEELAQTRIYWQKEKKMVLTMMSGDVNIWPEYKNRTIFDITNNLSIVILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 270 - protein, 342 aa, Synthetic construct
  REGION 1..342: note = CD80_190-Fc
VIHVTKEVKEVATLSCGHNVSVEELAQYRIYWQKEKKMVLTMMSGDLNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 271 - protein, 342 aa, Synthetic construct
  REGION 1..342: note = CD80_191-Fc
VIHVTKEVKEYATLSCGHNVSVEELAQYRIYWQKEKKMVLTMMSGDLNIWPEYKNRTIFDITNNLSILILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 272 - protein, 342 aa, Synthetic construct
  REGION 1..342: note = CD80_192-Fc
VIHVTKNVKEVATLSCGHNVSVEELAQTRIYWQKDKKMVLTMMSGDMNIWPEYKNRTIFDITNNLSIVILALRPSDEGTYECVVLKYEKDAFKREHLAEVRLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 273 - protein, 342 aa, Synthetic construct
  REGION 1..342: note = CD80_193-Fc
VIHVTKEVKEYATLSCGHNVSVEELAQTRIYWQKEKKMVLTWMSGDMNIWPEYKNRTIFDIANNLSIVILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 274 - protein, 342 aa, Synthetic construct
  REGION 1..342: note = CD80_194-Fc
VIHVTKEVKEYATLSCGHNVSVEELAQTRIYWQKEKKMVLTWMSGDMNIWPEYKNRTIFDIANNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 275 - protein, 342 aa, Synthetic construct
  REGION 1..342: note = CD80_195-Fc
VIHVTKEVKEYATLSCGHNVSVEELAQTRIYWQKEKKMVLTWMSGDMNIWPEYKNRTIYDITNNLSINILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 276 - protein, 342 aa, Synthetic construct
  REGION 1..342: note = CD80_196-Fc
VIHVTKEVKEYATLSCGHNVSVEELAQTRIYWQKEKKMVLTWMSGDMNIWPKYKNRTIFDIANNLSIVILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 277 - protein, 342 aa, Synthetic construct
  REGION 1..342: note = CD80_197-Fc
VIHVTKEVKEYATLSCGHNVSVEELAQTRIYWQKDKKMVLTMMSGDMNIWPEYKNRTIFDITNNLSIVILALRPSDEGTYECVVLKQEKDAFKREHLAEVRLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 278 - protein, 342 aa, Synthetic construct
  REGION 1..342: note = CD80_198-Fc
VIHVTKEVKEVATLSCGYNVSVEELEQTSIYWQKDKKMVLTMMSGDLNIWPEYKNRTIFDITNNLSIMILGLRPSDGGTYECVVLKYEKGAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 279 - protein, 342 aa, Synthetic construct
  REGION 1..342: note = CD80_199-Fc
VIHVTKEVKGVATLSCGYNVSVEELAQTRIYWQKEKKMVLTMMSGDMNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 280 - protein, 342 aa, Synthetic construct
  REGION 1..342: note = CD80_200-Fc
VIHVTKEVNRVATLSCGVNVSVEELAQYRIYWQKEKKMVLTMMSGDMNIWPEYKNRTIFDITNNLSIVILGLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 281 - protein, 342 aa, Synthetic construct
  REGION 1..342: note = CD80_201-Fc
VIHVTKEVKGVATLSCGYNVSVEELAQYRIYWQKEKKMVLTMMSGDWNIWPEYKNRTIFDISNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 282 - protein, 342 aa, Synthetic construct
  REGION 1..342: note = CD80_202-Fc
VIHVTKHVKEVATLSCGLNISVEELAQYRIYWQKEKKMVLTMMSGSMNIWPEYKNRTIFDITNNLSIVILGLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 283 - protein, 342 aa, Synthetic construct
  REGION 1..342: note = CD80_203-Fc
VIHVTKEVNAWATLSCGFNVSVEELAQHRIYWQKEKKMVLTMMSGDLNIWPEYKNRTIFDIENNLSIVILALDPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 284 - protein, 342 aa, Synthetic construct
  REGION 1..342: note = CD80_204-Fc
VIHVTKEVKEVATLSCGVNISVEELAQYRIYWQKGKKMVLTMMSGDVNIWPEYKNRTIFDITNNLSIVILALEPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 285 - protein, 342 aa, Synthetic construct
  REGION 1..342: note = CD80_205-Fc
VIHVTKEVKGVATLSCGHNVSVEELAQTRIYWQKEKKMVLTMMSGKMNIWPEYKNRTIFDITNNLSIVILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 286 - protein, 342 aa, Synthetic construct
  REGION 1..342: note = CD80_206-Fc
VIHVTKEVKGVATLSCGTNVSVEELATTRIYWQKEKKMVLTMMSGELNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 287 - protein, 342 aa, Synthetic construct
  REGION 1..342: note = CD80_207-Fc
VIHVTKEVKEVATLSCGHNVSVEELAQTRIYWQKDKKMVLTMMSGDMNIWPEYKNRTIFDITNNLSITILALRPSDEGTYECVVLKYEKDAFKREHLAEVKLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 288 - protein, 342 aa, Synthetic construct
  REGION 1..342: note = CD80_208-Fc
VIHVTKEVKEYATLSCGHNVSVEELAQTRIYWQKGKKMVLTGMSGDMNIWPEYKNRTISDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 289 - protein, 342 aa, Synthetic construct
  REGION 1..342: note = CD80_209-Fc
VIHVTKEVKEYATLSCGHNVSVEELAQRRIYWQKGKKMVLTMMSGDLNIWPEYKNRTISDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 290 - protein, 342 aa, Synthetic construct
  REGION 1..342: note = CD80_210-Fc
VIHVTKNVKEVATLSCGHNVSVEELAQTRIYWQKDKKMVLTMMSGDMNIWPEYKNRTISDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 291 - protein, 342 aa, Synthetic construct
  REGION 1..342: note = CD80_211-Fc
VIHVTKEVKEYATLSCGHNVSVEELAQRRIYWQKGKKMVLTMMSGDLNIWPEYKNRTIFDITNNLSIVILGLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 292 - protein, 342 aa, Synthetic construct
  REGION 1..342: note = CD80_212-Fc
VIHVTKEVRAVATLSCGHNVSVEELAQTRIYWQKGKKMVLTMMSGDMNIWPEYKNRTIFDITNNLSITILALRPSDEGTYECVVLKYEKDAFKREHLAEVKLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 293 - protein, 342 aa, Synthetic construct
  REGION 1..342: note = CD80_213-Fc
VIHVTKEVKEYATLSCGHNVSVEELAQTRIYWQKEKKMVLTMMSGDMNIWPEYKNRTIFDITNNLSITILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 294 - protein, 342 aa, Synthetic construct
  REGION 1..342: note = CD80_214-Fc
VIHVTKEVKGVATLSCGHNVSVEELAQTRIYWQKGKKMVLTMMSGEVNIWPEYKNRTIFDITNNLSIMILALRPSDEGTYECVVLKYEKDAFKREHLAEVKLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 295 - protein, 342 aa, Synthetic construct
  REGION 1..342: note = CD80_215-Fc
VIHVTKEVRAVATLSCGHNVSVEELAQTRIYWQKGKKMVLTMMSGDMNIWPEYKNRTIFDITNNLSILILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 296 - protein, 342 aa, Synthetic construct
  REGION 1..342: note = CD80_216-Fc
VIHVTKEVKEWATLSCGHNVSVEELAQYRIYWQKEKKMVLTMMSGVMNIWPEYKNRTIFDITNNLSIVILALEPSDEGTYECVVLKYEKDALKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 297 - protein, 342 aa, Synthetic construct
  REGION 1..342: note = CD80_217-Fc
VIHVTKEVNGVATLSCGHNVSVEELAQTRIYWQKEKKMVLTMMSGDMNIWPEYKNRTIFDITNNLSIVILALRPSDEGTYECVVLKKEKDAFKREHLAEVQLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 298 - protein, 342 aa, Synthetic construct
  REGION 1..342: note = CD80_218-Fc
VIHVTKEVKEWATLSCGHNVSVEELAQYRIYWQKEKKMVLTMMSGVMNIWPEYKNRTIFDITNNLSITILALTPSDEGTYECVVLKNEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 299 - protein, 342 aa, Synthetic construct
  REGION 1..342: note = CD80_219-Fc
VIHVTKEVKSYATLSCGHNVSVEELAQTRIYWQKEKKMVLTRMSGDMNIWPEYKNRTIFDITNNLSIVILVLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 300 - protein, 342 aa, Synthetic construct
  REGION 1..342: note = CD80_220-Fc
VIHVTKEVKEVATLSCGFNVSVEELAQVRIYWQKEKKMVLTMMSGDLNIWPEYKNRTIFDITNNLSISILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 301 - protein, 342 aa, Synthetic construct
  REGION 1..342: note = CD80_221-Fc
VIHVTKEVKGVATLSCGHNVSVEELSQYRIYWQKEKKMVLTMMSGDMNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 302 - protein, 342 aa, Synthetic construct
  REGION 1..342: note = CD80_222-Fc
VIHVTKEVKEVATLSCGHNVSVEELAQTRIYWQKDKKMVLTMMSGQMNIWPEYKNRTIFDITNNLSIVILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 303 - protein, 342 aa, Synthetic construct
  REGION 1..342: note = CD80_223-Fc
VIHVTKEVKSVATLSCGHNVSVEELAQTRIYWQKEKKMVLTMMSGDMNIWPEYKNRTIFDITNNLSIMILALRPSDEGTYECVVLKPEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 304 - protein, 342 aa, Synthetic construct
  REGION 1..342: note = CD80_224-Fc
VIHVTKEVNEWATLSCGHNVSVEELAQTRIYWQKEKKMVLTMMSGDLNIWPEYKNRTIFDITNNLSITILALTPSDEGTYECVVLKNEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 305 - protein, 342 aa, Synthetic construct
  REGION 1..342: note = CD80_225-Fc
VIHVTKEVKEYATLSCGHNVSVEELAQTRIYWQKEKKMVLTMMSGDLNIWPEYKNRTIFDITNNLSITILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 306 - protein, 342 aa, Synthetic construct
  REGION 1..342: note = CD80_226-Fc
VIHVTKEVREVATLSCGHNVSVEELTQYRIYWQKAKKMVLTMMSGDLNIWPEYKNRTIFDITNNLSIVILGLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 307 - protein, 342 aa, Synthetic construct
  REGION 1..342: note = CD80_227-Fc
VIHVTKEVKEWATLSCGHNVSVEELAQHRIYWQKEKKMVLTMMSGQMNIWPEYKNRTIFDITNNLSILILALRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 308 - protein, 342 aa, Synthetic construct
  REGION 1..342: note = CD80_228-Fc
VIHVTKQVKEYATLSCGHNVSVEELAQTHIYWQKEKKMVLTMMSGDLNIWPEYKNRTIFDITNNLSITILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 309 - protein, 342 aa, Synthetic construct
  REGION 1..342: note = CD80_229-Fc
VIHVTKEVKEYATLSCGHNVSVEELAQTRIYWQKEKKMVLTMMSGDVNIWPEYKNRTIFDITNNLSIVILGLRPSDEGTYECVVEKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 310 - protein, 342 aa, Synthetic construct
  REGION 1..342: note = CD80_230-Fc
VIHVTKEVKGVATLSCGHNVSVEELAFTRIYWQKEKKMVLTMMSGNMNIWPEYKNRTIFDITNNLSIVILGLRPSDEGTYECVVLKYEKGAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 311 - protein, 342 aa, Synthetic construct
  REGION 1..342: note = CD80_231-Fc
VIHVTKEVKEWATLSCGHNVSVEELAQYRIYWQKEKKMVLTMMSGDLNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 312 - protein, 342 aa, Synthetic construct
  REGION 1..342: note = CD80_232-Fc
VIHVTKEVKEFATLSCGHNVSVEELAQYRIYWQKEKKMVLTMMSGDLNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 313 - protein, 342 aa, Synthetic construct
  REGION 1..342: note = CD80_233-Fc
VIHVTKEVKEYATLSCGHNVSVEELAQYRIYWQKEKKMVLTMMSGDLNIWPEYKNRTIFDITNNLSIMILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 314 - protein, 342 aa, Synthetic construct
  REGION 1..342: note = CD80_234-Fc
VIHVTKEVKEYATLSCGHNVSVEELAQYRIYWQKEKKMVLTMMSGDLNIWPEYKNRTIFDITNNLSILILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 315 - protein, 342 aa, Synthetic construct
  REGION 1..342: note = CD80_235-Fc
VIHVTKEVKEYATLSCGHNVSVEELAQTRIYWQKEKKMVLTMMSGDLNIWPEYKNRTIFDITNNLSIVILGLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 316 - protein, 342 aa, Synthetic construct
  REGION 1..342: note = CD80_236-Fc
VIHVTKEVKEYATLSCGHNVSVEELAQYRIYWQKEKKMVLTMMSGDLNIWPEYKNRTIFDITNNLSIVILGLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 317 - protein, 342 aa, Synthetic construct
  REGION 1..342: note = CD80_237-Fc
VIHVTKEVKEYATLSCGHNVSVEELAQTRIYWQKEKKMVLTMMSGDLNIWPEYKNRTIFDITNNLSIMILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 318 - protein, 342 aa, Synthetic construct
  REGION 1..342: note = CD80_238-Fc
VIHVTKEVKEYATLSCGHNVSVEELAQTRIYWQKEKKMVLTMMSGEVNIWPEYKNRTIFDITNNLSIMILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 319 - protein, 342 aa, Synthetic construct
  REGION 1..342: note = CD80_239-Fc
VIHVTKEVKEYATLSCGHNVSVEELAQYRIYWQKEKKMVLTMMSGEVNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 320 - protein, 342 aa, Synthetic construct
  REGION 1..342: note = CD80_240-Fc
VIHVTKEVKEYATLSCGHNVSVEELAQYRIYWQKEKKMVLTMMSGEVNIWPEYKNRTIFDITNNLSIMILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 321 - protein, 342 aa, Synthetic construct
  REGION 1..342: note = CD80_241-Fc
VIHVTKEVKSFATLSCGHNVSVEELAQYRIYWQKEKKMVLTMMSGDLNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 322 - protein, 342 aa, Synthetic construct
  REGION 1..342: note = CD80_242-Fc
VIHVTKEVKEFATLSCGHNVSVEELAQYRIYWQKEKKMVLTMMSGDLNIWPEYKNRTIFDISNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 323 - protein, 342 aa, Synthetic construct
  REGION 1..342: note = CD80_243-Fc
VIHVTKEVKEYATLSCGHNVSVEELAQTRIYWQKEKKMVLTMMSGDLNIWPEYKNRTIFDITNNLSIVILGLRPSDEGTYECVVLKREKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 324 - protein, 342 aa, Synthetic construct
  REGION 1..342: note = CD80_244-Fc
VIHVTKEVKEYATLSCGHNVSVEELAQYRIYWQKEKKMVLTMMSGDLNIWPEYKNRTIFDITNNLSIVILALRPSDEGTYECVVLKREKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 325 - protein, 7 aa, Synthetic construct
  REGION 1..7: note = GSG4S AA Sequence
GSGGGGS

SEQ ID NO: 326 - protein, 228 aa, Synthetic construct
  REGION 1..228: note = IgG4 Fc
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 327 - protein, 443 aa, Synthetic construct
  REGION 1..443: note = WT CD80 ECD-Fc
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 328 - protein, 5 aa, Synthetic construct
  REGION 1..5: note = linker
GGGGS

SEQ ID NO: 329 - protein, 10 aa, Synthetic construct
  REGION 1..10: note = linker
GGGGSGGGGS

SEQ ID NO: 330 - protein, 15 aa, Synthetic construct
  REGION 1..15: note = linker
GGGGSGGGGSGGGGS

SEQ ID NO: 331 - protein, 20 aa, Synthetic construct
  REGION 1..20: note = linker
GGGGSGGGGSGGGGSGGGGS

SEQ ID NO: 332 - protein, 25 aa, Synthetic construct
  REGION 1..25: note = linker
GGGGSGGGGSGGGGSGGGGSGGGGS

SEQ ID NO: 333 - protein, 7 aa, Synthetic construct
  REGION 1..7: note = linker
GGGGSSA

SEQ ID NO: 334 - protein, 13 aa, Synthetic construct
  REGION 1..13: note = linker
GGGGSGGGGSAAA

SEQ ID NO: 335 - protein, 12 aa, Synthetic construct
  REGION 1..12: note = linker
GSGGGGSGGGGS

SEQ ID NO: 336 - protein, 464 aa, Synthetic construct
  REGION 1..464: note = CD80-Fc-CD80_336
VIHVTKEVKGWATLSCGHNVSVEELAQYRIYWQKEKKMVLTMMSGDLNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSVIHVTKEVKGWATLSCGYNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 337 - protein, 464 aa, Synthetic construct
  REGION 1..464: note = CD80-CD80-Fc_337
VIHVTKEVKGWATLSCGHNVSVEELAQYRIYWQKEKKMVLTMMSGDLNIWPEYKNRTIFDITNNLSIVILALRPSDEGTYECVVLKYEKDAFKREHLAEVTLSVKADGGGGSGGGGSGGGGSVIHVTKEVKGWATLSCGYNVSVEELAQYRIYWQKEKKMVLTMMSGDLNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 338 - protein, 462 aa, Synthetic construct
  REGION 1..462: note = CD80-Fc-CD80_338
VIHVTKEVKGWATLSCGHNVSVEELAQYRIYWQKEKKMVLTMMSGDLNIWPEYKNRTIFDITNNLSIVILALRPSDEGTYECVVLKYEKDAFKREHLAEVTLSVKADGSGGGGSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSVIHVTKEVKGWATLSCGYNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 339 - protein, 458 aa, Synthetic construct
  REGION 1..458: note = CD80-Fc-CD80_339
VIHVTKEVKEYATLSCGHNVSVEELAQYRIYWQKEKKMVLTMMSGDLNIWPEYKNRTIFDITNNLSIVILALRPSDEGTYECVVLKYEKDAFKREHLAEVTLSVKADGSGGGGSPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSVIHVTKEVKEYATLSCGYNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 340 - protein, 464 aa, Synthetic construct
  REGION 1..464: note = CD80-CD80-Fc_340
VIHVTKEVKEYATLSCGHNVSVEELAQYRIYWQKEKKMVLTMMSGDLNIWPEYKNRTIFDITNNLSIVILALRPSDEGTYECVVLKYEKDAFKREHLAEVTLSVKADGGGGSGGGGSGGGGSVIHVTKEVKEYATLSCGYNVSVEELAQYRIYWQKEKKMVLTMMSGDLNIWPEYKNRTIFDITNNLSIVILALRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 341 - protein, 458 aa, Synthetic construct
  REGION 1..458: note = CD80-Fc-CD80_341
VIHVTKEVKEYATLSCGHNVSVEELAQTRIYWQKDKKMVLTMMSGDMNIWPEYKNRTIFDITNNLSIVILALRPSDEGTYECVVLKQEKDAFKREHLAEVRLSVKADGSGGGGSPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSVIHVTKEVKEYATLSCGYNVSVEELAQTRIYWQKDKKMVLTMMSGDMNIWPEYKNRTIFDITNNLSIVILALRPSDEGTYECVVLKQEKDAFKREHLAEVRLSVKAD

SEQ ID NO: 342 - protein, 464 aa, Synthetic construct
  REGION 1..464: note = CD80-CD80-Fc_342
VIHVTKEVKEYATLSCGHNVSVEELAQTRIYWQKDKKMVLTMMSGDMNIWPEYKNRTIFDITNNLSIVILALRPSDEGTYECVVLKQEKDAFKREHLAEVRLSVKADGGGGSGGGGSGGGGSVIHVTKEVKEYATLSCGYNVSVEELAQTRIYWQKDKKMVLTMMSGDMNIWPEYKNRTIFDITNNLSIVILALRPSDEGTYECVVLKQEKDAFKREHLAEVRLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 343 - protein, 232 aa, Synthetic construct
  REGION 1..232: note = IgG1 Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 344 - protein, 231 aa, Synthetic construct
  REGION 1..231: note = Fc C220S/L234A/L235E/G237A/K447del
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 345 - protein, 232 aa, Synthetic construct
  REGION 1..232: note = Fc C220S/L234A/L235E/G237A/E356D/M358L
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - protein, 231 aa, Synthetic construct
  REGION 1..231: note = Fc (E356D and M358L)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 347 - protein, 232 aa, Synthetic construct
  REGION 1..232: note = Fc (C5S (C220S), R77C, (R292C), N82G (N297G), V87C (V302C))
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 232 aa, Synthetic construct
  REGION 1..232: note = Fc with C220S/L234A/L235E/G237A
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 231 aa, Synthetic construct
  REGION 1..231: note = Fc with C220S/E233P/L234V/L235A/G236del/S267K
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 350 - protein, 231 aa, Synthetic construct
  REGION 1..231: note = Fc (C5S (C220S), R77C, (R292C), N82G (N297G), V87C (V302C), L232del (K447del))
EPKSSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 351 - protein, 231 aa, Synthetic construct
  REGION 1..231: note = Fc with C220S/L234A/L235E/G237A/K447del
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 352 - protein, 230 aa, Synthetic construct
  REGION 1..230: note = Fc with C220S/E233P/L234V/L235A/G236del/S267K/K447del
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 353 - protein, 235 aa, Synthetic construct
  REGION 1..235: note = IgG2 Fc
TKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 354 - protein, 229 aa, Synthetic construct
  REGION 1..229: note = IgG4 Fc
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 355 - protein, 229 aa, Synthetic construct
  REGION 1..229: note = IgG4 Fc S228P
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 356 - protein, 227 aa, Synthetic construct
  REGION 1..227: note = Fc
DKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 357 - protein, 232 aa, Synthetic construct
  REGION 1..232: note = Fc
DPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 358 - protein, 227 aa, Synthetic construct
  REGION 1..227: note = Fc
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 359 - protein, 232 aa, Synthetic construct
  REGION 1..232: note = Fc
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 360 - protein, 522 aa, Synthetic construct
  REGION 1..522: note = single chain Fc with effectorless mutation
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSGGGGSGGGGSGGGGSGGGGSGGGGSGGGGSGGGGSEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 361 - protein, 222 aa, Synthetic construct
  REGION 1..222: note = IgG2 Fc
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 362 - protein, 226 aa, Synthetic construct
  REGION 1..226: note = IgG1 Fc effectorless (L234A/L235E/G237A) and K447del
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 363 - protein, 5 aa, Synthetic construct
  REGION 1..5: note = hinge
EPKSC

SEQ ID NO: 364 - protein, 5 aa, Synthetic construct
  REGION 1..5: note = hinge
EPKSS

SEQ ID NO: 365 - protein, 60 aa, Synthetic construct
  REGION 1..60: note = anti-CD19 scFv
DIQMTQTTSSLSASLGDRVTISCRASQDISKYLNWYQQKPDGTVKLLIYHTSRLHSGVPS

SEQ ID NO: 366 - protein, 60 aa, Synthetic construct
  REGION 1..60: note = CD8-derived hinge and transmembrane domain
RFSGSGSGTDYSLTISNLEQEDIATYFCQQGNTLPYTFGGGTKLEITGSTSGSGKPGSGE

SEQ ID NO: 367 - protein, 60 aa, Synthetic construct
  REGION 1..60: note = CD8-derived hinge and transmembrane domain
GSTKGEVKLQESGPGLVAPSQSLSVTCTVSGVSLPDYGVSWIRQPPRKGLEWLGVIWGSE

SEQ ID NO: 368 - protein, 60 aa, Synthetic construct
  REGION 1..60: note = CD8 hinge and transmembrane domain
TTYYNSALKSRLTIIKDNSKSQVFLKMNSLQTDDTAIYYCAKHYYYGGSYAMDYWGQGTS

SEQ ID NO: 369 - protein, 5 aa, Synthetic construct
  REGION 1..5: note = CD3zeta intracellular signaling domain
VTVSS

SEQ ID NO: 370 - protein, 71 aa, Synthetic construct
  REGION 1..71: note = 4-1BB-derived costimulatory domain
KPTTTPAPRPPTPAPTIASQPLSLRPEASRPAAGGAVHTRGLDFASDIYIWAPLAGTCGVLLLSLVITLYC

SEQ ID NO: 371 - protein, 60 aa, Synthetic construct
  REGION 1..60: note = CD28-derived costimulatory domain
AKPTTTPAPRPPTPAPTIASQPLSLRPEACRPAAGGAVHTRGLDFACDIYIWAPLAGTCG

SEQ ID NO: 372 - protein, 9 aa, Synthetic construct
  REGION 1..9: note = CD28-derived costimulatory domain 2
VLLLSLVIT

SEQ ID NO: 373 - protein, 68 aa, Synthetic construct
  REGION 1..68: note = CD28-derived costimulatory domain 3
KPTTTPAPRPPTPAPTIASQPLSLRPEACRPAAGGAVHTRGLDFACDIYIWAPLAGTCGVLLLSLVIT

SEQ ID NO: 374 - protein, 112 aa, Synthetic construct
  REGION 1..112: note = T2A protein
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 375 - protein, 42 aa, Synthetic construct
  REGION 1..42: note = T2A
KRGRKKLLYIFKQPFMRPVQTTQEEDGCSCRFPEEEEGGCEL

SEQ ID NO: 376 - protein, 40 aa, Synthetic construct
  REGION 1..40: note = T2A
SKRSRLLHSDYMNMTPRRPGPTRKHYQPYAPPRDFAAYRS

SEQ ID NO: 377 - protein, 41 aa, Synthetic construct
  REGION 1..41: note = Protein
RSKRSRLLHSDYMNMTPRRPGPTRKHYQPYAPPRDFAAYRS

SEQ ID NO: 378 - protein, 44 aa, Synthetic construct
  REGION 1..44: note = T2A
FWVRSKRSRLLHSDYMNMTPRRPGPTRKHYQPYAPPRDFAAYRS

SEQ ID NO: 379 - protein, 20 aa, Synthetic construct
  REGION 1..20: note = Nucleotide
SGEGRGSLLTCGDVEENPGP

SEQ ID NO: 380 - protein, 18 aa, Synthetic construct
  REGION 1..18: note = P2A protein
EGRGSLLTCGDVEENPGP

SEQ ID NO: 381 - protein, 21 aa, Synthetic construct
  REGION 1..21: note = Blue fluorescent protein
GSGEGRGSLLTCGDVEENPGP

SEQ ID NO: 382 - protein, 63 aa, Synthetic construct
  REGION 1..63: note = Blue fluorescent protein
GGCAGTGGCGAGGGCAGAGGAAGTCTGCTAACATGCGGTGACGTCGAGGAGAATCCTGGCCCA

SEQ ID NO: 383 - protein, 22 aa, Synthetic construct
  REGION 1..22: note = eGFP
GSGATNFSLLKQAGDVEENPGP

SEQ ID NO: 384 - protein, 60 aa, Synthetic construct
  REGION 1..60: note = Green fluorescent protein
SELIKENMHMKLYMEGTVDNHHFKCTSEGEGKPYEGTQTMRIKVVEGGPLPFAFDILATS

SEQ ID NO: 385 - protein, 60 aa, Synthetic construct
  REGION 1..60: note = eGFP
FLYGSKTFINHTQGIPDFFKQSFPEGFTWERVTTYEDGGVLTATQDTSLQDGCLIYNVKI

SEQ ID NO: 386 - protein, 60 aa, Synthetic construct
  REGION 1..60: note = antiCD19x28z chimeric antigen receptor: protein
RGVNFTSNGPVMQKKTLGWEAFTETLYPADGGLEGRNDMALKLVGGSHLIANIKTTYRSK

SEQ ID NO: 387 - protein, 52 aa, Synthetic construct
  REGION 1..52: note = 2nd Gen CAR (without signal sequence or T2A or BFP)
KPAKNLKMPGVYYVDYRLERIKEANNETYVEQHEVAVARYCDLPSKLGHKLN

SEQ ID NO: 388 - protein, 232 aa, Synthetic construct
  REGION 1..232: note = 2nd Gen CAR with T2A
SRSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLWEAFTETLYPADGGLEGRNDMALKLVGGSHLIANIKTTYRSKKPAKNLKMPGVYYVDYRLERIKEANNETYVEQHEVAARYCDLPSKLGHKLN

SEQ ID NO: 389 - protein, 59 aa, Synthetic construct
  REGION 1..59: note = 2nd Gen CAR with CD28-derived costimulatory domain (without signal sequence or T2A or BFP)
SKGEELFTGVVPILVELDGDVNGHKFSVSGEGEGDATYGKLTLKFICTTGKLPVPWPTL

SEQ ID NO: 390 - protein, 60 aa, Synthetic construct
  REGION 1..60: note = antiCD19 chimeric antigen receptor CAR-2
VTTLTYGVQCFSRYPDHMKQHDFFKSAMPEGYVQERTIFFKDDGNYKTRAEVKFEGDTLV

SEQ ID NO: 391 - protein, 60 aa, Synthetic construct
  REGION 1..60: note = antiCD19 chimeric antigen receptor CAR-2
NRIELKGIDFKEDGNILGHKLEYNYNSHNVYIMADKQKNGIKVNFKIRHNIEDGSVQLAD

SEQ ID NO: 392 - protein, 58 aa, Synthetic construct
  REGION 1..58: note = anti-CD19 chimeric antigen receptor CAR-2
HYQQNTPIGDGPVLLPDNHYLSTQSALSKDPNEKRDHMVLLEFVTAAGITHGMDELYK

SEQ ID NO: 393 - protein, 59 aa, Synthetic construct; identical to SEQ ID NO: 389
  REGION 1..59: note = antiCD19 chimeric antigen receptor CAR-1

SEQ ID NO: 394 - protein, 60 aa, Synthetic construct
  REGION 1..60: note = antiCD19 chimeric antigen receptor CAR-1
VTTFSYGVQCFSRYPDHMKQHDFFKSAMPEGYVQERTIFFKDDGNYKTRAEVKFEGDTLV

SEQ ID NO: 395 - protein, 60 aa, Synthetic construct; identical to SEQ ID NO: 391
  REGION 1..60: note = Anti-CD19 scFv

SEQ ID NO: 396 - protein, 18 aa, Synthetic construct
  REGION 1..18: note = HSA signal peptide
MKWVTFISLLFLFSSAYS

SEQ ID NO: 397 - protein, 22 aa, Synthetic construct
  REGION 1..22: note = Ig kappa light chain
MDMRAPAGIFGFLLVLFPGYRS

SEQ ID NO: 398 - protein, 19 aa, Synthetic construct
  REGION 1..19: note = human azurocidin preprotein signal sequence
MTRLTVLALLAGLLASSRA

SEQ ID NO: 399 - protein, 19 aa, Synthetic construct
  REGION 1..19: note = IgG heavy chain signal peptide
MELGLSWIFLLAILKGVQC

SEQ ID NO: 400 - protein, 19 aa, Synthetic construct
  REGION 1..19: note = IgG heavy chain signal peptide
MELGLRWVFLVAILEGVQC

SEQ ID NO: 401 - protein, 19 aa, Synthetic construct
  REGION 1..19: note = IgG heavy chain signal peptide
MKHLWFFLLLVAAPRWVLS

SEQ ID NO: 402 - protein, 19 aa, Synthetic construct
  REGION 1..19: note = IgG heavy chain signal peptide
MDWTWRILFLVAAATGAHS

SEQ ID NO: 403 - protein, 19 aa, Synthetic construct
  REGION 1..19: note = IgG heavy chain signal peptide
MDWTWRFLFVVAAATGVQS

SEQ ID NO: 404 - protein, 19 aa, Synthetic construct
  REGION 1..19: note = IgG heavy chain signal peptide
MEFGLSWLFLVAILKGVQC

SEQ ID NO: 405 - protein, 19 aa, Synthetic construct
  REGION 1..19: note = IgG heavy chain signal peptide
MEFGLSWVFLVALFRGVQC

SEQ ID NO: 406 - protein, 26 aa, Synthetic construct
  REGION 1..26: note = IgG heavy chain signal peptide
MDLLHKNMKHLWFFLLLVAAPRWVLS

SEQ ID NO: 407 - protein, 22 aa, Synthetic construct
  REGION 1..22: note = IgG Kappa light chain signal sequences:
MDMRVPAQLLGLLLLWLSGARC

SEQ ID NO: 408 - protein, 22 aa, Synthetic construct
  REGION 1..22: note = IgG Kappa light chain signal sequences:
MKYLLPTAAAGLLLLAAQPAMA

SEQ ID NO: 409 - protein, 17 aa, Synthetic construct
  REGION 1..17: note = Gaussia luciferase
MGVKVLFALICIAVAEA

SEQ ID NO: 410 - protein, 18 aa, Synthetic construct
  REGION 1..18: note = Human albumin
MKWVTFISLLFLFSSAYS

SEQ ID NO: 411 - protein, 18 aa, Synthetic construct
  REGION 1..18: note = Human chymotrypsinogen
MAFLWLLSCWALLGTTFG

SEQ ID NO: 412 - protein, 14 aa, Synthetic construct
  REGION 1..14: note = Human interleukin-2
MQLLSCIALILALV

SEQ ID NO: 413 - protein, 15 aa, Synthetic construct
  REGION 1..15: note = Human trypsinogen-2
MNLLLILTFVAAAVA

SEQ ID NO: 414 - protein, 465 aa, Synthetic construct
  REGION 1..465: note = (CD80 H6771 IgV) G(G4S)3 (CD80 H6771 IgV) GSG4S Fc4.1
VIHVTKEVKEYATLSCGHNVSVEELAQYRIYWQKEKKMVLTMMSGDLNIWPEYKNRTIFDITNNLSIVILGLRPSDEGTYECVVLKYEKDAFKREHLAEVTLSVKADGGGGGSGGGGSGGGGSVIHVTKEVKEYATLSCGYNVSVEELAQYRIYWQKEKKMVLTMMSGDLNIWPEYKNRTIFDITNNLSIVILGLRPSDEGTYECVVLKYEKDAFKREHLAEVTLSVKADGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 415 - protein, 107 aa, synthetic construct
  REGION 1..107: note = CD80_415
VIHVTKEVKGWATLSCGYNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 416 - protein, 107 aa, synthetic construct
  REGION 1..107: note = CD80_416
VIHVTKEVKEYATLSCGYNVSVEELAQYRIYWQKEKKMVLTMMSGDLNIWPEYKNRTIFDITNNLSIVILALRPSDEGTYECVVLKYEKDAFKREHLAEVTLSVKAD

SEQ ID NO: 417 - protein, 107 aa, synthetic construct
  REGION 1..107: note = CD80_417
VIHVTKEVKEYATLSCGYNVSVEELAQYRIYWQKEKKMVLTMMSGDLNIWPEYKNRTIFDITNNLSIVILGLRPSDEGTYECVVLKYEKDAFKREHLAEVTLSVKAD

(19) United States
(12) Patent Application Publication
(10) Pub. No.: US 2025/0243260 A1
(43) Pub. Date: Jul. 31, 2025

(54) IMMUNOMODULATORY PROTEINS OF VARIANT CD80 POLYPEPTIDES, CELL THERAPIES AND RELATED METHODS AND USES

(71) Applicant: Alpine Immune Sciences, Inc., Seattle, WA (US)

(72) Inventors: Mark F. MAURER, Seattle, WA (US); Daniel William DEMONTE, Seattle, WA (US)

(73) Assignee: Alpine Immune Sciences, Inc., Seattle, WA (US)

(21) Appl. No.: 18/844,376

(22) PCT Filed: Mar. 6, 2023
(86) PCT No.: PCT/US2023/063809

§ 371 Date: Sep. 5, 2024

Related U.S. Application Data
(60) Provisional application No. 63/317,521, filed on Mar. 7, 2022.
(60) Provisional application No. 63/317,523, filed on Mar. 7, 2022.

(51) Int. Cl. C07K 14/705; A61K 38/00; A61P 35/00

(52) U.S. Cl. CPC C07K 14/70532; A61P 35/00; A61K 38/00; C07K 2319/30

(57) ABSTRACT

Provided are immunomodulatory proteins containing an engineered variant CD80 extracellular domain that exhibits improved PD-L1 binding. The immunomodulatory proteins include variant CD80 domains with amino acid substitutions in the IgV domain. Among the provided immunomodulatory proteins are variant CD80-Fc fusion proteins. Also provided are cell therapies, such as T cell therapies, engineered with secretable or transmembrane immunomodulatory proteins containing the variant CD80 disclosed herein. In some aspects, the provided immunomodulatory proteins are capable of antagonizing PD-1/PD-L1 in addition to providing CD28 costimulation in a PD-L1-dependent fashion. Also provided are nucleic acid molecules encoding the immunomodulatory proteins. The immunomodulatory proteins provide therapeutic utility for a variety of immunological diseases, disorders, or conditions, such as cancer. Also provided are compositions and methods for making and using such immunomodulatory proteins as well as compositions and methods for making and using such cell therapies.

### CROSS REFERENCE TO RELATED APPLICATIONS

This application claims priority to U.S. provisional application 63/317,521 entitled “IMMUNOMODULATORY PROTEINS OF VARIANT CD80 POLYPEPTIDES AND RELATED METHODS AND USES”, filed Mar. 7, 2022, and to U.S. provisional application 63/317,523, entitled “SECRETABLE AND TRANSMEMBRANE IMMUNOMODULATORY PROTEINS OF VARIANT CD80 POLYPEPTIDES AND RELATED CELL THERAPIES”, filed Mar. 7, 2022, the contents of each of which are incorporated by reference in their entirety for all purposes.

### REFERENCE TO AN ELECTRONIC SEQUENCE LISTING

The present application is being filed along with a Sequence Listing in electronic format. The Sequence Listing is provided as a file entitled 761612004040SeqList.xml, created Mar. 6, 2023, which is 663,369 bytes in size. The information in the electronic format of the Sequence Listing is incorporated by reference in its entirety.

### FIELD

The present disclosure provides immunomodulatory proteins containing an engineered variant CD80 extracellular domain that exhibits improved PD-L1 binding. The present disclosure also provides cell therapies, such as T cell therapies, engineered with secretable or transmembrane immunomodulatory proteins containing an engineered variant CD80 extracellular domain that exhibits improved PD-L1 binding. The immunomodulatory proteins include variant CD80 domains with amino acid substitutions in the IgV domain. Among the provided immunomodulatory proteins are variant CD80-Fc fusion proteins. In some aspects, the provided immunomodulatory proteins are capable of antagonizing PD-1/PD-L1 in addition to providing CD28 costimulation in a PD-L1-dependent fashion. The present disclosure also provides nucleic acid molecules encoding the immunomodulatory proteins. The immunomodulatory proteins provide therapeutic utility for a variety of immunological diseases, disorders, or conditions, such as cancer. In some embodiments, the cell therapy also is engineered with an antigen receptor such as a recombinant T cell receptor (TCR) or chimeric antigen receptor (CAR). The provided secretable and transmembrane immunomodulatory proteins may improve the potency and efficacy of cell therapies. Compositions and methods for making and using such proteins or cell therapies are provided.

### BACKGROUND

Modulation of the immune response by intervening in the processes that occur in the immunological synapse (IS) formed by and between antigen-presenting cells (APCs) or target cells and lymphocytes is of increasing medical interest. Mechanistically, cell surface proteins in the IS can involve the coordinated and often simultaneous interaction of multiple protein targets with a single protein to which they bind. IS interactions occur in close association with the junction of two cells, and a single protein in this structure can interact with both a protein on the same cell (cis) as well as a protein on the associated cell (trans), likely at the same time. Although therapeutics are known that can modulate the IS, improved therapeutics are needed. Provided are immunomodulatory proteins that meet such needs. Also provided are cell therapies that meet such needs.

### SUMMARY

The present application in one aspect provides immunomodulatory proteins containing an engineered variant CD80 extracellular domain that exhibits improved PD-L1 binding. In some aspects, the immunomodulatory proteins include variant CD80 domains with amino acid substitutions in the IgV domain. In some aspects, the provided immunomodulatory proteins are capable of antagonizing PD-1/PD-L1 in addition to providing CD28 costimulation in a PD-L1-dependent fashion. Also provided herein are variant CD80-Fc fusion proteins, nucleic acid molecules encoding the immunomodulatory proteins, compositions and methods for making and using such immunomodulatory proteins. In some aspects, the immunomodulatory proteins provide therapeutic utility for a variety of immunological diseases, disorders, or conditions, such as cancer.

The present application in another aspect provides an immunomodulatory protein comprising at least one variant CD80 extracellular domain polypeptide, wherein the variant CD80 extracellular domain polypeptide comprises an amino acid substitution at position 11 in the sequence set forth in SEQ ID NO:2 or a portion thereof comprising an IgV domain, wherein the variant CD80 extracellular domain polypeptide exhibits increased binding to PD-L1 compared to wild-type CD80 set forth in SEQ ID NO:2 or the portion thereof comprising the IgV domain.

The present application in another aspect provides an immunomodulatory protein comprising at least one variant CD80 extracellular domain polypeptide, wherein the variant CD80 extracellular domain polypeptide comprises 2 to 10 amino acid substitutions at positions in the sequence set forth in SEQ ID NO:2 or a portion thereof comprising an IgV domain, wherein at least one substitution is at a position selected from among 9, 10, or 11, wherein the variant CD80 extracellular domain polypeptide exhibits increased binding to PD-L1 compared to wild-type CD80 set forth in SEQ ID NO:2 or the portion thereof comprising the IgV domain. In some embodiments, the variant CD80 extracellular domain polypeptide comprises an amino acid substitution at position 11 and the substitution is to an aromatic amino acid residue. In some embodiments, the aromatic amino acid residues is selected from the group consisting of tyrosine (Y), tryptophan (W) or phenylalanine (F), optionally wherein the amino acid substitution is V11Y, V11F, or V11W.

In some of any of the provided embodiments, the variant CD80 extracellular domain polypeptide comprises the amino acid substitution V11Y.

In some of any of the provided embodiments, the variant CD80 extracellular domain polypeptide comprises the amino acid substitutions V11Y/M47V/F59S/L85E, V11Y/M47V/L85E, V11Y/M42I/M47V/A71G, V11Y/T28Y/M47L/L85E, E10S/V11F/T28Y/M47L/T62S, E10G/V11W/V22S/T28P/A71G/E81K/Y87R, V11Y/T28Y/L85E/Y87I, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E10G/V11W/M47V/L85E, V11Y/T28Y/M47L/V68L/L85E, V11Y/M42W/T62A/L85E, V11Y/M42W/T62A, V11Y/M42W/F59Y/V68N, V11Y/M42W/E52K/T62A/L85E, V11Y/E35D/Y87Q/T101R, K9N/E10A/V11W/H18F/T28H/M47L/T62E/R73D, V11Y/E35G/M42G/F59S, V11Y/T28R/E35G/M47L/F59S, V11Y/T28R/E35G/M47L/A71G, V11Y/V68T, V11W/T28Y/D46V/R73E/F92L, V11W/T28Y/D46V/V68T/R73T/Y87N, E10S/V11Y/M42R/A71V, K9N/V11W/M47L/V68T/R73T/Y87N, V11Y/M47L/V68T, V11W/T28H/D46Q/V68L/L85E, E7Q/V11Y/R29H/M47L/V68T, V11Y/M47V/A71G/L85E, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

In some of any of the provided embodiments, the variant CD80 extracellular domain polypeptide comprises an amino acid substitution at position 9 and is substitution to a polar uncharged amino acid residue. In some embodiments, the polar uncharged amino acid is selected from the group consisting of serine (S), asparagine (N), glutamine (Q), threonine (T).

In some of any of the provided embodiments, the variant CD80 extracellular domain polypeptide comprises the amino acid substitution K9S or K9N. In some embodiments, the variant CD80 extracellular domain polypeptide comprise an amino acid substitution at position 9 and the substitution is to another basic amino acid. In some embodiments, the other basic amino acid is selected from the group consisting of arginine or histidine. In some embodiments, the variant CD80 extracellular domain polypeptide comprises the amino acid substitution is K9R.

In some of any of the provided embodiments, the variant CD80 extracellular domain polypeptide comprises the amino acid substitutions K9N/E10G/N63S/L85E, K9N/E10G/E35G/D46E/M47V/V68M, K9R/E10S/V11Y/M42L/F59M/V68M/L85E, K9S/E10R/V11Y/M47L/A71G, K9N/E10R/H18V/T28Y/A71G, K9N/E10A/V11W/H18F/T28H/M47L/T62E/R73D, K9R/E10A/E35G/V68T/T101K, K9R/E10A/E35G/V68L/L85E, K9N/E10G/Y87K/T101Q, K9N/V11W/M47L/V68T/R73T/Y87N, K9R/A26T/T28Y/E35A/M47L/A71G.

In some embodiments, the variant CD80 extracellular domain comprises an amino acid substitution at position 10 and the substitution is to a nonpolar amino acid. In some embodiments, the nonpolar amino acid is glycine, alanine or valine. In some embodiments, the variant CD80 extracellular domain comprises the amino acid substitution E10G or E10A. In some embodiments, the variant CD80 extracellular domain comprises an amino acid substitution at position 10 and the amino acid substitution is selected from the group consisting of E10G, E10S, E10R, and E10A.

In some of any of the provided embodiments, the variant CD80 extracellular domain polypeptide comprises the amino acid substitutions K9N/E10G/N63S/L85E, E10G/E35G/D46E/M47V/V68M, K9N/E10G/E35G/D46E/M47V/V68M, K9R/E10S/V11Y/M42L/F59M/V68M/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, E10G/V11W/T28Y/M47L, K9S/E10R/V11Y/M47L/A71G, E10G/V11W/E35G/M47L/A71G/Y87R, E10G/V11W/M47V/L85E, E10G/H18Y, K9N/E10R/H18V/T28Y/A71G, E10G/H18Y/T28Y/M47W/T62S, K9N/E10A/V11W/H18F/T28H/M47L/T62E/R73D, E10G/D46K/L85E, E10G/H18T/Q27T/D46E/M47L, K9R/E10A/E35G/V68T/T101K, E10G/E35G/D46E/M47V/V68M/T101K, K9R/E10A/E35G/V68L/L85E, K9N/E10G/Y87K/T101Q, E10S/V11Y/M42R/A71V, E10G/A26S/T28Y, E10S/V68M/Y87P, E10G/Q27F/D46N/A71G/D90G, or E10S/V11F/T28Y/M47L.

In some of any of the provided embodiments, the variant CD80 extracellular domain polypeptide further comprises an amino acid substitution at position 47 in the sequence set forth in SEQ ID NO:2 or the portion thereof comprising an IgV domain. In some embodiments, the amino acid substitution at position 47 is to another hydrophobic amino acid. In some embodiments, the hydrophobic amino acid is selected from the group consisting of valine, leucine, isoleucine or proline. In some embodiments, the variant CD80 extracellular domain comprises the amino acid substitution M47L or M47V. In some embodiments, the variant CD80 extracellular domain polypeptide comprises the amino acid substitutions V11W/M47L, V11F/M47L, V11Y/M47L, V11W/M47V, or V11Y/M47V.

In some of any of the provided embodiments, the variant CD80 extracellular domain polypeptide comprises the amino acid substitutions V11Y/M47V/F59S/L85E, V11Y/M47V/L85E, V11Y/M42I/M47V/A71G, V11Y/T28Y/M47L/L85E, E10S/V11F/T28Y/M47L/T62S, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E10G/V11W/M47V/L85E, V11Y/T28Y/M47L/V68L/L85E, K9N/E10A/V11W/H18F/T28H/M47L/T62E/R73D, V11Y/T28R/E35G/M47L/F59S, V11Y/T28R/E35G/M47L/A71G, K9N/V11W/M47L/V68T/R73T/Y87N, V11Y/M47L/V68T, E7Q/V11Y/R29H/M47L/V68T, V11Y/M47V/A71G/L85E, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

In some of any of the provided embodiments, the variant CD80 extracellular domain polypeptide further comprises an amino acid substitution at position 28 in the sequence set forth in SEQ ID NO:2 or the portion thereof comprising an IgV domain.

In some of any of the provided embodiments, the variant CD80 extracellular domain polypeptide comprises an amino acid substitution selected from T28Y, T28P, T28H, T28R, or T28V. In some embodiments, the amino acid substitution is T28Y.

In some of any of the provided embodiments, the variant CD80 extracellular domain polypeptide comprises the amino acid substitutions V11W/T28Y/M47L, V11F/T28Y/M47L, V11Y/T28Y/M47L, V11W/T28Y/M47V, V11F/T28Y/M47V, or V11Y/T28Y/M47V.

In some of any of the provided embodiments, the variant CD80 extracellular domain polypeptide comprises the amino acid substitutions V11Y/T28Y/M47L.

In some of any of the provided embodiments, the variant CD80 extracellular domain polypeptide further comprises an amino acid substitution at position 68, optionally wherein the amino acid substitution is V68M, V68L, V68N, V68T, or V68S.

In some of any of the provided embodiments, the variant CD80 extracellular domain polypeptide comprises the amino acid substitutions V11Y/T28Y/M47L/V68M.

In some of any of the provided embodiments, the variant CD80 extracellular domain polypeptide comprises the amino acid substitutions V11Y/T28Y/M47L/V68L.

In some of any of the provided embodiments, the variant CD80 extracellular domain polypeptide comprises the amino acid substitutions E10G/V11W/T28Y/M47L.

In some of any of the provided embodiments, the variant CD80 extracellular domain polypeptide further comprises an amino acid substitution at position 7 in the sequence set forth in SEQ ID NO:2 or a portion thereof comprising an IgV domain.

In some of any of the provided embodiments, the variant CD80 extracellular domain polypeptide further comprises an amino acid substitution at position 18 in the sequence set forth in SEQ ID NO:2 or a portion thereof comprising an IgV domain. In some of any of the provided embodiments, the variant CD80 extracellular domain polypeptide comprises amino acid substitutions E10G/V11W/H18Y/T28Y/M47L, V11Y/H18Y/T28Y/M47L or V11Y/H18Y/T28Y/M47L/A71G.

The present application in another aspect provides an immunomodulatory protein comprising at least one variant CD80 extracellular domain polypeptide, wherein the variant CD80 extracellular domain polypeptide comprises an amino acid substitution at position 7 in the sequence set forth in SEQ ID NO:163 or a portion thereof comprising the IgV domain, wherein the variant CD80 extracellular domain polypeptide exhibits increased binding to PD-L1 compared to the wild-type CD80 extracellular domain polypeptide set forth in SEQ ID NO:2 or the portion thereof comprising the IgV domain.

In some of any of the provided embodiments, the variant CD80 extracellular domain polypeptide comprises an amino acid substitution at position 7 and the substitution is to a polar uncharged amino acid residue. In some embodiments, the polar uncharged amino acid is selected from the group consisting of serine (S), asparagine (N), glutamine (Q), threonine (T).

In some of any of the provided embodiments, the variant CD80 extracellular domain polypeptide comprises the amino acid substitution E7Q, E7N or E7S. In some embodiments, the variant CD80 extracellular domain polypeptide comprises an amino acid substitution at position 7 and the substitution is to a basic amino acid. In some embodiments, the basic amino acid is selected from the group consisting of arginine, histidine or lysine.

In some of any of the provided embodiments, the variant CD80 extracellular domain polypeptide comprises the amino acid substitution E7H or E7K.

In some of any of the provided embodiments, the variant CD80 extracellular domain polypeptide comprises the amino acid substitutions E7S/H18I/V20L/A26K/M47L/A71N, E7K/V11W/N63H/A71G/Y87K, E7N/E35D/T101R, E7H/H18L/V20I/T28Y/D46S/A71G, E7N/E35D/F59S, or E7Q/V11Y/R29H/M47L/V68T.

In some of any of the provided embodiments, the immunomodulatory protein further comprises an amino acid substitution at position 101 in the sequence set forth in SEQ ID NO:2 or a portion thereof comprising an IgV domain.

The present application in another aspect provides an immunomodulatory protein comprising at least one variant CD80 extracellular domain polypeptide, wherein the variant CD80 extracellular domain polypeptide comprises an amino acid substitution at position 101 in the sequence set forth in SEQ ID NO:2 or a portion thereof comprising an IgV domain, wherein the variant CD80 extracellular domain polypeptide exhibits increased binding to PD-L1 compared to wild-type CD80 set forth in SEQ ID NO:2 or the portion thereof comprising the IgV domain. In some embodiments, the variant CD80 extracellular domain polypeptide comprises an amino acid substitution at position 101 and the substitution is to a charged amino acid residue. In some embodiments, the charged amino acid residue is basic and the amino acid substitution is to a histidine (H), lysine (K) or arginine (R). In some embodiments, the variant CD80 extracellular domain polypeptide comprises an amino acid substitution T101K or T101R. In some embodiments, the charged amino acid residue is acidic and the amino acid substitution is to aspartate (D), glutamate (E), asparagine (N) or glutamine (Q). In some embodiments, the variant CD80 extracellular domain polypeptide comprises an amino acid substitution T101Q.

In some of any of the provided embodiments, the variant CD80 extracellular domain polypeptide comprises the amino acid substitutions E7N/E35D/T101R, V11Y/E35D/Y87Q/T101R, E35D/V68T/T101K, K9R/E10A/E35G/V68T/T101K, E10G/E35G/D46E/M47V/V68M/T101K, or K9N/E10G/Y87K/T101Q.

In some of any of the provided embodiments, the variant CD80 extracellular domain polypeptide comprises the amino acid substitutions V11Y/E35D/Y87Q/T101R.

In some of any of the provided embodiments, the immunomodulatory protein further comprises an additional amino acid substitution at a different position wherein the amino acid substitution is selected from the group consisting of E7S, E7K, E7N, E7H, E7Q, K9N, K9R, K9S, E10G, E10S, E10R, E10A, V11Y, V11F, V11W, H18I, H18Y, H18F, H18V, H18L, H18T, V20L, V20I, V22S, A26K, A26G, A26Q, A26E, A26S, A26T, Q27F, Q27T, T28Y, T28P, T28H, T28R, T28V, R29S, R29H, E35G, E35D, E35A, M42I, M42L, M42G, M42W, M42R, D46E, D46S, D46K, D46V, D46Q, D46N, M47V, M47L, M47R, M47W, E52K, F59S, F59M, F59Y, T62S, T62A, T62E, N63S, N63I, N63H, V68M, V68L, V68N, V68T, V68S, A71G, A71N, A71V, R73D, R73E, R73T, E77G, E81K, L85E, L85Q, Y87R, Y87I, Y87K, Y87Q, Y87N, Y87P, D90G, F92L, T101R, T101K, or T101Q, or a conservative amino acid substitution of any of the foregoing.

The present application in another aspect provides an immunomodulatory protein comprising at least one variant CD80 extracellular domain polypeptide, wherein the variant CD80 extracellular domain polypeptide comprises one or more amino acid substitutions in the sequence set forth in SEQ ID NO:2 or a portion thereof comprising the IgV domain, wherein the one or more amino acid substitutions is selected from E7S, E7K, E7N, E7H, E7Q, K9N, K9R, K9S, E10G, E10S, E10A, V11Y, V11F, V11W, V20I, V22S, Q27F, Q27T, T28P, T28H, T28R, T28V, R29S, E35A, M42L, M42G, M42W, M42R, D46S, D46K, D46Q, M47R, M47W, E52K, F59S, T62S, T62A, N63I, N63H, V68N, V68T, V68S, A71N, A71V, R73D, R73E, R73T, L85Q, Y87R, Y87I, Y87K, Y87P, T101R, T101K, and T101Q, wherein the variant CD80 polypeptide exhibits increased binding to PD-L1 compared to wild-type CD80 comprising the sequence set forth in SEQ ID NO:2 or the portion thereof comprising the IgV domain.

In some of any of the provided embodiments, the variant CD80 extracellular domain polypeptide comprises 2, 3, 4, 5, 6, 7, 8, 9 or 10 amino acid substitutions compared to SEQ ID NO:2 or the portion thereof comprising the IgV domain.

In some of any of the provided embodiments, the immunomodulatory protein comprises no more than 4 amino acid substitutions compared to SEQ ID NO:2 or the portion thereof comprising the IgV domain.

In some of any of the provided embodiments, the immunomodulatory protein comprises 2, 3 or 4 amino acid substitutions compared to SEQ ID NO:2 or the portion thereof comprising the IgV domain.

The present application in another aspect provides an immunomodulatory polypeptide comprising at least one variant CD80 extracellular domain polypeptide, wherein the variant CD80 extracellular domain polypeptide comprises the sequence set forth in SEQ ID NO:2 or a portion thereof comprising an IgV domain, in which is contained amino acid substitutions selected from V11Y/M47V/F59S/L85E, V11Y/M47V/L85E, V11Y/M42I/M47V/A71G, K9N/E10G/N63S/L85E, E10G/E35G/D46E/M47V/V68M, K9N/E10G/E35G/D46E/M47V/V68M, E7S/H18I/V20L/A26K/M47L/A71N, K9R/E10S/V11Y/M42L/F59M/V68M/L85E, V11Y/T28Y/M47L/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, V11Y/T28Y/L85E/Y87I, H18F/M42G/F59Y/V68N, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E35D/V68L/L85E, E10G/V11W/M47V/L85E, T28Y/M47L, V11Y/T28Y/M47L/V68L/L85E, E7N/E35D/T101R, V11Y/M42W/T62A/L85E, V11Y/M42W/T62A, V11Y/M42W/F59Y/V68N, V11Y/M42W/E52K/T62A/L85E, V11Y/E35D/Y87Q/T101R, H18Y/A26E/R29S/E35D/M47L/V68M/A71G/E77G/D90G, E10G/H18Y, K9N/E10R/H18V/T28Y/A71G, E10G/H18Y/T28Y/M47W/T62S, E7H/H18L/V20I/T28Y/D46S/A71G, K9N/E10A/V11W/H18F/T28H/M47L/T62E/R73D, H18V/V20I/T28Y/E35G/M47V/R73E, E10G/D46K/L85E, E10G/H18T/Q27T/D46E/M47L, E35D/V68T/T101K, V11Y/E35G/M42G/F59S, V11Y/T28R/E35G/M47L/F59S, E7N/E35D/F59S, V11Y/T28R/E35G/M47L/A71G, K9R/E10A/E35G/V68T/T101K, V11Y/V68T, E10G/E35G/D46E/M47V/V68M/T101K, K9R/E10A/E35G/V68L/L85E, V11W/T28Y/D46V/R73E/F92L, K9N/E10G/Y87K/T101Q, V11W/T28Y/D46V/V68T/R73T/Y87N, E10S/V11Y/M42R/A71V, H18F/T28V/M47L/V68S, E10G/A26S/T28Y, E35D/D46Q/L85E, E10S/V68M/Y87P, K9N/V11W/M47L/V68T/R73T/Y87N, V11Y/M47L/V68T, K9R/A26T/T28Y/E35A/M47L/A71G, V11W/T28H/D46Q/V68L/L85E, E7Q/V11Y/R29H/M47L/V68T, V11Y/M47V/A71G/L85E, E10G/Q27F/D46N/A71G/D90G, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

In some of any of the provided embodiments, the variant CD80 extracellular domain polypeptide exhibits at least 85% sequence identity to the sequence set forth in SEQ ID No: 2 or the portion thereof comprising the IgV domain.

In some of any of the provided embodiments, the variant CD80 extracellular domain polypeptide exhibits at least 90% sequence identity to the sequence set forth in SEQ ID No: 2 or the portion thereof comprising the IgV domain.

In some of any of the provided embodiments, the variant CD80 extracellular domain polypeptide exhibits at least 95% sequence identity to the sequence set forth in SEQ ID No: 2 or the portion thereof comprising the IgV domain.

In some of any of the provided embodiments, the variant CD80 extracellular domain polypeptide exhibits at least 97% sequence identity to the sequence set forth in SEQ ID No: 2 or the portion thereof comprising the IgV domain.

In some of any of the provided embodiments, the portion of SEQ ID NO:2 comprising the IgV domain comprises amino acids 1-101 of SEQ ID NO:2 and has a length of no more than 110 amino acids.

In some of any of the provided embodiments, the portion of SEQ ID NO:2 comprising the IgV domain is set forth in SEQ ID NO:163.

In some of any of the provided embodiments, the portion of SEQ ID NO:2 comprising the IgV domain is set forth as amino acids 1-107 of SEQ ID NO:2 (SEQ ID NO:164).

In some of any of the provided embodiments, the variant CD80 extracellular domain polypeptide comprises the sequence of amino acids set forth in any of SEQ ID NOS: 165-244 or a sequence of amino acids that exhibits at least 95% sequence identity to any of SEQ ID NOS: 165-244.

In some of any of the provided embodiments, the variant CD80 extracellular domain polypeptide comprises the sequence of amino acids set forth in any of SEQ ID NOS: 165-244.

In some of any of the provided embodiments, the variant CD80 extracellular domain polypeptide is set forth in any one of SEQ ID NOS: 165-244.

In some of any of the provided embodiments, the variant CD80 extracellular domain polypeptide is set forth in SEQ ID NO:180.

In some of any of the provided embodiments, the variant CD80 extracellular domain polypeptide is set forth in SEQ ID NO:185.

In some of any of the provided embodiments, the variant CD80 extracellular domain polypeptide is set forth in SEQ ID NO:197.

In some of any of the provided embodiments, the variant CD80 extracellular domain polypeptide is set forth in SEQ ID NO:233.

In some of any of the provided embodiments, the variant CD80 extracellular domain polypeptide is set forth in SEQ ID NO:234.

In some of any of the provided embodiments, the immunomodulatory protein comprises a heterologous moiety that is linked to the at least one variant CD80 polypeptide, optionally via a linker. In some embodiments, the heterologous moiety is a half-life extending moiety, a multimerization domain, a targeting moiety that binds to a molecule on the surface of a cell, or a detectable label. In some embodiments, the half-life extending moiety comprises a multimerization domain, albumin, an albumin-binding polypeptide, Pro/Ala/Ser (PAS), a C-terminal peptide (CTP) of the beta subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some of any of the provided embodiments, the immunomodulatory protein is a variant CD80-Fc fusion protein comprising the at least one variant polypeptide and an Fc region of an immunoglobulin. In some embodiments, the at least one variant CD80 polypeptide is linked to the Fc region via a linker, optionally a peptide linker. In some embodiments, the linker comprises a peptide linker and the peptide linker is selected from GGGGS (G4S; SEQ ID NO: 328), GSGGGGS (SEQ ID NO: 325), GGGGSGGGGS (2×GGGGS; SEQ ID NO: 329), GGGGSGGGGSGGGGS (3×GGGGS; SEQ ID NO: 330), GGGGSGGGGSGGGGSGGGGS (4×GGGGS, SEQ ID NO:331), GGGGSGGGGSGGGGSGGGGSGGGGS (5×GGGGS, SEQ ID NO: 332), GGGGSSA (SEQ ID NO: 333), or GSGGGGSGGGGS (SEQ ID NO:335) or combinations thereof.

In some of any of the provided embodiments, the immunoglobulin Fc is an IgG1 Fc domain, or is a variant Fc domain that exhibits reduced binding affinity to an Fc receptor and/or reduced effector function, optionally as compared to a wild-type IgG1 Fc domain. In some embodiments, the immunoglobulin Fc is a variant IgG1 Fc domain comprising one or more amino acid substitutions selected from L234A, L234V, L235A, L235E, G237A, S267K, R292C, N297G, and V302C, by EU numbering. In some embodiments, the immunoglobulin Fc region comprises the amino acid substitutions L234A, L235E an G237A by EU numbering, optionally wherein the Fc region is set forth in any of SEQ ID NOS: 344, 345, 348 or 351.

In some of any of the provided embodiments, the immunoglobulin Fc region comprises the sequence set forth in SEQ ID NO:344.

In some of any of the provided embodiments, the immunoglobulin Fc is an IgG4 Fc domain, optionally comprising the amino acid substitution S228P.

In some of any of the provided embodiments, the immunoglobulin Fc region comprises the sequence set forth in SEQ ID NO:326.

In some of any of the provided embodiments, the variant CD80-Fc fusion protein comprises the structure: variant CD80 polypeptide (vCD80)-Linker-Fc region.

In some of any of the provided embodiments, the variant CD80-Fc fusion protein comprises the sequence of amino acids set forth in any one of SEQ ID NOS: 245-324 or a sequence that exhibits at least 85%, at least 90%, at least 95% or at least 97% sequence identity to any one of SEQ ID NOS: 245-324.

In some of any of the provided embodiments, the variant CD80-Fc fusion protein comprises the structure: (vCD80)-Linker-Fc region-Linker-(vCD80).

In some of any of the provided embodiments, the variant CD80-Fc fusion protein comprises the sequence of amino acids set forth in any one of SEQ ID NOS: 336, 338, 339 or 341, or a sequence that exhibits at least 85%, at least 90%, at least 95% or at least 97% sequence identity to any one of SEQ ID NOS: 336, 338, 339 or 341.

In some of any of the provided embodiments, the variant CD80-Fc fusion protein comprises the structure: (vCD80)-Linker-(vCD80)-Linker-Fc region.

In some of any of the provided embodiments, the variant CD80-Fc fusion protein comprises the sequence of amino acids set forth in SEQ ID NO: 340 or 342, or a sequence that exhibits at least 85%, at least 90%, at least 95% or at least 97% sequence identity to SEQ ID NO: 340 or 342.

In some of any of the provided embodiments, the immunomodulatory protein is a homodimer comprising two identical copies of the variant CD80-Fc fusion protein.

In some of any of the provided embodiments, the PD-L1 is human PD-L1.

In some of any of the provided embodiments, the binding affinity of the variant CD80 extracellular domain to PD-L1 is increased greater than 1.1-fold compared to wild-type CD80 set forth in SEQ ID NO:2 or the portion comprising the IgV domain. In some embodiments, the binding affinity is increased greater than 1.5-fold, greater than 2-fold, greater than 3-fold, greater than 4-fold, greater than 5-fold, greater than 6-fold, greater than 7-fold, greater than 8-fold, greater than 9-fold or greater than 10-fold.

In some of any of the provided embodiments, the binding affinity is determined by Mean Fluorescence Intensity (MFI) as measured by flow cytometry in a cell-based binding assay for a PD-L1-expressing cell.

In some of any of the provided embodiments, the immunomodulatory protein blocks binding of PD-L1 to PD-1.

In some of any of the provided embodiments, the variant CD80 extracellular polypeptide exhibit a Koff for binding to PD-L1 of less than 50×10-3 s-1.

In some of any of the provided embodiments, the variant CD80 extracellular polypeptide has a Koff for binding to PD-L1 of at or about or less than 40×10-3 s-1, 30×10-3 s-1, 20×10-3 s-1, 15×10-3 s-1, 10×10-3 s-1, 5×10-3 s-1, or 1×10-3 s-1.

In some of any of the provided embodiments, the variant CD80 extracellular polypeptide has a Koff for binding to PD-L1 of between 1×10-3 s-1 and 50×10-3 s-1, 1×10-3 s-1 and 30×10-3 s-1, 1×10-3 s-1 and 20×10-3 s-1, 1×10-3 s-1 and 15×10-3 s-1, 1×10-3 s-1 and 10×10-3 s-1, 1×10-3 s-1 and 5×10-3 s-1, 5×10-3 s-1 and 50×10-3 s-1, 5×10-3 s-1 and 30×10-3 s-1, 5×10-3 s-1 and 20×10-3 s-1, 5×10-3 s-1 and 15×10-3 s-1, 5×10-3 s-1 and 10×10-3 s-1, 10×10-3 s-1 and 50×10-3 s-1, 10×10-3 s-1 and 30×10-3 s-1, 10×10-3 s-1 and 20×10-3 s-1, 1×10-3 s-1 and 15×10-3 s-1, 15×10-3 s-1 and 50×10-3 s-1, 15×10-3 s-1 and 30×10-3 s-1, 15×10-3 s-1 and 20×10-3 s-1, 20×10-3 s-1 and 50×10-3 s-1, 20×10-3 s-1 and 30×10-3 s-1, or 30×10-3 s-1 and 50×10-3 s-1.

In some of any of the provided embodiments, the variant CD80 polypeptide binds CD28, optionally with a binding affinity is 0.8-fold to 30-fold of the binding affinity of wild-type CD80 set forth in SEQ ID NO:2 or the portion thereof comprising the IgV domain.

In some of any of the provided embodiments, the variant CD80 polypeptide exhibits increased binding to CD28 compared to wild-type CD80 set forth in SEQ ID NO:2 or the portion thereof comprising the IgV domain.

In some of any of the provided embodiments, the binding affinity of the variant CD80 extracellular domain to CD80 is increased greater than 1.1-fold compared to wild-type CD80 set forth in SEQ ID NO:2 or the portion comprising the IgV domain. In some embodiments, the binding affinity is increased greater than 1.5-fold, greater than 2-fold, greater than 3-fold, greater than 4-fold, greater than 5-fold, greater than 6-fold, greater than 7-fold, greater than 8-fold, greater than 9-fold or greater than 10-fold.

In some of any of the provided embodiments, the binding affinity is determined by Mean Fluorescence Intensity (MFI) as measured by flow cytometry in a cell-based binding assay for a CD28-expressing cell.

In some of any of the provided embodiments, the immunomodulatory protein exhibits CD28 agonism, optionally as determined in a T reporter assay. In some embodiments, the CD28 agonism is PD-L1 dependent, optionally as determined in a T cell reporter assay in the presence of PD-L1 expressing cells.

In some of any of the provided embodiments, the immunomodulatory protein blocks binding of CTLA-4 to its ligand CD80 or CD86.

In some of any of the provided embodiments, the immunomodulatory protein is a soluble protein.

In some of any of the provided embodiments, the immunomodulatory protein that is a purified protein.

Also provided herein are cell therapies, such as such as immune cells for example T cell therapies, engineered with any of the provided immunomodulatory proteins as a secretable or transmembrane immunomodulatory protein containing a variant CD80 extracellular domain that exhibits improved PD-L1 binding. The immunomodulatory protein of the provided cell therapies can include any of the above immunomodulatory proteins, including containing any of the above described variant CD80 polypeptides. In some aspects, the immunomodulatory proteins of the immune cells include variant CD80 domains with amino acid substitutions in the IgV domain. In some aspects, cell therapy also is engineered with an antigen receptor such as a recombinant T cell receptor (TCR) or chimeric antigen receptor (CAR). In some aspects, the provided secretable and transmembrane immunomodulatory proteins may improve the potency and efficacy of cells of the cell therapy. Also provided herein are compositions and methods for making and using such cell therapies.

The present application in another aspect provides an immune cell comprising an immunomodulatory protein comprising at least one variant CD80 extracellular domain polypeptide. In some embodiments, the variant CD80 extracellular domain polypeptide comprises an amino acid substitution at position 11 in the sequence set forth in SEQ ID NO:2 or a portion thereof comprising an IgV domain. In some embodiments, the variant CD80 extracellular domain polypeptide exhibits increased binding to PD-L1 compared to wild-type CD80 set forth in SEQ ID NO:2 or the portion thereof comprising the IgV domain.

The present application in another aspect provides an immune cell comprising an immunomodulatory protein comprising at least one variant CD80 extracellular domain polypeptide. In some embodiments, the variant CD80 extracellular domain polypeptide comprises 2 to 10 amino acid substitutions at positions in the sequence set forth in SEQ ID NO:2 or a portion thereof comprising an IgV domain. In some embodiments at least one substitution is at a position selected from among 9, 10, or 11. In some embodiments the variant CD80 extracellular domain polypeptide exhibits increased binding to PD-L1 compared to wild-type CD80 set forth in SEQ ID NO:2 or the portion thereof comprising the IgV domain. In some embodiments, the variant CD80 extracellular domain polypeptide comprises an amino acid substitution at position 11 and the substitution is to an aromatic amino acid residue. In some embodiments, the aromatic amino acid residues are selected from the group consisting of tyrosine (Y), tryptophan (W) or phenylalanine (F), optionally wherein the amino acid substitution is V11Y, V11F, or V11W.

The present application in another aspect provides an immune cell comprising an immunomodulatory protein comprising at least one variant CD80 extracellular domain polypeptide. In some aspects, the variant CD80 extracellular domain polypeptide comprises an amino acid substitution at position 7 in the sequence set forth in SEQ ID NO:163 or a portion thereof comprising the IgV domain. In some aspects the variant CD80 extracellular domain polypeptide exhibits increased binding to PD-L1 compared to the wild-type CD80 extracellular domain polypeptide set forth in SEQ ID NO:2 or the portion thereof comprising the IgV domain.

In some of any embodiments, the immune cell comprises any of the immunomodulatory proteins provided herein. In some of any of the provided embodiments, the immune cell further comprises a T cell receptor (TCR) or a chimeric antigen receptor (CAR).

In some of any of the provided embodiments, the immune cell is a lymphocyte. In some embodiments, the lymphocyte is a T cell.

In some of any of the provided embodiments, the immunomodulatory protein is a transmembrane protein expressed on the surface of the immune cell. In some of any of the provided embodiments, the immunomodulatory protein is secretable from the immune cell.

The present application in another aspect provides a nucleic acid molecule(s) encoding the immunomodulatory protein described in any of the provided embodiments herein. In some embodiments, the nucleic acid molecule is a synthetic nucleic acid. In some embodiments, the nucleic acid molecule provided herein is a cDNA.

The present application in another aspect provides a vector, comprising the nucleic acid molecule described in any of the provided embodiments herein. In some embodiments, the vector is an expression vector. In some embodiments, the vector is a mammalian expression vector or a viral vector.

The present application in another aspect provides a method of producing an immunomodulatory protein comprising introducing the nucleic acid molecule described in any of the embodiments herein into a host cell under conditions to express the protein in the cell, and isolating or purifying the protein from the cell.

In some of any of the provided embodiments, provided herein is a purified immunomodulatory protein produced by the method described herein.

In some of any of the provided embodiments, provided herein is a pharmaceutical composition comprising the immunomodulatory protein described herein. In some embodiments, the method comprises a pharmaceutically acceptable excipient. In some embodiments, the pharmaceutical composition is sterile.

Also provided herein is a pharmaceutical composition comprising the immune cell disclosed herein. In some embodiments, the pharmaceutical composition comprises a pharmaceutically acceptable excipient. In some embodiments, the pharmaceutical composition is sterile.

In some of any of the provided embodiments, provided herein is an article of manufacture comprising the pharmaceutical composition disclosed herein in a vial or container. In some embodiments, the vial or container is sealed.

In some of any of the provided embodiments, provided herein is a kit comprising the pharmaceutical composition disclosed herein, and instructions for use.

The present application in another aspect provides a method of stimulating an immune response in a subject, comprising administering an immunomodulatory protein disclosed herein or the pharmaceutical composition disclosed herein to a subject in need thereof. In some embodiments, stimulating the immune response treats a disease or condition in the subject.

Also provided herein is a method of stimulating an immune response in a subject, comprising administering the immune cell disclosed herein or the pharmaceutical composition disclosed herein to a subject in need thereof. In some embodiments, stimulating the immune response treats a disease or condition in the subject.

The present application in another aspect provides a method of treating a disease or condition in a subject, the method comprising administering the immunomodulatory protein disclosed herein or the pharmaceutical composition disclosed herein to a subject having the disease or condition. In some embodiments, the disease or condition is a cancer.

Also provided herein is a method of treating a disease or condition in a subject, the method comprising administering the immune cell of any of claims disclosed herein or the pharmaceutical composition disclosed herein to a subject having the disease or condition. In some embodiments, the disease or condition is a cancer.

In some of any of the provided embodiments, the subject has a PD-L1-expressing tumor.

In some of any of the provided embodiments, prior to the administering, the method comprises selecting a subject having an PD-L1-expressing tumor.

### BRIEF DESCRIPTION OF THE DRAWINGS

FIG. 1A depicts a CD80 vIgD/PD-L1 ECD structure and identifies that certain amino acids, such as amino acids 7-11, are residues involved in the interaction between CD80 and PD-L1 within 4 Å of each other.

FIG. 1B depicts modeling of a wildtype (WT) CD80 vIgD/PD-L1 structure compared to a variant CD80 vIgD/PD-L1 structure, in which the variant CD80 contains an amino acid substitution at position 11 to an aromatic residue.

FIG. 2A depicts a surface plasmon resonance (SPR) sensorgram of wildtype CD80 ECD-Fc binding to PD-L1.

FIG. 2B depicts SPR sensorgrams of certain variant CD80 IgV-Fc binding to PD-L1. Compared to FIG. 2A, the results demonstrate increased binding to PD-L1 compared to wild-type CD80-Fc and with a relatively slow PD-L1 off-rate.

FIG. 3A-3D depict binding activity as measured by the median fluorescent intensity of single cells across a range of PD-L1 receptor densities. FIG. 3A depicts binding of exemplary variant CD80 IgV-Fc molecules, including dimers and tandem formats, to K562/OKT3/PDL1. FIG. 3B depicts binding of exemplary variant CD80 IgV-Fc, including dimers and tandem formats, to HCC827. FIG. 3C depicts binding of exemplary variant CD80 IgV-Fc, including dimers and tandem formats, to SCC152/PDL1. FIG. 3D depicts binding of exemplary variant CD80 IgV-Fc, including dimers and tandem formats, to A704 cells.

FIG. 4A-4C depict PD-1/PD-L1 blockade by exemplary variant CD80 IgV-Fc molecules, including dimers and tandem formats, as determined by relative light units (RLU) using Jurkat/PD-1/SHP2 reporter cells in the presence of PD-L1-expressing target cells that also were engineered to express membrane-bound anti-CD3 (OKT3) single-chain Fc. FIG. 4A depicts blockade activity in the presence of K562/OKT3/PD-L1 target cells. FIG. 4B depicts blockade activity in the presence of HCC827/OKT3 targets cells. FIG. 4C depicts blockade activity in the presence of SCC152/PD-L1/OKT3.

FIG. 5A-5D depict PD-L1-dependent CD28 costimulation as measured by IL-2 secretion by primary human T cells when incubated with PD-L1-expressing target cells (K562, HCC827, SCC152 and A704 cells) in the presence of exemplary variant CD80 IgV-Fc molecules, including dimers and tandem formats. FIG. 5A depicts IL-2 secretion in co-cultures of T cells and K562-PD-L1^{hi} cells. FIG. 5B depicts IL-2 secretion in co-cultures of T cells and HCC827-PD-L1^{hi} cells. FIG. 5C depicts IL-2 secretion in co-cultures of T cells and SCC152-PD-L1^{hi} cells. FIG. 5D depicts IL-2 secretion in co-cultures of T cells and A704-PD-L1^{low} cells.

FIG. 6 depicts mean tumor volume over time in the low hPD-L1 MC38 mouse model following treatment with exemplary variant CD80 IgV-Fc molecules, including dimers and tandem formats.

FIG. 7 depicts mean tumor volume over time in the low hPD-L1 MC38 mouse model following treatment with different doses of the exemplary variant CD80 IgV-Fc dimers a CD80_234-Fc.

### DETAILED DESCRIPTION

Provided herein are immunomodulatory proteins that contain variants (also called mutants) of at least one CD80 extracellular domain (CD80 vIgDs) that exhibit improved binding to PD-L1. In some embodiments, the binding affinity to PD-L1 is increased compared to a wild-type or unmodified CD80. In some embodiments, the variants exhibit a slow PD-L1 off-rate for binding to PD-L1 and, in some aspects, improved pharmacokinetics. In some embodiments, the immunomodulatory proteins are purified proteins. In some embodiments, the immunomodulatory proteins may be expressed in cells, such as T cells, as secretable immunomodulatory proteins or transmembrane immunomodulatory proteins.

Also provided herein are cell therapies (e.g., T cell therapies) engineered with a secretable or transmembrane immunomodulatory protein of any of the provided immunomodulatory proteins that contain variants of at least one CD80 extracellular domain that exhibit improved binding to PD-L1 as provided herein. In some embodiments, the immunomodulatory proteins provided herein can be engineered into an immune cell, such as a T cell, to improve response and activation of the immune cell, such as by immune modulation via provided a costimulatory signal and/or blocking of an inhibitory signal.

The provided variant extracellular domain of CD80 may be composed of a binding portion containing the IgV domain in which is contained one or more amino acid substitutions, compared to an unmodified or wild-type CD80 polypeptide. The provided immunomodulatory proteins may be fusion proteins of a variant CD80 extracellular domain or binding portion thereof containing the IgV domain and a multimerization domain, such as an immunoglobulin Fc. For example, provided herein are variant CD80-Fc fusion proteins. Among the provided immunomodulatory proteins are proteins that bind PD-L1 and CD28 to both antagonize PD-1/PD-L1 interactions and to provide PD-L1 dependent costimulation of CD28. In some cases, the provided immunomodulatory proteins may also antagonize or block interactions of CTLA-4 with its ligands CD80/CD86. In some embodiments, the immunomodulatory proteins provided herein can be used for the treatment of diseases, disorders or conditions that are associated with a dysregulated immune response, such as associated with cancer.

In some embodiments, the variant CD80 polypeptides exhibit increased binding affinity to PD-L1 compared to the unmodified or wild-type CD80 not containing one or more substitutions. In some embodiments, the variant CD80 polypeptides retain binding affinity to CD28 compared to the unmodified or wild-type CD80 not containing one or more substitutions. In some embodiments, the variant CD80 polypeptides exhibit increased binding affinity to CD28 compared to the unmodified or wild-type CD80 not containing the one or more substitutions.

In some embodiments, the variant CD80 polypeptides and immunomodulatory proteins modulate an immunological immune response, such as increase an immune response. In some embodiments, the variant CD80 polypeptides and immunomodulatory proteins provided herein can be used for the treatment of diseases or conditions that are associated with a dysregulated immune response.

In some embodiments, the provided variant CD80 polypeptides modulate T cell activation, expansion, differentiation, and survival via interactions with costimulatory signaling molecules. In general, antigen specific T-cell activation generally requires two distinct signals. The first signal is provided by the interaction of the T-cell receptor (TCR) with major histocompatibility complex (MHC) associated antigens present on antigen presenting cells (APCs). The second signal is costimulatory, e.g., a CD28 costimulatory signal, to TCR engagement and necessary to avoid T-cell apoptosis or anergy.

In some embodiments, under normal physiological conditions, the T cell-mediated immune response is initiated by antigen recognition by the T cell receptor (TCR) and is regulated by a balance of co-stimulatory and inhibitory signals (e.g., immune checkpoint proteins). The immune system relies on immune checkpoints to prevent autoimmunity (i.e., self-tolerance) and to protect tissues from excessive damage during an immune response, for example during an attack against a pathogenic infection. In some cases, however, these immunomodulatory proteins can be dysregulated in diseases and conditions, including tumors, as a mechanism for evading the immune system.

In some embodiments, among known T-cell costimulatory receptors is CD28, which is the T-cell costimulatory receptor for the ligands B7-1 (CD80) and B7-2 (CD86) both of which are present on APCs. These same ligands can also bind to the inhibitory T-cell receptor CTLA-4 (cytotoxic T-lymphocyte-associated protein 4) with greater affinity than for CD28; the binding to CTLA-4 acts to down-modulate the immune response.

CD80, which is expressed by the majority of antigen presenting cells, binds not only CD28, but also CTLA-4 and PD-L1, thereby playing a central role in T cell regulation. Specifically, although CD80 has a well-documented role modulating T cell responses via the CTLA-4/CD28 coreceptors, it recently has been demonstrated to bind PD-L1 in cis on the surface of antigen presenting cells (APC) (Butte M. J., et al. Immunity 2007; 27(1): 111-22; Chaudhri A., et al. Cancer Immunol Res 2018; 6(8): 921-929). PD-L1 is one of two ligands for the inhibitory immune receptor, programmed death 1 (PD-1). The interaction of PD-L1 with PD-1 negatively regulates immune activity by promoting T cell inactivation and down-modulating T cell activity. PD-1 expression on T cells may be induced after T cells have been activated as a strategy to prevent over-activity of T cells. Many tumor cells express PD-L1 on their surface, potentially leading to PD-1/PD-L1 interactions and the inhibition of T cell responses against the tumor. The binding of CD80 to PD-L1 can block the interaction between PD-L1 and PD-1, and thereby prevent inhibition of T cell responses, e.g., at the site of a tumor, and effectively potentiate or enhance the immune response. At the same time, however, CD80 might also be available to bind to CD28 or CTLA4 receptors, and be involved in inducing or inhibiting T cell responses. Thus, in some cases, interactions of CD80 with PD-L1, CD28, and CTLA-4 can yield overlapping and complementary effects. In some embodiments, CD28 and PD-L1 may play complementary roles in modeling an immune response. Recent data suggest that CD80, when bound to PD-L1 in cis, appears to retain the ability to costimulate CD28 while blocking PD-L1 engagement with PD-1 (Zhao Y., et al., Cell Rep 2018; 24(2): 379-390; Zhao Y., et al., Immunity 2019; 51(6): 1059-1073), suggesting that the relative expression levels of each of these receptors likely contributes to the overall costimulatory/coinhibitory activity of an APC (Sugiura D. M., et al., Science 2019; 364(6440): 558-566).

Immune checkpoint inhibitors (CPI) targeting the CTLA-4/CD80/CD86 and PD-1/PD-L1 pathways have demonstrated significant clinical activity in many cancers, either as monotherapy or in combination with current standard of care regimens (Ribas A., et al., Science 2018; 359(6382): 1350-1355). Yet, despite recent advancements in immuno-oncology achieved through T cell checkpoint blockade targeting these pathways, the majority of treated patients either fail to achieve objective responses or develop resistance to therapy and experience disease progression. For instance, although impressive clinical outcomes are achieved for some cancers, many patients fail to respond to CPI entirely, or the observed response lacks durability due to the development of acquired resistance (Jenkins R. W., et al., Br J Cancer 2018; 118(1): 9-16). Primary and acquired resistance to immunotherapy is the subject of intensive research, with several proposed tumor-intrinsic and extrinsic mechanisms implicated, including increased expression of metabolic mediators, impaired antigen presentation and T cell activation, recruitment of immunosuppressive cells, modulation of immune checkpoints, and impaired IFNγ signaling, among others (Schoenfeld A. J., et al., Cancer Cell 2020; 37(4): 443-455). To overcome these obstacles, rationally designed combinations of drugs targeting multiple inhibitory and costimulatory pathways may be required. In support of this concept, preclinical data demonstrating the distinct and synergistic mechanisms of CTLA-4 and PD-1 blockade (Wei S. C., Cell 2017; 170(6): 1120-1133; Intlekofer A. M., et al., J Leukoc Biol 2013; 94(1): 25-39) have translated into improved clinical activity and patient outcomes in randomized trials in melanoma and renal cell carcinoma (Wolchok J. D., et al., N Engl J Med 2017; 377(14): 1345-1356; Motzer R. J., et al., N Engl J Med 2018; 378(14): 1277-1290).

More recently, several groups have demonstrated that CTLA-4 and PD-1 inhibition works either directly or indirectly through suppression of CD28 costimulatory signaling (Rowshanravan B., et al., Blood 2018; 131(1):58-67; Hui E., et al., Science 2017; 355(6332): 1428-1433; Kamphorst A. O., et al., Science 2017; 355(6332): 1423-1427) and that lack of sufficient T cell costimulation in the tumor microenvironment may be involved in primary or acquired resistance to CPI (Scarpa M., et al., BMC Cancer 2016; 16: 388; Jiang P., et al., Nat Med 2018; 24(10): 1550-1558; Pinto M. L., et al., Front Immunol 2019; 10: 1875; Feng X.-Y., et al., Future Oncology 2019; 15(5): 473-483; Beckerman K. E., et. al., JCI Insight 2020; 5(16)). Patients responsive to checkpoint blockade generate and maintain a productive adaptive anti-tumor immune response via increased CD28 costimulatory signaling leading to improved T cell priming and effector functions as well as reversal of T cell exhaustion. This raises the possibility that an approach combining CPI with CD28 costimulation should be more potent than CPI alone. However, there is a need for improved therapies.

The provided embodiments are based on the recognition that PD-L1 binding improvements can be engineered into the CD80 extracellular domain. By increasing PD-L1 binding, it was possible to generate a CD80 vIgD:PD-L1 complex revealing residues involved in the interaction with PD-L1. Targeting these residues and others for mutagenesis using a rational design strategy further identified individual positions and mutations to selectively engineer a CD80 IgV domain for increased affinity for PD-L1 relative to wild-type CD80. Moreover, as exemplified in examples herein, a selection strategy also was employed to identify variants with a relatively slow off-rate for PD-L1 binding of less than less than 50×10⁻³ s⁻¹, such as less than less than 20×10⁻³ s⁻¹. The improvements in binding to PD-L1 could be achieved while maintaining the ability to bind both CD28 and CTLA-4. In some cases, binding to CD28 also was increased.

In some embodiments, the provided immunomodulatory proteins can mediate CD28 agonism. For example, when fused to an exemplary Fc domain (e.g. Fc domain with weak or no effector function), the immunomodulatory proteins containing a provided variant extracellular domain also demonstrated the ability to induce PD-L1-dependent CD28 costimulation. In some cases, CD28 agonism is mediated by certain variant CD80 polypeptides exhibiting increased binding to PD-L1 to thereby facilitate tethering or crosslinking of the variant CD80 molecule to a surface at the immune synapse for interaction with CD28, thereby facilitating T cell activation by providing a costimulatory signal. This activity, designated herein as PD-L1-dependent CD28 costimulation, is due, in some aspects, to the ability of a variant CD80 polypeptide to bind both PD-L1 and CD28 in a non-competitive manner and/or by provision of a multivalent (e.g. dimeric or tetravalent) format of a variant CD80 polypeptide.

Thus, in some aspects, the provided molecules exhibit, in a single active domain, both checkpoint antagonistic and costimulatory activity to provide an immunomodulatory protein capable of antagonizing CTLA-4 and/or PD-1 inhibitory receptors and delivering a PD-L1-dependent T cell costimulatory signal via CD28. In some embodiments, the provided immunomodulatory proteins can be used as a therapy, such as a therapy as a purified protein (e.g. Fc-fusion protein) or as a cell therapy, in oncology indications. Enhancement of immunological activity has clinical significance for treatment of certain disease indications or conditions, such as cancer and viral infections. In some cases, however, existing therapies to intervene and alter the costimulatory effects of both receptors are constrained by the spatial orientation requirements as well as size limitations imposed by the confines of the immunological synapse. In some aspects, existing therapeutic drugs, including antibody drugs, may not be able to interact simultaneously with the multiple target proteins involved in modulating these interactions. In addition, in some cases, existing therapeutic drugs may only have the ability to antagonize, but not agonize, an immune response. Additionally, pharmacokinetic differences between drugs that independently target one or the other of these two receptors can create difficulties in properly maintaining a desired blood concentration of such drug combinations throughout the course of treatment. The provided variant CD80 polypeptides and immunomodulatory proteins address such problems.

In some embodiments, the provided immunomodulatory proteins are able to armor cell therapies by providing to them a secretable or transmembrane bound molecule to provide for independent activation of the CD28 costimulatory receptor while also, in some cases, antagonizing inhibitory signals. In some embodiments, the secretable or transmembrane immunomodulatory proteins provided herein can promote immune cell (e.g., T cell) proliferation, cytotoxicity and cytokine production and thereby improve antigen-specific responses of the cell therapy. In some embodiments, existing cell therapies are deficient in one or more of these respects.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in the specification and the appended claims, the singular forms “a,” “an,” and “the” include plural referents unless the context clearly indicates otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

The term “affinity-modified” as used in the context of a domain of a protein means a mammalian protein having an altered amino acid sequence in an extracellular domain or a specific binding portion thereof (relative to the corresponding wild-type parental or unmodified domain) such that it has an increased or decreased binding activity, such as binding affinity, to at least one of its binding partners (alternatively “counter-structures”) compared to the parental wild-type or unmodified (i.e., non-affinity modified domain) protein. The domain may be an immunoglobulin superfamily domain (IgSF domain), such as an IgV domain. Included in this context is an affinity modified CD80 IgSF domain, such as an affinity modified CD80 IgV domain. In some embodiments, the affinity-modified domain can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions, in a wild-type or unmodified domain. An increase or decrease in binding activity, e.g. binding affinity, can be determined using well known binding assays, including flow cytometry. Larsen et al., American Journal of Transplantation, Vol 5: 443-453 (2005). See also, Linsley et al., Immunity, 1: 7930801 (1994). An increase in a protein's binding activity, e.g. affinity, to its binding partner(s) is to a value at least 10% greater than that of the wild-type control and in some embodiments, at least 20%, 30%, 40%, 50%, 100%, 200%, 300%, 500%, 1000%, 5000%, or 10000% greater than that of the wild-type control value. A decrease in a protein's binding activity, e.g. affinity, to at least one of its binding partners is to a value no greater than 90% of the control but no less than 10% of the wild-type control value, and in some embodiments no greater than 80%, 70% 60%, 50%, 40%, 30%, or 20% but no less than 10% of the wild-type control value. An affinity-modified protein is altered in primary amino acid sequence of the extracellular domain or a specific binding portion thereof by substitution, addition, or deletion of amino acid residues. The term “affinity-modified” is not to be construed as imposing any condition for any particular starting composition or method by which the affinity-modified protein was created. Thus, an affinity-modified protein is not limited to wild-type protein domains that are then transformed to an affinity-modified domain by any particular process of affinity modification. An affinity-modified domain polypeptide can, for example, be generated starting from wild-type mammalian domain sequence information, then modeled in silico for binding to its binding partner, and finally recombinantly or chemically synthesized to yield the affinity-modified domain composition of matter. In but one alternative example, an affinity-modified domain can be created by site-directed mutagenesis of a wild-type domain. Thus, an affinity modified domain denotes a product and not necessarily a product produced by any given process. A variety of techniques including recombinant methods, chemical synthesis, or combinations thereof, may be employed.

The term “allogeneic” as used herein means a cell or tissue that is removed from one organism and then infused or adoptively transferred into a genetically dissimilar organism of the same species. In some embodiments of the invention, the species is murine or human.

The term “autologous” as used herein means a cell or tissue that is removed from the same organism to which it is later infused or adoptively transferred. An autologous cell or tissue can be altered by, for example, recombinant DNA methodologies, such that it is no longer genetically identical to the native cell or native tissue which is removed from the organism. For example, a native autologous T-cell can be genetically engineered by recombinant DNA techniques to become an autologous engineered cell expressing a transmembrane immunomodulatory protein and/or chimeric antigen receptor (CAR), which in some cases involves engineering a T-cell or TIL (tumor infiltrating lymphocyte). The engineered cells are then infused into a patient from whom the native T-cell was isolated. In some embodiments, the organism is human or murine.

As used herein, “bind,” “bound” or grammatical variations thereof refers to the participation of a molecule in any attractive interaction with another molecule, resulting in a stable association in which the two molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs.

As used herein, binding activity refer to characteristics of a molecule, e.g. a polypeptide, relating to whether or not, and how, it binds one or more binding partners. A binding activity can include any measure of binding of one molecule for a binding partner. Binding activities include the ability to bind the binding partner(s), the affinity with which it binds to the binding partner (e.g. high affinity), the avidity with which it binds to the binding partner, the strength of the bond with the binding partner and/or specificity or selectivity for binding with the binding partner.

The term “binding affinity” as used herein means the specific binding affinity of a protein for its binding partner (i.e., its counter-structure) under specific binding conditions. The binding affinity refers to the strength of the interaction between two or more molecules, such as binding partners, typically the strength of the noncovalent interactions between two binding partners. An increase or attenuation in binding affinity of an immunomodulatory protein containing an affinity-modified domain (e.g. variant CD80 IgV domain) to a binding partner is determined relative to the binding affinity of a protein containing the unmodified domain (e.g. wild-type CD80 extracellular domain or specific portion containing the IgV domain). Methods for determining binding affinity, or relative binding affinity, are known in art, solid-phase ELISA immunoassays, ForteBio Octet, Biacore measurements or flow cytometry. See, for example, Larsen et al., American Journal of Transplantation, vol. 5: 443-453 (2005); Linsley et al., Immunity, Vol 1 (9): 793-801 (1994). In some embodiments, binding affinity can be measured by flow cytometry, such as based on a Mean Fluorescence Intensity (MFI) in a flow binding assay. In some embodiments, a variant CD80, such as containing an affinity modified IgSF domain (e.g. variant IgV domain), specifically binds to PD-L1 measured by flow cytometry with a binding affinity that yields a Mean Fluorescence Intensity (MFI) value at least 1.2-fold, 1.5-fold, 2.0-fold, 3.0-fold, 4.0-fold, 5.0-fold, 6.0-fold, 7.0-fold, 8.0-fold, 9.0-fole or 10.0-fold greater than an unmodified CD80 control in a binding assay such as described in Example 3.

The term “binding avidity” as used herein means the specific binding avidity, of a protein for its binding partner (i.e., its counter-structure) under specific binding conditions. In biochemical kinetics avidity refers to the accumulated strength of multiple affinities of individual non-covalent binding interactions, such as between a protein for its binding partner (i.e., its counter-structure). As such, avidity is distinct from affinity, which describes the strength of a single interaction.

The term “biological half-life” refers to the amount of time it takes for a substance, such as an immunomodulatory protein containing a variant CD80 polypeptide of the present invention, to lose half of its pharmacologic or physiologic activity or concentration. Biological half-life can be affected by elimination, excretion, degradation (e.g., enzymatic) of the substance, or absorption and concentration in certain organs or tissues of the body. In some embodiments, biological half-life can be assessed by determining the time it takes for the blood plasma concentration of the substance to reach half its steady state level (“plasma half-life”). Conjugates that can be used to derivatize and increase the biological half-life of polypeptides of the invention are known in the art and include, but are not limited to, immunoglobulin Fc, polyethylene glycol (PEG), hydroxyethyl starch (HES), XTEN (extended recombinant peptides; see, WO2013130683), human serum albumin (HSA), bovine serum albumin (BSA), lipids (acylation), and poly-Pro-Ala-Ser (PAS), polyglutamic acid (glutamylation).

The term “binding partner” or “cognate binding partner” (also used interchangeably with “counter-structure”) in reference to a polypeptide, such as in reference to an IgSF domain of a variant CD80, refers to at least one molecule (typically a native mammalian protein) to which the referenced polypeptide specifically binds under specific binding conditions. In some aspects, a variant CD80 containing an affinity modified IgSF domain specifically binds to at least one counter-structure of the corresponding native or wildtype CD80 (e.g. PD-L1, CD28 or CTLA-4) but with increased or attenuated affinity. A “cell surface binding partner” is a cognate binding partner expressed on a mammalian cell surface. Typically, the cell surface binding partner is a transmembrane protein. In some embodiments, the cell surface binding partner is a receptor, or a ligand of a receptor expressed on and by cells, such as mammalian cells, forming the immunological synapse, for example immune cells.

The term “chimeric antigen receptor” or “CAR” as used herein refers to an artificial (i.e., man-made) transmembrane protein expressed on a mammalian cell containing at least an ectodomain, a transmembrane, and an endodomain. Optionally, the CAR protein includes a “spacer” which covalently links the ectodomain to the transmembrane domain. A spacer is often a polypeptide linking the ectodomain to the transmembrane domain via peptide bonds. The CAR is typically expressed on a mammalian lymphocyte. In some embodiments, the CAR is expressed on a mammalian cell such as a T-cell or a tumor infiltrating lymphocyte (TIL). A CAR expressed on a T-cell is referred to herein as a “CAR T-cell” or “CAR-T.” In some embodiments the CAR-T is a T helper cell, a cytotoxic T-cell, a natural killer T-cell, a memory T-cell, a regulatory T-cell, or a gamma delta T-cell. When used clinically in, e.g., adoptive cell transfer, a CAR-T with antigen binding specificity to the patient's tumor is typically engineered to express on a native T-cell obtained from the patient. The engineered T-cell expressing the CAR is then infused back into the patient. The CAR-T is thus often an autologous CAR-T although allogeneic CAR-Ts are included within the scope of the invention. The ectodomain of a CAR contains an antigen binding region, such as an antibody or antigen binding fragment thereof (e.g., scFv), that specifically binds under physiological conditions with a target antigen, such as a tumor specific antigen Upon specific binding a biochemical chain of events (i.e., signal transduction) results in modulation of the immunological activity of the CAR-T. Thus, for example, upon specific binding by the antigen binding region of the CAR-T to its target antigen can lead to changes in the immunological activity of the T-cell activity as reflected by changes in cytotoxicity, proliferation or cytokine production. Signal transduction upon CAR-T activation is achieved in some embodiments by the CD3-zeta chain (“CD3-z”) which is involved in signal transduction in native mammalian T-cells. CAR-Ts can further contain multiple signaling domains such as CD28, 41BB or OX40, to further modulate immunomodulatory response of the T-cell. CD3-z contains a conserved motif known as an immunoreceptor tyrosine-based activation motif (ITAM) which is involved in T-cell receptor signal transduction.

As used herein, “conjugate,” “conjugation” or grammatical variations thereof refers the joining or linking together of two or more compounds resulting in the formation of another compound, by any joining or linking methods known in the art. It can also refer to a compound which is generated by the joining or linking together two or more compounds. For example, a variant CD80 polypeptide linked directly or indirectly to one or more chemical moieties or polypeptide is an exemplary conjugate. Such conjugates include fusion proteins, those produced by chemical conjugates and those produced by any other methods.

The term “competitive binding” as used herein means that a protein is capable of specifically binding to at least two cognate binding partners but that specific binding of one cognate binding partner inhibits, such as prevents or precludes, simultaneous binding of the second cognate binding partner. Thus, in some cases, it is not possible for a protein to bind the two cognate binding partners at the same time. Generally, competitive binders contain the same or overlapping binding site for specific binding but this is not a requirement. In some embodiments, competitive binding causes a measurable inhibition (partial or complete) of specific binding of a protein to one of its cognate binding partners due to specific binding of a second cognate binding partner. A variety of methods are known to quantify competitive binding such as ELISA (enzyme linked immunosorbent assay) assays.

The term “conservative amino acid substitution” as used herein means an amino acid substitution in which an amino acid residue is substituted by another amino acid residue having a side chain R group with similar chemical properties (e.g., charge or hydrophobicity). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

The term, “corresponding to” with reference to positions of a protein, such as recitation that nucleotides or amino acid positions “correspond to” nucleotides or amino acid positions in a disclosed sequence, such as set forth in the Sequence Listing, refers to nucleotides or amino acid positions identified upon alignment with the disclosed sequence based on structural sequence alignment or using a standard alignment algorithm, such as the GAP algorithm. For example, corresponding residues can be determined by alignment of a reference sequence with the sequence of wild-type CD80 set forth in SEQ ID NO: 2 (ECD domain) or set forth in SEQ ID NO: 163 or 164 (IgV domain) by structural alignment methods as described herein. By aligning the sequences, one skilled in the art can identify corresponding residues, for example, using conserved and identical amino acid residues as guides.

As used herein, domain (typically a sequence of three or more, generally 5 or 7 or more amino acids, such as 10 to 200 amino acid residues) refers to a portion of a molecule, such as a protein or encoding nucleic acid, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as binding activity. A protein can have one, or more than one, distinct domains. For example, a domain can be identified, defined or distinguished by homology of the primary sequence or structure to related family members, such as homology to motifs. In another example, a domain can be distinguished by its function, such as an ability to interact with a biomolecule, such as a cognate binding partner. A domain independently can exhibit a biological function or activity such that the domain independently or fused to another molecule can perform an activity, such as, for example binding. A domain can be a linear sequence of amino acids or a non-linear sequence of amino acids. Many polypeptides contain a plurality of domains. Such domains are known, and can be identified by those of skill in the art. For exemplification herein, definitions are provided, but it is understood that it is well within the skill in the art to recognize particular domains by name. If needed appropriate software can be employed to identify domains.

The term “ectodomain,” “extracellular domain,” or “ECD,” which are used interchangeably herein, refers to a region of a membrane protein, such as a transmembrane protein, that lies outside the vesicular membrane (e.g., the space outside of a cell), when a full-length form of the membrane protein is expressed from a cell. For purposes herein, it is understood that reference to the ECD refers to sequences and domains that make up this region and do not require that a protein that contains an ECD is a membrane protein or that the domain is present outside a cell. For example, a soluble immunomodulatory protein can contain ECD sequences of a membrane protein fused to another moiety, such as a multimerization domain, for example an Fc region. Ectodomains often interact with specific ligands or specific cell surface receptors, such as via a binding domain that specifically binds to the ligand or cell surface receptor. Ectodomains of members of the IgSF superfamily contain an IgSF domain such as typically an IgV domain and, in some cases, and IgC domain. Thus, reference to an ECD herein includes a full-length sequence of an ECD of a membrane protein as well as specific-binding fragments thereof containing an IgV domain that bind to a ligand or cognate binding partner.

The term “endodomain” as used herein refers to the region found in some membrane proteins, such as transmembrane proteins, that extend into the interior space defined by the cell surface membrane. In mammalian cells, the endodomain is the cytoplasmic region of the membrane protein. In cells, the endodomain interacts with intracellular constituents and can be play a role in signal transduction and thus, in some cases, can be an intracellular signaling domain. The endodomain of a cellular transmembrane protein is alternately referred to as a cytoplasmic domain, which, in some cases, can be a cytoplasmic signaling domain.

The terms “effective amount” or “therapeutically effective amount” refer to a quantity and/or concentration of a therapeutic composition, such as containing an immunomodulatory protein or Fc fusion protein, that when administered ex vivo (by contact with a cell from a patient) or in vivo (by administration into a patient) either alone (i.e., as a monotherapy) or in combination with additional therapeutic agents, yields a statistically significant decrease in disease progression as, for example, by ameliorating or eliminating symptoms and/or the cause of the disease. An effective amount for treating a disease, condition or disorder, such as a cancer, may be an amount that relieves, lessens, or alleviates at least one symptom or biological response or effect associated with a disease or disorder, prevents progression of the disease or disorder, or improves physical functioning of the patient. In some embodiments, “effective amount” or “therapeutically effective amount” refer to a quantity and/or concentration of a cell therapy composition.

As used herein, a fusion protein refers to a polypeptide encoded by a nucleic acid sequence containing a coding sequence for two or more proteins, in some cases 2, 3, 4, 5 or more protein, in which the coding sequences are in the same reading frame such that when the fusion construct is transcribed and translated in a host cell, the protein is produced containing the two or more proteins. Each of the two or more proteins can be adjacent to another protein in the construct or separated by a linker polypeptide that contains, 1, 2, 3, or more, but typically fewer than 20, 15, 10, 9, 8, 7, or 6 amino acids. The protein product encoded by a fusion construct is referred to as a fusion polypeptide. An example of a fusion protein in accord with the provided embodiments is an Fc fusion protein containing an affinity-modified domain (e.g. a variant of a CD80 extracellular domain or portion thereof containing an IgV domain) that is linked to an immunoglobulin Fc domain.

The term “engineered cell” as used herein refers to a mammalian cell that has been genetically modified by human intervention such as by recombinant DNA methods or viral transduction. In some embodiments, the cell is an immune cell, such as a lymphocyte (e.g., T cell, B cell, NK cell) or an antigen presenting cell (e.g., dendritic cell). The cell can be a primary cell from a patient or can be a cell line. In some embodiments, an engineered cell of the invention contains a variant CD80 of the invention engineered to modulate immunological activity of a T-cell expressing CD28, PD-L1 and/or CTLA-4, or an APC expressing PD-L1, to which the variant CD80 polypeptide specifically binds. In some embodiments, the variant CD80 is a transmembrane immunomodulatory protein (hereinafter referred to as “TIP”) containing the extracellular domain or a portion thereof containing the IgV domain linked to a transmembrane domain (e.g., a CD80 transmembrane domain) and, optionally, an intracellular signaling domain. In some cases, the TIP is formatted as a chimeric receptor containing a heterologous cytoplasmic signaling domain or endodomain. In some embodiments, an engineered cell is capable of expressing and secreting an immunomodulatory protein as described herein. Among provided engineered cells also are cells further containing an engineered T-cell receptor (TCR) or chimeric antigen receptor (CAR).

The term “engineered T-cell” as used herein refers to a T-cell such as a T helper cell, cytotoxic T-cell (alternatively, cytotoxic T lymphocyte or CTL), natural killer T-cell, regulatory T-cell, memory T-cell, or gamma delta T-cell, that has been genetically modified by human intervention such as by recombinant DNA methods or viral transduction methods. An engineered T-cell contains a variant CD80 transmembrane immunomodulatory protein (TIP) or secreted immunomodulatory protein (SIP) of the present invention that is expressed on the T-cell and is engineered to modulate immunological activity of the engineered T-cell itself, or a mammalian cell to which the variant CD80 expressed on the T-cell specifically binds.

The term “engineered T-cell receptor” or “engineered TCR” refers to a T-cell receptor (TCR) engineered to specifically bind with a desired affinity to a major histocompatibility complex (MHC)/peptide target antigen that is selected, cloned, and/or subsequently introduced into a population of T-cells, often used for adoptive immunotherapy. In contrast to engineered TCRs, CARs are engineered to bind target antigens in an MHC independent manner.

The term “expressed on” as used herein is used in reference to a protein expressed on the surface of a cell, such as a mammalian cell. Thus, the protein is expressed as a membrane protein. In some embodiments, the expressed protein is a transmembrane protein. In some embodiments, the protein is conjugated to a small molecule moiety such as a drug or detectable label. Proteins expressed on the surface of a cell can include cell-surface proteins such as cell surface receptors that are expressed on mammalian cells.

The term “half-life extending moiety” refers to a moiety of a polypeptide fusion or chemical conjugate that extends the half-life of a protein circulating in mammalian blood serum compared to the half-life of the protein that is not so conjugated to the moiety. In some embodiments, half-life is extended by greater than or greater than about 1.2-fold, 1.5-fold, 2.0-fold, 3.0-fold, 4.0-fold, 5.0-fold, or 6.0-fold. In some embodiments, half-life is extended by more than 6 hours, more than 12 hours, more than 24 hours, more than 48 hours, more than 72 hours, more than 96 hours or more than 1 week after in vivo administration compared to the protein without the half-life extending moiety. The half-life refers to the amount of time it takes for the protein to lose half of its concentration, amount, or activity. Half-life can be determined for example, by using an ELISA assay or an activity assay. Exemplary half-life extending moieties include an Fc domain, a multimerization domain, polyethylene glycol (PEG), hydroxyethyl starch (HES), XTEN (extended recombinant peptides; see, WO2013130683), human serum albumin (HSA), bovine serum albumin (BSA), lipids (acylation), and poly-Pro-Ala-Ser (PAS), and polyglutamic acid (glutamylation).

An Fc (fragment crystallizable) region or domain of an immunoglobulin molecule (also termed an Fc polypeptide) corresponds largely to the constant region of the immunoglobulin heavy chain, and is responsible for various functions, including the antibody's effector function(s). The Fc domain contains part or all of a hinge domain of an immunoglobulin molecule plus a CH2 and a CH3 domain. In some cases for inclusion in a provided fusion protein, all or a portion of the Fc hinge sequence may be deleted. Hence, reference to an Fc domain herein refers to such sequence with or without a hinge sequence. The Fc domain can form a dimer of two polypeptide chains joined by one or more disulfide bonds. The Fc domain may be an Fc domain from an IgG molecule, such as an IgG1, IgG2 or IgG4. In some embodiments, the Fc domain exhibits reduced or weak Fc effector activity via Fc receptor (e.g. FcγRI) binding. In some embodiments, the Fc is a variant Fc that exhibits reduced (e.g., reduced greater than 30%, 40%, 50%, 60%, 70%, 80%, 90% or more) activity to facilitate an effector function. In some embodiments, reference to amino acid substitutions in an Fc region is by EU numbering system unless described with reference to a specific SEQ ID NO. EU numbering is known and is according to the most recently updated IMGT Scientific Chart (IMGT®, the international ImMunoGeneTics information System®, http://www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

An immunoglobulin Fc fusion (“Fc-fusion”), such as an immunomodulatory Fc fusion protein, is a molecule comprising one or more polypeptides (or one or more small molecules) operably linked to an Fc region of an immunoglobulin. An Fc-fusion may comprise, for example, the Fc region linked to a variant CD80 extracellular domain polypeptide containing an IgV domain. An immunoglobulin Fc region may be linked indirectly or directly to one or more variant CD80 polypeptides. Various linkers are known in the art and can optionally be used to link an Fc to a fusion partner to generate an Fc-fusion. Fc-fusions of identical species can be dimerized to form Fc-fusion homodimers. Fc-fusions of non-identical species (e.g. knob into hole engineering) may be used to form Fc-fusion heterodimers. In some embodiments, the Fc is a mammalian Fc such as a murine, rabbit or human Fc.

The term “host cell” refers to a cell that can be used to express a protein encoded by a recombinant expression vector. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include Chinese hamster ovary (CHO) cells or their derivatives such as Veggie CHO, DG44, Expi CHO, or CHOZN and related cell lines which grow in serum-free media or CHO strain DX-B11, which is deficient in DHFR. In some embodiments, a host cell can be a mammalian cell (e.g., a human cell, a monkey cell, a hamster cell, a rat cell, a mouse cell, or an insect cell).

The term “immunological synapse” or “immune synapse” (abbreviated “IS”) as used herein means the interface between a mammalian cell that expresses MHC I (major histocompatibility complex) or MHC II, such as an antigen-presenting cell or tumor cell, and a mammalian lymphocyte such as an effector T cell or Natural Killer (NK) cell.

The term “immunoglobulin” (abbreviated “Ig”) as used herein refers to a mammalian immunoglobulin protein including any of the five human classes of antibody: IgA (which includes subclasses IgA1 and IgA2), IgD, IgE, IgG (which includes subclasses IgG1, IgG2, IgG3, and IgG4), and IgM. The term is also inclusive of immunoglobulins that are less than full-length, whether wholly or partially synthetic (e.g., recombinant or chemical synthesis) or naturally produced, such as antigen binding fragment (Fab), variable fragment (Fv) containing V_{H} and V_{L}, the single chain variable fragment (scFv) containing V_{H} and V_{L} linked together in one chain, as well as other antibody V region fragments, such as Fab′, F(ab)₂, F(ab′)₂, dsFv diabody, Fc, and Fd polypeptide fragments. Bispecific antibodies, homobispecific and heterobispecific, are included within the meaning of the term.

The term “immunoglobulin superfamily” or “IgSF” as used herein means the group of cell surface and soluble proteins that are involved in the recognition, binding, or adhesion processes of cells. Molecules are categorized as members of this superfamily based on shared structural features with immunoglobulins (i.e., antibodies); they all possess a domain known as an immunoglobulin domain or fold. Members of the IgSF include cell surface antigen receptors, co-receptors and co-stimulatory molecules of the immune system, molecules involved in antigen presentation to lymphocytes, cell adhesion molecules, certain cytokine receptors and intracellular muscle proteins. They are commonly associated with roles in the immune system. Proteins in the immunological synapse are often members of the IgSF. IgSF can also be classified into “subfamilies” based on shared properties such as function. Such subfamilies typically are composed of from 4 to 30 IgSF members.

The terms “IgSF domain” or “immunoglobulin domain” or “Ig domain” as used herein refers to a structural domain of IgSF proteins. Ig domains are named after the immunoglobulin molecules. They contain about 70-110 amino acids and are categorized according to their size and function. Ig-domains possess a characteristic Ig-fold, which has a sandwich-like structure formed by two sheets of antiparallel beta strands. Interactions between hydrophobic amino acids on the inner side of the sandwich and highly conserved disulfide bonds formed between cysteine residues in the B and F strands stabilize the Ig-fold. One end of the Ig domain has a section called the complementarity determining region that is important for the specificity of antibodies for their ligands. The Ig like domains can be classified (into classes) as: IgV, IgC1, IgC2, or IgI. Most Ig domains are either variable (IgV) or constant (IgC). IgV domains with 9 beta strands are generally longer than IgC domains with 7 beta strands. Ig domains of some members of the IgSF resemble IgV domains in the amino acid sequence, yet are similar in size to IgC domains. These are called IgC2 domains, while standard IgC domains are called IgC1 domains. The extracellular domain of wild-type CD80 contains two Ig domains: IgV and IgC. In some embodiments, the variant CD80 extracellular domain of immunomodulatory proteins provided herein may be a full extracellular domain containing the IgV and IgC domain or a specific binding fragment or portion thereof containing the IgV domain

The term “immunological activity” as used herein in the context of mammalian lymphocytes such as T-cells refers to one or more cell survival, cell proliferation, cytokine production (e.g., interferon-gamma), or T-cell cytotoxicity activities. In some cases, an immunological activity can mean their expression of cytokines, such as chemokines or interleukins. Assays for determining enhancement or suppression of immunological activity include the MLR (mixed lymphocyte reaction) assays measuring interferon-gamma cytokine levels in culture supernatants (Wang et al., Cancer Immunol Res. 2014 September: 2(9):846-56), SEB (staphylococcal enterotoxin B) T cell stimulation assay (Wang et al., Cancer Immunol Res. 2014 September: 2(9):846-56), and anti-CD3 T cell stimulation assays (Li and Kurlander, J Transl Med. 2010: 8: 104). Since T cell activation is associated with secretion of IFN-gamma cytokine, detecting IFN-gamma levels in culture supernatants from these in vitro human T cell assays can be assayed using commercial ELISA kits (Wu et al, Immunol Lett 2008 Apr. 15; 117(1): 57-62). Induction of an immune response results in an increase in immunological activity relative to quiescent lymphocytes. An immunomodulatory protein, such as a variant CD80 polypeptide containing an affinity modified IgSF domain, as provided herein can in some embodiments increase IFN-gamma (interferon-gamma) expression in a primary T-cell assay relative to a wild-type IgSF member or IgSF domain control. In some embodiments, in assaying for the ability of an immunomodulatory protein or affinity modified IgSF domain of the invention to increase IFN-gamma expression in a primary T-cell assay, a co-immobilization assay can be used. In a co-immobilization assay, a T-cell receptor signal, provided in some embodiments by anti-CD3 antibody, is used in conjunction with a co-immobilized affinity modified IgSF domain, such as a variant CD80, to determine the ability to increase IFN-gamma expression relative to a wild-type IgSF domain control. In some embodiments, a reporter T cell assay may be used as described in Example 4.

An “immunomodulatory protein” or “immunomodulatory polypeptide” is a protein that modulates immunological activity. By “modulation” or “modulating” an immune response is meant that immunological activity is either enhanced or suppressed. Such modulation includes any induction, or alteration in degree or extent, of immunological activity of an immune cell, such as a T cell. For example, soluble Fc fusion proteins herein, including multidomain immunomodulatory proteins provided herein, may induce or stimulate immunological activity of T cells. An immunomodulatory protein can be a single polypeptide chain or a multimer (dimers or higher order multimers) of at least two polypeptide chains covalently bonded to each other by, for example, interchain disulfide bonds. Thus, monomeric, dimeric, and higher order multimeric proteins are within the scope of the defined term. Multimeric proteins can be homomultimeric (of identical polypeptide chains) or heteromultimeric (of different polypeptide chains). An immunomodulatory protein can comprise a variant CD80 polypeptide.

The term “lymphocyte” as used herein means any of three subtypes of white blood cell in a mammalian immune system. They include natural killer cells (NK cells) (which function in cell-mediated, cytotoxic innate immunity), T cells (for cell-mediated, cytotoxic adaptive immunity), and B cells (for humoral, antibody-driven adaptive immunity). T cells include: T helper cells, cytotoxic T-cells, natural killer T-cells, memory T-cells, regulatory T-cells, or gamma delta T-cells. Innate lymphoid cells (ILC) are also included within the definition of lymphocyte.

The term “membrane protein” as used herein means a protein that, under physiological conditions, is attached directly or indirectly to a lipid bilayer. A lipid bilayer that forms a membrane can be a biological membrane such as a eukaryotic (e.g., mammalian) cell membrane or an artificial (i.e., man-made) membrane such as that found on a liposome. Attachment of a membrane protein to the lipid bilayer can be by way of covalent attachment, or by way of non-covalent interactions such as hydrophobic or electrostatic interactions. A membrane protein can be an integral membrane protein or a peripheral membrane protein. Membrane proteins that are peripheral membrane proteins are non-covalently attached to the lipid bilayer or non-covalently attached to an integral membrane protein. A peripheral membrane protein forms a temporary attachment to the lipid bilayer such that under the range of conditions that are physiological in a mammal, peripheral membrane protein can associate and/or disassociate from the lipid bilayer. In contrast to peripheral membrane proteins, integral membrane proteins form a substantially permanent attachment to the membrane's lipid bilayer such that under the range of conditions that are physiological in a mammal, integral membrane proteins do not disassociate from their attachment to the lipid bilayer. A membrane protein can form an attachment to the membrane by way of one layer of the lipid bilayer (monotopic), or attached by way of both layers of the membrane (polytopic). An integral membrane protein that interacts with only one lipid bilayer is an “integral monotopic protein”. An integral membrane protein that interacts with both lipid bilayers is an “integral polytopic protein” alternatively referred to herein as a “transmembrane protein”.

As used herein, modification is in reference to modification of a sequence of amino acids of a polypeptide or a sequence of nucleotides in a nucleic acid molecule and includes a change in amino acids or nucleotides, respectively, of the sequence. The amino acid modification or change may be a deletion, insertion, or replacement (substitution) of amino acids or nucleotides, respectively. Methods of modifying a polypeptide are routine to those of skill in the art, such as by using recombinant DNA methodologies.

The term, a “multimerization domain” refers to a sequence of amino acids that promotes the formation of a multimer of two or more polypeptides. A multimerization domain includes sequences that promote stable interaction of a polypeptide molecule with one or more additional polypeptide molecules, each containing a complementary multimerization domain (e.g. a first multimerization domain and a second multimerization domain), which can be the same or a different multimerization domain. The interactions between complementary multimerization domains, e.g. interaction between a first multimerization domain and a second multimerization domain, form a stable protein-protein interaction to produce a multimer of the polypeptide molecule with the additional polypeptide molecule. In some cases, the multimerization domain is the same and interacts with itself to form a stable protein-protein interaction between two polypeptide chains. Generally, a polypeptide is joined directly or indirectly to the multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3 or IgG4 subtypes, IgA, IgE, IgD and IgM and modified forms thereof.

The terms “nucleic acid” and “polynucleotide” are used interchangeably to refer to a polymer of nucleic acid residues (e.g., deoxyribonucleotides or ribonucleotides) in either single- or double-stranded form. Unless specifically limited, the terms encompass nucleic acids containing known analogues of natural nucleotides and that have similar binding properties to it and are metabolized in a manner similar to naturally-occurring nucleotides. Unless otherwise indicated, a particular nucleic acid sequence also implicitly encompasses conservatively modified variants thereof (e.g., degenerate codon substitutions) and complementary nucleotide sequences as well as the sequence explicitly indicated (a “reference sequence”). Specifically, degenerate codon substitutions may be achieved by generating sequences in which the third position of one or more selected (or all) codons is substituted with mixed-base and/or deoxyinosine residues. The term nucleic acid or polynucleotide encompasses cDNA or mRNA encoded by a gene.

The term “non-competitive binding” as used herein means the ability of a protein to specifically bind simultaneously to at least two cognate binding partners. Thus, the protein is able to bind to at least two different cognate binding partners at the same time, although the binding interaction need not be for the same duration such that, in some cases, the protein is specifically bound to only one of the cognate binding partners. In some embodiments, the binding occurs under specific binding conditions. In some embodiments, the simultaneous binding is such that binding of one cognate binding partner does not substantially inhibit simultaneous binding to a second cognate binding partner. In some embodiments, non-competitive binding means that binding a second cognate binding partner to its binding site on the protein does not displace the binding of a first cognate binding partner to its binding site on the protein. Methods of assessing non-competitive binding are well known in the art such as the method described in Perez de La Lastra et al., Immunology, 1999 April: 96(4): 663-670. In some cases, in non-competitive interactions, the first cognate binding partner specifically binds at an interaction site that does not overlap with the interaction site of the second cognate binding partner such that binding of the second cognate binding partner does not directly interfere with the binding of the first cognate binding partner. Thus, any effect on binding of the cognate binding partner by the binding of the second cognate binding partner is through a mechanism other than direct interference with the binding of the first cognate binding partner. For example, in the context of enzyme-substrate interactions, a non-competitive inhibitor binds to a site other than the active site of the enzyme. Non-competitive binding encompasses uncompetitive binding interactions in which a second cognate binding partner specifically binds at an interaction site that does not overlap with the binding of the first cognate binding partner but binds to the second interaction site only when the first interaction site is occupied by the first cognate binding partner.

The terms “in operable combination,” “in operable order” and “operably linked” as used herein refer to the linkage of nucleic acid sequences in such a manner or orientation that the segments are arranged so that they function in concert for their intended purposes. In some embodiments, the term refers to linkage of nucleic acids to produce a nucleic acid molecule capable of directing the transcription of a given gene and/or to produce a desired protein molecule that is functional. For example, segments of a DNA sequence, e.g. a coding sequence and a regulatory sequence(s), are linked in such a way as to permit gene expression when the appropriate molecules (e.g. transcriptional activator proteins) are bound to the regulatory sequence.

The term “pharmaceutical composition” refers to a composition suitable for pharmaceutical use in a mammalian subject, often a human. A pharmaceutical composition typically comprises an effective amount of an active agent (e.g., an immunomodulatory polypeptide comprising a variant CD80) and a carrier, excipient, or diluent. The carrier, excipient, or diluent is typically a pharmaceutically acceptable carrier, excipient or diluent, respectively. In some embodiments, the pharmaceutical composition typically comprises an effective amount of cells engineered with the immunomodulatory polypeptide comprise the variant CD80.

The terms “polypeptide” and “protein” are used interchangeably herein and refer to a molecular chain of two or more amino acids linked through peptide bonds. The terms do not refer to a specific length of the product. Thus, “peptides,” and “oligopeptides,” are included within the definition of polypeptide. The terms include post-translational modifications of the polypeptide, for example, glycosylation, acetylation, phosphorylation and the like. The terms also include molecules in which one or more amino acid analogs or non-canonical or unnatural amino acids that can be synthesized, or expressed recombinantly using known protein engineering techniques. In addition, proteins can be derivatized as described herein by well-known organic chemistry techniques.

The term “purified” as applied to nucleic acids, such as encoding immunomodulatory proteins, or proteins (e.g. immunomodulatory proteins), generally denotes a nucleic acid or protein or polypeptide that is substantially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or protein is at least about 50% pure, usually at least about 75%, 80%, 85%, 90%, 95%, 96%, 99% or more pure (e.g., percent by weight or on a molar basis).

The term “recombinant” indicates that the material (e.g., a nucleic acid or a polypeptide) has been artificially (i.e., non-naturally) altered by human intervention. The alteration can be performed on the material within, or removed from, its natural environment or state. For example, a “recombinant nucleic acid” is one that is made by recombining nucleic acids, e.g., during cloning, affinity modification, DNA shuffling or other well-known molecular biological procedures. A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid or that is otherwise altered by genetic engineering, such as by introducing into the cell a nucleic acid molecule encoding a recombinant protein, such as a transmembrane immunomodulatory protein provided herein. Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. The terms “in operable combination,” “in operable order” and “operably linked” as used herein refer to the linkage of nucleic acid sequences in such a manner or orientation that a nucleic acid molecule capable of directing the transcription of a given gene and/or the synthesis of a desired protein molecule is produced.

The term “recombinant expression vector” as used herein refers to a DNA molecule containing a desired coding sequence and appropriate nucleic acid sequences necessary for the expression of the operably linked coding sequence in a particular host cell. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the recombinant expression vector, operably linked to the coding sequence for the recombinant protein, such as a recombinant fusion protein, so that the expressed fusion protein can be secreted by the recombinant host cell, for easier isolation of the fusion protein from the cell, if desired. The term includes the vector as a self-replicating nucleic acid structure as well as the vector incorporated into the genome of a host cell into which it has been introduced. Among the vectors are viral vectors, such as lentiviral vectors.

The term “sequence identity” as used herein refers to the sequence identity between genes or proteins at the nucleotide or amino acid level, respectively. “Sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. Methods for the alignment of sequences for comparison are well known in the art, such methods include GAP, BESTFIT, BLAST, BLAST-2, ALIGN or Megalign (DNASTAR) software, FASTA and TFASTA. The BLAST algorithm calculates percent sequence identity and performs a statistical analysis of the similarity between the two sequences. The software for performing BLAST analysis is publicly available through the National Center for Biotechnology Information (NCBI) website. In some cases, a percent sequence identity can be determined as the percentage of amino acid residues (or nucleotide residues) in a candidate sequence that are identical with the amino acid residues (or nucleotide residues) in a reference sequence, after aligning the sequences and introducing gaps, if necessary, to achieve the maximum percent sequence identity. Reference to sequence identity includes sequence identity across the full length of each of the sequences being compared. Those skilled in the art can determine appropriate parameters for aligning sequences, including any algorithms needed to achieve maximal alignment over the full length of the sequences being compared.

The term “soluble” as used herein in reference to proteins means that the protein is not a membrane protein or is not anchored in a cell membrane. A protein can be constructed as a soluble protein by inclusion of only an extracellular domain or a portion thereof and without a transmembrane domain. In some cases, solubility of a protein can be improved by linkage or attachment, directly or indirectly via a linker, to an Fc domain or other half-life extending molecule, which, in some cases, also can improve the stability and/or half-life of the protein. In some aspects, a soluble protein is an Fc fusion protein.

The term “specifically binds” as used herein means the ability of a protein, under specific binding conditions, to bind to a target protein such that its affinity or avidity is at least 5 times as great, but optionally 10, 20, 30, 40, 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule but may specifically bind to more than one target molecule. In some cases, a specifically binding protein may bind to a protein that has similarity in structural conformation with the target protein (e.g., paralogs or orthologs). Those of skill will recognize that specific binding to a molecule having the same function in a different species of animal (i.e., ortholog) or to a molecule having a substantially similar epitope as the target molecule (e.g., paralog) is possible and does not detract from the specificity of binding which is determined relative to a statistically valid collection of unique non-targets (e.g., random polypeptides). Thus, an immunomodulatory protein of the invention, or each individual binding domain thereof, may specifically bind to more than one distinct species of target molecule due to cross-reactivity. Solid-phase ELISA immunoassays, ForteBio Octet or Biacore measurements can be used to determine specific binding between two proteins. Generally, interactions between two binding proteins have dissociation constants (Kd) less than about 1×10⁻⁵ M, and often as low as about 1×10⁻¹² M. In certain aspects of the present disclosure, interactions between two binding proteins have dissociation constants of less than about 1×10⁻⁶ M, 1×10⁻⁷ M, 1×10⁻⁸ M, 1×10⁻⁹ M, 1×10⁻¹⁰ M, or 1×10⁻¹¹ M or less.

The term “specific binding fragment” or “fragment” or “binding portion” as used herein in reference to a protein means a polypeptide that is shorter than a full-length protein or a specific domain or region thereof and that specifically binds in vitro and/or in vivo to a binding partner of the full-length protein or of the specific domain or region. A specific finding fragment or binding portion is in reference to a fragment or portion of a full length extracellular domain of a polypeptide or a binding domain of a polypeptide, but that still binds to a binding partner of the binding domain. For example, a specific binding fragment is in reference to a fragment of a full-length CD80 extracellular domain (e.g. containing IgV and IgC domains), but that still binds to a binding partner of CD80. In some embodiments, the specific binding fragment or binding portion is at least 20%, 30%, 40%, 50%, 60%, 70%, 75%, 80%, 85%, 90%, 95%, 96%, 97%, 98%, or 99% the sequence length of the full-length sequence of the extracellular domain. In some embodiments, the specific binding fragment can have an amino acid length of at least 50 amino acids, such as at least 60, 70, 80, 90, 100, or 110 amino acids. For instance, a specific binding fragment or binding portion of an extracellular domain generally includes an IgV domain.

The terms “surface expresses” or “surface expression” in reference to a mammalian cell expressing a polypeptide means that the polypeptide is expressed as a membrane protein. In some embodiments, the membrane protein is a transmembrane protein.

As used herein, a “subject” is a mammal, such as a human or other animal, and typically is human. The subject can be male or female and can be any suitable age, including infant, juvenile, adolescent, adult, and geriatric subjects.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

The term “targeting moiety” as used herein refers to a composition that is covalently or non-covalently attached to, or physically encapsulates, a polypeptide comprising the variant CD80. The targeting moiety has specific binding affinity for a desired counter-structure such as a cell surface receptor (e.g., the B7 family member PD-L1), or a tumor antigen such as tumor specific antigen (TSA) or a tumor associated antigen (TAA). Typically, the desired counter-structure is localized on a specific tissue or cell-type. Targeting moieties include: antibodies, antigen binding fragment (Fab), variable fragment (Fv) containing V_{H} and V_{L}, the single chain variable fragment (scFv) containing V_{H} and V_{L}linked together in one chain, as well as other antibody V region fragments, such as Fab′, F(ab)₂, F(ab′)₂, dsFv diabody, nanobodies, soluble receptors, receptor ligands, affinity matured receptors or ligands, as well as small molecule (<500 Dalton) compositions (e.g., specific binding receptor compositions). Targeting moieties can also be attached covalently or non-covalently to the lipid membrane of liposomes that encapsulate a polypeptide of the present invention.

The terms “treating,” “treatment,” or “therapy” of a disease or disorder as used herein mean slowing, stopping or reversing the disease or disorders progression, as evidenced by decreasing, cessation or elimination of either clinical or diagnostic symptoms, by administration of an immunomodulatory protein either alone or in combination with another compound as described herein. “Treating,” “treatment,” or “therapy” also means a decrease in the severity of symptoms in an acute or chronic disease or disorder or a decrease in the relapse rate. As used herein in the context of cancer, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS). “Preventing,” “prophylaxis,” or “prevention” of a disease or disorder as used in the context of this invention refers to the administration of an immunomodulatory protein, either alone or in combination with another compound, to prevent the occurrence or onset of a disease or disorder or some or all of the symptoms of a disease or disorder or to lessen the likelihood of the onset of a disease or disorder.

The term “tumor specific antigen” or “TSA” as used herein refers to a counter-structure that is present primarily on tumor cells of a mammalian subject but generally not found on normal cells of the mammalian subject. A tumor specific antigen need not be exclusive to tumor cells but the percentage of cells of a particular mammal that have the tumor specific antigen is sufficiently high or the levels of the tumor specific antigen on the surface of the tumor are sufficiently high such that it can be targeted by anti-tumor therapeutics, such as immunomodulatory polypeptides of the invention, and provide prevention or treatment of the mammal from the effects of the tumor. In some embodiments, in a random statistical sample of cells from a mammal with a tumor, at least 50% of the cells displaying a TSA are cancerous. In other embodiments, at least 60%, 70%, 80%, 85%, 90%, 95%, or 99% of the cells displaying a TSA are cancerous.

The term “variant” (also “modified” or mutant,” which can be used interchangeably) as used in reference to a variant CD80 protein means a protein, such as a mammalian (e.g., human or murine) protein created by human intervention. The variant CD80 is a polypeptide having an altered or modified amino acid sequence, such as by one or more amino acid substitutions, deletions, additions or combinations thereof, relative to an unmodified or wild-type protein or to a domain thereof. For purposes herein, the variant CD80 contains at least one affinity modified domain, whereby one or more of the amino acid differences occurs in the IgV domain. A variant CD80 polypeptide can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions. A variant CD80 polypeptide generally exhibits at least 50%, 60%, 70%, 80%, 85%, 86%, 87%, 88%, 89%, 90%, 91%, 92%, 93%, 94%, 95%, 96%, 97%, 98%, 99% or more sequence identity to a corresponding form of a wild-type or unmodified protein, such as an extracellular domain of wild-type CD80 set forth in SEQ ID NO:2 or binding portion thereof containing the IgV domain set forth in SEQ ID NO:163 or SEQ ID NO:164. Non-naturally occurring amino acids as well as naturally occurring amino acids are included within the scope of permissible substitutions or additions. A variant protein is not limited to any particular method of making and includes, for example, chemical synthesis, recombinant DNA techniques, or combinations thereof. A variant protein of the invention specifically binds to at least one or more binding partners. In some embodiments, the altered amino acid sequence results in an altered (i.e., increased or decreased) binding activity, such as binding affinity or avidity, to the one or more binding partners. In some embodiments, by virtue of the altered binding activity or affinity, the altered IgV domain is an affinity modified IgSF domain. A variant protein may thus be an “affinity-modified” protein as described herein.

The term “wild-type” or “natural,” “native” or “unmodified” with reference to a protein, which are used interchangeably, as used herein is used in connection with biological materials such as nucleic acid molecules, proteins, host cells, and the like, that are found in nature or not modified by human intervention.

### II. Variant CD80 Immunomodulatory Proteins and Engineered Cells

Provided herein are variant CD80 immunomodulatory proteins that contain at least one variant extracellular domain of CD80. In particular embodiments, the provided variant immunomodulatory proteins, including fusion proteins as described (e.g. CD80-Fc fusion proteins), are purified proteins. In some embodiments, such immunomodulatory protein, including fusion proteins (CD80-Fc fusion proteins) provide a protein-based therapeutic for use in modulating immune responses, including for the treatment of cancer, bacterial infections or viral infections.

In some embodiments, also provided herein are engineered immune cells which express the immunomodulatory variant CD80 polypeptides (alternatively, “engineered cells”). In some embodiments, the expressed immunomodulatory variant CD80 polypeptide is a transmembrane protein and is surface expressed. In some embodiments, the expressed immunomodulatory variant CD80 polypeptide is expressed and secreted from the immune cell. In some embodiments, the provided immune cells are engineered with a variant CD80 immunomodulatory proteins that contain at least one variant extracellular domain of CD80.

The variant extracellular domain of CD80 may include an extracellular domain or a specific binding portion thereof that contains an IgV domain in which is contained one or more amino acid modifications, such as one or more substitutions (alternatively, “mutations” or “replacements”), deletions or additions relative to unmodified CD80 polypeptide (e.g. wild-type CD80) or a portion of a wild-type or unmodified CD80 extracellular domain or the specific binding portion thereof containing the IgV domain. By virtue of containing modifications (e.g. amino acid substitutions) in the IgV domain, which is an immunoglobulin superfamily (IgSF) domain, the provided variant CD80 polypeptide is or comprises a variant IgSF domain (hereinafter called “vIgD”) in which the one or more amino acid modifications (e.g., substitutions) is in the IgV domain. In some embodiments, the at least one variant extracellular domain of CD80 is an IgV only molecule in which the IgV domain is the only IgSF domain of the CD80 polypeptide of the immunomodulatory protein.

In some embodiments, the variant CD80 polypeptide, or an immunomodulatory protein containing the same, exhibits altered (e.g. increased) binding affinity for one or more of a CD80 cognate binding partner, PD-L1, CD28 or CTLA-4. In some embodiments, by virtue of the altered binding activity or affinity, the altered IgV domain is an affinity modified IgSF domain. Hence, the provided variant CD80 polypeptides include a vIgD that is an affinity-modified IgSF domains, such that the variant CD80 polypeptide exhibits altered (increased or decreased) binding activity or affinity for one or more cognate binding partners, PD-L1, CD28 or CTLA-4, compared to a wild-type or unmodified CD80 polypeptide containing the IgSF domain. In some embodiments, the binding affinity is increased for PD-L1. In some embodiments, among provided variant CD80 polypeptides, or immunomodulatory proteins containing the same, also are those that exhibit a relatively slow PD-L1 off-rate of less than less than 50×10³ s⁻¹, such as less than less than 20×10⁻³ s⁻¹.

The provided variant CD80 immunomodulatory proteins include soluble fusion proteins thereof in which the variant CD80 extracellular domain is linked to another moiety, such as an immunoglobulin Fc or other multimerization domain, half-life extending moiety or targeting moiety. Among provided immunomodulatory proteins is a variant CD80-Fc fusion protein. In some embodiments, provided is a variant CD80-Fc fusion protein containing (1) a variant CD80 polypeptide composed of a variant CD80 extracellular domain containing an IgV domain in which is contained one or more amino acid substitutions compared to the extracellular domain or a binding portion thereof containing the IgV domain of wild-type CD80, and (2) an Fc domain, wherein the variant CD80 extracellular domain of the variant CD80-Fc fusion protein exhibits increased binding to PD-L1 compared to a wild-type CD80-Fc containing the extracellular domain or a binding portion thereof containing the IgV domain of wild-type CD80. The variant CD80 polypeptide can be linked directly or indirectly (e.g. via a peptide linker) to the Fc domain.

Generally, each of the various attributes of polypeptides or immunomodulatory proteins are separately disclosed below (e.g. affinity of CD80 for PD-L1, CD28 or CTLA-4, PD-L1 off rate, number of variations per polypeptide chain, number of linked polypeptide chains, the number and nature of amino acid alterations per variant CD80, etc.). However, as will be clear to the skilled artisan, any particular polypeptide can comprise a combination of these independent attributes. It is understood that reference to amino acids, including to a specific sequence set forth as a SEQ ID NO used to describe domain organization of an IgSF domain are for illustrative purposes and are not meant to limit the scope of the embodiments provided. It is understood that polypeptides and the description of domains thereof are theoretically derived based on homology analysis and alignments with similar molecules. Thus, the exact locus can vary, and is not necessarily the same for each protein. Hence, the specific IgSF domain, such as specific IgV domain or IgC domain, can be several amino acids (such as one, two, three or four) longer or shorter.

Further, various embodiments of the invention as discussed below are frequently provided within the meaning of a defined term as disclosed above. The embodiments described in a particular definition are therefore to be interpreted as being incorporated by reference when the defined term is utilized in discussing the various aspects and attributes described herein. Thus, the headings, the order of presentation of the various aspects and embodiments, and the separate disclosure of each independent attribute is not meant to be a limitation to the scope of the present disclosure.

### A. Variant CD80 Polypeptides

In some embodiments, the variant CD80 polypeptide is or includes an extracellular domain or a specific binding portion thereof containing an IgV domain in which is contained one or more amino acid modifications, such as one or more substitutions (alternatively, “mutations” or “replacements”), deletions or additions in an immunoglobulin superfamily (IgSF) domain (IgD) relative to a wild-type or unmodified CD80 polypeptide or a portion of a wild-type or unmodified CD80 containing the IgD or a specific binding fragment thereof. Thus, a provided variant CD80 polypeptide is or comprises a variant IgD (hereinafter called “vIgD”) in which the one or more amino acid modifications (e.g., substitutions) is in an IgD. In particular embodiments, the vIgD is a variant IgV domain. In some embodiments, the one or more amino acids modifications, such as one or more amino acid substitutions, is in the IgV domain. In some embodiments, the variant CD80 polypeptide is a variant CD80 polypeptide composed of an extracellular domain portion of CD80 containing the entire extracellular domain (ECD) or a specific binding portion thereof containing the IgV domain in which is contained the one or more amino acid modifications, such as one or more substitutions. In some embodiments, the variant CD80 extracellular domain comprises or consists essentially of the extracellular domain or a specific binding portion thereof containing the IgV domain. In some embodiments, the variant CD80 extracellular domain comprises or consists essentially of the IgV domain.

In some embodiments, the variant CD80 polypeptide is modified (e.g. by one or more amino acid substitutions) in the IgV domain relative to the sequence of an unmodified CD80 sequence. In some embodiments, the unmodified CD80 sequence is a wild-type CD80.

In some embodiments, the variant CD80 extracellular domain polypeptide comprises one or more amino acid substitutions described herein in the sequence of a wild-type CD80 extracellular domain or a portion thereof comprising an IgV domain. In some embodiments, the unmodified CD80 is a wild-type CD80 sequence that is a mammalian CD80 sequence. In some embodiments, the wild-type CD80 sequence can be a mammalian CD80 that includes, but is not limited to, human, mouse, cynomolgus monkey, or rat. In some embodiments, the wild-type CD80 sequence is human. In some embodiments, the wild-type CD80 has the sequence set forth in SEQ ID NO:1 or is a portion thereof containing the extracellular domain or a specific binding portion thereof containing the IgV domain. The extracellular domain of an exemplary human CD80 sequence is set forth in SEQ ID NO:2.

In some embodiments, the unmodified CD80 sequence is set forth in SEQ ID NO:2. In some embodiments, the variant CD80 extracellular domain contains one or more amino acid substitutions described herein in the sequence of an unmodified CD80 extracellular domain set forth in SEQ ID NO:2.

SEQ ID NO: 2
VIHVTKEVKEVATLSCGHNVSVEELAQTRIYWQKEKKMVLTMMSG
DMNIWPEYKNRTIFDITNNLSIVILALRPSDEGTYECVVLKYEKD
AFKREHLAEVTLSVKADFPTPSISDFEIPTSNIRRIICSTSGGFP
EPHLSWLENGEELNAINTTVSQDPETELYAVSSKLDFNMTTNHSF
MCLIKYGHLRVNQTFNWNTTKQEHFPDN

In some embodiments, the unmodified CD80 sequence has (i) the sequence of amino acids set forth in SEQ ID NO:2, (ii) a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO:2, or (iii) is a fragment or portion of (i) or (ii) containing an IgV domain. In some embodiments, the variant CD80 extracellular domain contains one or more amino acid substitutions described herein in the sequence of an unmodified CD80 extracellular domain that has (i) the sequence of amino acids set forth in SEQ ID NO:2, (ii) a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO:2, or (iii) is a fragment or portion of (i) or (ii) containing an IgV domain. In some embodiments, the wild-type or unmodified extracellular domain of CD80 is capable of binding one or more CD80 binding proteins, such as one or more of CTLA-4, PD-L1 or CD28.

In some embodiments, the unmodified CD80 sequence is an extracellular domain sequence of a wild-type CD80 that is a portion of the ECD that contains an IgV domain relative to the sequence of amino acids of a wild-type CD80. In some embodiments, the IgV domain portion may be a sequence of amino acids that lacks the full sequence of the extracellular domain of CD80 but that contains the IgV domain. In some embodiments, the IgV domain portion is a portion of the extracellular domain of CD80 that includes the IgV domain but lacks the IgC domain. In some embodiments, the portion of the ECD that contains an IgV domain has a sequence of amino acids that is at least 100 amino acids in length and less than 115 amino acids in length. In some embodiments, the IgV domain portion has a length of 101 amino acids, 102 amino acids, 103 amino acids, 104 amino acids, 105 amino acids, 106 amino acids, 107 amino acids, 108 amino acids, 109 amino acids or 110 amino acids, 111 amino acids, 112 amino acids, 113 amino acids, 114 amino acids, 115 amino acids. In some embodiments, the unmodified CD80 sequence is a specific binding fragment or portion of SEQ ID NO:2 containing the IgV domain. In some embodiments, the IgV domain includes amino acids 1-101 of SEQ ID NO:2. In some embodiments, the IgV domain includes amino acids 1-101 of SEQ ID NO:2 and has a length of no more than 110 amino acids. In some embodiments, the extracellular domain portion containing a specific binding portion containing the IgV domain contains an amino acid sequence that is at least about 85%, 86%, 87%, 88%, 89%, 90%, 91%, 92%, 93%, 94%, 95%, 96%, 97%, 98%, 99% of the length of the IgV domain set forth as amino acids 1-101, 1-104, 3-104 or 1-107 of SEQ ID NO: 2. In some embodiments, the IgV portion includes amino acids 1-107 of SEQ ID NO:12. In some embodiments, the IgV portion is set forth as amino acids 1-107 of SEQ ID NO:2.

In some embodiments, the unmodified CD80 sequence is a specific binding fragment or portion of SEQ ID NO:2 containing the IgV domain. In some embodiments, the unmodified CD80 sequence is a portion of the ECD set forth in SEQ ID NO:163 (corresponding to amino acids 1-101 of SEQ ID NO:2). In some embodiments, the unmodified CD80 is an extracellular domain portion that is or consists of the sequence set forth in SEQ ID NO:163. In some embodiments, the variant CD80 extracellular domain contains one or more amino acid substitutions described herein in the sequence of an unmodified CD80 extracellular domain portion set forth in SEQ ID NO:163.

SEQ ID NO: 163
VIHVTKEVKEVATLSCGHNVSVEELAQTRIYWQKEKKMVLTMMSG
DMNIWPEYKNRTIFDITNNLSIVILALRPSDEGTYECVVLKYEKD
AFKREHLAEVT

In some embodiments, the unmodified CD80 sequence has (i) the sequence of amino acids set forth in SEQ ID NO:163, or (ii) a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO:163. In some embodiments, the variant CD80 extracellular domain contains one or more amino acid substitutions described herein in the sequence of an unmodified CD80 extracellular domain that has (i) the sequence of amino acids set forth in SEQ ID NO:163, or (ii) a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO:163. In some embodiments, the wild-type or unmodified extracellular domain of CD80 is capable of binding one or more CD80 binding proteins, such as one or more of CTLA-4, PD-L1 or CD28.

In some embodiments, the unmodified CD80 sequence is a portion of the ECD of CD80 set forth in SEQ ID NO:164 (corresponding to amino acids 1-107 of SEQ ID NO:2). In some embodiments, the unmodified CD80 is an extracellular domain portion that is or consists of the sequence set forth in SEQ ID NO:164. In some embodiments, the variant CD80 extracellular domain contains one or more amino acid substitutions described herein in the sequence of an unmodified CD80 extracellular domain portion set forth in SEQ ID NO:164.

SEQ ID NO: 164
VIHVTKEVKEVATLSCGHNVSVEELAQTRIYWQKEKKMVLTMMSG
DMNIWPEYKNRTIFDITNNLSIVILALRPSDEGTYECVVLKYEKD
AFKREHLAEVTLSVKAD

In some embodiments, the unmodified CD80 sequence has (i) the sequence of amino acids set forth in SEQ ID NO:164, or (ii) a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO:164. In some embodiments, the variant CD80 extracellular domain contains one or more amino acid substitutions described herein in the sequence of an unmodified CD80 extracellular domain that has (i) the sequence of amino acids set forth in SEQ ID NO:164, or (ii) a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO:164. In some embodiments, the wild-type or unmodified extracellular domain of CD80 is capable of binding one or more CD80 binding proteins, such as one or more of CTLA-4, PD-L1 or CD28.

Unless stated otherwise, as indicated throughout the present disclosure, the amino acid modifications(s) are designated by amino acid position number corresponding to the numbering of positions of the unmodified ECD sequence set forth in SEQ ID NO:2 or, where applicable, the unmodified IgV sequence set forth in SEQ ID NO: 163 or 164. It is within the level of a skilled artisan to identify the corresponding position of a modification, e.g., amino acid substitution, in a CD80 polypeptide, including portion thereof containing an IgSF domain (e.g., IgV) thereof, such as by alignment of a reference sequence with SEQ ID NO:2 or SEQ ID NO:163 or SEQ ID NO:164. For instance, a skilled artisan readily understands that the numbering of aligned residues in the sequences set forth in SEQ ID NO:2, 163 and 164 are the same. In the listing of modifications throughout this disclosure, the amino acid position is indicated in the middle, with the corresponding unmodified (e.g., wild-type) amino acid listed before the number and the identified variant amino acid substitution listed after the number. If the modification is a deletion of the position, a “del” is indicated, and if the modification is an insertion at the position, an “ins” is indicated. In some cases, an insertion is listed with the amino acid position indicated in the middle, with the corresponding unmodified (e.g., wild-type) amino acid listed before and after the number and the identified variant amino acid insertion listed after the unmodified (e.g., wild-type) amino acid.

The wild-type or unmodified CD80 sequence does not necessarily have to be used as a starting composition to generate variant CD80 polypeptides described herein. Therefore, use of the term “substitution” does not imply that the provided embodiments are limited to a particular method of making variant CD80 polypeptides. Variants CD80 polypeptides can be made, for example, by de novo peptide synthesis and thus does not necessarily require a “substitution” in the sense of altering a codon to encode for the substitution. This principle also extends to the terms “addition” and “deletion” of an amino acid residue which likewise do not imply a particular method of making. The means by which the variant CD80 polypeptides are designed or created is not limited to any particular method. In some embodiments, however, a wild-type or unmodified CD80 encoding nucleic acid is mutagenized from wild-type or unmodified CD80 genetic material and screened for desired specific binding affinity and/or induction of IFN-gamma expression or other functional activity according to the methods disclosed in the Examples or other methods known to a skilled artisan. In some embodiments, a variant CD80 polypeptide is synthesized de novo utilizing protein or nucleic acid sequences available at any number of publicly available databases and then subsequently screened. The National Center for Biotechnology Information provides such information and its website is publicly accessible via the internet as is the UniProtKB database as discussed previously.

In some embodiments, the variant CD80 polypeptide has up to 1, 2, 3, 4, 5, 6, 7, 8, 9, 10, 11, 12, 13, 14, 15, 16, 17, 18, 19, or 20 amino acid modifications (e.g., substitutions) compared to the unmodified CD80 polypeptide. The modifications (e.g., substitutions) can be in the IgV domain. In some embodiments, the variant CD80 polypeptide has up to 1, 2, 3, 4, 5, 6, 7, 8, 9, 10, 11, 12, 13, 14, 15, 16, 17, 18, 19, or 20 amino acid modifications (e.g., substitutions) in the IgV domain. In some embodiments, additional modifications (e.g., substitutions) also may be present in the IgC domain.

In some embodiments, the variant CD80 polypeptide has at least about 85%, 86%, 86%, 88%, 89%, 90%, 91%, 92%, 93%, 94%, 95%, 96%, 97%, 98%, or 99% sequence identity with the wild-type or unmodified CD80 polypeptide or specific binding fragment thereof.

In some embodiments, the variant CD80 polypeptide has at least about 85%, 86%, 86%, 88%, 89%, 90%, 91%, 92%, 93%, 94%, 95%, 96%, 97%, 98%, or 99% sequence identity with the wild-type or unmodified CD80 polypeptide set forth in SEQ ID NO:2.

In some embodiments, the variant CD80 polypeptide has at least about 85%, 86%, 86%, 88%, 89%, 90%, 91%, 92%, 93%, 94%, 95%, 96%, 97%, 98%, or 99% sequence identity with the wild-type or unmodified CD80 polypeptide set forth in SEQ ID NO:163.

In some embodiments, the variant CD80 polypeptide has at least about 85%, 86%, 86%, 88%, 89%, 90%, 91%, 92%, 93%, 94%, 95%, 96%, 97%, 98%, or 99% sequence identity with the wild-type or unmodified CD80 polypeptide set forth in SEQ ID NO:164.

In some embodiments, the variant CD80 polypeptide has one or more amino acid modifications (e.g., substitutions) in an unmodified CD80 or specific binding fragment there of corresponding to position(s) 7, 9, 10, 11, 18, 20, 22, 26, 27, 28, 29, 35, 42, 46, 47, 52, 59, 62, 63, 68, 71, 73, 77, 81, 85, 87, 90, 92, or 101. In some embodiments, at least one of the amino acid modifications (e.g., substitutions) in an unmodified CD80 or specific binding fragment there is at a position corresponding to position(s) 7, 9, 10, 11, 28 or 101. In some embodiments, at least one of the amino acid modifications (e.g., substitutions) in an unmodified CD80 or specific binding fragment there is at a position corresponding to position(s) 9, 10, or 11.

In some embodiments, the variant CD80 polypeptide has a modification, e.g., amino acid substitution, at any 2 or more of the foregoing positions, such as 2, 3, 4, 5, 6, 7, 8, 9, 10, 11, 12, 13, 14, 15 or more of the positions, compared to the corresponding extracellular domain or portion thereof of the unmodified (e.g. wild-type CD80). In some embodiments, the variant CD80 polypeptide has 2-10 amino acid modifications, e.g. amino acid substitutions, compared to the unmodified CD80 polypeptide. In some embodiments, the number of amino acid modifications is 2, 3, 4, 5, 6, 7, 8, 9 or 10, compared to the unmodified CD80 polypeptide. In some embodiments, the variant CD80 polypeptide contains no more than 4 amino acid modifications (e.g. amino acid substitutions). In some embodiments the variant CD80 polypeptide contains 4 amino acid modifications (e.g. amino acid substitutions). In some embodiments, the variant CD80 polypeptide contains 3 amino acid modifications (e.g. amino acid substitutions). The amino acid modifications may be any combination of amino acid substitution as described herein. Variant CD80 polypeptides containing a combination of multiple amino acid substitutions are denoted herein in some cases with a/between substitutions. For instance, the exemplary variant CD80 extracellular domain polypeptide V11Y/T28Y/M47L refers to a polypeptide containing the three amino acid substitutions V11Y, T28Y and M47L. In some embodiments, any of such amino acid modifications (e.g. amino acid substitutions) as described are in an unmodified CD80 polypeptide set forth in SEQ ID NO:2 or in a portion thereof comprising an IgV domain. For instance, in some embodiments, any of such amino acid modifications (e.g. amino acid substitutions) as described are in an unmodified CD80 polypeptide set forth in SEQ ID NO:163. In some embodiments, any of such amino acid modifications (e.g. amino acid substitutions) as described are in an unmodified CD80 polypeptide set forth in SEQ ID NO:164.

In some embodiments, the variant CD80 polypeptide has one or more amino acid substitution selected from among E7S, E7K, E7N, E7H, E7Q, K9N, K9R, K9S, E10G, E10S, E10R, E10A, V11Y, V11F, V11W, H18I, H18Y, H18F, H18V, H18L, H18T, V20L, V20I, V22S, A26K, A26G, A26Q, A26E, A26S, A26T, Q27F, Q27T, T28Y, T28P, T28H, T28R, T28V, R29S, R29H, E35G, E35D, E35A, M42I, M42L, M42G, M42W, M42R, D46E, D46S, D46K, D46V, D46Q, D46N, M47V, M47L, M47R, M47W, E52K, F59S, F59M, F59Y, T62S, T62A, T62E, N63S, N63I, N63H, V68M, V68L, V68N, V68T, V68S, A71G, A71N, A71V, R73D, R73E, R73T, E77G, E81K, L85E, L85Q, Y87R, Y87I, Y87K, Y87Q, Y87N, Y87P, D90G, F92L, T101R, T101K, or T101Q, or a conservative amino acid substitution of any of the foregoing. In some embodiments, the variant CD80 polypeptide has one or more amino acid substitution selected from among E7S, E7K, E7N, E7H, E7Q, K9N, K9R, K9S, E10G, E10S, E10A, V11Y, V11F, V11W, V20I, V22S, Q27F, Q27T, T28P, T28H, T28R, T28V, R29S, E35A, M42L, M42G, M42W, M42R, D46S, D46K, D46Q, M47R, M47W, E52K, F59S, T62S, T62A, N63I, N63H, V68N, V68T, V68S, A71N, A71V, R73D, R73E, R73T, L85Q, Y87R, Y87I, Y87K, Y87P, T101R, T101K, and T101Q, or a conservative amino acid substitution of any of the foregoing. In some embodiments, at least one amino acid substitution is E7S, E7K, E7N, E7H, E7Q, K9N, K9R, K9S, E10G, E10S, E10R, E10A, V11Y, V11F, V11W, T28P, T28H, T28R, T28V, T101R, T101K, or T101Q.

In some embodiments, a conservative amino acid modification, e.g. substitution is any amino acid that falls in the same class of amino acids as the substituted amino acids, other than the reference (e.g., unmodified) or wild-type amino acid. The classes of amino acids are aliphatic (glycine, alanine, valine, leucine, and isoleucine), hydroxyl or sulfur-containing (serine, cysteine, threonine, and methionine), cyclic (proline), aromatic (phenylalanine, tyrosine, tryptophan), basic (histidine, lysine, and arginine), and acidic/amide (aspartate, glutamate, asparagine, and glutamine).

In some embodiments, the variant CD80 polypeptide includes at least one amino acid substitution at position 7. In some embodiments, the amino acid substitution at position 7 is E7S, E7K, E7N, E7H, E7Q, or is an amino acid substitution that is a conservative mutation of any of the foregoing. In some embodiments, the substituted amino acid at position 7 is a polar uncharged amino acid. In some embodiments, the polar uncharged amino acid is serine (Ser, S), asparagine (Asn, N), glutamine (Gln, Q), threonine (Thr, T). In some embodiments, the substituted amino acid at position 7 is a serine (Ser, S). In some embodiments, the amino acid substitution is E7S. In some embodiments, the substituted amino acid at position 7 is asparagine (Asn, N). In some embodiments, the amino acid substitution is E7N. In some embodiments, the substituted amino acid at position 7 is glutamine (Gln, Q). In some embodiments, the amino acid substitution is E7Q. In some embodiments, the substituted amino acid at position 7 is a basic amino acid. In some embodiments, the basic amino acid is lysine (Lys, K), arginine (Arg, R) or histidine (His, H). In some embodiments the substituted amino acid at position 7 is lysine (Lys, K). In some embodiments, the amino acid substitution is E7K. In some embodiments, the substituted amino acid at position 7 is histidine (His, H). In some embodiments, the amino acid substitution is E7H.

In some embodiments, the variant extracellular domain polypeptide comprises the amino acid substitutions E7S/H18I/V20L/A26K/M47L/A71N, E7K/V11W/N63H/A71G/Y87K, E7N/E35D/T101R, E7H/H18L/V20I/T28Y/D46S/A71G, E7N/E35D/F59S, or E7Q/V11Y/R29H/M47L/V68T.

In some embodiments, the variant CD80 polypeptide includes at least one amino acid substitution at position 9. In some embodiments, the amino acid substitution at position 9 is K9N, K9R, K9S, or is an amino acid substitution that is a conservative mutation of any of the foregoing. In some embodiments, the substituted amino acid at position 9 is a polar uncharged amino acid. In some embodiments, the polar uncharged amino acid is serine (Ser, S), asparagine (Asn, N), glutamine (Gln, Q), threonine (Thr, T). In some embodiments, the substituted amino acid at position 9 is a serine (Ser, S). In some embodiments, the amino acid substitution is K9S. In some embodiments, the substituted amino acid at position 9 is an asparagine (Asn, N). In some embodiments, the amino acid substitution is K9N. In some embodiments, the substituted amino acid at position 9 is glutamine (Gln, Q). In some embodiments, the amino acid substitution is K9Q. In some embodiments, the substituted amino acid at position 9 is threonine (Thr, T). In some embodiments, the amino acid substitution is K9T. In some embodiments, the substituted amino acid at position 9 is to a basic amino acid, which in some cases is other than lysine (K). In some embodiments, the basic amino acid is arginine (Arg, R) or histidine (His, H). In some embodiments, the substituted amino acid at position 9 is an arginine (Arg, R). In some embodiments, the amino acid substitution is K9R. In some embodiments, the substituted amino acid at position 9 is histidine (His, H). In some embodiments, the amino acid substitution is K9H.

In some embodiments, the variant CD80 extracellular domain polypeptide comprises the amino acid substitutions K9N/E10G/N63S/L85E, K9N/E10G/E35G/D46E/M47V/V68M, K9R/E10S/V11Y/M42L/F59M/V68M/L85E, K9S/E10R/V11Y/M47L/A71G, K9N/E10R/H18V/T28Y/A71G, K9N/E10A/V11W/H18F/T28H/M47L/T62E/R73D, K9R/E10A/E35G/V68T/T101K, K9R/E10A/E35G/V68L/L85E, K9N/E10G/Y87K/T101Q, K9N/V11W/M47L/V68T/R73T/Y87N, K9R/A26T/T28Y/E35A/M47L/A71G.

In some embodiments, the variant CD80 polypeptide includes at least one amino acid substitution at position 10. In some embodiments, the amino acid substitution at position 10 is E10G, E10S, E10R or E10A. In some embodiments, the substituted amino acid at position 10 is a nonpolar amino acid with an aliphatic group. In some embodiments, the nonpolar amino acid is glycine (Gly, G), alanine (Ala, A), Valine (Val, V), leucine (Leu, L), methionine (Met, M) or isoleucine (Ile, I). In some embodiments, the substituted amino acid at position 10 is glycine (Gly, G). In some embodiments, the amino acid substitution is E10G. In some embodiments, the substituted amino acid at position 10 is alanine (Ala, A). In some embodiments, the amino acid substitution is E10A.

In some embodiments, the variant CD80 extracellular domain polypeptide comprises the amino acid substitutions K9N/E10G/N63S/L85E, E10G/E35G/D46E/M47V/V68M, K9N/E10G/E35G/D46E/M47V/V68M, K9R/E10S/V11Y/M42L/F59M/V68M/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, E10G/V11W/T28Y/M47L, K9S/E10R/V11Y/M47L/A71G, E10G/V11W/E35G/M47L/A71G/Y87R, E10G/V11W/M47V/L85E, E10G/H18Y, K9N/E10R/H18V/T28Y/A71G, E10G/H18Y/T28Y/M47W/T62S, K9N/E10A/V11W/H18F/T28H/M47L/T62E/R73D, E10G/D46K/L85E, E10G/H18T/Q27T/D46E/M47L, K9R/E10A/E35G/V68T/T101K, E10G/E35G/D46E/M47V/V68M/T101K, K9R/E10A/E35G/V68L/L85E, K9N/E10G/Y87K/T101Q, E10S/V11Y/M42R/A71V, E10G/A26S/T28Y, E10S/V68M/Y87P, E10G/Q27F/D46N/A71G/D90G, or E10S/V11F/T28Y/M47L.

In some embodiments, the variant CD80 polypeptide includes at least one amino acid substitution at position 11. In some embodiments, the amino acid substitution at position 11 is V11Y, V11F, V11W, or is an amino acid substitution that is a conservative mutation of any of the foregoing. In some embodiments, the substituted amino acid at position 11 is an aromatic amino acid. In some embodiments, the aromatic amino acid is a tyrosine (Tyr, Y), tryptophan (Trp, W), or phenylalanine (Phe, F). In some embodiments, the substituted amino acid at position 11 is a tyrosine (Tyr, Y). In some embodiments, the amino acid substitution is V11Y. In some embodiments, the substituted amino acid at position 11 is a tryptophan (Trp, W). In some embodiments, the amino acid substitution is V11W. In some embodiments, the substituted amino acid at position 11 is a phenylalanine (Phe, F). In some embodiments, the amino acid substitution is V11F.

In some embodiments, the variant CD80 extracellular domain polypeptide comprises the amino acid substitutions V11Y/M47V/F59S/L85E, V11Y/M47V/L85E, V11Y/M42I/M47V/A71G, V11Y/T28Y/M47L/L85E, E10S/V11F/T28Y/M47L/T62S, E10G/V11W/V22S/T28P/A71G/E81K/Y87R, V11Y/T28Y/L85E/Y87I, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E10G/V11W/M47V/L85E, V11Y/T28Y/M47L/V68L/L85E, V11Y/M42W/T62A/L85E, V11Y/M42W/T62A, V11Y/M42W/F59Y/V68N, V11Y/M42W/E52K/T62A/L85E, V11Y/E35D/Y87Q/T101R, K9N/E10A/V11W/H18F/T28H/M47L/T62E/R73D, V11Y/E35G/M42G/F59S, V11Y/T28R/E35G/M47L/F59S, V11Y/T28R/E35G/M47L/A71G, V11Y/V68T, V11W/T28Y/D46V/R73E/F92L, V11W/T28Y/D46V/V68T/R73T/Y87N, E10S/V11Y/M42R/A71V, K9N/V11W/M47L/V68T/R73T/Y87N, V11Y/M47L/V68T, V11W/T28H/D46Q/V68L/L85E, E7Q/V11Y/R29H/M47L/V68T, V11Y/M47V/A71G/L85E, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

In some embodiments, the variant CD80 polypeptide includes at least one amino acid substitution at position 18. In some embodiments, the amino acid substitution at position 18 is H18Y or is an amino acid substitution that is a conservative mutation of any of the foregoing. In some embodiments, the amino acid substitution at position 18 is H18Y.

In some embodiments, the variant CD80 polypeptide includes at least one amino acid substitution at position 28. In some embodiments, the amino acid substitution at position 28 is T28Y, T28P, T28H, T28R, or is an amino acid substitution that is a conservative mutation of any of the foregoing. In some embodiments, the amino acid substitution is T28Y. In some embodiments, the amino acid substitution is T28P. In some embodiments, the substituted amino acid at position 28 is a basic amino acid. In some embodiments, the basic amino acid is lysine (Lys, K), arginine (Arg, R), histidine (His, H). In some embodiments, the amino acid substitution is T28H. In some embodiments, the amino acid substitution is T28R.

In some embodiments, the variant CD80 polypeptide includes at least two amino acid substitutions in which one is at position 11 such as any as described and another is at position 28 such as any as described. In some embodiments, the variant CD80 polypeptide includes at least two amino acid substitutions characterized in that (1) one amino acid substitution is selected from V11Y, V11F, or V11W; and (2) one amino acid substitution is selected from T28Y, T28P, T28H, T28R. In some embodiments, the variant CD80 polypeptide includes the amino acid substitutions V11Y and T28Y.

In some embodiments, the variant CD80 polypeptide includes at least one amino acid substitution at position 101. In some embodiments, the amino acid substitution at position 101 is T101R, T101K, or T101Q, or is an amino acid substitution that is a conservative mutation of any of the foregoing. In some embodiments, the substituted amino acid at position 101 is a polar uncharged amino acid. In some embodiments, the polar uncharged amino acid is serine (Ser, S), asparagine (Asn, N), glutamine (Gln, Q), threonine (Thr, T). In some embodiments, the substituted amino acid at position 101 is a glutamine (Gln, Q). In some embodiments, the amino acid substitution is E7Q. In some embodiments, the substituted amino acid at position 101 is a basic amino acid. In some embodiments, the basic amino acid is lysine (Lys, K), arginine (Arg, R) or histidine (His, H). In some embodiments the substituted amino acid at position 101 is lysine (Lys, K). In some embodiments, the amino acid substitution is T101K. In some embodiments, the substituted amino acid at position 101 is arginine (Arg, R). In some embodiments, the amino acid substitution is T101R.

In some embodiments, the variant CD80 extracellular domain polypeptide comprises the amino acid substitutions E7N/E35D/T101R, V11Y/E35D/Y87Q/T101R, E35D/V68T/T101K, K9R/E10A/E35G/V68T/T101K, E10G/E35G/D46E/M47V/V68M/T101K, or K9N/E10G/Y87K/T101Q.

In some embodiments of any of the provided variant CD80 polypeptides, the variant CD80 extracellular domain polypeptide may further comprise an amino acid substitution at one of more of positions 18, 26, 35, 46, 47, 68, 71, 85, 87 or 903. In some embodiments, the provided variant CD80 extracellular domain may contain an amino acid substitution at one or more positions 7, 9, 10, 11, 28 or 101, such as any described above, and may further comprise one or more amino acid substitution at a position selected from the group consisting of 18, 26, 35, 46, 47, 68, 71, 85, 87 or 90. In some embodiments, the one or more further amino acid substitutions is selected from the group consisting of H18I, H18Y, H18F, H18V, H18L, H18T, A26K, A26G, A26Q, A26E, A26S, A26T, E35G, E35D, E35A, D46E, D46S, D46K, D46V, D46Q, D46N, M47V, M47L, M47R, M47W, V68M, V68L, V68N, V68T, V68S, A71G, A71N, A71V, L85E, L85Q, Y87R, Y87I, Y87K, Y87Q, Y87N, Y87P, or D90G.

In particular embodiments, the variant CD80 extracellular domain polypeptide further comprises an amino acid substitution at position 47. In some embodiments, the amino acid substitution is M47V, M47L, M47R, or M47W, or is an amino acid substitution that is a conservative mutation of any of the foregoing. In some embodiments, the amino acid substitution is M47R. In some embodiments, the amino acid substitution is M47W. In some embodiments, the substituted amino acid at position 47 is non-polar amino acid with an aliphatic group. In some embodiments, the substituted amino acid is glycine (Gly, G), Alanine (Ala, A), Valine (Val, V), Leucine (Leu, L), Methionine (Met, M) or Isoleucine (Ile, I). In some embodiments the substituted amino acid is Leucine (Leu, L). In some embodiments, the amino acid substitution is M47L. In some embodiments, the substituted amino acid is Valine (Val, V). In some embodiments, the amino acid substitution is M47V.

In some embodiments, the variant CD80 polypeptide includes at least two amino acid substitutions in which one is at position 11 such as any as described and another is at position 47 such as any as described. In some embodiments, the variant CD80 polypeptide includes at least two amino acid substitutions characterized in that (1) one amino acid substitution is selected from V11Y, V11F, or V11W; and (2) one amino acid substitution is selected from M47V, M47L, M47R, or M47W. In some embodiments, the variant CD80 polypeptide includes at least two amino acid substitutions characterized in that (1) one amino acid substitution is selected from V11Y, V11F or V11W; and (2) one amino acid substitution is selected from M47V or M47L. In some embodiments, the variant CD80 polypeptide includes the amino acid substitutions V11W and M47L (V11W/M47L). In some embodiments, the variant CD80 polypeptide includes the amino acid substitutions V11W and M47V (V11W/M47V). In some embodiments, the variant CD80 polypeptide includes the amino acid substitutions V11F and M47L (V11F/M47L). In some embodiments, the variant CD80 polypeptide includes the amino acid substitutions V11F and M47V (V11F/M47V). In some embodiments, the variant CD80 polypeptide includes the amino acid substitutions V11Y and M47L (V11Y/M47L). In some embodiments, the variant CD80 polypeptide includes the amino acid substitutions V11Y and M47V (V11Y/M47V).

In some embodiments, the variant CD80 polypeptide includes at least three amino acid substitutions in which one is at position 11 such as any as described, another is at position 28 such as any as described and a further is at position 47 such as any as described. In some embodiments, the variant CD80 polypeptide includes at least three amino acid substitutions characterized in that (1) one amino acid substitution is selected from V11Y, V11F, or V11W; (2) one amino acid substitution is selected from T28Y, T28P, T28H, T28R; and (3) one amino acid substitution is selected from M47V, M47L, M47R, or M47W. In some embodiments, the variant CD80 polypeptide includes at least three amino acid substitutions characterized in that (1) one amino acid substitution is selected from V11Y, V11F, or V11W; (2) one amino acid substitution is T28Y; and (3) one amino acid substitution is selected from M47V or M47L. In some embodiments, the variant CD80 polypeptide includes the amino acid substitutions V11W/T28Y/M47L. In some embodiments, the variant CD80 polypeptide includes the amino acid substitutions V11F/T28Y/M47L. In some embodiments, the variant CD80 polypeptide includes the amino acid substitutions V11Y/T28Y/M47L. In some embodiments, the variant CD80 polypeptide includes the amino acid substitutions V11W/T28Y/M47V. In some embodiments, the variant CD80 polypeptide includes the amino acid substitutions V11F/T28Y/M47V. In some embodiments, the variant CD80 polypeptide includes the amino acid substitutions In some embodiments, the variant CD80 polypeptide includes the amino acid substitutions V11F/T28Y/M47V. In some embodiments, the variant CD80 polypeptide has the amino acid substitutions E10G/V11W/T28Y/M47L. In some embodiments, the variant CD80 polypeptide has the amino acid substitutions V11Y/T28Y/M47L/V68M. In some embodiments, the variant CD80 polypeptide has the amino acid substitutions V11Y/T28Y/M47L/V68L.

In particular embodiments, the variant CD80 extracellular domain polypeptide further comprises an amino acid substitution at position 68. In some embodiments, the amino acid substitution is V68M, V68L, V68N, V68T, V68S, or is an amino acid substitution that is a conservative mutation of any of the foregoing. In some embodiments, the substituted amino acid at position 68 is non-polar amino acid with an aliphatic group. In some embodiments, the substituted amino acid is glycine (Gly, G), Alanine (Ala, A), Leucine (Leu, L), Methionine (Met, M) or Isoleucine (Ile, I). In some embodiments the substituted amino acid is Leucine (Leu, L). In some embodiments, the amino acid substitution is V68L. In some embodiments, the substituted amino acid is Methionine (Met, M). In some embodiments, the amino acid substitution is V68M. In some embodiments, the substituted amino acid at position 68 is a polar uncharged amino acid. In some embodiments, the polar uncharged amino acid is Serine (Ser, S), Threonine (Thr, T), Cysteine (Cys, C), Proline (Pro, P), Asparagine (Asn, N) or Glutamine (Gln, Q). In some embodiments the substituted amino acid is Asparagine (Asn, N). In some embodiments, the amino acid substitution is V68N. In some embodiments the substituted amino acid is Threonine (Thr, T). In some embodiments, the amino acid substitution is V68T. In some embodiments the substituted amino acid is Serine (Ser, S). In some embodiments, the amino acid substitution is V68S.

In some embodiments, the variant CD80 polypeptide includes at least three amino acid substitutions in which one is at position 11 such as any as described, another is at position 47 such as any as described and a further is at position 68 such as any as described. In some embodiments, the variant CD80 polypeptide includes at least three amino acid substitutions characterized in that (1) one amino acid substitution is selected from V11Y, V11F, or V11W; (2) one amino acid substitution is selected from M47V, M47L, M47R, or M47W; and (2) one amino acid substitution is selected from V68M, V68L, V68N, V68T, V68S. In some embodiments, the variant CD80 polypeptide includes at least three amino acid substitutions characterized in that (1) one amino acid substitution is selected from V11Y, V11F or V11W; (2) one amino acid substitution is selected from M47V or M47L; and (3) one amino acid substitution is V68M or V68L. In some embodiments, the variant CD80 polypeptide includes the amino acid substitutions V11Y/M47L/V68M. In some embodiments, the variant CD80 polypeptide includes at least four amino acid substitutions in which one is at position 11 such as any as described, another is at position 28 such as any as described, an additional is at position 47 such as any as described and a further is at position 68 such as any as described. In some embodiments, the variant CD80 polypeptide includes at least four amino acid substitutions characterized in that (1) one amino acid substitution is selected from VIlY, V11F, or V11W; (2) one amino acid substitution is selected from T28Y, T28P, T28H, T28R; (3) one amino acid substitution is selected from M47V, M47L, M47R, or M47W; and (4) one amino acid substitution is selected from V68M, V68L, V68N, V68T, V68S. In some embodiments, the variant CD80 polypeptide includes at least four amino acid substitutions characterized in that (1) one amino acid substitution is selected from V11Y, V11F, or V11W; (2) one amino acid substitution is T28Y; (3) one amino acid substitution is selected from M47V or M47L; and (4) one amino acid substitution is V68M or V68L. In some embodiments, the variant CD80 polypeptide includes the amino acid substitutions V11Y/T28Y/M47L/V68M. In some embodiments, the variant CD80 polypeptide includes the amino acid substitutions V11Y/T28Y/M47L/V68L.

In some embodiments, the variant extracellular domain polypeptide comprises amino acid substitutions selected from the group consisting of V11Y/M47V/F59S/L85E, V11Y/M47V/L85E, V11Y/M42I/M47V/A71G, K9N/E10G/N63S/L85E, E10G/E35G/D46E/M47V/V68M, K9N/E10G/E35G/D46E/M47V/V68M, E7S/H18I/V20L/A26K/M47L/A71N, K9R/E10S/V11Y/M42L/F59M/V68M/L85E, V11Y/T28Y/M47L/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, V11Y/T28Y/L85E/Y87I, H18F/M42G/F59Y/V68N, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E35D/V68L/L85E, E10G/V11W/M47V/L85E, T28Y/M47L, V11Y/T28Y/M47L/V68L/L85E, E7N/E35D/T101R, V11Y/M42W/T62A/L85E, V11Y/M42W/T62A, V11Y/M42W/F59Y/V68N, V11Y/M42W/E52K/T62A/L85E, V11Y/E35D/Y87Q/T101R, H18Y/A26E/R29S/E35D/M47L/V68M/A71G/E77G/D90G, E10G/H18Y, K9N/E10R/H18V/T28Y/A71G, E10G/H18Y/T28Y/M47W/T62S, E7H/H18L/V20I/T28Y/D46S/A71G, K9N/E10A/V11W/H18F/T28H/M47L/T62E/R73D, H18V/V20I/T28Y/E35G/M47V/R73E, E10G/D46K/L85E, E10G/H18T/Q27T/D46E/M47L, E35D/V68T/T101K, V11Y/E35G/M42G/F59S, V11Y/T28R/E35G/M47L/F59S, E7N/E35D/F59S, V11Y/T28R/E35G/M47L/A71G, K9R/E10A/E35G/V68T/T101K, V11Y/V68T, E10G/E35G/D46E/M47V/V68M/T101K, K9R/E10A/E35G/V68L/L85E, V11W/T28Y/D46V/R73E/F92L, K9N/E10G/Y87K/T101Q, V11W/T28Y/D46V/V68T/R73T/Y87N, E10S/V11Y/M42R/A71V, H18F/T28V/M47L/V68S, E10G/A26S/T28Y, E35D/D46Q/L85E, E10S/V68M/Y87P, K9N/V11W/M47L/V68T/R73T/Y87N, V11Y/M47L/V68T, K9R/A26T/T28Y/E35A/M47L/A71G, V11W/T28H/D46Q/V68L/L85E, E7Q/V11Y/R29H/M47L/V68T, V11Y/M47V/A71G/L85E, E10G/Q27F/D46N/A71G/D90G, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, and V11Y/T28Y/M47L/Y87R.

In some of any of the provided embodiments, the variant CD80 polypeptide contains the combination of amino acid substitutions listed in Table E2.A and E2.B. Table E2.A and Table E2.B also provides exemplary sequences by reference to SEQ ID NO of the reference (e.g., unmodified) CD80 polypeptide, and exemplary variant CD80 polypeptides. As indicated, the exact locus or residues corresponding to a given domain can vary, such as depending on the methods used to identify or classify the domain. Also, in some cases, adjacent N- and/or C-terminal amino acids of a given domain (e.g. IgV) also can be included in a sequence of a variant CD80 polypeptide, such as to ensure proper folding of the domain when expressed. Thus, it is understood that the exemplification of the SEQ ID NOs in Table E2.A and Table E2.B is not to be construed as limiting. For example, the particular domain, such as the ECD domain or a portion thereof containing the IgV only, of a variant CD80 polypeptide can be several amino acids longer or shorter, such as 1-10, e.g., 1, 2, 3, 4, 5, 6 or 7 amino acids longer or shorter, than the sequence of amino acids set forth in the respective SEQ ID NO.

In some of any of the provided embodiments, the amino acid substitution(s) is in an unmodified CD80 polypeptide set forth in SEQ ID NO:2.

In some of any of the provided embodiments, the amino acid substitution(s) is in an unmodified CD80 polypeptide set forth in SEQ ID NO:163.

In some of any of the provided embodiments, the amino acid substitution(s) is in an unmodified CD80 polypeptide set forth in SEQ ID NO:164.

In some embodiments, the variant CD80 polypeptide is a variant extracellular domain (ECD) domain polypeptide containing an IgV domain with amino acid substitutions as described, such as a variant CD80 polypeptide comprising the sequence set forth in any one of SEQ ID NOS: 165-244. In some embodiments, the variant CD80 polypeptide comprises a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 165-244. It is understood that reference herein to sequence identity with reference to a variant CD80 polypeptide sequence refers to a sequence that retains the amino acid substitution(s) present in the referenced SEQ ID NO of the variant CD80 polypeptide.

In some embodiments, the variant CD80 polypeptide is a variant extracellular domain (ECD) domain polypeptide that is a portion of the ECD that contains the IgV domain comprising the sequence set forth in any one of SEQ ID NOS: 165-244 but lacks an IgC domain of a full-length extracellular domain.

In some embodiments, the variant CD80 polypeptide consists or consists essentially of the sequence set forth in any one of SEQ ID NOS: 165-244. In some embodiments, the variant CD80 polypeptide consists or consists essentially of a polypeptide sequence that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 165-244.

In some embodiments, the variant CD80 polypeptide has the sequence of amino acids set forth in any one of SEQ ID NOS: 165-244.

In some embodiments, the variant CD80 polypeptide comprises the sequence set forth in SEQ ID NO:180. In some embodiments, the variant CD80 polypeptide consists essentially of the sequence set forth in SEQ ID NO:180. In some embodiments, the variant CD80 polypeptide consists of the sequence set forth in SEQ ID NO:180. In some embodiments, the variant CD80 polypeptide has the sequence of amino acids set forth in SEQ ID NO: 180.

In some embodiments, the variant CD80 polypeptide comprises the sequence set forth in SEQ ID NO:185. In some embodiments, the variant CD80 polypeptide consists essentially of the sequence set forth in SEQ ID NO:185. In some embodiments, the variant CD80 polypeptide consists of the sequence set forth in SEQ ID NO:185. In some embodiments, the variant CD80 polypeptide has the sequence of amino acids set forth in SEQ ID NO: 185.

In some embodiments, the variant CD80 polypeptide comprises the sequence set forth in SEQ ID NO:197. In some embodiments, the variant CD80 polypeptide consists essentially of the sequence set forth in SEQ ID NO:197. In some embodiments, the variant CD80 polypeptide consists of the sequence set forth in SEQ ID NO:197. In some embodiments, the variant CD80 polypeptide has the sequence of amino acids set forth in SEQ ID NO: 197.

In some embodiments, the variant CD80 polypeptide comprises the sequence set forth in SEQ ID NO:233. In some embodiments, the variant CD80 polypeptide consists essentially of the sequence set forth in SEQ ID NO:233. In some embodiments, the variant CD80 polypeptide consists of the sequence set forth in SEQ ID NO:233. In some embodiments, the variant CD80 polypeptide has the sequence of amino acids set forth in SEQ ID NO: 233.

In some embodiments, the variant CD80 polypeptide comprises the sequence set forth in SEQ ID NO:234. In some embodiments, the variant CD80 polypeptide consists essentially of the sequence set forth in SEQ ID NO:234. In some embodiments, the variant CD80 polypeptide consists of the sequence set forth in SEQ ID NO:234. In some embodiments, the variant CD80 polypeptide has the sequence of amino acids set forth in SEQ ID NO: 234.

In some embodiments, the variant CD80 polypeptide comprises the sequence set forth in SEQ ID NO:415. In some embodiments, the variant CD80 polypeptide consists essentially of the sequence set forth in SEQ ID NO:415. In some embodiments, the variant CD80 polypeptide consists of the sequence set forth in SEQ ID NO:415. In some embodiments, the variant CD80 polypeptide has the sequence of amino acids set forth in SEQ ID NO: 415.

In some embodiments, the variant CD80 polypeptide comprises the sequence set forth in SEQ ID NO:416. In some embodiments, the variant CD80 polypeptide consists essentially of the sequence set forth in SEQ ID NO:416. In some embodiments, the variant CD80 polypeptide consists of the sequence set forth in SEQ ID NO:416. In some embodiments, the variant CD80 polypeptide has the sequence of amino acids set forth in SEQ ID NO:416.

In some embodiments, the variant CD80 polypeptide comprises the sequence set forth in SEQ ID NO:417. In some embodiments, the variant CD80 polypeptide consists essentially of the sequence set forth in SEQ ID NO:417. In some embodiments, the variant CD80 polypeptide consists of the sequence set forth in SEQ ID NO:417. In some embodiments, the variant CD80 polypeptide has the sequence of amino acids set forth in SEQ ID NO:417.

In some embodiments, the variant CD80 polypeptide is encoded by a sequence of nucleotides set forth in any one of SEQ ID NOS: 3-82. In some embodiments, the variant CD80 polypeptide is encoded by a sequence of nucleotides that exhibits at least about 90% identity, at least about 91% identity, at least about 92% identity, at least about 93% identity, at least about 94% identity, at least about 95% identity, such as at least about 96% identity, 97% identity, 98% identity, or 99% identity to any one of SEQ ID NOS: 3-82. Also provided herein is a nucleic acid containing the sequence set forth in any of SEQ ID NOS: 3-82 or a sequence that exhibits at least 90% identity, at least 91% identity, at least 92% identity, at least 93% identity, at least 94% identity, at least 95% identity, such as at least 96% identity, 97% identity, 98% identity, or 99% identity to any one of SEQ ID NOS: 3-82. In some embodiments, also provided herein is a nucleic acid sequence set forth in any of SEQ ID NOS: 3-82 or a sequence that exhibits at least 90% identity, at least 91% identity, at least 92% identity, at least 93% identity, at least 94% identity, at least 95% identity, such as at least 96% identity, 97% identity, 98% identity, or 99% identity to any one of SEQ ID NOS: 3-82. In some embodiments, also provided herein is a nucleic acid sequence set forth in any of SEQ ID NOS: 3-82

In some embodiments, the variant CD80 polypeptide is encoded by the sequence set forth in SEQ ID NO:23. In some embodiments, provided herein is a nucleic acid sequence containing the sequence set forth in SEQ ID NO:23. In some embodiments, provided herein is a nucleic acid set forth in SEQ ID NO:23.

In some embodiments, the variant CD80 polypeptide is encoded by the sequence set forth in SEQ ID NO:71. In some embodiments, provided herein is a nucleic acid sequence containing the sequence set forth in SEQ ID NO:71. In some embodiments, provided herein is a nucleic acid set forth in SEQ ID NO:71.

In some embodiments, the variant CD80 polypeptide is encoded by the sequence set forth in SEQ ID NO:72. In some embodiments, provided herein is a nucleic acid sequence containing the sequence set forth in SEQ ID NO:72. In some embodiments, provided herein is a nucleic acid set forth in SEQ ID NO:72.

### B. Fusion Proteins

In some embodiments, also provided herein are variant CD80 fusion proteins in which any of the above variant CD80 polypeptides are linked or fused to at least one other protein molecule. In some embodiments, the other protein molecule may be a multimerization domain, a half-life extending moiety or a targeting moiety.

In some embodiments, the variant CD80 polypeptide is linked directly to the other protein. In some embodiments, the variant CD80 polypeptide is linked indirectly to the other protein via a linker. In some embodiments, the linker is a peptide linker. In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length. In some embodiments, the linker is (in one-letter amino acid code): GGGGS (“4GS”; SEQ ID NO: 328) or multimers of the 4GS linker, such as repeats of 2, 3, 4, or 5 4GS linkers. In some embodiments, the peptide linker is the peptide linker is (GGGGS)₂ (SEQ ID NO: 329), (GGGGS)₃ (SEQ ID NO: 330), (GGGGS)₄ (SEQ ID NO: 331) or (GGGGS)₅(SEQ ID NO: 332). In some embodiments, the linker also can include a series of alanine residues alone or in addition to another peptide linker (such as a 4GS linker or multimer thereof). In some embodiments, the linker (in one-letter amino acid code) is GSGGGGS (SEQ ID NO: 325) or GGGGSSA (SEQ ID NO: 333). In some embodiments, the linker is GS(G4S)₂ (SEQ ID NO: 335). In some examples, the linker is a 2×GGGGS followed by three alanines (GGGGSGGGGSAAA; SEQ ID NO:334). In some examples, the linker is set forth in SEQ ID NO: 335.

Exemplary fusion proteins are provided in the following sections.

1. Multimeric Fusion Proteins (e.g. Fc Fusions)

In some embodiments, also provided herein are variant CD80 fusion sequences in which any of the above variant CD80 extracellular domain sequence is linked or fused to a multimerization domain, such as any described herein.

Interaction of two or more polypeptides of the immunomodulatory proteins can be facilitated by their linkage, either directly or indirectly, to any moiety or other polypeptide that are themselves able to interact to form a stable structure. For example, separate encoded polypeptide chains can be joined by multimerization, whereby multimerization of the polypeptides is mediated by a multimerization domain. Typically, the multimerization domain provides for the formation of a stable protein-protein interaction between a first polypeptide and a second polypeptide.

In some embodiments, the two or more individual polypeptides of the immunomodulatory proteins can be joined by multimerization, such as joined as dimeric, trimeric, tetrameric, or pentameric molecules. In some cases, the individual polypeptides are the same. For example, a trimeric molecule can be formed from three copies of the same individual polypeptide. In other examples, a tetrameric molecule is generated from four copies of the same individual polypeptides. In further examples, a pentameric molecule is generated from five copies of the same individual polypeptides. The multimerization domain may be one that facilities dimerization, trimerization, tetramerization, or pentamerization of the polypeptide chains.

In some embodiments, the immunomodulatory protein forms a multimer, e.g., a dimer. In some embodiments, the dimer is a homodimer in which the two polypeptides of the immunomodulatory protein are the same. In some embodiments, the dimer is a heterodimer in which the two polypeptides of the immunomodulatory protein are different.

In some embodiments, a multimerization domain includes any capable of forming a stable protein-protein interaction. The multimerization domains can interact via an immunoglobulin sequence (e.g. Fc domain; see e.g., International Patent Pub. Nos. WO 93/10151 and WO 2005/063816 US; U.S. Pub. No. 2006/0024298; U.S. Pat. No. 5,457,035); leucine zipper (e.g. from nuclear transforming proteins fos and jun or the proto-oncogene c-myc or from General Control of Nitrogen (GCN4)) (ee e.g., Busch and Sassone-Corsi (1990) Trends Genetics, 6:36-40; Gentz et al., (1989) Science, 243:1695-1699); a hydrophobic region; a hydrophilic region; or a free thiol which forms an intermolecular disulfide bond between the chimeric molecules of a homo- or heteromultimer. In addition, a multimerization domain can include an amino acid sequence comprising a protuberance complementary to an amino acid sequence comprising a hole, such as is described, for example, in U.S. Pat. No. 5,731,168; International Patent Pub. Nos. WO 98/50431 and WO 2005/063816; Ridgway et al. (1996) Protein Engineering, 9:617-621. Such a multimerization region can be engineered such that steric interactions not only promote stable interaction, but further promote the formation of heterodimers over homodimers from a mixture of chimeric monomers. Generally, protuberances are constructed by replacing small amino acid side chains from the interface of the first polypeptide with larger side chains (e.g., tyrosine or tryptophan). Compensatory cavities of identical or similar size to the protuberances are optionally created on the interface of the second polypeptide by replacing large amino acid side chains with smaller ones (e.g., alanine or threonine). Exemplary multimerization domains are described below.

The variant CD80 polypeptide sequence can be joined anywhere, but typically via its N- or C-terminus, to the N- or C-terminus of a multimerization domain to form a chimeric polypeptide. The linkage can be direct or indirect via a linker. Also, the chimeric polypeptide can be a fusion protein or can be formed by chemical linkage, such as through covalent or non-covalent interactions. For example, when preparing a chimeric polypeptide containing a multimerization domain, nucleic acid encoding all or part of a variant CD80 polypeptide sequence can be operably linked to nucleic acid encoding the multimerization domain sequence, directly or indirectly or optionally via a linker domain. In some cases, the construct encodes a chimeric protein where the C-terminus of the variant CD80 polypeptide sequence is joined to the N-terminus of the multimerization domain. In some instances, a construct can encode a chimeric protein where the N-terminus of the variant CD80 polypeptide sequence is joined to the N- or C-terminus of the multimerization domain.

A polypeptide multimer contains two chimeric proteins created by linking, directly or indirectly, two of the same or different variant CD80 polypeptide sequences directly or indirectly to a multimerization domain. In some examples, where the multimerization domain is a polypeptide, a gene fusion encoding the CD80 polypeptide sequence and multimerization domain is inserted into an appropriate expression vector. The resulting chimeric or fusion protein can be expressed in host cells transformed with the recombinant expression vector, and allowed to assemble into multimers, where the multimerization domains interact to form multivalent polypeptides. Chemical linkage of multimerization domains to the variant CD80 polypeptide can be effected using heterobifunctional linkers.

The resulting chimeric polypeptides, such as fusion proteins, and multimers formed therefrom, can be purified by any suitable method such as, for example, by affinity chromatography over Protein A or Protein G columns. Where two nucleic acid molecules encoding different polypeptides are transformed into cells, formation of homo- and heterodimers will occur. Conditions for expression can be adjusted so that heterodimer formation is favored over homodimer formation.

In some embodiments, the multimerization domain is an Fc region of an immunoglobulin.

In some embodiments, the multimerization domain is an immunoglobulin Fc region, in which the fusion protein is a variant CD80-Fc composed of (1) a variant CD80 sequence containing or consisting of any of the provided variant CD80 polypeptide sequences; and (2) an immunoglobulin Fc region. Thus, among provided embodiments are variant CD80-Fc fusion proteins composed of (1) a variant CD80-Fc sequence containing or consisting of any of the above described variant CD80 polypeptide sequences; and (2) an immunoglobulin Fc region.

In some embodiments, the multimerization domain is an immunoglobulin Fc region, in which the fusion protein is a variant CD80-Fc composed of (1) a variant CD80 sequence containing or consisting of the sequence set forth in any one of SEQ ID NOS: 165-244; and (2) an immunoglobulin Fc region. Thus, among provided embodiments are variant CD80-Fc fusion proteins composed of (1) a variant CD80-Fc sequence containing or consisting of the sequence set forth in any one of SEQ ID NOS: 165-244; and (2) an immunoglobulin Fc region. In some embodiments, provided is a variant CD80-Fc fusion protein composed of (1) a variant CD80 sequence set forth in any one of SEQ ID NOS: 165-244; and (2) an immunoglobulin Fc region.

In some embodiments, the variant CD80 polypeptide of the Fc fusion protein comprises the sequence set forth in SEQ ID NO:180. In some embodiments, the variant CD80 polypeptide of the Fc fusion protein consists essentially of the sequence set forth in SEQ ID NO:180. In some embodiments, the variant CD80 polypeptide of the Fc fusion protein consists of the sequence set forth in SEQ ID NO:180. In some embodiments, the variant CD80 polypeptide of the Fc fusion protein is set forth in SEQ ID NO: 180.

In some embodiments, the variant CD80 polypeptide of the Fc fusion protein comprises the sequence set forth in SEQ ID NO:185. In some embodiments, the variant CD80 polypeptide of the Fc fusion protein consists essentially of the sequence set forth in SEQ ID NO:185. In some embodiments, the variant CD80 polypeptide of the Fc fusion protein consists of the sequence set forth in SEQ ID NO:185. In some embodiments, the variant CD80 polypeptide of the Fc fusion protein is set forth in SEQ ID NO: 185.

In some embodiments, the variant CD80 polypeptide of the Fc fusion protein comprises the sequence set forth in SEQ ID NO:197. In some embodiments, the variant CD80 polypeptide of the Fc fusion protein consists essentially of the sequence set forth in SEQ ID NO:197. In some embodiments, the variant CD80 polypeptide of the Fc fusion protein consists of the sequence set forth in SEQ ID NO:197. In some embodiments, the variant CD80 polypeptide of the Fc fusion protein is set forth in SEQ ID NO: 197.

In some embodiments, the variant CD80 polypeptide of the Fc fusion protein comprises the sequence set forth in SEQ ID NO:233. In some embodiments, the variant CD80 polypeptide of the Fc fusion protein consists essentially of the sequence set forth in SEQ ID NO:233. In some embodiments, the variant CD80 polypeptide of the Fc fusion protein consists of the sequence set forth in SEQ ID NO:233. In some embodiments, the variant CD80 polypeptide of the Fc fusion protein is set forth in SEQ ID NO: 233.

In some embodiments, the variant CD80 polypeptide comprises the sequence set forth in SEQ ID NO:234. In some embodiments, the variant CD80 polypeptide consists essentially of the sequence set forth in SEQ ID NO:234. In some embodiments, the variant CD80 polypeptide consists of the sequence set forth in SEQ ID NO:234. In some embodiments, the variant CD80 polypeptide is set forth in SEQ ID NO: 234.

In provided embodiments of a variant CD80-Fc, the immunoglobulin Fc region can be a wild-type Fc of an immunoglobulin, such as an IgG1 Fc. In some cases, the Fc region can be a variant Fc that lacks effector function (also called “effectorless Fc”). Exemplary Fc regions and variants thereof in provided variant CD80-Fc fusion proteins are described below.

In some embodiments, the Fc is murine or human Fc. In some embodiments, the Fc is a mammalian or human IgG1, IgG2, IgG3, or IgG4 Fc regions.

In some embodiments, the Fc region is or comprises the sequence set forth in any one of SEQ ID NOs: 326, 343, 344, 345, 346, 347, 348, 349, 350, 351, 352, 353, 354, 355, 356, 357, 358, 359, 360, 361 or 362. In some embodiments, the Fc region is or is derived from an IgG1, such as set forth in any one of SEQ ID NOS: 343, 344, 345, 346, 347, 348, 349, 350, 351, 352, 356, 357, 358, 359, 360, or 362. In some embodiments, the Fc region is or is derived from an IgG2, such as any set forth in SEQ ID NO: 353 or 361. In some embodiments, the Fc region is or is derived from an IgG4, such as any set forth in SEQ ID NO: 326, 354, or 355. In some embodiments, an Fc region in Fc fusion proteins provided herein also can include an Fc region that exhibits at least about 85%, 86%, 87%, 88%, 89%, 90%, 91%, 92%, 93%, 94%, 95%, 96%, 97%, 98%, 99% to any of the above Fc regions.

In some embodiments, the Fc is derived from IgG1, such as human IgG1. In some embodiments, the Fc is an IgG1 Fc set forth in SEQ ID NO: 343 having an allotype containing residues Glu (E) and Met (M) at positions 356 and 358 by EU numbering. In some embodiments, the Fc comprises the amino acid sequence set forth in SEQ ID NO: 343 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 343. In other embodiments, the Fc is an IgG1 Fc that contains amino acids of the human G1 ml allotype, such as residues containing Asp (D) and Leu (L) at positions 356 and 358, e.g. as set forth in SEQ ID NO:346. Thus, in some cases, an Fc provided herein can contain amino acid substitutions E356D and M358L to reconstitute residues of allotype G1 m1. In some embodiments, the Fc comprises the amino acid sequence set forth in SEQ ID NO: 346 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 346.

In some embodiments, the Fc region has the amino acid sequence set forth in SEQ ID NO:346. EPKSSDKTHTCPPCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPAPIEKTISKA KGQPREPQVYTLPPSRDELTKNQVSLTCLVKGFYPSDIAVEWESNGQPENNYKTTPPVLDSD GSFFLYSKLTVDKSRWQQGNVFSCSVMHEALHNHYTQKSLSLSPG (SEQ ID NO:346)

In some embodiments, the Fc comprises the sequence set forth in SEQ ID NO: 356. In some embodiments, the Fc comprises the sequence set forth in SEQ ID NO:357. In some embodiments, an Fc region used in a construct provided herein can further lack a C-terminal lysine residue.

In some embodiments, the Fc is derived from IgG2, such as human IgG2. In some embodiments, the Fc comprises the amino acid sequence set forth in SEQ ID NO: 353 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 353. In some embodiments, the Fc region is an IgG2 Fc region that has the sequence set forth in SEQ ID NO: 353. In some embodiments, the Fc comprises the amino acid sequence set forth in SEQ ID NO: 361 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 361. In some embodiments, the Fc region is an IgG2 Fc region that has the sequence set forth in SEQ ID NO: 361.

In some embodiments, the Fc is derived from IgG4, such as human IgG4. In some embodiments, a CD80-Fc with an IgG4 Fc may exhibit FcR-dependent CD28 costimulation via IgG4 Fc. Thus, the provided CD80-Fc containing an IgG4 Fc may exhibit both PD-L1-dependent CD28 costimulation and FcR-dependent CD29 costimulation. In some embodiments, the FcR-dependent CD28 costimulation of such CD80-Fc fusion proteins can increase CD28 costimulation in tumors even with low or no PD-L1 expression, thereby increasing the pool of potential responder subjects.

In some embodiments, the IgG4 Fc comprises the amino acid sequence set forth in SEQ ID NO: 354 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 354. In some embodiments, the IgG4 Fc is a stabilized Fc in which the CH3 domain of human IgG4 is substituted with the CH3 domain of human IgG1 and which exhibits inhibited aggregate formation, an antibody in which the CH3 and CH2 domains of human IgG4 are substituted with the CH3 and CH2 domains of human IgG1, respectively, or an antibody in which arginine at position 409 indicated in the EU index proposed by Kabat et al. of human IgG4 is substituted with lysine and which exhibits inhibited aggregate formation (see e.g. U.S. Pat. No. 8,911,726). In some embodiments, the Fc is an IgG4 containing the S228P mutation, which has been shown to prevent recombination between a therapeutic antibody and an endogenous IgG4 by Fab-arm exchange (see e.g. Labrijin et al. (2009) Nat. Biotechnol., 27(8): 767-71.) In some embodiments, the IgG4 Fc comprises the amino acid sequence set forth in SEQ ID NO: 355 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 355. In some embodiments, the Fc region is an IgG4 Fc region set forth in SEQ ID NO:355. In some embodiments, the IgG4 Fc comprises the amino acid sequence set forth in SEQ ID NO: 326 or a sequence of amino acids that exhibits at least about 85%, 86%, 87%, 88%, 89%, 90%, 91%, 92%, 93%, 94%, 95%, 96%, 97%, 98%, 99% or more sequence identity to SEQ ID NO: 326.

In some embodiments, the Fc region is an IgG4 Fc region set forth in SEQ ID NO:326. ESKYGPPCPPCPAPEFLGGPSVFLFPPKPKDTLMISRTPEVTCVVVDVSQEDPEVQFNWYVDG VEVHNAKTKPREEQFNSTYRVVSVLTVLHQDWLNGKEYKCKVSNKGLPSSIEKTISKAKGQP REPQVYTLPPSQEEMTKNQVSLTCLVKGFYPSDIAVEWESNGQPENNYKTTPPVLDSDGSFFL YSRLTVDKSRWQEGNVFSCSVMHEALHNHYTQKSLSLSLG (SEQ ID NO:326)

In some embodiments, the Fc region is a variant Fc region in which a wild-type Fc is modified by one or more amino acid substitutions to reduce effector activity or to render the Fc inert for Fc effector function. Exemplary effectorless or inert mutations include those described herein.

In some embodiments, the Fc region contains one or more modifications that alter (e.g. reduce) one or more of its normal functions. In general, the Fc region is responsible for effector functions, such as complement-dependent cytotoxicity (CDC) and antibody-dependent cell cytotoxicity (ADCC), in addition to the antigen-binding capacity, which is the main function of immunoglobulins. Additionally, the FcRn sequence present in the Fc region plays the role of regulating the IgG level in serum by increasing the in vivo half-life by conjugation to an in vivo FcRn receptor. In some embodiments, such functions can be reduced or altered in an Fc for use with the provided Fc fusion proteins.

In some embodiments, one or more amino acid modifications may be introduced into the Fc region, thereby generating an Fc region variant. In some embodiments, the Fc region variant has decreased effector function. There are many examples of changes or mutations to Fc sequences that can alter effector function. For example, WO 00/42072, WO2006019447, WO2012125850, WO2015/107026, US2016/0017041 and Shields et al. J Biol. Chem. 9(2): 6591-6604 (2001) describe exemplary Fc variants with improved or diminished binding to FcRs. The contents of those publications are specifically incorporated herein by reference.

In some embodiments, the provided immunomodulatory proteins comprise an Fc region that exhibits reduced effector functions, which makes it a desirable candidate for applications in which the half-life of the immunomodulatory protein in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the immunomodulatory protein lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the immunomodulatory protein is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Immunomodulatory proteins with reduced effector function include those with substitution of one or more of Fc region residues 238, 265, 269, 270, 297, 327 and 329 by EU numbering (U.S. Pat. No. 6,737,056). Such Fc mutants include Fc mutants with substitutions at two or more of amino acid positions 265, 269, 270, 297 and 327 by EU numbering, including the so-called “DANA” Fc mutant with substitution of residues 265 and 297 to alanine (U.S. Pat. No. 7,332,581).

In some embodiments, the Fc region of immunomodulatory proteins has an Fc region in which any one or more of amino acids at positions 234, 235, 236, 237, 238, 239, 270, 297, 298, 325, and 329 (indicated by EU numbering) are substituted with different amino acids compared to the native Fc region. Such alterations of Fc region include, for example, alterations such as deglycosylated chains (N297A and N297Q), IgG1-N297G, IgG1-L234A/L235A, IgG1-L234A/L235E/G237A, IgG1-A325A/A330S/P331S, IgG1-C226S/C229S, IgG1-C226S/C229S/E233P/L234V/L235A, IgG1-E233P/L234V/L235A/G236del/S267K, IgG1-L234F/L235E/P331S, IgG1-S267E/L328F, IgG2-V234A/G237A, IgG2-H268Q/V309L/A330S/A331S, IgG4-L235A/G237A/E318A, and IgG4-L236E described in Current Opinion in Biotechnology (2009) 20 (6), 685-691; alterations such as G236R/L328R, L235G/G236R, N325A/L328R, and N325LL328R described in WO 2008/092117; amino acid insertions at positions 233, 234, 235, and 237 (indicated by EU numbering); and alterations at the sites described in WO 2000/042072.

Certain Fc variants with improved or diminished binding to FcRs are described. (See, e.g., U.S. Pat. No. 6,737,056; WO 2004/056312, WO2006019447 and Shields et al., J. Biol. Chem. 9(2): 6591-6604 (2001).)

In some embodiments, there is provided an immunomodulatory protein comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.) or WO2015107026. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434 by EU numbering, e.g., substitution of Fc region residue 434 (U.S. Pat. No. 7,371,826).

In some embodiments, the Fc region of the immunomodulatory protein comprises one or more amino acid substitutions C220S, C226S and/or C229S by EU numbering. In some embodiments, the Fc region of the immunomodulatory protein comprises one or more amino acid substitutions R292C and V302C. See also Duncan & Winter, Nature 322:738-40 (1988); U.S. Pat. Nos. 5,648,260; 5,624,821; and WO 94/29351 concerning other examples of Fc region variants.

In some embodiments, alterations are made in the Fc region that result in diminished C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, the variant Fc region comprising the one or more amino acid modifications (e.g amino acid substitutions) is derived from a wild-type IgG1, such as a wild-type human IgG1. In some embodiments, the wild-type IgG1 Fc can be the Fc set forth in SEQ ID NO: 343 having an allotype containing residues Glu (E) and Met (M) at positions 356 and 358 by EU numbering. In some embodiments, the variant Fc region is derived from the amino acid sequence set forth in SEQ ID NO: 343. In other embodiments, the wild-type IgG1 Fc contains amino acids of the human G1 m1 allotype, such as residues containing Asp (D) and Leu (L) at positions 356 and 358, e.g. as set forth in SEQ ID NO:346. Thus, in some cases, the variant Fc is derived from the amino acid sequence set forth in SEQ ID NO:346.

In some embodiments, the Fc region lacks the C-terminal lysine corresponding to position 232 of the wild-type or unmodified Fc set forth in SEQ ID NO: 343 or 346 (corresponding to K447del by EU numbering).

In some embodiments, the variant Fc region comprises a C5S amino acid modification of the wild-type or unmodified Fc region by numbering of SEQ ID NO:343 (corresponding to C220S by EU numbering).

In some embodiments, the Fc region is a variant Fc that contains at least one amino acid substitution that is N82G by numbering of SEQ ID NO: 343 (corresponding to N297G by EU numbering). In some embodiments, the Fc further contains at least one amino acid substitution that is R77C or V87C by numbering of SEQ ID NO: 343 (corresponding to R292C or V302C by EU numbering). In some embodiments, the variant Fc region further comprises a C5S amino acid modification by numbering of SEQ ID NO: 343 (corresponding to C220S by EU numbering). For example, in some embodiments, the variant Fc region comprises the following amino acid modifications: N297G and one or more of the following amino acid modifications C220S, R292C or V302C by EU numbering (corresponding to N82G and one or more of the following amino acid modifications C5S, R77C or V87C with reference to SEQ ID NO:343), e.g., the Fc region comprises the sequence set forth in SEQ ID NO:347.

In some embodiments, the variant Fc contains the amino acid substitutions L234A/L235E/G237A, by EU numbering. In some embodiments, the variant Fc contains the amino acid substitutions A330S/P331S, by EU numbering. In some embodiments, the variant Fc contains the amino acid substitutions L234A/L235E/G237A/A330S/P331S (Gross et al. (2001) Immunity 15:289).

In some embodiments, the variant Fc comprises the sequence set forth in SEQ ID NO: 358. In some embodiments, the variant Fc comprises the sequence set forth in SEQ ID NO:359. In some embodiments, an Fc region used in a construct provided herein can further lack a C-terminal lysine residue.

In some embodiments, the Fc region is a variant Fc that includes mutations L234A, L235E and G237A by EU numbering. In some embodiments, a wild-type Fc is further modified by the removal of one or more cysteine residue, such as by replacement of the cysteine residues to a serine residue at position 220 (C220S) by EU numbering. Exemplary inert Fc regions having reduced effector function are set forth in SEQ ID NO: 348 and SEQ ID NO:345, which are based on allotypes set forth in SEQ ID NO:343 or SEQ ID NO: 346, respectively. In some embodiments, an Fc region can further lack a C-terminal lysine residue. In some embodiments, the variant Fc region comprises one or more of the amino acid modifications C220S, L234A, L235E or G237A, e.g. the Fc region comprises the sequence set forth in SEQ ID NO:344, 345, 348 or 351. In some embodiments, the variant Fc comprises has the sequence set forth in SEQ ID NO: 344. In some embodiments, the variant Fc comprises has the sequence set forth in SEQ ID NO: 345. In some embodiments, the variant Fc comprises has the sequence set forth in SEQ ID NO: 348. In some embodiments, the variant Fc comprises has the sequence set forth in SEQ ID NO: 351.

In some embodiments, the Fc region is a variant Fc that has the sequence set forth in SEQ ID NO:344.

(SEQ ID NO: 73)
EPKSSDKTHTCPPCPAPEAEGAPSVFLFPPKPKDTLMISRTPEVT
CVVVDVSHEDPEVKENWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPG

In some embodiments, the Fc region is an IgG1 Fc but does not contain a hinge sequence. In some embodiments, the IgG1 Fc region does not contain the hinge sequence EPKSC (SEQ ID NO:363). In some embodiments, the IgG1 Fc region does not contain a hinge sequence EPKSS (SEQ ID NO: 364).

In some embodiments, the Fc region is a variant Fc that has the sequence set forth in SEQ ID NO: 362.

(SEQ ID NO: 362)
DKTHTCPPCPAPEAEGAPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
G

In some embodiments, the Fc region is a variant Fc region that comprises one or more of the amino acid modifications C220S, L235P, L234V, L235A, G236del or S267K, e.g. the Fc region comprises the sequence set forth in SEQ ID NO:349. In some embodiments, the Fc region lacks the C-terminal lysine corresponding to position 232 of the wild-type or unmodified Fc set forth in SEQ ID NO: 343 (corresponding to K447del by EU numbering).

In some embodiments, the Fc region is a variant Fc region that comprises one or more of the amino acid modifications C220S, R292C, N297G, V302C. In some embodiments, the Fc region lacks the C-terminal lysine corresponding to position 232 of the wild-type or unmodified Fc set forth in SEQ ID NO: 343 (corresponding to K447del by EU numbering). An exemplary variant Fc region is set forth in SEQ ID NO: 350.

In some embodiments, the variant Fc region comprises one or more of the amino acid modifications C220S/E233P/L234V/L235A/G236del/S267K. In some embodiments, the Fc region lacks the C-terminal lysine corresponding to position 232 of the wild-type or unmodified Fc set forth in SEQ ID NO: 343 (corresponding to K447del by EU numbering). An exemplary variant Fc region is set forth in SEQ ID NO: 352.

Examples of such Fc regions for inclusion in an immunomodulatory polypeptide are set forth in Table 1.

TABLE 1
Exemplary IgG1 Fc Regions, wild-type or variant (effectorless)

| | 356E/358M | 356D/358L |
|---|---|---|
| | allotype | allotype |
| Fc mutations (EU numbering) | SEQ ID NO | SEQ ID NO |
| (wild-type) | 343 | 346 (with |
| | | C220S, |
| | | K447del) |
| C220S, R292C, N297G, V302C | 347 | |
| C220S, R292C, N297G, V302C, K447del | 350 | |
| C220S, L234A, L235E, G237A | 348 | 345 |
| C220S, L234A, L235E, G237A, K447del | 351 | 344 |
| L234A, L235E, G237A, K447del, with | | 362 |
| deletion of hinge | | |
| C220S, L235P, L234V, L235A, G236del, | 349 | |
| S267K | | |
| C220S/E233P/L234V/L235A/G236del/ | 352 | |
| S267K/K447del | | |
| L234A, L235E, G237A, A330S, P331S | | 359 |
| L234A, L235E, G237A, A330S, P331S, | | 358 |
| with deletion of hinge | | |

In some embodiments, the Fc region is a variant Fc region containing any combination of the Fc mutations in Table 1. In some embodiments, the Fc region is a variant Fc region having the sequence set forth in any one of the SEQ ID NOs in Table 1.

For example, a variant Fc region may be an effectorless Fc that exhibits reduced effector activity compared to a wild-type IgG1 set forth in SEQ ID NO:343 or SEQ ID NO:346. In some embodiments, the variant Fc comprises the sequence of amino acids set forth in any of SEQ ID NOS:344, 345, 347, 348, 349, 350, 351, or 352 or a sequence of amino acids that exhibits at least about 85%, 86%, 87%, 88%, 89%, 90%, 91%, 92%, 93%, 94%, 95%, 96%, 97%, 98%, 99% or more sequence identity to any of SEQ ID NOS: 344, 345, 347, 348, 349, 350, 351, or 352. In some embodiments, the variant Fc has the sequence set forth in SEQ ID NO: 344. In embodiments, when produced and expressed from cells, the provided immunomodulatory protein (e.g. variant CD80-Fc fusion) is a homodimer containing two identical polypeptide chains.

In some embodiments, the variant TACI polypeptide is directly linked to the multimerization domain (Fc region). In some embodiments, a variant CD80 polypeptide sequence is joined to the multimerization domain (e.g. Fc region) via a linker, such as a peptide linker. In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length.

In some embodiments, the linker is (in one-letter amino acid code): GGGGS (“4GS”; SEQ ID NO: 328) or multimers of the 4GS linker, such as repeats of 2, 3, 4, or 5 4GS linkers. In some embodiments, the peptide linker is the peptide linker is (GGGGS)₂ (SEQ ID NO: 329), (GGGGS)₃ (SEQ ID NO: 330), (GGGGS)₄ (SEQ ID NO: 331) or (GGGGS)₅(SEQ ID NO: 332). In some embodiments, the linker also can include a series of alanine residues alone or in addition to another peptide linker (such as a 4GS linker or multimer thereof). In some embodiments, the linker (in one-letter amino acid code) is GSGGGGS (SEQ ID NO: 325) or GGGGSSA (SEQ ID NO: 333). In some embodiments, the linker is GS(G4S)₂ (SEQ ID NO: 335). In some examples, the linker is a 2×GGGGS followed by three alanines (GGGGSGGGGSAAA; SEQ ID NO:334). In some examples, the linker is set forth in SEQ ID NO: 335.

In some embodiments, the variant CD80-Fc fusion protein has the structure variant CD80 polypeptide (vCD80)-Linker-Fc region. In some embodiments, the immunomodulatory protein is a homodimer of two identical copies of the variant CD80-Fc fusion protein. For instance, interactions between Fc regions of the two identical polypeptide fusions form covalent disulfide bonds to result in a dimeric molecule containing two identical variant CD80 polypeptides.

In some embodiments, there is provided a variant CD80-Fc fusion protein containing in order a variant CD80 polypeptide, e.g. any as described above, a linker and an Fc region. In some embodiments, the variant CD80 polypeptide of the variant CD80-Fc fusion is a variant CD80 polypeptide, such as any as described. In some embodiments, the variant CD80 polypeptide of the variant CD80-Fc fusion is set forth in any one of SEQ ID NOS: 165-244. The linker may be any as described. In some embodiments, the linker is GSGGGGS (SEQ ID NO: 325). In some embodiments, the linker is GS(G4S)₂ (SEQ ID NO: 335). The Fc region may be any Fc region as described. In some embodiments, the Fc region is a variant IgG1 Fc set forth in SEQ ID NO: 344. In some embodiments, the Fc region is a IgG4 Fc set forth in SEQ ID NO: 326.

In some embodiments, the variant CD80-Fc fusion protein has the sequence of amino acids set forth in any one of SEQ ID NOS: 245-324. In some embodiments, the variant CD80-Fc fusion protein consists or consists essentially of the sequence of amino acids set forth in any one of SEQ ID NOS: 245-324. In some embodiments, the variant CD80-Fc fusion protein is set forth in any one of SEQ ID NOS: 245-324.

In some embodiments, the variant CD80-Fc fusion protein is encoded by a sequence of nucleotides set forth in any one of SEQ ID NOS: 83-162. Also provided herein is a sequence of nucleotides encoding a variant CD80-Fc fusion protein in which the sequence of nucleotides comprises the sequence set forth in any one of SEQ ID NOS: 83-162. In some embodiments, the sequence of nucleotides encoding the variant CD80-Fc fusion protein consists or consists essentially of the sequence of amino acids set forth in any one of SEQ ID NOS: 83-162. In some embodiments, the sequence of nucleotides encoding the variant CD80-Fc fusion protein is set forth in any one of SEQ ID NOS: 83-162.

In some embodiments, the variant CD80-Fc fusion protein has the sequence set forth in SEQ ID NO:265.

(SEQ ID NO: 265)
VIHVTKEVKEYATLSCGHNVSVEELAQYRIYWQKEKKMVLTMMSG
DLNIWPEYKNRTIFDITNNLSIVILALRPSDEGTYECVVLKYEKD
AFKREHLAEVTLSVKADGSGGGGSESKYGPPCPPCPAPEFLGGPS
VFLFPPKPKDTLMISRTPEVTCVVVDVSQEDPEVQFNWYVDGVEV
HNAKTKPREEQFNSTYRVVSVLTVLHQDWLNGKEYKCKVSNKGLP
SSIEKTISKAKGQPREPQVYTLPPSQEEMTKNQVSLTCLVKGFYP
SDIAVEWESNGQPENNYKTTPPVLDSDGSFFLYSRLTVDKSRWQE
GNVFSCSVMHEALHNHYTQKSLSLSLG

In some embodiments, the variant CD80-Fc fusion is encoded by the sequence set forth in SEQ ID NO:103.

In some embodiments, the variant CD80-Fc fusion protein has the sequence set forth in SEQ ID NO:313.

(SEQ ID NO: 313)
VIHVTKEVKEYATLSCGHNVSVEELAQYRIYWQKEKKMVLTMMSG
DLNIWPEYKNRTIFDITNNLSIMILALRPSDEGTYECVVLKYEKD
AFKREHLAEVTLSVKADGSGGGGSESKYGPPCPPCPAPEFLGGPS
VFLFPPKPKDTLMISRTPEVTCVVVDVSQEDPEVQFNWYVDGVEV
HNAKTKPREEQFNSTYRVVSVLTVLHQDWLNGKEYKCKVSNKGLP
SSIEKTISKAKGQPREPQVYTLPPSQEEMTKNQVSLTCLVKGFYP
SDIAVEWESNGQPENNYKTTPPVLDSDGSFFLYSRLTVDKSRWQE
GNVFSCSVMHEALHNHYTQKSLSLSLG

In some embodiments, the variant CD80-Fc fusion is encoded by the sequence set forth in SEQ ID NO:151.

In some embodiments, the variant CD80-Fc fusion protein has the sequence set forth in SEQ ID NO:314.

(SEQ ID NO: 314)
VIHVTKEVKEYATLSCGHNVSVEELAQYRIYWQKEKKMVLTMMSG
DLNIWPEYKNRTIFDITNNLSILILALRPSDEGTYECVVLKYEKD
AFKREHLAEVTLSVKADGSGGGGSESKYGPPCPPCPAPEFLGGPS
VFLFPPKPKDTLMISRTPEVTCVVVDVSQEDPEVQFNWYVDGVEV
HNAKTKPREEQFNSTYRVVSVLTVLHQDWLNGKEYKCKVSNKGLP
SSIEKTISKAKGQPREPQVYTLPPSQEEMTKNQVSLTCLVKGFYP
SDIAVEWESNGQPENNYKTTPPVLDSDGSFFLYSRLTVDKSRWQE
GNVFSCSVMHEALHNHYTQKSLSLSLG

In some embodiments, the variant CD80-Fc fusion is encoded by the sequence set forth in SEQ ID NO:152.

In some embodiments, the variant CD80-Fc fusion protein contains multiple copies of a variant TACI-polypeptide sequence, such as 2, 3 or 4 variant CD80 polypeptide sequences. In some embodiments, the variant CD80-Fc fusion proteins contains two variant CD80 polypeptide sequences. In some cases, the variant CD80 polypeptide sequences may be linked directly or may be linked indirectly via a linker, such as a peptide linker including any as described. In such an example, one of the variant CD80 polypeptide sequence is joined or linked to the Fc region, such as either to the N- or C-terminus of the Fc region. In other cases, the variant CD80 polypeptide sequences may be separated from each other by the Fc region and each joined individually to the N- or C-terminus of the Fc region. The linkage to the Fc region may be direct or may be indirect via a linker, such as a peptide linker including any as described.

In some embodiments, the variant CD80 polypeptide sequences may be arranged in order in the fusion protein in tandem (hereinafter called a “tandem” Fc fusion construct). In some embodiments, the variant CD80-Fc fusion protein has the structure: (vCD80)-Linker-(vCD80)-Linker-Fc region. In some embodiments, the immunomodulatory protein is a tetravalent molecule that is a homodimer of two identical copies of the variant CD80-Fc fusion protein. For instance, interactions between Fc regions of the two identical polypeptide fusions form covalent disulfide bonds to result in a dimeric molecule containing four variant CD80 polypeptides.

In some embodiments, there is provided a variant CD80-Fc fusion protein containing in order a variant CD80 polypeptide, e.g. any as described above; a linker; another variant CD80 polypeptide, e.g. any as described; and an Fc region. In some embodiments, each variant CD80 polypeptide of the variant CD80-Fc fusion is a variant CD80 polypeptide, such as any as described. In some embodiments, each variant CD80 polypeptide of the variant CD80 Fc fusion is a variant CD80 set forth in any one of SEQ ID NOS: 165-244. The linkers may be any as described. In some embodiments, the linker is GSGGGGS (SEQ ID NO: 325). The Fc region may be any Fc region as described. In some embodiments, the Fc region is a variant IgG1 Fc set forth in SEQ ID NO: 344. In some embodiments, the Fc region is a IgG4 Fc set forth in SEQ ID NO: 326. In some embodiments, the variant CD80-Fc fusion protein comprises the sequence set forth in SEQ ID NO: 337.

In some embodiments, the variant CD80-Fc fusion protein comprises the sequence set forth in SEQ ID NO: 340.

In some embodiments, the variant CD80-Fc fusion protein comprises the sequence set forth in SEQ ID NO: 342.

In some embodiments, the variant CD80-Fc fusion protein comprises SEQ ID NO: 414.

In some embodiments, the variant CD80 polypeptide sequences may be separated in the fusion protein by the Fc region in which the Fc region is positioned between the two variant CD80 polypeptide sequences (hereinafter called a “barbell” Fc fusion construct). In some embodiments, the variant CD80-Fc fusion protein has the structure: (vCD80)-Linker-Fc region-Linker-(vCD80). In some embodiments, the linkers may be the same or different. In some embodiments, the immunomodulatory protein is a tetravalent molecule that is a homodimer of two identical copies of the variant CD80-Fc fusion protein. For instance, interactions between Fc regions of the two identical polypeptide fusions form covalent disulfide bonds to result in a dimeric molecule containing four variant CD80 polypeptides.

In some embodiments, there is provided a variant CD80-Fc fusion protein containing in order a variant CD80 polypeptide, e.g. any as described above; a linker; an Fc region; a linker; and another variant CD80 polypeptide, e.g. any as described. In some embodiments, each variant CD80 polypeptide of the variant CD80-Fc fusion is a variant CD80 polypeptide, such as any as described. In some embodiments, each variant CD80 polypeptide of the variant CD80 Fc fusion is a variant CD80 polypeptide set forth in any one of SEQ ID NOS: 165-244. The linkers may be any as described, and may be the same of different. In some embodiments, the first linker is GSGGGGS (SEQ ID NO: 325) and the second linker is (GGGGS)₄ (SEQ ID NO: 331). The Fc region may be any Fc region as described. In some embodiments, the Fc region is a variant IgG1 Fc set forth in SEQ ID NO: 344. In some embodiments, the Fc region is a IgG4 Fc set forth in SEQ ID NO: 326.

In some embodiments, the variant CD80-Fc fusion protein comprises the sequence set forth in SEQ ID NO: 336.

In some embodiments, the variant CD80-Fc fusion protein comprises the sequence set forth in SEQ ID NO: 338.

In some embodiments, the variant CD80-Fc fusion protein comprises the sequence set forth in SEQ ID NO: 339.

In some embodiments, the variant CD80-Fc fusion protein comprises the sequence set forth in SEQ ID NO: 341.

In some embodiments, there is a provided a variant CD80-Fc fusion protein that is a dimer formed by two identical variant CD80 polypeptides as described linked to an Fc domain. In some embodiments, identical species (also referred to as copies) of any of the provided variant CD80-Fc fusion polypeptides will be dimerized to create a homodimer. In some embodiments, the dimer is a homodimer in which the two variant CD80-Fc polypeptides are the same. For generating a homodimeric Fc molecule, the Fc region is one that is capable of forming a homodimer with a matched Fc region by co-expression of the individual Fc regions in a cell. In some embodiments, dimerization is mediated by covalent disulfide bond(s) formed between the Fc regions of the polypeptide fusions.

Also provided are nucleic acid molecules encoding the immunomodulatory protein. In some embodiments, for production of immunomodulatory protein, a nucleic acid molecule encoding the immunomodulatory protein is inserted into an appropriate expression vector. The resulting immunomodulatory protein can be expressed in host cells transformed with the expression where assembly between Fc domains occurs by interchain disulfide bonds formed between the Fc moieties to yield dimeric, such as divalent, immunomodulatory proteins.

Also provided are nucleic acid molecules encoding the variant CD80-Fc fusion protein. In some embodiments, for production of an Fc fusion protein, a nucleic acid molecule encoding a variant CD80-Fc fusion protein is inserted into an appropriate expression vector. The resulting variant CD80-Fc fusion protein can be expressed in host cells transformed with the expression where assembly between Fc domains occurs by interchain disulfide bonds formed between the Fc moieties to yield dimeric variant CD80-Fc fusion proteins. The resulting Fc fusion proteins can be easily purified by affinity chromatography over Protein A or Protein G columns.

In embodiments, when produced and expressed from a cell, the provided immunomodulatory protein, such as a variant CD80-Fc, is a homodimer containing two identical polypeptide chains.

2. Fusions with an Effector Moiety

In some embodiments, the variant CD80 polypeptides provided herein can be conjugated with or fused with a moiety, such as an effector moiety, such as another protein, directly or indirectly, to form a fusion protein (“IgSF conjugate”). In some embodiments, the fusion is direct. In some embodiments, the fusion is indirect, such as via a linker. In some embodiments, the attachment can be covalent or non-covalent, e.g., via a biotin-streptavidin non-covalent interaction.

In some embodiments, the moiety can be a targeting moiety, a small molecule drug (non-polypeptide drug of less than 500 Daltons molar mass), a toxin, a cytostatic agent, a cytotoxic agent, an immunosuppressive agent, a radioactive agent suitable for diagnostic purposes, a radioactive metal ion for therapeutic purposes, a prodrug-activating enzyme, an agent that increases biological half-life, or a diagnostic or detectable agent.

In some embodiments, the effector moiety is a therapeutic agent, such as a cancer therapeutic agent, which is either cytotoxic, cytostatic or otherwise provides some therapeutic benefit. In some embodiments, the effector moiety is a targeting moiety or agent, such as an agent that targets a cell surface antigen, e.g., an antigen on the surface of a tumor cell. In some embodiments, the effector moiety is a label, which can generate a detectable signal, either directly or indirectly. In some embodiments, the effector moiety is a toxin. In some embodiments, the effector moiety is a protein, peptide, nucleic acid, small molecule or nanoparticle.

In some embodiments, 1, 2, 3, 4, 5 or more effector moieties, which can be the same or different, are conjugated, linked or fused to the variant polypeptide or protein to form an IgSF conjugate. In some embodiments, such effector moieties can be attached to the variant polypeptide or immunomodulatory protein using various molecular biological or chemical conjugation and linkage methods known in the art and described below. In some embodiments, linkers such as peptide linkers, cleavable linkers, non-cleavable linkers or linkers that aid in the conjugation reaction, can be used to link or conjugate the effector moieties to the variant polypeptide or immunomodulatory protein.

In some embodiments, the IgSF conjugate comprises the following components: (protein or polypeptide), (L)_{q} and (effector moiety)ₘ, wherein the protein or polypeptide is any of the described variant polypeptides or immunomodulatory proteins capable of binding one or more cognate counter structure ligands as described; L is a linker for linking the protein or polypeptide to the moiety; m is at least 1; q is 0 or more; and the resulting IgSF conjugate binds to the one or more counter structure ligands. In particular embodiments, m is 1 to 4 and q is 0 to 8.

In some embodiments, there is provided an IgSF conjugate comprising a variant polypeptide or immunomodulatory protein provided herein conjugated with a targeting agent that binds to a cell surface molecule, for example, for targeted delivery of the variant polypeptide or immunomodulatory protein to a specific cell. In some embodiments, the targeting agent is a molecule(s) that has the ability to localize and bind to a molecule present on a normal cell/tissue and/or tumor cell/tumor in a subject. In other words, IgSF conjugates comprising a targeting agent can bind to a ligand (directly or indirectly), which is present on a cell, such as a tumor cell. The targeting agents of the invention contemplated for use include antibodies, polypeptides, peptides, aptamers, other ligands, or any combination thereof, that can bind a component of a target cell or molecule.

In some embodiments, the targeting agent binds a tumor cell(s) or can bind in the vicinity of a tumor cell(s) (e.g., tumor vasculature or tumor microenvironment) following administration to the subject. The targeting agent may bind to a receptor or ligand on the surface of the cancer cell. In another aspect of the invention, a targeting agent is selected which is specific for a noncancerous cells or tissue. For example, a targeting agent can be specific for a molecule present normally on a particular cell or tissue. Furthermore, in some embodiments, the same molecule can be present on normal and cancer cells. Various cellular components and molecules are known. For example, if a targeting agent is specific for EGFR, the resulting IgSF conjugate can target cancer cells expressing EGFR as well as normal skin epidermal cells expressing EGFR. Therefore, in some embodiments, an IgSF conjugate of the invention can operate by two separate mechanisms (targeting cancer and non-cancer cells).

In various aspects of the invention disclosed herein an IgSF conjugate of the invention comprises a targeting agent which can bind/target a cellular component, such as a tumor antigen, a bacterial antigen, a viral antigen, a mycoplasma antigen, a fungal antigen, a prion antigen, an antigen from a parasite. In some aspects, a cellular component, antigen or molecule can each be used to mean, a desired target for a targeting agent. For example, in various embodiments, a targeting agent is specific for or binds to a component, which includes but is not limited to, epidermal growth factor receptor (EGFR, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family; platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family; TRK receptor family; ephrin (EPH) receptor family; AXL receptor family; leukocyte tyrosine kinase (LTK) receptor family; TIE receptor family, angiopoietin 1,2; receptor tyrosine kinase-like orphan receptor (ROR) receptor family, e.g., ROR1; CD171 (L1CAM); B7-H6 (NCR3LG1); CD80, tumor glycosylation antigen, e.g., sTn or Tn, such as sTn Ag of MUC1; LHR (LHCGR); phosphatidylserine, discoidin domain receptor (DDR) family; RET receptor family; KLG receptor family; RYK receptor family; MuSK receptor family; Transforming growth factor-α (TGF-α) receptors, TGF-β; Cytokine receptors, Class I (hematopoietin family) and Class II (interferon/IL-10 family) receptors, tumor necrosis factor (TNF) receptor superfamily (TNFRSF), death receptor family; cancer-testis (CT) antigens, lineage-specific antigens, differentiation antigens, alpha-actinin-4, ARTCl, breakpoint cluster region-Abelson (Bcr-abl) fusion products, B-RAF, caspase-5 (CASP-5), caspase-8 (CASP-8), β-catenin (CTNNBI), cell division cycle 27 (CDC27), cyclin-dependent kinase 4 (CDK4), CDKN2A, COA-I, dek-can fusion protein, EFTUD-2, Elongation factor 2 (ELF2), Ets variant gene 6/acute myeloid leukemia 1 gene ETS (ETC6-AML1) fusion protein, fibronectin (FN), e.g., the extradomain A (EDA) of fibronectin, GPNMB, low density lipid receptor/GDP-L fucose: β-D-galactose 2-α-L-fucosyltransferase (LDLR/FUT) fusion protein, HLA-A2. arginine to isoleucine exchange at residue 170 of the α-helix of the α2-domain in the HLA-A2gene (HLA-A*201-R170I), HLA-A1 1, heat shock protein 70-2 mutated (HSP70-2M), K1AA0205, MART2, melanoma ubiquitous mutated 1, 2, 3 (MUM-I, 2, 3), prostatic acid phosphatase (PAP), neo-PAP, Myosin class I, NFYC, OGT, OS-9, pml-RARa fusion protein, PRDX5, PTPRK, K-ras (KRAS2), N-ras (NRAS), HRAS, RBAF600, SIRT2, SNRPD1, SYT-SSX1 or -SSX2 fusion protein, Triosephosphate Isomerase, BAGE, BAGK-1, BAGE-2,3,4,5, GAGE-1,2,3,4,5,6,7,8, GnT-V (aberrant N-acetyl glucosaminyl transferase V, MGAT5), HERV-K-MEL, KK-LC, KM-HN-I, LAGE, LAGE-I, CTL-recognized antigen on melanoma (CAMEL), MAGE-A1 (MAGE-I), MAGE-A2, MAGE-A3, MAGE-A4, MAGE-A5, MAGE-A6, MAGE-A8, MAGE-A9, MAGE-AlO, MAGE-AI 1, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gplOO, gplOO/Pmell7 (SILV), tyrosinase (TYR), TRP-I, HAGE, NA-88, NY-ESO-I, NY-ESO-1/LAGE-2, SAGE, Spl7, SSX-1,2,3,4, TRP2-INT2, carcino-embryonic antigen (CEA), Kallikrein 4, mammaglobin-A, OA1, prostate specific antigen (PSA), TRP-1/gp75, TRP-2, adipophilin, interferon inducible protein absent in melanoma 2 (AIM-2), BING-4, CPSF, cyclin Dl, epithelial cell adhesion molecule (Ep-CAM), EphA3, fibroblast growth factor-5 (FGF-5), glycoprotein 250 (gp250), EGFR (ERBBI), HER-2/neu (ERBB2), interleukin 13 receptor α2 chain (IL13Rα2), IL-6 receptor, intestinal carboxyl esterase (iCE), alpha-feto protein (AFP), M-CSF, mdm-2, MUC1, p53 (TP53), PBF, PRAME, PSMA, RAGE-I, RNF43, RU2AS, SOXlO, STEAP1, survivin (BIRC5), human telomerase reverse transcriptase (hTERT), telomerase, Wilms' tumor gene (WTl), SYCP1, BRDT, SPANX, XAGE, ADAM2, PAGE-5, LIPI, CTAGE-I, CSAGE, MMA1, CAGE, BORIS, HOM-TES-85, AF15ql4, HCA661, LDHC, MORC, SGY-I, SPOl 1, TPX1, NY-SAR-35, FTHL17, NXF2, TDRD1, TEX15, FATE, TPTE, immunoglobulin idiotypes, Bence-Jones protein, estrogen receptors (ER), androgen receptors (AR), CD40, CD30, CD20, CD 19, CD33, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), β-human chorionic gonadotropin, β-2 microglobulin, squamous cell carcinoma antigen, neuron-specific enolase, heat shock protein gp96, GM2, sargramostim, CTLA-4, 707 alanine proline (707-AP), adenocarcinoma antigen recognized by T cells 4 (ART-4), carcinoembryonic antigen peptide-1 (CAP-I), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr virus (EBV) proteins (EBV latent membrane proteins—LMP1, LMP2; others), Hepatitis B or C virus proteins, and HIV proteins.

In some embodiments, an IgSF conjugate, through its targeting agent, will bind a cellular component of a tumor cell, tumor vasculature or tumor microenvironment, thereby promoting killing of targeted cells via modulation of the immune response, (e.g., by activation of co-stimulatory molecules or inhibition of negative regulatory molecules of immune cell activation), inhibition of survival signals (e.g., growth factor or cytokine or hormone receptor antagonists), activation of death signals, and/or immune-mediated cytotoxicity, such as through antibody dependent cellular cytotoxicity. Such IgSF conjugates can function through several mechanisms to prevent, reduce or eliminate tumor cells, such as to facilitate delivery of conjugated effector moieties to the tumor target, such as through receptor-mediated endocytosis of the IgSF conjugate; or such conjugates can recruit, bind, and/or activate immune cells (e.g., NK cells, monocytes/macrophages, dendritic cells, T cells, B cells). Moreover, in some instances one or more of the foregoing pathways may operate upon administration of one or more IgSF conjugates of the invention.

In some embodiments, an IgSF conjugate, through its targeting agent, will be localized to, such as bind to, a cellular component of a tumor cell, tumor vasculature or tumor microenvironment, thereby modulating cells of the immune response in the vicinity of the tumor. In some embodiments, the targeting agent facilitates delivery of the conjugated IgSF (e.g., vIgD) to the tumor target, such as to interact with its cognate binding partner to alter signaling of immune cells (e.g., NK cells, monocytes/macrophages, dendritic cells, T cells, B cells) bearing the cognate binding partner. In some embodiments, localized delivery mediates an antagonizing or blocking activity of the CTLA-4 inhibitory receptor. In some embodiments, localized delivery agonizes the CTLA-4 inhibitory receptor, which, in some cases, can occur where there is proximal clustering of an activating receptor.

In some embodiments, the targeting agent is an immunoglobulin. As used herein, the term “immunoglobulin” includes natural or artificial mono- or polyvalent antibodies including, but not limited to, polyclonal, monoclonal, multispecific, human, humanized or chimeric antibodies, single chain antibodies, Fab fragments, F(ab′) fragments, fragments produced by a Fab expression library, single chain Fv (scFv); anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, e.g., molecules that contain an antigen binding site that immunospecifically binds an antigen. The immunoglobulin molecules of the invention can be of any type (e.g., IgG, IgE, IgM, IgD, IgA, and IgY), class (e.g., IgG1, IgG2, IgG3, IgG4, IgA1, and IgA2) or subclass of immunoglobulin molecule.

In some embodiments, an IgSF conjugate, through its antibody targeting moiety, will bind a cellular component of a tumor cell, tumor vasculature or tumor microenvironment, thereby promoting apoptosis of targeted cells via modulation of the immune response, (e.g., by activation of co-stimulatory molecules or inhibition of negative regulatory molecules of immune cell activation), inhibition of survival signals (e.g., growth factor or cytokine or hormone receptor antagonists), activation of death signals, and/or immune-mediated cytotoxicity, such as through antibody dependent cellular cytotoxicity. Such IgSF conjugates can function through several mechanisms to prevent, reduce or eliminate tumor cells, such as to facilitate delivery of conjugated effector moieties to the tumor target, such as through receptor-mediated endocytosis of the IgSF conjugate; or such conjugates can recruit, bind, and/or activate immune cells (e.g., NK cells, monocytes/macrophages, dendritic cells, T cells, B cells).

In some embodiments, an IgSF conjugate, through its antibody targeting moiety, will bind a cellular component of a tumor cell, tumor vasculature or tumor microenvironment, thereby modulating the immune response (e.g., by activation of co-stimulatory molecules or inhibition of negative regulatory molecules of immune cell activation). In some embodiments, such conjugates can recognize, bind, and/or modulate (e.g., inhibit or activate) immune cells (e.g., NK cells, monocytes/macrophages, dendritic cells, T cells, B cells).

Antibody targeting moieties of the invention include antibody fragments that include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Also included in the invention are Fc fragments, antigen-Fc fusion proteins, and Fc-targeting moiety conjugates or fusion products (Fc-peptide, Fc-aptamer). The antibody targeting moieties of the invention may be from any animal origin including birds and mammals. In one aspect, the antibody targeting moieties are human, murine (e.g., mouse and rat), donkey, sheep, rabbit, goat, guinea pig, camel, horse, or chicken. Further, such antibodies may be humanized versions of animal antibodies. The antibody targeting moieties of the invention may be monospecific, bispecific, trispecific, or of greater multispecificity.

In various embodiments, an antibody/targeting moiety recruits, binds, and/or activates immune cells (e.g., NK cells, monocytes/macrophages, dendritic cells) via interactions between Fc (in antibodies) and Fc receptors (on immune cells) and via the conjugated variant polypeptides or immunomodulatory proteins provided herein. In some embodiments, an antibody/targeting moiety recognizes or binds a tumor agent via and localizes to the tumor cell the conjugated variant polypeptides or immunomodulatory proteins provided herein to facilitate modulation of immune cells in the vicinity of the tumor.

Examples of antibodies which can be incorporated into IgSF conjugates include but are not limited to antibodies such as Cetuximab (IMC-C225; Erbitux®), Trastuzumab (Herceptin®), Rituximab (Rituxan®; MabThera®), Bevacizumab (Avastin®), Alemtuzumab (Campath®; Campath-1H®; Mabcampath®), Panitumumab (ABX-EGF; Vectibix®), Ranibizumab (Lucentis®), Ibritumomab, Ibritumomab tiuxetan, (Zevalin®), Tositumomab, Iodine 1131 Tositumomab (BEXXAR®), Catumaxomab (Removab®), Gemtuzumab, Gemtuzumab ozogamicine (Mylotarg®), Abatacept (CTLA4-Ig; Orencia®), Belatacept (L104EA29YIg; LEA29Y; LEA), Ipilimumab (MDX-010; MDX-101), Tremelimumab (ticilimumab; CP-675,206), PRS-010, PRS-050, Aflibercept (VEGF Trap, AVE005), Volociximab (M200), F200, MORAb-009, SS1P (CAT-5001), Cixutumumab (IMC-A12), Matuzumab (EMD72000), Nimotuzumab (h-R3), Zalutumumab (HuMax-EGFR), Necitumumab IMC-11F8, mAb806/ch806, Sym004, mAb-425, Panorex® (17-1A) (murine monoclonal antibody); Panorex @(17-1A) (chimeric murine monoclonal antibody); IDEC-Y2B8 (murine, anti-CD20 MAb); BEC2 (anti-idiotypic MAb, mimics the GD epitope) (with BCG); Oncolym (Lym-1 monoclonal antibody); SMART M195 Ab, humanized 13′ I LYM-I (Oncolym), Ovarex (B43.13, anti-idiotypic mouse MAb); MDX-210 (humanized anti-HER-2 bispecific antibody); 3622W94 MAb that binds to EGP40 (17-1A) pancarcinoma antigen on adenocarcinomas; Anti-VEGF, Zenapax (SMART Anti-Tac (IL-2 receptor); SMART MI95 Ab, humanized Ab, humanized); MDX-210 (humanized anti-HER-2 bispecific antibody); MDX-447 (humanized anti-EGF receptor bispecific antibody); NovoMAb-G2 (pancarcinoma specific Ab); TNT (chimeric MAb to histone antigens); TNT (chimeric MAb to histone antigens); Gliomab-H (Monoclon s—Humanized Abs); GNI-250 Mab; EMD-72000 (chimeric-EGF antagonist); LymphoCide (humanized LL2 antibody); and MDX-260 bispecific, targets GD-2, ANA Ab, SMART IDlO Ab, SMART ABL 364 Ab or ImmuRAIT-CEA. As illustrated by the forgoing list, it is conventional to make antibodies to a particular target epitope.

In some embodiments, the antibody targeting moiety is a full-length antibody, or antigen-binding fragment thereof, containing an Fc domain. In some embodiments, the variant polypeptide or immunomodulatory protein is conjugated to the Fc portion of the antibody targeting moiety, such as by conjugation to the N-terminus of the Fc portion of the antibody.

In some embodiments, the vIgD is linked, directly or indirectly, to the N- or C-terminus of the light and/or heavy chain of the antibody. In some embodiments, linkage can be via a peptide linker, such as any described above. In some embodiments, the antibody conjugate can be produced by co-expression of the heavy and light chain of the antibody in a cell.

In one aspect of the invention, the targeting agent is an aptamer molecule. For example, in some embodiments, the aptamer is comprised of nucleic acids that function as a targeting agent. In various embodiments, an IgSF conjugate of the invention comprises an aptamer that is specific for a molecule on a tumor cell, tumor vasculature, and/or a tumor microenvironment. In some embodiments, the aptamer itself can comprise a biologically active sequence, in addition to the targeting module (sequence), wherein the biologically active sequence can induce an immune response to the target cell. In other words, such an aptamer molecule is a dual use agent. In some embodiments, an IgSF conjugate of the invention comprises conjugation of an aptamer to an antibody, wherein the aptamer and the antibody are specific for binding to separate molecules on a tumor cell, tumor vasculature, tumor microenvironment, and/or immune cells.

The term “aptamer” includes DNA, RNA or peptides that are selected based on specific binding properties to a particular molecule. For example, an aptamer(s) can be selected for binding a particular gene or gene product in a tumor cell, tumor vasculature, tumor microenvironment, and/or an immune cell, as disclosed herein, where selection is made by methods known in the art and familiar to one of skill in the art.

In some aspects of the invention the targeting agent is a peptide. For example, the variant polypeptides or immunomodulatory proteins provided herein can be conjugated to a peptide which can bind with a component of a cancer or tumor cells. Therefore, such IgSF conjugates of the invention comprise peptide targeting agents which binds to a cellular component of a tumor cell, tumor vasculature, and/or a component of a tumor microenvironment. In some embodiments, targeting agent peptides can be an antagonist or agonist of an integrin. Integrins, which comprise an alpha and a beta subunit, include numerous types well known to a skilled artisan.

In one embodiment, the targeting agent is Vvβ. Integrin Vvβ3 is expressed on a variety of cells and has been shown to mediate several biologically relevant processes, including adhesion of osteoclasts to bone matrix, migration of vascular smooth muscle cells, and angiogenesis. Suitable targeting molecules for integrins include RGD peptides or peptidomimetics as well as non-RGD peptides or peptidomimetics (see, e.g., U.S. Pat. Nos. 5,767,071 and 5,780,426) for other integrins such as V4.βi (VLA-4), V4-P7 (see, e.g., U.S. Pat. No. 6,365,619; Chang et al, Bioorganic & Medicinal Chem Lett, 12:159-163 (2002); Lin et al., Bioorganic & Medicinal Chem Lett, 12:133-136 (2002)), and the like.

In some embodiments, there is provided an IgSF conjugate comprising a variant polypeptide or immunomodulatory protein provided herein conjugated with a therapeutic agent. In some embodiments, the therapeutic agent includes, for example, daunomycin, doxorubicin, methotrexate, and vindesine (Rowland et al., Cancer Immunol. Immunother. 21:183-187, 1986). In some embodiments, the therapeutic agent has an intracellular activity. In some embodiments, the IgSF conjugate is internalized and the therapeutic agent is a cytotoxin that blocks the protein synthesis of the cell, therein leading to cell death. In some embodiments, the therapeutic agent is a cytotoxin comprising a polypeptide having ribosome-inactivating activity including, for example, gelonin, bouganin, saporin, ricin, ricin A chain, bryodin, diphtheria toxin, restrictocin, Pseudomonas exotoxin A and variants thereof. In some embodiments, where the therapeutic agent is a cytotoxin comprising a polypeptide having a ribosome-inactivating activity, the IgSF conjugate must be internalized upon binding to the target cell in order for the protein to be cytotoxic to the cells.

In some embodiments, there is provided an IgSF conjugate comprising a variant polypeptide or immunomodulatory protein provided herein conjugated with a toxin. In some embodiments, the toxin includes, for example, bacterial toxins such as diphtheria toxin, plant toxins such as ricin, small molecule toxins such as geldanamycin (Mandler et al., J. Nat. Cancer Inst. 92(19):1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition.

In some embodiments, there is provided an IgSF conjugate comprising a variant polypeptide or immunomodulatory protein provided herein conjugated with a label, which can generate a detectable signal, indirectly or directly. These IgSF conjugates can be used for research or diagnostic applications, such as for the in vivo detection of cancer. The label is preferably capable of producing, either directly or indirectly, a detectable signal. For example, the label may be radio-opaque or a radioisotope, such as 3H, 14C, 32P, 355, 1231, 1251, 1311; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the label is a radioactive atom for scintigraphic studies, for example 99Tc or 1231, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983). In some embodiments, the IgSF conjugate is detectable indirectly. For example, a secondary antibody that is specific for the IgSF conjugate and contains a detectable label can be used to detect the IgSF conjugate.

The IgSF conjugates may be prepared using any methods known in the art. See, e.g., WO 2009/067800, WO 2011/133886, and U.S. Patent Application Publication No. 2014322129, incorporated by reference herein in their entirety.

The variant polypeptides or immunomodulatory proteins of an IgSF conjugate may be “attached to” the effector moiety by any means by which the variant polypeptides or immunomodulatory proteins can be associated with, or linked to, the effector moiety. For example, the variant polypeptides or immunomodulatory proteins of an IgSF conjugate may be attached to the effector moiety by chemical or recombinant means. Chemical means for preparing fusions or conjugates are known in the art and can be used to prepare the IgSF conjugate. The method used to conjugate the variant polypeptides or immunomodulatory proteins and effector moiety must be capable of joining the variant polypeptides or immunomodulatory proteins with the effector moiety without interfering with the ability of the variant polypeptides or immunomodulatory proteins to bind to their one or more counter structure ligands.

The variant polypeptides or immunomodulatory proteins of an IgSF conjugate may be linked indirectly to the effector moiety. For example, the variant polypeptides or immunomodulatory proteins of an IgSF conjugate may be directly linked to a liposome containing the effector moiety of one of several types. The effector moiety(s) and/or the variant polypeptides or immunomodulatory proteins may also be bound to a solid surface.

In some embodiments, the variant polypeptides or immunomodulatory proteins of an IgSF conjugate and the effector moiety are both proteins and can be conjugated using techniques well known in the art. There are several hundred crosslinkers available that can conjugate two proteins. (See for example “Chemistry of Protein Conjugation and Crosslinking,” 1991, Shans Wong, CRC Press, Ann Arbor). The crosslinker is generally chosen based on the reactive functional groups available or inserted on the variant polypeptides or immunomodulatory proteins and/or effector moiety. In addition, if there are no reactive groups, a photoactivatable crosslinker can be used. In certain instances, it may be desirable to include a spacer between the variant polypeptides or immunomodulatory proteins and the effector moiety. Crosslinking agents known to the art include the homobifunctional agents: glutaraldehyde, dimethyladipimidate and Bis(diazobenzidine) and the heterobifunctional agents: m Maleimidobenzoyl-N-Hydroxysuccinimide and Sulfo-m Maleimidobenzoyl-N-Hydroxysuccinimide.

In some embodiments, the variant polypeptides or immunomodulatory proteins of an IgSF conjugate may be engineered with specific residues for chemical attachment of the effector moiety. Specific residues used for chemical attachment of molecule known to the art include lysine and cysteine. The crosslinker is chosen based on the reactive functional groups inserted on the variant polypeptides or immunomodulatory proteins, and available on the effector moiety.

An IgSF conjugate may also be prepared using recombinant DNA techniques. In such a case a DNA sequence encoding the variant polypeptides or immunomodulatory proteins is fused to a DNA sequence encoding the effector moiety, resulting in a chimeric DNA molecule. The chimeric DNA sequence is transfected into a host cell that expresses the fusion protein. The fusion protein can be recovered from the cell culture and purified using techniques known in the art.

Examples of attaching an effector moiety, which is a label, to the variant polypeptides or immunomodulatory proteins include the methods described in Hunter, et al., Nature 144:945 (1962); David, et al., Biochemistry 13:1014 (1974); Pain, et al., J. Immunol. Meth. 40:219 (1981); Nygren, J. Histochem. and Cytochem. 30:407 (1982); Wensel and Meares, Radioimmunoimaging And Radioimmunotherapy, Elsevier, N.Y. (1983); and Colcher et al., “Use Of Monoclonal Antibodies As Radiopharmaceuticals For The Localization Of Human Carcinoma Xenografts In Athymic Mice”, Meth. Enzymol., 121:802-16 (1986).

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as 99Tc or 1231, 186Re, 188Re and 11Iln can be attached via a cysteine residue in the peptide. Yttrium-90 can be attached via a lysine residue. The IODOGEN method (Fraker et al., Biochem. Biophys. Res. Commun. 80:49-57 (1978)) can be used to incorporate iodine-123. “Monoclonal Antibodies in Immunoscintigraphy” (Chatal, CRC Press 1989) describes other methods in detail.

Conjugates of the variant polypeptides or immunomodulatory proteins and a cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-p-isothiocyanatobenzyl-3-methyldiethylenetriaminepentaacetic acid (MX-DTPA) is an exemplary chelating agent for conjugation of radionucleotide to the antibody. See, e.g., WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The IgSF conjugates of the invention expressly contemplate, but are not limited to, drug conjugates prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL, U.S.A). See pages 467-498, 2003-2004 Applications Handbook and Catalog.

### C. Cells and Engineering Cells

Provided herein are engineered cells expressing any of the provided immunomodulatory polypeptide. In some embodiments, the immunomodulatory protein is any as described in Sections I.A and I.B. In some embodiments, the engineered cells express on their surface any of the provided transmembrane immunomodulatory polypeptides. In some embodiments, the engineered cells express and are capable of or are able to secrete the immunomodulatory protein from the cells under conditions suitable for secretion of the protein. In some embodiments, the immunomodulatory protein is expressed on a lymphocyte such as a tumor infiltrating lymphocyte (TIL), T-cell or NK cell, or on a myeloid cell. In some embodiments, the engineered cells are antigen presenting cells (APCs). In some embodiments, the engineered cells are engineered mammalian T-cells or engineered mammalian antigen presenting cells (APCs). In some embodiments, the engineered T-cells or APCs are human or murine cells.

In some embodiments, engineered T-cells include, but are not limited to, T helper cell, cytotoxic T-cell (alternatively, cytotoxic T lymphocyte or CTL), natural killer T-cell, regulatory T-cell, memory T-cell, or gamma delta T-cell. In some embodiments, the engineered T cells are CD4+ or CD8+. In addition to the signal of the MHC, engineered T-cells also require a co-stimulatory signal. Inn some embodiments, engineered T cells also can be modulated by inhibitory signals, which, in some cases, is provided by a variant CD80 transmembrane immunomodulatory polypeptide expressed in membrane bound form as discussed previously.

In some embodiments, the engineered APCs include, for example, MHC II expressing APCs such as macrophages, B cells, and dendritic cells, as well as artificial APCs (aAPCs) including both cellular and acellular (e.g., biodegradable polymeric microparticles) aAPCs. Artificial APCs (aAPCs) are synthetic versions of APCs that can act in a similar manner to APCs in that they present antigens to T-cells as well as activate them. Antigen presentation is performed by the MHC (Class I or Class II). In some embodiments, in engineered APCs such as aAPCs, the antigen that is loaded onto the MHC is, in some embodiments, a tumor specific antigen or a tumor associated antigen. The antigen loaded onto the MHC is recognized by a T-cell receptor (TCR) of a T cell, which, in some cases, can express CTLA-4, CD28, PD-L1 or other molecules recognized by the variant CD80 polypeptides provided herein. Materials which can be used to engineer an aAPC include: poly (glycolic acid), poly(lactic-co-glycolic acid), iron-oxide, liposomes, lipid bilayers, sepharose, and polystyrene.

In some embodiments, the immunomodulatory polypeptides, such as transmembrane immunomodulatory polypeptides or secretable immunomodulatory polypeptides, can be incorporated into engineered cells, such as engineered T cells or engineered APCs, by a variety of strategies such as those employed for recombinant host cells. A variety of methods to introduce a DNA construct into primary T cells are known in the art. In some embodiments, viral transduction or plasmid electroporation are employed. In typical embodiments, the nucleic acid molecule encoding the immunomodulatory protein, or the expression vector, comprises a signal peptide that localizes the expressed transmembrane immunomodulatory proteins to the cellular membrane or for secretion. In some embodiments, a nucleic acid encoding a transmembrane immunomodulatory protein of the invention is sub-cloned into a viral vector, such as a retroviral vector, which allows expression in the host mammalian cell. The expression vector can be introduced into a mammalian host cell and, under host cell culture conditions, the immunomodulatory protein is expressed on the surface or is secreted.

In an exemplary example, primary T-cells can be purified ex vivo (CD4 cells or CD8 cells or both) and stimulated with an activation protocol consisting of various TCR/CD28 agonists, such as anti-CD3/anti-CD28 coated beads. After a 2 or 3 day activation process, a recombinant expression vector containing an immunomodulatory polypeptide can be stably introduced into the primary T cells through art standard lentiviral or retroviral transduction protocols or plasmid electroporation strategies. Cells can be monitored for immunomodulatory polypeptide expression by, for example, flow cytometry using anti-epitope tag or antibodies that cross-react with native parental molecule and polypeptides comprising variant CD80. T-cells that express the immunomodulatory polypeptide can be enriched through sorting with anti-epitope tag antibodies or enriched for high or low expression depending on the application.

### 1. Transmembrane Immunomodulatory Proteins

In some embodiments, an immunomodulatory polypeptide comprising a variant CD80 can be a membrane bound protein. As described in more detail below, the immunomodulatory polypeptide can be a transmembrane immunomodulatory polypeptide comprising a variant CD80 in which is contained: an ectodomain containing at least one affinity modified IgSF domain (IgV or IgC), a transmembrane domain and, optionally, a cytoplasmic domain. In some embodiments, the transmembrane immunomodulatory protein can be expressed on the surface of an immune cell, such as a mammalian cell, including on the surface of a lymphocyte (e.g., T cell or NK cell) or antigen presenting cell. In some embodiments, the transmembrane immunomodulatory protein is expressed on the surface of a mammalian T-cell, including such T-cells as: a T helper cell, a cytotoxic T-cell (alternatively, cytotoxic T lymphocyte or CTL), a natural killer T-cell, a regulatory T-cell, a memory T-cell, or a gamma delta T-cell. In some embodiments, the mammalian cell is an antigen presenting cell (APC). Typically, but not exclusively, the ectodomain (alternatively, “extracellular domain”) of comprises the one or more amino acid variations (e.g., amino acid substitutions) of the variant CD80 of the invention. Thus, for example, in some embodiments a transmembrane protein will comprise an ectodomain that comprises one or more amino acid substitutions of a variant CD80 of the invention.

In some embodiments, the engineered cells express a variant CD80 polypeptides are transmembrane immunomodulatory polypeptides (TIPs) that can be a membrane protein such as a transmembrane protein. In typical embodiments, the ectodomain of a membrane protein comprises an extracellular domain or IgSF domain thereof of a variant CD80 provided herein in which is contained one or more amino acid substitutions in at least one IgSF domain as described. The transmembrane immunomodulatory proteins provided herein further contain a transmembrane domain linked to the ectodomain. In some embodiments, the transmembrane domain results in an encoded protein for cell surface expression on a cell. In some embodiments, the transmembrane domain is linked directly to the ectodomain. In some embodiments, the transmembrane domain is linked indirectly to the ectodomain via one or more linkers or spacers. In some embodiments, the transmembrane domain contains predominantly hydrophobic amino acid residues, such as leucine and valine.

In some embodiments, a full-length transmembrane anchor domain can be used to ensure that the TIPs will be expressed on the surface of the engineered cell, such as engineered T cell. Conveniently, this could be from a particular native protein that is being affinity modified (e.g., CD80 or other native IgSF protein), and simply fused to the sequence of the first membrane proximal domain in a similar fashion as the native IgSF protein (e.g., CD80). In some embodiments, the transmembrane immunomodulatory protein comprises a transmembrane domain of the corresponding wild-type or unmodified IgSF member, such as a transmembrane domain contained in the sequence of amino acids set forth in SEQ ID NO:395. In some embodiments, the membrane bound form comprises a transmembrane domain of the corresponding wild-type or unmodified polypeptide, such as corresponding to residues 243-263 of SEQ ID NO:395.

In some embodiments, the transmembrane domain is a non-native transmembrane domain that is not the transmembrane domain of native CD80. In some embodiments, the transmembrane domain is derived from a transmembrane domain from another non-CD80 family member polypeptide that is a membrane-bound or is a transmembrane protein. In some embodiments, a transmembrane anchor domain from another protein on T cells can be used. In some embodiments, the transmembrane domain is derived from CD8. In some embodiments, the transmembrane domain can further contain an extracellular portion of CD8 that serves as a spacer domain. An exemplary CD8 derived transmembrane domain is set forth in SEQ ID NO: 366, 367, or 368 or a portion thereof containing the CD8 transmembrane domain. In some embodiments, the transmembrane domain is a synthetic transmembrane domain.

In some embodiments, the transmembrane immunomodulatory protein further contains an endodomain, such as a cytoplasmic signaling domain, linked to the transmembrane domain. In some embodiments, the cytoplasmic signaling domain induces cell signaling. In some embodiments, the endodomain of the transmembrane immunomodulatory protein comprises the cytoplasmic domain of the corresponding wild-type or unmodified polypeptide, such as a cytoplasmic domain contained in the sequence of amino acids set forth in SEQ ID NO:395. In some embodiments, the membrane bound form comprises an endodomain of the corresponding wild-type or unmodified polypeptide, such as corresponding to residues 264-288 of SEQ ID NO:395.

In some embodiments, a provided transmembrane immunomodulatory protein that is or comprises a variant CD80 comprises a sequence of amino acids that exhibits at least 85%, 86%, 87%, 88%, 89%, 90%, 91%, 92%, 93%, 94%, 95%, 96%, 97%, 98% or 99% sequence identity to SEQ ID NO: 1 and contains an ectodomain comprising at least one affinity-modified CD80 IgSF domain as described and a transmembrane domain. In some embodiments, the transmembrane immunomodulatory protein contains any one or more amino acid substitutions in an IgSF domain (e.g., IgV domain) as described. In some embodiments, the transmembrane immunomodulatory protein can further comprise a cytoplasmic domain as described. In some embodiments, the transmembrane immunomodulatory protein can further contain a signal peptide. In some embodiments, the signal peptide is the native signal peptide of wild-type IgSF member, such as contained in the sequence of amino acids set forth in SEQ ID NO:395. In some embodiments, signal peptide is the signal peptide of the corresponding wild-type or unmodified polypeptide, such as corresponding to residues 1-34 of SEQ ID NO:395.

Also provided is a nucleic acid molecule encoding such transmembrane immunomodulatory proteins. In some embodiments, a nucleic acid molecule encoding a transmembrane immunomodulatory protein comprises a nucleotide sequence that encodes a sequence of amino acids that exhibits at least 85%, 86%, 87%, 88%, 89%, 90%, 91%, 92%, 93%, 94%, 95%, 96%, 97%, 98% or 99% sequence identity to SEQ ID NOS: 1 and contains an ectodomain comprising at least one affinity-modified IgSF domain as described, a transmembrane domain and, optionally, a cytoplasmic domain. In some embodiments, the nucleic acid molecule can further comprise a sequence of nucleotides encoding a signal peptide. In some embodiments, the signal peptide is the native signal peptide of the corresponding wild-type IgSF member, such as corresponding to residues 1-34 of SEQ ID NO:395.

In some embodiments, provided are CAR-related transmembrane immunomodulatory proteins in which the endodomain of a transmembrane immunomodulatory protein comprises a cytoplasmic signaling domain that comprises at least one ITAM (immunoreceptor tyrosine-based activation motif)-containing signaling domain. ITAM is a conserved motif found in a number of protein signaling domains involved in signal transduction of immune cells, including in the CD3-zeta chain (“CD3-z”) involved in T-cell receptor signal transduction. In some embodiments, the endodomain comprises at CD3-zeta signaling domain. In some embodiments, the CD3-zeta signaling domain comprises the sequence of amino acids set forth in SEQ ID NO: 333 or a sequence of amino acids that exhibits at least 85%, 86%, 87%, 88%, 89%, 90%, 91%, 92%, 93%, 94%, 95%, 96%, 97%, 98% or 99% to SEQ ID NO:369 and retains the activity of T cell signaling. In some embodiments, the endodomain of a CAR-related transmembrane immunomodulatory protein can further comprise a costimulatory signaling domain to further modulate immunomodulatory responses of the T-cell. In some embodiments, the costimulatory signaling domain is CD28, ICOS, 41BB or OX40. In some embodiments, the costimulatory signaling domain is a derived from CD28 or 4-1BB and comprises the sequence of amino acids set forth in any of SEQ ID NOS: 370-373 or a sequence of amino acids that exhibits at least 85%, 86%, 87%, 88%, 89%, 90%, 91%, 92%, 93%, 94%, 95%, 96%, 97%, 98% or 99% to SEQ ID NO:370-373 and retains the activity of T cell costimulatory signaling. In some embodiments, the provided CAR-related transmembrane immunomodulatory proteins have features of CARs to stimulate T cell signaling upon binding of an affinity modified IgSF domain to a cognate binding partner or counter structure. In some embodiments, upon specific binding by the affinity-modified IgSF domain to its counter structure can lead to changes in the immunological activity of the T-cell activity as reflected by changes in cytotoxicity, proliferation or cytokine production.

In some embodiments, the transmembrane immunomodulatory protein does not contain an endodomain capable of mediating cytoplasmic signaling. In some embodiments, the transmembrane immunomodulatory protein lacks the signal transduction mechanism of the wild-type or unmodified polypeptide and therefore does not itself induce cell signaling. In some embodiments, the transmembrane immunomodulatory protein lacks an intracellular (cytoplasmic) domain or a portion of the intracellular domain of the corresponding wild-type or unmodified polypeptide, such as a cytoplasmic signaling domain contained in the sequence of amino acids set forth in SEQ ID NO:395 corresponding to residues 264-288 of SEQ ID NO:395. In some embodiments, the transmembrane immunomodulatory protein does not contain an ITIM (immunoreceptor tyrosine-based inhibition motif), such as contained in certain inhibitory receptors, including inhibitory receptors of the IgSF family (e.g., PD-1 or TIGIT). Thus, in some embodiments, the transmembrane immunomodulatory protein only contains the ectodomain and the transmembrane domain, such as any as described.

### 2. Secretable Immunomodulatory Proteins

In some embodiments, the CD80 variant immunomodulatory polypeptide containing any one or more of the amino acid mutations as described herein, is secretable, such as when expressed from a cell. Such a variant CD80 immunomodulatory protein does not comprise a transmembrane domain. In some embodiments, the variant CD80 immunomodulatory protein is not conjugated to a half-life extending moiety (such as an Fc domain or a multimerization domain). In some embodiments, the variant CD80 immunomodulatory protein comprises a signal peptide, e.g., an antibody signal peptide or other efficient signal sequence to get domains outside of cell. When the immunomodulatory protein comprises a signal peptide and is expressed by an engineered cell, the signal peptide causes the immunomodulatory protein to be secreted by the engineered cell. Generally, the signal peptide, or a portion of the signal peptide, is cleaved from the immunomodulatory protein with secretion. The immunomodulatory protein can be encoded by a nucleic acid (which can be part of an expression vector). In some embodiments, the immunomodulatory protein is expressed and secreted by a cell (such as an immune cell, for example a primary immune cell).

Thus, in some embodiments, there are provided variant CD80 immunomodulatory proteins that further comprises a signal peptide. In some embodiments, provided herein is a nucleic acid molecule encoding the variant CD80 immunomodulatory protein operably connected to a secretion sequence encoding the signal peptide.

A signal peptide is a sequence on the N-terminus of an immunomodulatory protein that signals secretion of the immunomodulatory protein from a cell. In some embodiments, the signal peptide is about 5 to about 40 amino acids in length (such as about 5 to about 7, about 7 to about 10, about 10 to about 15, about 15 to about 20, about 20 to about 25, or about 25 to about 30, about 30 to about 35, or about 35 to about 40 amino acids in length).

In some embodiments, the signal peptide is a native signal peptide from the corresponding wild-type CD80. In some embodiments, the signal peptide is the native signal peptide of the corresponding wild-type CD80, such as corresponding to residues 1-34 of SEQ ID NO:395.

In some embodiments, the signal peptide is a non-native signal peptide. For example, in some embodiments, the non-native signal peptide is a mutant native signal peptide from the corresponding wild-type CD80, and can include one or more (such as 2, 3, 4, 5, 6, 7, 8, 9, or 10 or more) substitutions insertions or deletions. In some embodiments, the non-native signal peptide is a signal peptide or mutant thereof of a family member from the same IgSF family as the wild-type IgSF family member. In some embodiments, the non-native signal peptide is a signal peptide or mutant thereof from an IgSF family member from a different IgSF family that the wild-type IgSF family member. In some embodiments, the signal peptide is a signal peptide or mutant thereof from a non-IgSF protein family, such as a signal peptide from an immunoglobulin (such as IgG heavy chain or IgG-kappa light chain), a cytokine (such as interleukin-2 (IL-2), or CD33), a serum albumin protein (e.g., HSA or albumin), a human azurocidin preprotein signal sequence, a luciferase, a trypsinogen (e.g., chymotrypsinogen or trypsinogen) or other signal peptide able to efficiently secrete a protein from a cell. Exemplary signal peptides include any described in the Table 2.

TABLE 2
Exemplary Signal Peptides

| SEQ ID NO | Signal Peptide | Peptide Sequence |
|---|---|---|
| SEQ ID NO: 396 | HSA signal peptide | MKWVTFISLLFLFSSAYS |
| SEQ ID NO: 397 | Ig kappa light chain | MDMRAPAGIFGFLLVLFPGYRS |
| SEQ ID NO: 398 | human azurocidin preprotein | MTRLTVLALLAGLLASSRA |
| | signal sequence | |
| SEQ ID NO: 399 | IgG heavy chain signal peptide | MELGLSWIFLLAILKGVQC |
| SEQ ID NO: 400 | IgG heavy chain signal peptide | MELGLRWVFLVAILEGVQC |
| SEQ ID NO: 401 | IgG heavy chain signal peptide | MKHLWFFLLLVAAPRWVLS |
| SEQ ID NO: 402 | IgG heavy chain signal peptide | MDWTWRILFLVAAATGAHS |
| SEQ ID NO: 403 | IgG heavy chain signal peptide | MDWTWRFLFVVAAATGVQS |
| SEQ ID NO: 404 | IgG heavy chain signal peptide | MEFGLSWLFLVAILKGVQC |
| SEQ ID NO: 405 | IgG heavy chain signal peptide | MEFGLSWVFLVALFRGVQC |
| SEQ ID NO: 406 | IgG heavy chain signal peptide | MDLLHKNMKHLWFFLLLVAAPRW |
| | | VLS |
| SEQ ID NO: 407 | IgG Kappa light chain signal | MDMRVPAQLLGLLLLWLSGARC |
| | sequences: | |
| SEQ ID NO: 408 | IgG Kappa light chain signal | MKYLLPTAAAGLLLLAAQPAMA |
| | sequences: | |
| SEQ ID NO: 409 | Gaussia luciferase | MGVKVLFALICIAVAEA |
| SEQ ID NO: 410 | Human albumin | MKWVTFISLLFLFSSAYS |
| SEQ ID NO: 411 | Human chymotrypsinogen | MAFLWLLSCWALLGTTFG |
| SEQ ID NO: 412 | Human interleukin-2 | MQLLSCIALILALV |
| SEQ ID NO: 413 | Human trypsinogen-2 | MNLLLILTFVAAAVA |

In some embodiments of a secretable variant CD80 immunomodulatory protein, the immunomodulatory protein comprises a signal peptide when expressed, and the signal peptide (or a portion thereof) is cleaved from the immunomodulatory protein upon secretion.

In some embodiments, the engineered cells express variant CD80 polypeptides that are secreted from the cell. In some embodiments, such a variant CD80 polypeptide is encoded by a nucleic acid molecule encoding an immunomodulatory protein under the operable control of a signal sequence for secretion. In some embodiments, the encoded immunomodulatory protein is secreted when expressed from a cell. In some embodiments, the immunomodulatory protein encoded by the nucleic acid molecule does not comprise a transmembrane domain.

In some embodiments, the immunomodulatory protein encoded by the nucleic acid molecule does not comprise a half-life extending moiety (such as an Fc domain or a multimerization domain). In some embodiments, the immunomodulatory protein encoded by the nucleic acid molecule comprises a signal peptide. In some embodiments, a nucleic acid of the invention further comprises nucleotide sequence that encodes a secretory or signal peptide operably linked to the nucleic acid encoding the immunomodulatory protein, thereby allowing for secretion of the immunomodulatory protein.

In some embodiments, the secretable immunomodulatory protein is a fusion protein containing a multimerization domain, such as an Fc domain. In some embodiments, the secretable immunomodulatory protein is a variant CD80-Fc fusion protein. In some embodiments, the fusion protein is encoded by a nucleic acid expressed by the cell in which, upon expression by the cell, a dimer of the fusion protein is expressed and secreted. In some embodiments, the nucleic acid further comprises a nucleotide sequence that encodes a secretory or signal peptide operably linked to the nucleic acid encoding the fusion protein (e.g. CD80-Fc), thereby allowing for secretion of the fusion protein (e.g. CD80-Fc). Exemplary fusion proteins are described in the following subsection.

3. Co-Expression with Antigen Receptors

In some embodiments a cellular aAPC can be engineered to contain a TIP and TCR agonist which is used in adoptive cellular therapy. In some embodiments, a cellular aAPC can be engineered to contain a TIP and TCR agonist which is used in ex vivo expansion of human T cells, such as prior to administration, e.g., for reintroduction into the patient. In some aspects, the aAPC may include expression of at least one anti-CD3 antibody clone, e.g., such as, for example, OKT3 and/or UCHT1. In some aspects, the aAPCs may be inactivated (e.g., irradiated). In some embodiment, the TIP can include any variant IgSF domain that exhibits binding affinity for a cognate binding partner on a T cell.

In some embodiments, an immunomodulatory protein provided herein, such as a transmembrane immunomodulatory protein or a secretable immunomodulatory protein, is co-expressed or engineered into a cell that expresses an antigen-binding receptor, such as a recombinant receptor, such as a chimeric antigen receptor (CAR) or T cell receptor (TCR). In some embodiments, the engineered cell, such as an engineered T cell, recognizes a desired antigen associated with cancer, inflammatory and autoimmune disorders, or a viral infection. In specific embodiments, the antigen-binding receptor contains an antigen-binding moiety that specifically binds a tumor specific antigen or a tumor associated antigen. In some embodiments, the engineered T-cell is a CAR (chimeric antigen receptor) T-cell that contains an antigen-binding domain (e.g., scFv) that specifically binds to an antigen, such as a tumor specific antigen or tumor associated antigen. In some embodiments, the TIP protein is expressed in an engineered T-cell receptor cell or an engineered chimeric antigen receptor cell. In such embodiments, the engineered cell co-expresses the TIP and the CAR or TCR. In some embodiments, the SIP protein is expressed in an engineered T-cell receptor cell or an engineered chimeric antigen receptor cell. In such embodiments, the engineered cell co-expresses the SIP and the CAR or TCR.

Chimeric antigen receptors (CARs) are recombinant receptors that include an antigen-binding domain (ectodomain), a transmembrane domain and an intracellular signaling region (endodomain) that is capable of inducing or mediating an activation signal to the T cell after the antigen is bound. In some example, CAR-expressing cells are engineered to express an antigen-binding domain that is an antibody or antigen-binding fragment with specificity for a particular tumor antigen linked to an intracellular signaling part comprising an activating domain and, in some cases, a costimulatory domain. The costimulatory domain can be derived from, e.g., CD28, OX-40, 4-1BB/CD137, inducible T cell costimulator (ICOS), The activating domain can be derived from, e.g., CD3, such as CD3 zeta, epsilon, delta, gamma, or the like. In certain embodiments, the CAR is designed to have two, three, four, or more costimulatory domains. In some aspects, the antigen-binding domain is an antibody or antigen-binding fragment thereof, such as a single chain fragment (scFv). The CAR scFv can be designed to target an antigen expressed on a cell associated with a disease or condition, e.g., a tumor antigen. In some embodiments, the antigen is expressed on a tumor or cancer cell. Examples of tumor antigens include, but are not limited to, CD19, HER2, BCMA, or CD22. Example CAR+ T cell therapies and constructs are described in U.S. Patent Publication Nos. 2013/0287748, 2014/0227237, 2014/0099309, and 2014/0050708, and these references are incorporated by reference in their entirety.

In some embodiments, the antigen recognized by the CAR is CD19, which is a transmembrane protein expressed by cells in the B cell lineage, including all normal B cells and B cell malignances, including but not limited to NHL, CLL, and non-T cell ALL. In some embodiments, the CAR is an anti-CD19 CAR, such as a CAR containing an anti-CD19 scFv set forth in SEQ ID NO:365. In some embodiments, the CAR is an anti-CD19 CAR, such as a CAR containing an anti-CD19 scFv set forth in SEQ ID NO:393. Other exemplary CARs include anti-HER2 CARs, anti-BCMA CARs, anti-CD22 CARs and other CARs specific to tumor-associated antigens. For instance, in some embodiments, the CAR comprises an anti-HER scFv containing a variable heavy and light chains of trastuzumab. CAR-T cells are well known to a skilled artisan.

In some embodiments, the CAR further contains a spacer, a transmembrane domain, and an intracellular signaling domain or region comprising an ITAM signaling domain, such as a CD3zeta signaling domain. In some embodiments, the CAR further includes a costimulatory signaling domain. In some embodiments, the CAR contains an scFv antigen-binding domain, a CD8 hinge region, a transmembrane domain, and intracellular signaling domains derived from 4-1BB and CD3-zeta signaling domains.

In some embodiments, the spacer and transmembrane domain are the hinge and transmembrane domain derived from CD8, such as having an exemplary sequence set forth in SEQ ID NO: 366, 367, 368 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:366, 367, 368. In some embodiments, the endodomain comprises at CD3-zeta signaling domain. In some embodiments, the CD3-zeta signaling domain comprises the sequence of amino acids set forth in SEQ ID NO: 369 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO:369 and retains the activity of T cell signaling. In some embodiments, the endodomain of a CAR can further comprise a costimulatory signaling domain or region to further modulate immunomodulatory responses of the T-cell. In some embodiments, the costimulatory signaling domain is or comprises a costimulatory region, or is derived from a costimulatory region, of CD28, ICOS, 41BB or OX40. In some embodiments, the costimulatory signaling domain is a derived from CD28 or 4-1BB and comprises the sequence of amino acids set forth in any of SEQ ID NOS: 370-373 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO:370-373 and retains the activity of T cell costimulatory signaling.

In some embodiments, the construct encoding the CAR further encodes a second protein, such as a marker, e.g., detectable protein, separated from the CAR by a self-cleaving peptide sequence. In some embodiments, the self-cleaving peptide sequence is an F2A, T2A, E2A or P2A self-cleaving peptide. Exemplary sequences of a T2A self-cleaving peptide are set for the in any one of SEQ ID NOS: 374, 375, 376 or a sequence of amino acids that exhibits at least 85%, 86%, 87%, 88%, 89%, 90%, 91%, 92%, 93%, 94%, 95%, 96%, 97%, 98% or 99% or more sequence identity to any of SEQ ID NOS: 374, 375, 376. In some embodiments, the T2A is encoded by the sequence of nucleotides set forth in SEQ ID NO:377 or a sequence that exhibits at least 85%, 86%, 87%, 88%, 89%, 90%, 91%, 92%, 93%, 94%, 95%, 96%, 97%, 98% or 99% or more sequence identity to any of SEQ ID NO: 377. An exemplary sequence of a P2A self-cleaving peptide is set in SEQ ID NO: 378 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NOS: 378. In some cases, a nucleic acid construct that encodes more than one P2A self-cleaving peptide (such as a P2A1 and P2A2), in which the nucleotide sequence P2A1 and P2A2 each encode the P2A set forth in SEQ ID NO:378, the nucleotide sequence may be different to avoid recombination between sequences.

In some embodiments, the marker is a detectable protein, such as a fluorescent protein, e.g., a green fluorescent protein (GFP) or blue fluorescent protein (BFP). Exemplary sequences of a fluorescent protein marker are set forth in SEQ ID NO: 379, 380, 381, 382, 383, or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO: 379, 380, 381, 382, 383.

In some embodiments, the CAR has the sequence of amino acids set forth in any of SEQ ID NOS: 384, 385, 386, 387, 388, 389, 391, 392 or a sequence of amino acids that exhibits at least 85%, 86%, 87%, 88%, 89%, 90%, 91%, 92%, 93%, 94%, 95%, 96%, 97%, 98% or 99% or more sequence identity to any one of SEQ ID NOS: 384, 385, 386, 387, 388, 389, 391, 392. In some embodiments, the CAR is encoded by a sequence of nucleotides set forth in SEQ ID NO: 390 or 394 or a sequence of amino acids that exhibits at least 85%, 86%, 87%, 88%, 89%, 90%, 91%, 92%, 93%, 94%, 95%, 96%, 97%, 98% or 99% or more sequence identity to any one of SEQ ID NO: 390 or 394.

In another embodiment, the engineered T-cell possesses a TCR, including a recombinant or engineered TCR. In some embodiments, the TCR can be a native TCR. Those of skill in the art will recognize that generally native mammalian T-cell receptors comprise an alpha and a beta chain (or a gamma and a delta chain) involved in antigen specific recognition and binding. In some embodiments, the TCR is an engineered TCR that is modified. In some embodiments, the TCR of an engineered T-cell specifically binds to a tumor associated or tumor specific antigen presented by an APC.

In another embodiment, the engineered T-cell possesses a TCR, including a recombinant or engineered TCR. In some embodiments, the TCR can be a native TCR. Those of skill in the art will recognize that generally native mammalian T-cell receptors comprise an alpha and a beta chain (or a gamma and a delta chain) involved in antigen specific recognition and binding. In some embodiments, the TCR is an engineered TCR that is modified. In some embodiments, the TCR of an engineered T-cell specifically binds to a tumor associated or tumor specific antigen presented by an APC. In some embodiments, the TCR is an HPV16 E6 peptide (E6 TCR). In some embodiments, the TCR is an HPV16 E7 peptide (E7 TCR). Exemplary HPV TCRs include those described in International published PCT Appl. No. WO2015009606 or WO2015184228.

Upon immunomodulatory polypeptide expression the engineered T-cell can be assayed for appropriate function by a variety of means. The engineered CAR or TCR co-expression can be validated to show that this part of the engineered T cell was not significantly impacted by the expression of the immunomodulatory protein. Once validated, standard in vitro cytotoxicity, proliferation, or cytokine assays (e.g., IFN-gamma expression) can be used to assess the function of engineered T-cells. Exemplary standard endpoints are percent lysis of the tumor line, proliferation of the engineered T-cell, or IFN-gamma protein expression in culture supernatants. An engineered construct which results in statistically significant increased lysis of tumor line, increased proliferation of the engineered T-cell, or increased IFN-gamma expression over the control construct can be selected for. Additionally, non-engineered, such as native primary or endogenous T-cells could also be incorporated into the same in vitro assay to measure the ability of the immunomodulatory polypeptide construct expressed on the engineered cells, such as engineered T-cells, to modulate activity, including, in some cases, to activate and generate effector function in bystander, native T-cells. Increased expression of activation markers such as CD69, CD44, or CD62L could be monitored on endogenous T cells, and increased proliferation and/or cytokine production could indicate desired activity of the immunomodulatory protein expressed on the engineered T cells.

In some embodiments, the similar assays can be used to compare the function of engineered T cells containing the CAR or TCR alone to those containing the CAR or TCR and a TIP construct. Typically, these in vitro assays are performed by plating various ratios of the engineered T cell and a “tumor” cell line containing the cognate CAR or TCR antigen together in culture. Standard endpoints are percent lysis of the tumor line, proliferation of the engineered T cell, or IFN-gamma production in culture supernatants. An engineered immunomodulatory protein which resulted in statistically significant increased lysis of tumor line, increased proliferation of the engineered T cell, or increased IFN-gamma production over the same TCR or CAR construct alone can be selected for. Engineered human T cells can be analyzed in immunocompromised mice, like the NSG strain, which lacks mouse T, NK and B cells. Engineered human T cells in which the CAR or TCR binds a target counter-structure on the xenograft and is co-expressed with the TIP affinity modified IgSF domain can be adoptively transferred in vivo at different cell numbers and ratios compared to the xenograft. For example, engraftment of CD19+ leukemia tumor lines containing a luciferase/GFP vector can be monitored through bioluminescence or ex vivo by flow cytometry. In a common embodiment, the xenograft is introduced into the murine model, followed by the engineered T cells several days later. Engineered T cells containing the immunomodulatory protein can be assayed for increased survival, tumor clearance, or expanded engineered T cells numbers relative to engineered T cells containing the CAR or TCR alone. As in the in vitro assay, endogenous, native (i.e., non-engineered) human T cells could be co-adoptively transferred to look for successful epitope spreading in that population, resulting in better survival or tumor clearance.

In some embodiments, provided engineered T cells expressing a provided immunomodulatory protein exhibits one or more improved properties or activities compared to reference cells that have not been so engineered with an immunomodulatory protein (e.g. TIP) as described herein. In some embodiments, the reference cell, such as a reference T cells, reference CAR-engineered T cells, or reference TCR-engineered T cells, are cells that have been produced or engineered by similar ex vivo procedures but that do not express or have not been engineered to express the immunomodulatory protein. In some embodiments, the property or activity is associated with or related to T-cell function. In some embodiments, the one or more properties or activities include, but are not limited to, cellular proliferation, cytototoxic activity, cytokine production (e.g. IFN-gamma, IL-2 or TNF-alpha), and/or expression of one or more activation markers (e.g. CD69 or CD25). In some embodiments, the activity or property is increased by at least or at least about 1.2-fold, 1.4-fold, 1.5-fold, 1.6-fold, 1.7-fold, 1.8-fold, 1.9-fold, 2.0-fold, 2.5-fold, 3.0-fold, 4.0-fold, 5.0-fold, or more compared to the reference cell or reference cell composition.

### D. Exemplary Activity and Methods of Assessing Immune Modulation

In some embodiments, a variant CD80 polypeptide has a binding affinity for CTLA-4, PD-L1, or CD28 that differs from that of a wild-type or unmodified CD80 polypeptide control sequence as determined by, for example, solid-phase ELISA immunoassays, flow cytometry or surface plasmon resonance (Biacore) assays. In some embodiments, the variant CD80 polypeptide has an increased binding affinity for CTLA-4, PD-L1, and/or CD28. In some embodiments, the variant CD80 polypeptide has a decreased binding affinity for CD28, PD-L1, and/or CTLA-4, relative to a wild-type or unmodified CD80 polypeptide. The CD28, PD-L1 and/or the CTLA-4 can be a mammalian protein, such as a human protein or a murine protein. The altered, e.g. increased or decreased, binding activity or affinity for CTLA-4, PD-L1 and/or CD28 is conferred by one or more amino acid modifications (e.g. amino acid substitutions) as described.

In some embodiments, the one or more amino acid modifications (e.g. amino acid substitutions) of a variant CD80 polypeptide provided herein produces at least one affinity-modified IgSF domain of a CD80 extracellular domain (e.g., IgV) relative to an IgSF domain contained in a wild-type or unmodified CD80 polypeptide such that the variant CD80 polypeptide exhibits altered (increased or decreased) binding activity or affinity for one or more binding partners, CTLA-4, PD-L1, or CD28, compared to a wild-type or unmodified CD80 polypeptide. In some embodiments, a variant CD80 polypeptide has a binding affinity for CTLA-4, PD-L1, or CD28 that differs from that of a wild-type or unmodified CD80 polypeptide control sequence as determined by, for example, solid-phase ELISA immunoassays, flow cytometry or surface plasmon resonance (Biacore) assays. In some embodiments, the variant CD80 polypeptide has an increased binding affinity for CTLA-4, PD-L1, and/or CD28. In some embodiments, the variant CD80 polypeptide has a decreased binding affinity for CD28, PD-L1, and/or CTLA-4, relative to a wild-type or unmodified CD80 polypeptide. The CD28, PD-L1 and/or the CTLA-4 can be a mammalian protein, such as a human protein or a murine protein.

Binding affinities for each of the binding partners are independent; that is, in some embodiments, a variant CD80 polypeptide has an increased binding affinity for one, two or three of CD28, PD-L1, and CTLA-4, and/or a decreased binding affinity for one, two or three of CD28, PD-L1, and CTLA-4, relative to a wild-type or unmodified CD80 polypeptide.

In some embodiments, the variant CD80 polypeptide has an increased binding affinity for PD-L1, relative to a wild-type or unmodified CD80 polypeptide. In some embodiments, a variant CD80 polypeptide with increased or greater binding affinity to PD-L1 will have an increase in binding affinity relative to the wild-type or unmodified CD80 polypeptide control of at least about 5%, such as at least about 10%, 15%, 20%, 25%, 35%, or 50% for the PD-L1 binding partner. In some embodiments, the increase in binding affinity relative to the wild-type or unmodified CD80 polypeptide is more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 250-fold, 300-fold, 350-fold, 400-fold, or more. In such examples, the wild-type or unmodified CD80 polypeptide has the same sequence as the variant CD80 polypeptide except that it does not contain the one or more amino acid modifications (e.g., substitutions).

In some embodiments, the equilibrium dissociation constant (K_{d}) of any of the foregoing embodiments to PD-L1 can be at least 1×10⁻⁵ M, 1×10⁻⁶ M, 1×10⁻⁷ M, 1×10⁻⁸ M, 1×10⁻⁹ M, 1×10⁻¹⁰ M or 1×10⁻¹¹ M, or 1×10⁻¹² M.

In some embodiment, the provided immunomodulatory proteins (e.g. CD80-Fc fusion protein) exhibits increased binding to PD-L1 that is with a relatively slow off-rate. As shown here, immunomodulatory proteins composed of certain variant CD80 polypeptides revealed a vastly improved Koff rate compared to wild-type or unmodified CD80. Without wishing to be bound by theory, it is believed that a relatively slow Koff rate would be desirable to improve pharmacokinetic profile of the molecule, particularly in the tumor environment where PD-L1 is expressed. In some embodiments, the off rate, “Koff” or “kd” is, for example, as determined by surface plasmon resonance, such as BIACORE™. In some embodiments, the off-rate is less than 50×10 3 s⁻¹. In some embodiments, the off-rate of a provided immunomodulatory protein (e.g. CD80-Fc) for binding PD-L1 is at or about or less than 40×10⁻³ s⁻¹, 30×10⁻³ s⁻¹, 20×10⁻³ s⁻¹, 15×10⁻³ s⁻¹, 10×10⁻³ s⁻¹, 5×10⁻³ s⁻¹, or 1×10⁻³ s⁻¹. In some embodiments, the off-rate of a provided immunomodulatory protein for binding to PD-L1 is between 1×10⁻³ s⁻¹ and 50×10⁻³ s, 1×10⁻³ s⁻¹ and 30×10⁻³ s, 1×10⁻³ s⁻¹ and 20×10⁻³ s⁻¹, 1×10⁻³ s⁻¹ and 15×10⁻³ s⁻¹, 1×10⁻³ s⁻¹ and 10×10⁻³ s⁻¹, 1×10⁻³ s⁻¹ and 5×10⁻³ s⁻¹, 5×10⁻³ s⁻¹ and 50×10⁻³ s⁻¹, 5×10⁻³ s⁻¹ and 30×10⁻³ s⁻¹, 5×10⁻³ s⁻¹ and 20×10⁻³ s⁻¹, 5×10⁻³ s⁻¹ and 15×10⁻³ s⁻¹, 5×10⁻³ s⁻¹ and 10×10⁻³ s⁻¹, 10×10⁻³ s⁻¹ and 50×10⁻³ s⁻¹, 10×10⁻³ s⁻¹ and 30×10⁻³ s⁻¹, 10×10⁻³ s⁻¹ and 20×10⁻³ s⁻¹, 1×10⁻³ s⁻¹ and 15×10⁻³ s⁻¹, 15×10⁻³ s⁻¹ and 50×10⁻³ s⁻¹, 15×10⁻³ s⁻¹ and 30×10⁻³ s⁻¹, 15×10⁻³ s⁻¹ and 20×10⁻³ s⁻¹, 20×10⁻³ s⁻¹ and 50×10⁻³ s⁻¹, 20×10⁻³ s⁻¹ and 30×10⁻³ s⁻¹, or 30×10⁻³ s⁻¹ and 50×10⁻³ s⁻¹. In particular embodiments, the off-rate of a provided immunomodulatory protein for binding PD-L1 is between 10×10⁻³ s⁻¹ and 30×10⁻³ s⁻¹.

In some embodiments, the immunomodulatory protein (e.g. CD80-Fc fusion protein) antagonizes or blocks activity of PD-1/PD-L1 interactions and thereby reduces PD-1 inhibitory receptor activity. In some embodiments, the provided immunomodulatory proteins block the ability of PD-L1 to bind to its cognate inhibitory receptor binding partner PD-1. In some embodiments, the activity of the provided immunomodulatory proteins (e.g. CD80-Fc) to block PD-1/PD-L1 interactions can, in some cases, increase immune responses of T lymphocytes that express PD-1.

In some embodiments, the variant CD80 polypeptide has an increased binding affinity for CD28, relative to a wild-type or unmodified CD80 polypeptide. In some embodiments, a variant CD80 polypeptide with increased or greater binding affinity to CD28 will have an increase in binding affinity relative to the wild-type or unmodified CD80 polypeptide control of at least about 5%, such as at least about 10%, 15%, 20%, 25%, 35%, or 50% for the CD28 binding partner. In some embodiments, the increase in binding affinity relative to the wild-type or unmodified CD80 polypeptide is more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 250-fold, 300-fold, 350-fold, 400-fold, or more. In such examples, the wild-type or unmodified CD80 polypeptide has the same sequence as the variant CD80 polypeptide except that it does not contain the one or more amino acid modifications (e.g., substitutions).

In some embodiments, the equilibrium dissociation constant (K_{d}) of any of the foregoing embodiments to CD28 can be at least 1×10⁻⁵ M, 1×10⁻⁶ M, 1×10⁻⁷ M, 1×10⁻⁸ M, 1×10⁻⁹ M, 1×10⁻¹⁰ M or 1×10⁻¹¹ M, or 1×10⁻¹² M.

In some embodiments, the variant CD80 polypeptide has an increased binding affinity for PD-L1 and an increased binding affinity for CD28, relative to a wild-type or unmodified CD80 polypeptide. In some embodiments, the variant CD80 polypeptide has an increased binding affinity for PD-L1 and a decreased binding affinity for CD28, relative to a wild-type or unmodified CD80 polypeptide. In some embodiments, the variant CD80 polypeptide has an increased binding affinity for PD-L1 and exhibits similar binding affinity for CD28, relative to a wild-type or unmodified CD80 polypeptide.

In some embodiments, the variant CD80 polypeptide increases the signaling induced by CD28, upon binding, relative to a wild-type or unmodified CD80 polypeptide. In some embodiments, a variant CD80 polypeptide that stimulates or increases the signaling induced by CD28 will produce a signal that is at least 105%, 110%, 120%, 150%, 200%, 300%, 400%, or 500%, or more of the signal induced by the wild-type or unmodified CD80 polypeptide. In such examples, the wild-type or unmodified CD80 polypeptide has the same sequence as the variant CD80 polypeptide except that it does not contain the one or more amino acid modifications (e.g., substitutions).

In some embodiments, an immunomodulatory protein containing a variant CD80 polypeptide (e.g. CD80-Fc) that contains a variant CD80 polypeptide that exhibits increased binding to PD-L1 also binds to CD28. In some embodiments, the binding to CD28 also is increased relative to an unmodified or wild-type CD80. In particular embodiments, the ability of a variant CD80 polypeptide to bind both PD-L1 and CD28 is non-competitive such that both ligands can be bound simultaneously by the provided immunomodulatory protein (e.g. CD80-Fc). Also among provided embodiments are methods for mediating agonism of CD28 by PD-L1 dependent CD28 costimulation using variant CD80 polypeptides that exhibit increased binding to PD-L1 compared to unmodified or wild-type CD80 polypeptide. In some aspects, such methods can be used to increase an immune response in a subject administered the molecules, which, in some aspects, can treat a disease or condition in the subject, such as treatment of a tumor or cancer. Such PD-L1-dependent costimulation does not require an Fc with effector function and can be mediated by an Fc fusion protein containing an effector-less or inert Fc molecule. In some cases, such immunomodulatory proteins, e.g. CD80-Fc, also can facilitate promotion of an immune response in connection with the provided therapeutic methods by blocking the PD-L1/PD-1 interaction while also binding and co-stimulating a CD28 receptor on a localized T cell.

In some embodiments, a provided immunomodulatory protein is a CD80-Fc fusion in which the Fc domain binds to an FcR. In some embodiments, a variant CD80 polypeptide that is linked, directly or indirectly to an Fc that binds to an FcR, such as via an IgG4 Fc, may mediate CD28 agonism, which in some aspects may be independent of PD-L1 binding. There is provided methods for mediating agonism of CD28 by receptor-dependent CD28 costimulation using variant CD80 polypeptides provided herein the bind to CD28. In some embodiments, such agonism of CD28 may be useful to promote immunity in oncology, such as for treatment of tumors or cancer. In some cases, the variant CD80 polypeptides also bind CTLA-4 or PD-L1, such as exhibit increased binding to CTLA-4 or PD-L1, which may block inhibitory signaling by these checkpoint pathways. In some aspects, crosslinking the Fc receptor can initiate antibody-dependent cell cytotoxicity (ADCC)-mediated effector functions, and thereby effect depletion of target cells expressing the cognate binding partner, such as CTLA-4-expressing cells (e.g. CTLA-4-expressing T regulatory cells) or PD-L1-expressing cells (e.g. PD-Llhi tumors). The provided methods to modulate an immune response can be used to treat a disease or condition, such as a tumor or cancer. In some embodiments, the pharmaceutical composition can be used to inhibit growth of mammalian cancer cells (such as human cancer cells).

In some embodiments, the variant CD80 polypeptide has a decreased binding affinity for CTLA-4, relative to a wild-type or unmodified CD80 polypeptide. In some embodiments, a variant CD80 polypeptide with decreased or reduced binding affinity to CTLA-4 will have decrease in binding affinity relative to the wild-type or unmodified CD80 polypeptide control of at least 5%, such as at least about 10%, 15%, 20%, 30%, 40%, 50%, 60%, 70%, 80%, 90% or more for the CTLA-4. In some embodiments, the decrease in binding affinity relative to the wild-type or unmodified CD80 polypeptide is more than 1.2-fold, 1.5-fold, 2-fold, 3-fold, 4-fold, 5-fold, 6-fold, 7-fold, 8-fold, 9-fold, 10-fold, 20-fold, 30-fold 40-fold or 50-fold. In such examples, the wild-type or unmodified CD80 polypeptide has the same sequence as the variant CD80 polypeptide except that it does not contain the one or more amino acid modifications (e.g., substitutions).

In some embodiments, the variant CD80 polypeptide exhibits binding affinity to the ectodomain of human CTLA-4 which is no higher than the binding affinity of the unmodified or wild-type CD80 for the ectodomain of human CTLA-4.

In some embodiments, the equilibrium dissociation constant (K_{d}) of any of the foregoing embodiments to CTLA-4 can be at least 1×10⁻⁵ M, 1×10⁻⁶ M, 1×10⁻⁷ M, 1×10⁻⁸ M, 1×10⁻⁹ M, 1×10⁻¹⁰ M or 1×10⁻¹¹ M, or 1×10⁻¹² M.

In some embodiments, the immunomodulatory protein (e.g. CD80-Fc fusion protein) antagonizes or blocks activity of the CTLA-4 inhibitory receptor. In some embodiments, the provided immunomodulatory proteins block the ability of CTLA-4 to bind to its cognate binding partners CD80 or CD86. In some embodiments, the activity of the provided immunomodulatory proteins (e.g. CD80-Fc) to block activity of CTLA-4 can, in some cases, increase immune responses of T lymphocytes that express CTLA-4.

In some embodiments, the variant CD80 polypeptides or immunomodulatory proteins provided herein exhibit immunomodulatory activity to modulate T cell activation. In some embodiments, the provided variant CD80 polypeptides or immunomodulatory proteins modulate cytokine production, such as IFN-gamma, TNFα, or IL-2 expression, in a T cell assay relative to a wild-type or unmodified CD80 control. In some cases, modulation of IFN-gamma expression increases IFN-gamma expression relative to the control. In some cases, modulation of IFN-gamma expression decreases IFN-gamma expression relative to the control. Assays to determine specific binding and IFN-gamma expression are well-known in the art and include the MLR (mixed lymphocyte reaction) assays measuring interferon-gamma cytokine levels in culture supernatants (Wang et al., Cancer Immunol Res. 2014 September: 2(9):846-56), SEB (staphylococcal enterotoxin B) T cell stimulation assay (Wang et al., Cancer Immunol Res. 2014 September: 2(9):846-56), and anti-CD3 T cell stimulation assays (Li and Kurlander, J Transl Med. 2010: 8: 104).

In some embodiments, a variant CD80 polypeptide can in some embodiments increase or, in alternative embodiments, decrease IFN-gamma (interferon-gamma) expression in a primary T-cell assay relative to a wild-type CD80 control. In some embodiments, such activity may depend on the particular assay design and whether the variant CD80 polypeptide is provided in a form for antagonist activity or in a form for agonist activity. In some embodiments, a variant CD80 polypeptide or immunomodulatory protein is an antagonist of the inhibitory receptor, such as blocks an inhibitory signal from PD-1 or CTLA-4 in the cell that may occur to decrease response to an activating stimulus, e.g., CD3 and/or CD28 costimulatory signal or a mitogenic signal. In some embodiments, a variant CD80 polypeptide or immunomodulatory protein is an agonist of CD28 to increase an activating stimulus, e.g., CD3. Those of skill will recognize that different formats of the primary T-cell assay used to determine an increase or decrease in IFN-gamma expression exist.

In assaying for the ability of a variant CD80 to increase or decrease IFN-gamma expression in a primary T-cell assay, a Mixed Lymphocyte Reaction (MLR) assay can be used. In some embodiments, a variant CD80 polypeptide or immunomodulatory protein provided in antagonist form, such as soluble form, e.g., variant CD80-Fc or secretable immunomodulatory protein, block activity of the CTLA-4 inhibitory receptor or PD-L1 and thereby increase MLR activity in the assay, such as observed by increased production of IFN-gamma in the assay.

Alternatively, in assaying for the ability of a variant CD80 to modulate an increase or decrease IFN-gamma expression in a primary T-cell assay, a co-immobilization assay can be used. In a co-immobilization assay, a TCR signal, provided in some embodiments by anti-CD3 antibody, is used in conjunction with a co-immobilized variant CD80 polypeptide to determine the ability to increase or decrease IFN-gamma expression relative to a CD80 unmodified or wild-type control. In some embodiments, a variant CD80 polypeptide or immunomodulatory protein, e.g., a co-immobilized variant CD80 (e.g., CD80-Fc), increases IFN-gamma production in a co-immobilization assay.

In some embodiments, in assaying for the ability of a variant CD80 polypeptide or immunomodulatory protein to modulate an increase in IFN-gamma expression a T cell reporter assay can be used. In some embodiments, the T cell is a Jurkat T cell line or is derived from Jurkat T cell lines. In some embodiments, the reporter T cells also contain a reporter construct containing an inducible promoter responsive to T cell activation operably linked to a reporter. In some embodiments, the reporter is a fluorescent or luminescent reporter. In some embodiments, the reporter is luciferase. In some embodiments, the promoter is responsive to CD3 signaling. In some embodiments, the promoter is an NFAT promoter. In some embodiments, the promoter is responsive to costimulatory signaling, e.g., CD28 costimulatory signaling. In some embodiments, the promoter is an IL-2 promoter.

In aspects of a reporter assay, a reporter cell line is stimulated, such as by co-incubation with antigen presenting cells, such as for delivery a stimulatory signal to the reporter T cells. In some embodiments, the APCs are artificial APCs. Artificial APCs are well known to a skilled artisan. In some embodiments, artificial APCs are derived from one or more mammalian cell line, such as K562, CHO or 293 cells. In some embodiments, the artificial APCs are engineered to express an anti-CD3 antibody and, in some cases, a costimulatory ligand. In some embodiments, the artificial APC is generated to overexpress the cognate binding partner of the variant IgSF domain polypeptide. For example, in the case of a variant CD80, the APC is generated to overexpress the inhibitory ligand PD-L1. In some cases, the reporter cell line (e.g., Jurkat reporter cell) is generated to overexpress the ligand CD28. In some embodiments, the Jurkat reporter cells are co-incubated with artificial APCs in the presence of the variant CD80 polypeptide or immunomodulatory protein. In some embodiments, reporter expression is monitored, such as by determining the luminescence or fluorescence of the cells. Agonist or antagonist (blocking) activity of a cognate binding partner can be monitored.

In some embodiments, a variant CD80 polypeptide or immunomodulatory protein provided herein results in an increase in the reporter signal compared to the absence of the variant CD80 polypeptide or immunomodulatory protein. In certain embodiments provided herein, a variant CD80 polypeptide or immunomodulatory protein mediates CD28 agonism, such as PD-L1-dependent CD28 costimulation in which the reporter signal is observed in the presence of APCs expressing the PD-L1 ligand, thereby resulting in an increase of the reporter signal compared to the absence of the variant CD80 polypeptide or immunomodulatory protein.

Use of proper controls is known to those of skill in the art, however, in the aforementioned embodiments, a control typically involves use of the unmodified CD80, such as a wild-type of native CD80 isoform from the same mammalian species from which the variant CD80 was derived or developed. In some embodiments, the wild-type or native CD80 is of the same form or corresponding form as the variant. For example, if the variant CD80 is a soluble form containing a variant ECD fused to an Fc protein, then the control is a soluble form containing the wild-type or native ECD of CD80 fused to the Fc protein. Irrespective of whether the binding affinity and/or selectivity to either one or more of CTLA-4 and CD80 is increased or decreased, a variant CD80 in some embodiments will increase IFN-gamma expression and, in alternative embodiments, decrease IFN-gamma expression in a T-cell assay relative to a wild-type CD80 control.

In some embodiments, a variant CD80 polypeptide or immunomodulatory protein, increases IFN-gamma expression (i.e., protein expression) relative to a wild-type or unmodified CD80 control by at least: 5%, 10%, 20%, 30%, 40%, 50%, 60%, 70%, 80%, 90%, or higher. In other embodiments, a variant CD80 or immunomodulatory protein decreases IFN-gamma expression (i.e. protein expression) relative to a wild-type or unmodified CD80 control by at least: 5%, 10%, 20%, 30%, 40%, 50%, 60%, 70%, 80%, 90%, or higher. In some embodiments, the wild-type CD80 control is human CD80, such as would typically be used for a variant CD80 altered in sequence from that of a corresponding wild-type human CD80 sequence such as an CD80 sequence comprising the sequence of amino acids of SEQ ID NO: 2 or a portion thereof comprising the IgV domain, such as set forth in SEQ ID NO:163 or SEQ ID NO:164.

### III. Nucleic Acids, Vectors and Methods for Producing the Polypeptides

Provided herein are isolated or recombinant nucleic acids collectively referred to as “nucleic acids” which encode any of the immunomodulatory proteins provided herein. In some embodiments, nucleic acids provided herein, including all described below, are useful in recombinant production (e.g., expression) of immunomodulatory proteins provided herein. In some embodiments, nucleic acids provided herein, including all described below, are useful in expression of immunomodulatory proteins provided herein, such as variant CD80 fusion proteins provided herein. The nucleic acids provided herein can be in the form of RNA or in the form of DNA, and include mRNA, cRNA, recombinant or synthetic RNA and DNA, and cDNA. The nucleic acids provided herein are typically DNA molecules, and usually double-stranded DNA molecules. However, single-stranded DNA, single-stranded RNA, double-stranded RNA, and hybrid DNA/RNA nucleic acids or combinations thereof comprising any of the nucleotide sequences of the invention also are provided.

In some embodiments, the immunomodulatory protein comprises a signal peptide when expressed, and the signal peptide (or a portion thereof) is cleaved from the immunomodulatory protein upon secretion.

Also provided herein are recombinant expression vectors and recombinant host cells useful in producing the immunomodulatory proteins, such as variant CD80 fusion proteins provided herein.

In any of the above provided embodiments, the nucleic acids encoding the immunomodulatory polypeptides provided herein can be introduced into cells using recombinant DNA and cloning techniques. To do so, a recombinant DNA molecule encoding an immunomodulatory polypeptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidite method. Also, a combination of these techniques could be used. In some instances, a recombinant or synthetic nucleic acid may be generated through polymerase chain reaction (PCR). A DNA insert encoding an immunomodulatory protein can be cloned into an appropriate transduction/transfection vector as is known to those of skill in the art. Also provided are expression vectors containing the nucleic acid molecules.

In some embodiments, the expression vectors are capable of expressing the immunomodulatory proteins in an appropriate cell under conditions suited to expression of the protein. In some aspects, nucleic acid molecule or an expression vector comprises the DNA molecule that encodes the immunomodulatory protein operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

In some embodiments, expression of the immunomodulatory protein is controlled by a promoter or enhancer to control or regulate expression. The promoter is operably linked to the portion of the nucleic acid molecule encoding the variant polypeptide or immunomodulatory protein.

The resulting recombinant expression vector having the DNA molecule thereon is used to transform an appropriate host. This transformation can be performed using methods well known in the art. In some embodiments, a nucleic acid provided herein further comprises nucleotide sequence that encodes a secretory or signal peptide operably linked to the nucleic acid encoding an immunomodulatory polypeptide such that a resultant soluble immunomodulatory polypeptide is recovered from the culture medium, host cell, or host cell periplasm. In other embodiments, the appropriate expression control signals are chosen to allow for membrane expression of an immunomodulatory polypeptide. Furthermore, commercially available kits as well as contract manufacturing companies can also be utilized to make engineered cells or recombinant host cells provided herein.

In some embodiments, the resulting expression vector having the DNA molecule thereon is used to transform, such as transduce, an appropriate cell. The introduction can be performed using methods well known in the art. Exemplary methods include those for transfer of nucleic acids encoding the receptors, including via viral, e.g., retroviral or lentiviral, transduction, transposons, and electroporation. In some embodiments, the expression vector is a viral vector. In some embodiments, the nucleic acid is transferred into cells by lentiviral or retroviral transduction methods.

Any of a large number of publicly available and well-known mammalian host cells can be used in the preparing the polypeptides or engineered cells. The selection of a cell is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all cells can be equally effective for the expression of a particular DNA sequence.

In some embodiments, the host cell is a mammalian cell. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 (Chasin et al, Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658).

In some embodiments, the host cells can be a variety of eukaryotic cells, such as in yeast cells, or with mammalian cells such as Chinese hamster ovary (CHO) or HEK293 cells. In some embodiments, the host cell is a suspension cell and the polypeptide is engineered or produced in cultured suspension, such as in cultured suspension CHO cells, e.g. CHO-S cells. In some examples, the cell line is a CHO cell line that is deficient in DHFR (DHFR−), such as DG44 and DUXB11. In some embodiments, the cell is deficient in glutamine synthase (GS), e.g. CHO-S cells, CHOKI SV cells, and CHOZN((R)) GS−/− cells. In some embodiments, the CHO cells, such as suspension CHO cells, may be CHO-S-2H2 cells, CHO-S-clone 14 cells, or ExpiCHO-S cells.

In some embodiments, host cells can also be prokaryotic cells, such as with E. coli. The transformed recombinant host is cultured under polypeptide expressing conditions, and then purified to obtain a soluble protein. Recombinant host cells can be cultured under conventional fermentation conditions so that the desired polypeptides are expressed. Such fermentation conditions are well known in the art. Finally, the polypeptides provided herein can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, and affinity chromatography. Protein refolding steps can be used, as desired, in completing configuration of the mature protein. Finally, high performance liquid chromatography (HPLC) can be employed in the final purification steps.

In some embodiments, the recombinant vector is a viral vector. Exemplary recombinant viral vectors include a lentiviral vector genome, poxvirus vector genome, vaccinia virus vector genome, adenovirus vector genome, adenovirus-associated virus vector genome, herpes virus vector genome, and alpha virus vector genome. Viral vectors can be live, attenuated, replication conditional or replication deficient, non-pathogenic (defective), replication competent viral vector, and/or is modified to express a heterologous gene product, e.g., the variant immunomodulatory polypeptides provided herein. Vectors for generation of viruses also can be modified to alter attenuation of the virus, which includes any method of increasing or decreasing the transcriptional or translational load.

Exemplary viral vectors that can be used include modified vaccinia virus vectors (see, e.g., Guerra et al., J. Virol. 80:985-98 (2006); Tartaglia et al., AIDS Research and Human Retroviruses 8: 1445-47 (1992); Gheradi et al., J. Gen. Virol. 86:2925-36 (2005); Mayr et al., Infection 3:6-14 (1975); Hu et al., J. Virol. 75: 10300-308 (2001); U.S. Pat. Nos. 5,698,530, 6,998,252, 5,443,964, 7,247,615 and 7,368,116); adenovirus vector or adenovirus-associated virus vectors (see, e.g., Molin et al., J. Virol. 72:8358-61 (1998); Narumi et al., Am J. Respir. Cell Mol. Biol. 19:936-41 (1998); Mercier et al., Proc. Natl. Acad. Sci. USA 101:6188-93 (2004); U.S. Pat. Nos. 6,143,290; 6,596,535; 6,855,317; 6,936,257; 7,125,717; 7,378,087; 7,550,296); retroviral vectors including those based upon murine leukemia virus (MuLV), gibbon ape leukemia virus (GaLV), ecotropic retroviruses, simian immunodeficiency virus (SIV), human immunodeficiency virus (HIV), and combinations (see, e.g., Buchscher et al., J. Virol. 66:2731-39 (1992); Johann et al., J. Virol. 66: 1635-40 (1992); Sommerfelt et al., Virology 176:58-59 (1990); Wilson et al., J. Virol. 63:2374-78 (1989); Miller et al., J. Virol. 65:2220-24 (1991); Miller et al., Mol. Cell Biol. 10:4239 (1990); Kolberg, NIH Res. 4:43 1992; Cornetta et al., Hum. Gene Ther. 2:215 (1991)); lentiviral vectors including those based upon Human Immunodeficiency Virus (HIV-1), HIV-2, feline immunodeficiency virus (FIV), equine infectious anemia virus, Simian Immunodeficiency Virus (SIV), and maedi/visna virus (see, e.g., Pfeifer et al., Annu. Rev. Genomics Hum. Genet. 2: 177-211 (2001); Zufferey et al., J. Virol. 72: 9873, 1998; Miyoshi et al., J. Virol. 72:8150, 1998; Philpott and Thrasher, Human Gene Therapy 18:483, 2007; Engelman et al., J. Virol. 69: 2729, 1995; Nightingale et al., Mol. Therapy, 13: 1121, 2006; Brown et al., J. Virol. 73:9011 (1999); WO 2009/076524; WO 2012/141984; WO 2016/011083; McWilliams et al., J. Virol. 77: 11150, 2003; Powell et al., J. Virol. 70:5288, 1996) or any, variants thereof, and/or vectors that can be used to generate any of the viruses described above. In some embodiments, the recombinant vector can include regulatory sequences, such as promoter or enhancer sequences, that can regulate the expression of the viral genome, such as in the case for RNA viruses, in the packaging cell line (see, e.g., U.S. Pat. Nos. 5,385,839 and 5,168,062).

In some aspects, nucleic acids or an expression vector comprises a nucleic acid sequence that encodes the immunomodulatory protein operatively linked to appropriate expression control sequences. Methods of effecting this operative linking, either before or after the nucleic acid sequence encoding the immunomodulatory protein is inserted into the vector, are well known. Expression control sequences include promoters, activators, enhancers, operators, ribosomal binding sites, start signals, stop signals, cap signals, polyadenylation signals, and other signals involved with the control of transcription or translation. The promoter can be operably linked to the portion of the nucleic acid sequence encoding the immunomodulatory protein.

Transcriptional regulatory sequences include a promoter region sufficient to direct the initiation of RNA synthesis. Suitable eukaryotic promoters include the promoter of the mouse metallothionein I gene (Hamer et al, J. Molec. Appl Genet. 1:273 (1982)), the TK promoter of Herpes virus (McKnight, Cell 31:355 (1982)), the SV40 early promoter (Benoist et al, Nature 290:304 (1981)), the Rous sarcoma virus promoter (Gorman et al, Proc. Nat'l Acad. Sci. USA 79:6777 (1982)), the cytomegalovirus promoter (Foecking et al, Gene 45:101 (1980)), and the mouse mammary tumor virus promoter (see, generally, Etcheverry, “Expression of Engineered Proteins in Mammalian Cell Culture,” in Protein Engineering: Principles and Practice, Cleland et al. (eds.), pages 163-181 (John Wiley & Sons, Inc. 1996)). One useful combination of a promoter and enhancer is provided by a myeloproliferative sarcoma virus promoter and a human cytomegalovirus enhancer.

Alternatively, a prokaryotic promoter, such as the bacteriophage T3 RNA polymerase promoter, can be used to control production of an immunomodulatory protein in mammalian cells if the prokaryotic promoter is regulated by a eukaryotic promoter (Zhou et al, Mol Cell. Biol. 10:4529 (1990), and Kaufman et al, Nucl. Acids Res. 19:4485 (1991)).

An expression vector can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

For example, one suitable selectable marker is a gene that provides resistance to the antibiotic neomycin. In this case, selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A suitable amplifiable selectable marker is dihydrofolate reductase, which confers resistance to methotrexate. Other drug resistance genes (e.g., hygromycin resistance, multi-drug resistance, puromycin acetyltransferase) can also be used. Alternatively, markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

In some embodiments, polypeptides provided herein can also be made by synthetic methods. Solid phase synthesis is the preferred technique of making individual peptides since it is the most cost-effective method of making small peptides. For example, well known solid phase synthesis techniques include the use of protecting groups, linkers, and solid phase supports, as well as specific protection and deprotection reaction conditions, linker cleavage conditions, use of scavengers, and other aspects of solid phase peptide synthesis. Peptides can then be assembled into the polypeptides as provided herein.

### IV. Pharmaceutical Compositions

Provided herein are compositions containing any of the provided immunomodulatory proteins described herein. The pharmaceutical composition can further comprise a pharmaceutically acceptable excipient. For example, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, provided herein are compositions containing any of the provided engineered cells described herein. In some embodiments, provided are pharmaceutical compositions containing the T engineered cells expressing secretable or transmembrane immunomodulatory proteins as provided. In some embodiments, the pharmaceutical compositions and formulations include one or more optional pharmaceutically acceptable carrier or excipient. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Such a formulation containing engineered cells may, for example, be in a form suitable for intravenous infusion. A pharmaceutically acceptable carrier may be a pharmaceutically acceptable material, composition, or vehicle that is involved in carrying or transporting cells of interest from one tissue, organ, or portion of the body to another tissue, organ or portion of the body.

In some embodiments, the pharmaceutical composition is sterile.

In some embodiments, the pharmaceutical composition is a solid, such as a powder, capsule, or tablet. For example, the components of the pharmaceutical composition can be lyophilized. In some embodiments, the solid pharmaceutical composition is reconstituted or dissolved in a liquid prior to administration.

In some embodiments, the pharmaceutical composition is a liquid, for example immunomodulatory proteins dissolved in an aqueous solution (such as physiological saline or Ringer's solution). In some embodiments, the pH of the pharmaceutical composition is between about 4.0 and about 8.5 (such as between about 4.0 and about 5.0, between about 4.5 and about 5.5, between about 5.0 and about 6.0, between about 5.5 and about 6.5, between about 6.0 and about 7.0, between about 6.5 and about 7.5, between about 7.0 and about 8.0, or between about 7.5 and about 8.5).

In some embodiments, the pharmaceutical composition comprises a pharmaceutically-acceptable excipient, for example a filler, binder, coating, preservative, lubricant, flavoring agent, sweetening agent, coloring agent, a solvent, a buffering agent, a chelating agent, or stabilizer. Examples of pharmaceutically-acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically-acceptable binders include polyvinylpyrrolidone, starch, lactose, xylitol, sorbitol, maltitol, gelatin, sucrose, polyethylene glycol, methyl cellulose, or cellulose. Examples of pharmaceutically-acceptable coatings include hydroxypropyl methylcellulose (HPMC), shellac, corn protein zein, or gelatin. Examples of pharmaceutically-acceptable disintegrants include polyvinylpyrrolidone, carboxymethyl cellulose, or sodium starch glycolate. Examples of pharmaceutically-acceptable lubricants include polyethylene glycol, magnesium stearate, or stearic acid. Examples of pharmaceutically-acceptable preservatives include methyl parabens, ethyl parabens, propyl paraben, benzoic acid, or sorbic acid. Examples of pharmaceutically-acceptable sweetening agents include sucrose, saccharine, aspartame, or sorbitol. Examples of pharmaceutically-acceptable buffering agents include carbonates, citrates, gluconates, acetates, phosphates, or tartrates.

In some embodiments, the pharmaceutical composition further comprises an agent for the controlled or sustained release of the product, such as injectable microspheres, bio-erodible particles, polymeric compounds (polylactic acid, polyglycolic acid), beads, or liposomes.

In some embodiments, the pharmaceutical composition is sterile. Sterilization may be accomplished by filtration through sterile filtration membranes or radiation. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

A pharmaceutically acceptable carrier may be a pharmaceutically acceptable material, composition, or vehicle. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

In some embodiments, the pharmaceutical composition is administered to a subject. Generally, dosages and routes of administration of the pharmaceutical composition are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

Long-acting pharmaceutical compositions may be administered every 3 to 4 days, every week, or biweekly depending on the half-life and clearance rate of the particular formulation. The frequency of dosing will depend upon the pharmacokinetic parameters of the molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally.

A provided pharmaceutical formulation may, for example, be in a form suitable for intravenous infusion.

In some embodiments, the dosage of the pharmaceutical composition is a single dose or a repeated dose. In some embodiments, the doses are given to a subject once per day, twice per day, three times per day, or four or more times per day. In some embodiments, about 1 or more (such as about 2 or more, about 3 or more, about 4 or more, about 5 or more, about 6 or more, or about 7 or more) doses are given in a week. In some embodiments, multiple doses are given over the course of days, weeks, months, or years. In some embodiments, a course of treatment is about 1 or more doses (such as about 2 or more does, about 3 or more doses, about 4 or more doses, about 5 or more doses, about 7 or more doses, about 10 or more doses, about 15 or more doses, about 25 or more doses, about 40 or more doses, about 50 or more doses, or about 100 or more doses).

In some embodiments, an administered dose of the pharmaceutical composition is about 1 μg of protein per kg subject body mass or more (such as about 2 μg of protein per kg subject body mass or more, about 5 μg of protein per kg subject body mass or more, about 10 μg of protein per kg subject body mass or more, about 25 μg of protein per kg subject body mass or more, about 50 μg of protein per kg subject body mass or more, about 100 μg of protein per kg subject body mass or more, about 250 μg of protein per kg subject body mass or more, about 500 μg of protein per kg subject body mass or more, about 1 mg of protein per kg subject body mass or more, about 2 mg of protein per kg subject body mass or more, or about 5 mg of protein per kg subject body mass or more).

In some embodiments, for a pharmaceutical composition containing engineered cells, a therapeutic amount of a cell composition is administered. Typically, precise amounts of the compositions of the present invention to be administered can be determined by a physician with consideration of individual differences in age, weight, tumor size, extent of infection or metastasis, and condition of the patient (subject). It can generally be stated that a pharmaceutical composition comprising engineered cells, e.g., T cells, as described herein may be administered at a dosage of 10⁴ to 10⁹ cells/kg body weight, such as 10⁵ to 10⁶ cells/kg body weight, including all integer values within those ranges. Engineered cell compositions, such as T cell compositions, may also be administered multiple times at these dosages. The cells can be administered by using infusion techniques that are commonly known in immunotherapy (see, e.g., Rosenberg et al, New Eng. J. of Med. 319: 1676, 1988). The optimal dosage and treatment regime for a particular patient can readily be determined by one skilled in the art of medicine by monitoring the patient for signs of disease and adjusting the treatment accordingly.

### V. Therapeutic Applications

Provided herein are methods using the provided pharmaceutical compositions containing a variant CD80 polypeptide or immunomodulatory protein for modulating an immune response, including in connection with treating a disease or condition in a subject, such as in a human patient. Also provided herein are methods of using the provided pharmaceutical compositions containing an engineered cell in which is expressed the variant CD80 polypeptide or immunomodulatory protein containing the same as a secretable or transmembrane protein. The pharmaceutical compositions described herein (including pharmaceutical composition comprising the variant CD80 polypeptides or the immunomodulatory proteins) can be used in a variety of therapeutic applications, such as the treatment of a disease. In some embodiments, provided are methods of using the variant CD80 polypeptides or immunomodulatory proteins to increase an immune response in a subject. In some such aspects, increasing an immune response treats a disease or condition in the subject, such as a tumor or cancer.

In some embodiments, a pharmaceutical composition provided herein that stimulates or increases the immune response is administered, which can be useful, for example, in the treatment of cancer, viral infections, or bacterial infections. Among the provided methods are methods involving delivery of variant CD80 polypeptides or immunomodulatory proteins with increased affinity for PD-L1, which can antagonize signaling of an inhibitory receptor, such as block an inhibitory signal in the cell that may occur to decrease response to an activating stimulus, e.g., CD3 and/or CD28 costimulatory signal or a mitogenic signal. In some cases, the result of this can be to increase the immune response. In some embodiments, antagonism of PD-L1/PD-1, or in some cases also CTLA-4, by the provided variant CD80 polypeptides or immunomodulatory proteins may be useful to promote immunity in oncology, such as for treatment of tumors or cancers. In some embodiments, agonism of CD28, which can be dependent on or enhanced by CD80 co-binding PD-L1, may be useful to promote immunity in oncology, such as for treatment of tumors or cancers.

There is provided methods of increasing an immune response by delivery of a variant CD80 polypeptide that binds to PD-L1, such as binds PD-L1 with increased affinity compared to an unmodified or wildtype CD80 polypeptide. In some embodiments, the provided CD80 polypeptides are capable of binding the PD-L1 on a tumor cell or APC, thereby blocking the interaction of PD-L1 and the PD-1 inhibitory receptor to prevent the negative regulatory signaling that would have otherwise resulted from the PD-L1/PD-1 interaction. In some cases, the result of this can be to increase the immune response, which, in some aspects, can treat a disease or condition in the subject, such as treatment of a tumor or cancer.

Also among provided embodiments are methods for mediating agonism of CD28 by PD-L1 dependent CD28 costimulation using variant CD80 polypeptides or immunomodulatory proteins that exhibit increased binding to PD-L1 compared to unmodified or wild-type CD80 polypeptide. In some aspects, such methods can be used to increase an immune response in a subject administered the molecules, which, in some aspects, can treat a disease or condition in the subject, such as treatment of a tumor or cancer. In some cases, such variant CD80 polypeptides or immunomodulatory proteins also can facilitate promotion of an immune response in connection with the provided therapeutic methods by blocking the PD-L1/PD-1 interaction while also binding and co-stimulating a CD28 receptor on a localized T cell. In some cases, the variant CD80 polypeptides or immunomodulatory proteins also can block CTLA-4 activity.

In some embodiments, an immunomodulatory protein containing at least one variant CD80 polypeptide that is linked, directly or indirectly to an Fc is administered to a subject to mediate CD28 agonism. There is provided methods for mediating agonism of CD28 by receptor-dependent CD28 costimulation using variant CD80 polypeptides provided herein the bind to CD28. In some embodiments, such agonism of CD28 may be useful to promote immunity in oncology, such as for treatment of tumors or cancer. In some cases, the variant CD80 polypeptides also bind CTLA-4 or PD-L1, such as exhibit increased binding to PD-L1. The provided methods to modulate an immune response can be used to treat a disease or condition, such as a tumor or cancer. In some embodiments, the pharmaceutical composition can be used to inhibit growth of mammalian cancer cells (such as human cancer cells).

In provided embodiments, a method of treating cancer can include administering an effective amount of any of the pharmaceutical compositions described herein to a subject with cancer. The effective amount of the pharmaceutical composition can be administered to inhibit, halt, or reverse progression of cancers. Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the therapeutic composition into the patient. Thus, the present invention provides ex vivo and in vivo methods to inhibit, halt, or reverse progression of the tumor, or otherwise result in a statistically significant increase in progression-free survival (i.e., the length of time during and after treatment in which a patient is living with cancer that does not get worse), or overall survival (also called “survival rate;” i.e., the percentage of people in a study or treatment group who are alive for a certain period of time after they were diagnosed with or treated for cancer) relative to treatment with a control.

The cancers that can be treated by the pharmaceutical compositions and the treatment methods described herein include, but are not limited to, melanoma, bladder cancer, hematological malignancies (leukemia, lymphoma, myeloma), liver cancer, brain cancer, renal cancer, breast cancer, pancreatic cancer (adenocarcinoma), colorectal cancer, lung cancer (small cell lung cancer and non-small-cell lung cancer), spleen cancer, cancer of the thymus or blood cells (i.e., leukemia), prostate cancer, testicular cancer, ovarian cancer, uterine cancer, gastric carcinoma, a musculoskeletal cancer, a head and neck cancer, a gastrointestinal cancer, a germ cell cancer, or an endocrine and neuroendocrine cancer. In some embodiments, the cancer is Ewing's sarcoma. In some embodiments, the cancer is selected from melanoma, lung cancer, bladder cancer, and a hematological malignancy. In some embodiments, the cancer is a lymphoma, lymphoid leukemia, myeloid leukemia, cervical cancer, neuroblastoma, or multiple myeloma.

In some embodiments, the pharmaceutical composition is administered as a monotherapy (i.e., as a single agent) or as a combination therapy (i.e., in combination with one or more additional anticancer agents, such as a chemotherapeutic drug, a cancer vaccine, or an immune checkpoint inhibitor. In some embodiments, the pharmaceutical composition can also be administered with radiation therapy. In some aspects of the present disclosure, the immune checkpoint inhibitor is nivolumab, Tremelimumab, pembrolizumab, ipilimumab, or the like.

In some embodiments, the provided methods are for treating a subject that is or is suspected of having the disease or condition for which the therapeutic application is directed. In some cases, the subject for treatment can be selected prior to treatment based on one or more features or parameters, such as to determine suitability for the therapy or to identify or select subjects for treatment in accord with any of the provided embodiments, including treatment with any of the provided variant CD80 polypeptides or immunomodulatory proteins.

In some aspects, a subject is selected for treatment if at or immediately prior to the time of the administration of the pharmaceutical composition containing a variant CD80 polypeptide as described the subject has relapsed following remission after treatment with, or become refractory to, or is non-responsive to treatment with an antagonist of PD-1/PD-L1 or PD-1/PD-L2. In some embodiments, the antagonist is one that does not compete for binding to PD-L1 with a provided variant CD80 polypeptide to be used in the treatment methods. In some embodiments, the antagonist is an anti-PD-1 antibody. Exemplary anti-PD-1 antibodies are known and include, but are not limited to, nivolumab or pembrolizumab, or antigen binding fragments thereof.

In some embodiments, provided methods include diagnostic, prognostic or monitoring methods utilizing binding assays on various biological samples of patients having a disease or condition in which is known, suspected or that may be a candidate for treatment in accord with the provided embodiments. In some embodiments, the methods are carried out with reagents capable of detecting CD28, PD-L1 and/or CTLA-4 to select subjects having tumors or tumor cell infiltrates that express one or more binding partner of the variant CD80 polypeptide to be utilized in the therapeutic methods. Such reagents can be used as companion diagnostics for selecting subjects that are most likely to benefit from treatment with the provided molecules or pharmaceutical compositions and/or for predicting efficacy of the treatment.

In some embodiments, methods are provided for selecting subjects and/or predicting efficacy of treatment with provided therapies based on activity to antagonize PD-L1/PD-1 interaction and/or based on CD28 agonism, such as PD-L1-dependent CD28 costimulation, including in methods for increasing an immune response for treating a disease or condition and/or for treating a tumor or cancer. In some embodiments, a binding reagent is contacted with a sample from the subject. In some embodiments, the binding reagent is a PD-L1-binding reagent that specifically binds to PD-L1 on the surface of a cell, such as on the surface of a tumor cell or myeloid cells present in the tumor environment. In some embodiments, the binding reagent is a CD28-binding reagent that specifically binds to CD28 on the surface of a cell, such as on the surface of an infiltrating immune cell, such as a lymphocyte, e.g. a T cell. In some embodiments, the binding reagent can be an antibody or antigen-binding fragment, protein ligand or binding partner, an aptamer, an affimer, a peptide or a hapten. In some embodiments, such reagents can be used as a companion diagnostic for selecting or identifying subjects for treatment with a therapeutic agent or pharmaceutical composition provided herein containing a variant CD80 polypeptide that is or contains an IgSF domain (e.g. IgV) that exhibits increased binding to PD-L1 compared to the unmodified or wild-type CD80, including immunomodulatory proteins as provided. Included among such therapeutic agents are formats in which an extracellular portion of a CD80 variant polypeptide containing an affinity modified IgSF domain (e.g. IgV) is linked, directly or indirectly, to a multimerization domain, e.g. an Fc domain or region. In some embodiments, such a therapeutic agent is a variant CD80-Fc fusion protein.

In some embodiments, the binding reagent is an antibody or an antigen binding fragment thereof that specifically binds PD-L1. Various companion diagnostic reagents for detecting PD-L1, including intracellular or extracellular PD-L1, are known, e.g. Roach et al. (2016) Appl. Immunohistochem., Mol. Morphol., 24:392-397; Cogswell et al. (2017) Mol. Diagn. Ther. 21:85-93; International published patent application No. WO2015/181343 or WO2017/085307, or U.S. published patent application No. US2016/0009805 or US2017/0285037. Non limiting examples of anti-PD-L1 antibodies include, but are not limited to, mouse anti-PD-L1 clone 22C3 (Merck & Co.), rabbit anti-PD-L1 clone 28-8 (Bristol-Myers Squibb), rabbit anti-PD-L1 clones SP263 or SP142 (Spring Biosciences) and rabbit anti-PD-L1 antibody clone E1L3N. Such binding reagents can be used in histochemistry methods, including those available as Dako PD-L1 IHC 22C3 pharmDx assay, PD-L1 IHC 28-8 pharmDx assay, Ventana PD-L1 (SP263) assay, or Ventana PD-L1 (SP142) assay.

The binding reagent can be conjugated, such as fused, directly or indirectly to a detectable label for detection. In some cases, the binding reagent is linked or attached to a moiety that permits either direct detection or detection via secondary agents, such as via antibodies that bind to the reagent or a portion of the reagent and that are coupled to a detectable label. Exemplary detectable labels include, for example, chemiluminescent moieties, bioluminescent moieties, fluorescent moieties, radionuclides, and metals. Methods for detecting labels are well known in the art. Such a label can be detected, for example, by visual inspection, by fluorescence spectroscopy, by reflectance measurement, by flow cytometry, by X-rays, by a variety of magnetic resonance methods such as magnetic resonance imaging (MRI) and magnetic resonance spectroscopy (MRS). Methods of detection also include any of a variety of tomographic methods including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography, and ultrasonic tomography. Exemplary detectable labels include, for example, chemiluminescent moieties, bioluminescent moieties, fluorescent moieties, radionuclides, and metals. Among detectable labels are fluorescent probes or detectable enzymes, e.g. horseradish perioxidase.

The binding reagents can detect the binding partner, e.g. PD-L1, CD28 or CTLA-4, using any binding assay known to one of skill in the art including, in vitro or in vivo assays. Exemplary binding assays that can be used to assess, evaluate, determine, quantify and/or otherwise specifically detect expression or levels of a binding partner, e.g. PD-L1, CD28 or CTLA-4, in a sample include, but are not limited to, solid phase binding assays (e.g. enzyme linked immunosorbent assay (ELISA)), radioimmunoassay (RIA), immunoradiometric assay, fluorescence assay, chemiluminescent assay, bioluminescent assay, western blot and histochemistry methods, such as immunohistochemistry (IHC) or pseudo immunohistochemistry using a non-antibody binding agent. In solid phase binding assay methods, such as ELISA methods, for example, the assay can be a sandwich format or a competitive inhibition format. In other examples, in vivo imaging methods can be used. The binding assay can be performed on samples obtained from a patient body fluid, cell or tissue sample of any type, including from plasma, urine, tumor or suspected tumor tissues (including fresh, frozen, and fixed or paraffin embedded tissue), lymph node or bone marrow. In exemplary methods to select a subject for treatment in accord with the therapeutic methods provided herein, harvesting of the sample, e.g. tumor tissue, is carried out prior to treatment of the subject.

In some embodiments, the binding assay is a tissue staining assay to detect the expression or levels of a binding partner in a tissue or cell sample. Tissue staining methods include, but are not limited to, cytochemical or histochemical methods, such as immunohistochemistry (IHC) or histochemistry using a non-antibody binding agent (e.g. pseudo immunohistochemistry). Such histochemical methods permit quantitative or semi-quantitative detection of the amount of the binding partner in a sample, such as a tumor tissue sample. In such methods, a tissue sample can be contacted with a binding reagent, e.g. PD-L1 binding reagent, and in particular one that is detectably labeled or capable of detection, under conditions that permit binding to a tissue- or cell-associated binding partner.

A sample for use in the methods provided herein as determined by histochemistry can be any biological sample that is associated with the disease or condition, such as a tissue or cellular sample. For example, a tissue sample can be solid tissue, including a fresh, frozen and/or preserved organ or tissue sample or biopsy or aspirate, or cells. In some examples, the tissue sample is tissue or cells obtained from a solid tumor, such as primary and metastatic tumors, including but not limited to, breast, colon, rectum, lung, stomach, ovary, cervix, uterus, testes, bladder, prostate, thyroid and lung cancer tumors. In particular examples, the sample is a tissue sample from a cancer that is a late-stage cancer, a metastatic cancer, undifferentiated cancer, ovarian cancer, in situ carcinoma (ISC), squamous cell carcinoma (SCC), prostate cancer, pancreatic cancer, non-small cell lung cancer, breast cancer, colon cancer.

In some aspects, when the tumor is a solid tumor, isolation of tumor cells can be achieved by surgical biopsy. Biopsy techniques that can be used to harvest tumor cells from a subject include, but are not limited to, needle biopsy, CT-guided needle biopsy, aspiration biopsy, endoscopic biopsy, bronchoscopic biopsy, bronchial lavage, incisional biopsy, excisional biopsy, punch biopsy, shave biopsy, skin biopsy, bone marrow biopsy, and the Loop Electrosurgical Excision Procedure (LEEP). Typically, a non-necrotic, sterile biopsy or specimen is obtained that is greater than 100 mg, but which can be smaller, such as less than 100 mg, 50 mg or less, 10 mg or less or 5 mg or less; or larger, such as more than 100 mg, 200 mg or more, or 500 mg or more, 1 gm or more, 2 gm or more, 3 gm or more, 4 gm or more or 5 gm or more. The sample size to be extracted for the assay can depend on a number of factors including, but not limited to, the number of assays to be performed, the health of the tissue sample, the type of cancer, and the condition of the subject. The tumor tissue is placed in a sterile vessel, such as a sterile tube or culture plate, and can be optionally immersed in an appropriate medium.

In some embodiments, tissue obtained from the patient after biopsy is fixed, such as by formalin (formaldehyde) or glutaraldehyde, for example, or by alcohol immersion. For histochemical methods, the tumor sample can be processed using known techniques, such as dehydration and embedding the tumor tissue in a paraffin wax or other solid supports known to those of skill in the art (see Plenat et ah, (2001) Ann Pathol. January 21(1):29-47), slicing the tissue into sections suitable for staining, and processing the sections for staining according to the histochemical staining method selected, including removal of solid supports for embedding by organic solvents, for example, and rehydration of preserved tissue.

In some embodiments, histochemistry methods are employed. In some cases, the binding reagent is directly attached or linked to a detectable label or other moiety for direct or indirect detection. Exemplary detectable regents including, but are not limited to, biotin, a fluorescent protein, bioluminescent protein or enzyme. In other examples, the binding reagents are conjugated, e.g. fused, to peptides or proteins that can be detected via a labeled binding partner or antibody. In some examples, a binding partner can be detected by HC methods using a labeled secondary reagent, such as labeled antibodies, that recognize one or more regions, e.g. epitopes, of the binding reagent.

In some embodiments, the resulting stained specimens, such as obtained by histochemistry methods, are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of a binding partner, e.g. PD-L1, in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g. published U.S. patent Appl. No. US20100136549).

In some embodiments, a biological sample is detected for cells surface positive for a binding partner, e.g. PD-L1, CD28 or CTLA-4, if there is a detectable expression level of the binding partner (e.g. following contacting with the binding reagent and detection of bound binding reagent) in at least or at least about or about 1% of the cells, at least or at least about or about 5% of the cells, at least or at least about or about 10% of the cells, at least or at least about or about 20% of the cells, at least or at least about or about 40% of the cells or more.

In some embodiments, the biological sample is a tumor tissue sample comprising stromal cells, tumor cells or tumor infiltrating cells, such as tumor infiltrating immune cells, e.g. tumor infiltrating lymphocytes. In some embodiments, the tumor tissue sample is detected for cells surface positive for PD-L1 if there is a detectable expression level of the binding partner (e.g. following contacting with the binding reagent and detection of bound binding reagent) in at least or at least about or about 1% of the cells, at least or at least about or about 5% of the cells, at least or at least about or about 10% of the cells, at least or at least about or about 20% of the cells, at least or at least about or about 40% of the cells or more. In some embodiments, the cells are tumor cells or tumor infiltrating immune cells. In some embodiments, the tumor tissue sample is detected for cells surface positive for CD28 if there is a detectable expression level of the binding partner (e.g. following contacting with the binding reagent and detection of bound binding reagent) in at least or at least about or about 1% of the cells, at least or at least about or about 5% of the cells, at least or at least about or about 10% of the cells, at least or at least about or about 20% of the cells, at least or at least about or about 40% of the cells or more. In some embodiments, the cells are tumor infiltrating immune lymphocytes.

### VI. Articles of Manufacture and Kits

Also provided herein are articles of manufacture that comprise the pharmaceutical compositions described herein in suitable packaging. Suitable packaging for compositions described herein are known in the art, and include, for example, vials (such as sealed vials), vessels, ampules, bottles, jars, flexible packaging (e.g., sealed Mylar or plastic bags) or other bags, and the like. These articles of manufacture may further be sterilized and/or sealed.

Further provided are kits comprising the pharmaceutical compositions (or articles of manufacture) described herein, which may further comprise instruction(s) on methods of using the composition, such as uses described herein. The kits described herein may also include other materials desirable from a commercial and user standpoint, including other buffers, diluents, filters, needles, syringes, and package inserts with instructions for performing any methods described herein.

### VII. Exemplary Embodiments

Among the provided embodiments are:

1. An immunomodulatory protein comprising at least one variant CD80 extracellular domain polypeptide, wherein the variant CD80 extracellular domain polypeptide comprises an amino acid substitution at position 11 in the sequence set forth in SEQ ID NO:2 or a portion thereof comprising an IgV domain, wherein the variant CD80 extracellular domain polypeptide exhibits increased binding to PD-L1 compared to wild-type CD80 set forth in SEQ ID NO:2 or the portion thereof comprising the IgV domain.

2. An immunomodulatory protein comprising at least one variant CD80 extracellular domain polypeptide, wherein the variant CD80 extracellular domain polypeptide comprises 2 to 10 amino acid substitutions at positions in the sequence set forth in SEQ ID NO:2 or a portion thereof comprising an IgV domain, wherein at least one substitution is at a position selected from among 9, 10, or 11, wherein the variant CD80 extracellular domain polypeptide exhibits increased binding to PD-L1 compared to wild-type CD80 set forth in SEQ ID NO:2 or the portion thereof comprising the IgV domain.

3. The immunomodulatory protein of embodiment 1 or embodiment 2, wherein the variant CD80 extracellular domain polypeptide comprises an amino acid substitution at position 11 and the substitution is to an aromatic amino acid residue.

4. The immunomodulatory protein of embodiment 2, wherein the aromatic amino acid residues are selected from the group consisting of tyrosine (Y), tryptophan (W) or phenylalanine (F), optionally wherein the amino acid substitution is V11Y, V11F, or V11W

5. The immunomodulatory protein of any of embodiments 1-4, wherein the variant CD80 extracellular domain polypeptide comprises the amino acid substitution V11Y.

6. The immunomodulatory protein of any of embodiments 1-5, wherein the variant CD80 extracellular domain polypeptide comprises the amino acid substitutions V11Y/M47V/F59S/L85E, V11Y/M47V/L85E, V11Y/M42I/M47V/A71G, V11Y/T28Y/M47L/L85E, E10S/V11F/T28Y/M47L/T62S, E10G/V11W/V22S/T28P/A71G/E81K/Y87R, V11Y/T28Y/L85E/Y87I, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E10G/V11W/M47V/L85E, V11Y/T28Y/M47L/V68L/L85E, V11Y/M42W/T62A/L85E, V11Y/M42W/T62A, V11Y/M42W/F59Y/V68N, V11Y/M42W/E52K/T62A/L85E, V11Y/E35D/Y87Q/T101R, K9N/E10A/V11W/H18F/T28H/M47L/T62E/R73D, V11Y/E35G/M42G/F59S, V11Y/T28R/E35G/M47L/F59S, V11Y/T28R/E35G/M47L/A71G, V11Y/V68T, V11W/T28Y/D46V/R73E/F92L, V11W/T28Y/D46V/V68T/R73T/Y87N, E10S/V11Y/M42R/A71V, K9N/V11W/M47L/V68T/R73T/Y87N, V11Y/M47L/V68T, V11W/T28H/D46Q/V68L/L85E, E7Q/V11Y/R29H/M47L/V68T, V11Y/M47V/A71G/L85E, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

7. The immunomodulatory protein of embodiment 2, wherein the variant CD80 extracellular domain polypeptide comprises an amino acid substitution at position 9 and is substitution to a polar uncharged amino acid residue.

8. The immunomodulatory protein of embodiment 7, wherein the polar uncharged amino acid is selected from the group consisting of serine (S), asparagine (N), glutamine (Q), threonine (T).

9. The immunomodulatory protein of any of embodiments 2, 7 and 8, wherein the variant CD80 extracellular domain polypeptide comprises the amino acid substitution K9S or K9N.

10. The immunomodulatory protein of embodiment 2, wherein the variant CD80 extracellular domain polypeptide comprises an amino acid substitution at position 9 and the substitution is to another basic amino acid.

11. The immunomodulatory protein of embodiment 10, wherein the other basic amino acid is selected from the group consisting of arginine or histidine.

12. The immunomodulatory protein of any of embodiments 2, 10 and 11, wherein the variant CD80 extracellular domain polypeptide comprises the amino acid substitution is K9R.

13. The immunomodulatory protein of any of embodiments 2 and 7-12, wherein the variant CD80 extracellular domain polypeptide comprises the amino acid substitutions K9N/E10G/N63S/L85E, K9N/E10G/E35G/D46E/M47V/V68M, K9R/E10S/V11Y/M42L/F59M/V68M/L85E, K9S/E10R/V11Y/M47L/A71G, K9N/E10R/H18V/T28Y/A71G, K9N/E10A/V11W/H18F/T28H/M47L/T62E/R73D, K9R/E10A/E35G/V68T/T101K, K9R/E10A/E35G/V68L/L85E, K9N/E10G/Y87K/T101Q, K9N/V11W/M47L/V68T/R73T/Y87N, K9R/A26T/T28Y/E35A/M47L/A71G.

14. The immunomodulatory protein of embodiment 2, wherein the variant CD80 extracellular domain comprises an amino acid substitution at position 10 and the substitution is to a nonpolar amino acid.

15. The immunomodulatory protein of embodiment 14, wherein the nonpolar amino acid is glycine, alanine or valine.

16. The immunomodulatory protein of embodiment 2, 14 or 15, wherein the variant CD80 extracellular domain comprises the amino acid substitution E10G or E10A

17. The immunomodulatory protein of embodiment 2, wherein the variant CD80 extracellular domain comprises an amino acid substitution at position 10 and the amino acid substitution is selected from the group consisting of E10G, E10S, E10R, and E10A.

18. The immunomodulatory protein of any of embodiments 2 and 14-17, wherein the variant CD80 extracellular domain polypeptide comprises the amino acid substitutions K9N/E10G/N63S/L85E, E10G/E35G/D46E/M47V/V68M, K9N/E10G/E35G/D46E/M47V/V68M, K9R/E10S/V11Y/M42L/F59M/V68M/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, E10G/V11W/T28Y/M47L, K9S/E10R/V11Y/M47L/A71G, E10G/V11W/E35G/M47L/A71G/Y87R, E10G/V11W/M47V/L85E, E10G/H18Y, K9N/E10R/H18V/T28Y/A71G, E10G/H18Y/T28Y/M47W/T62S, K9N/E10A/V11W/H18F/T28H/M47L/T62E/R73D, E10G/D46K/L85E, E10G/H18T/Q27T/D46E/M47L, K9R/E10A/E35G/V68T/T101K, E10G/E35G/D46E/M47V/V68M/T101K, K9R/E10A/E35G/V68L/L85E, K9N/E10G/Y87K/T101Q, E10S/V11Y/M42R/A71V, E10G/A26S/T28Y, E10S/V68M/Y87P, E10G/Q27F/D46N/A71G/D90G, or E10S/V11F/T28Y/M47L.

19. The immunomodulatory protein of any of embodiments 1-5, 7-12 and 14-17, wherein the variant CD80 extracellular domain polypeptide further comprises an amino acid substitution at position 47 in the sequence set forth in SEQ ID NO:2 or the portion thereof comprising an IgV domain.

20. The immunomodulatory protein of embodiment 19, wherein the amino acid substitution at position 47 is to another hydrophobic amino acid.

21. The immunomodulatory protein of embodiment 20, wherein the hydrophobic amino acid is selected from the group consisting of valine, leucine, isoleucine or proline.

22. The immunomodulatory protein of any of embodiments 19-21, wherein the variant CD80 extracellular domain comprises the amino acid substitution M47L or M47V.

23. The immunomodulatory protein of any of embodiments 19-22, wherein the variant CD80 extracellular domain polypeptide comprises the amino acid substitutions V11W/M47L, V11F/M47L, V11Y/M47L, V11W/M47V, or V11Y/M47V.

24. The immunomodulatory protein of any of embodiments 19-23, wherein the variant CD80 extracellular domain polypeptide comprises the amino acid substitutions V11Y/M47V/F59S/L85E, V11Y/M47V/L85E, V11Y/M42I/M47V/A71G, V11Y/T28Y/M47L/L85E, E10S/V11F/T28Y/M47L/T62S, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E10G/V11W/M47V/L85E, V11Y/T28Y/M47L/V68L/L85E, K9N/E10A/V11W/H18F/T28H/M47L/T62E/R73D, V11Y/T28R/E35G/M47L/F59S, V11Y/T28R/E35G/M47L/A71G, K9N/V11W/M47L/V68T/R73T/Y87N, V11Y/M47L/V68T, E7Q/V11Y/R29H/M47L/V68T, V11Y/M47V/A71G/L85E, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

25. The immunomodulatory protein of any of embodiments 1-5, 7-12, 14-17 and 19-23, wherein the variant CD80 extracellular domain polypeptide further comprises an amino acid substitution at position 28 in the sequence set forth in SEQ ID NO:2 or the portion thereof comprising an IgV domain.

26. The immunomodulatory protein of any of embodiments 1-5, 7-12, 14-17, 19-23 and 25, wherein the variant CD80 extracellular domain polypeptide comprises an amino acid substitution selected from T28Y, T28P, T28H, T28R, or T28V.

27. The immunomodulatory protein of embodiment 26, wherein the amino acid substitution is T28Y.

28. The immunomodulatory protein of any of embodiments 19-23 and 25-27, wherein the variant CD80 extracellular domain polypeptide comprises the amino acid substitutions V11W/T28Y/M47L, V11F/T28Y/M47L, V11Y/T28Y/M47L, V11W/T28Y/M47V, V11F/T28Y/M47V, or V11Y/T28Y/M47V.

29. The immunomodulatory protein of any of embodiments 1-5, 7-12, 14-17, 19-28, wherein the variant CD80 extracellular domain polypeptide comprises the amino acid substitutions V11Y/T28Y/M47L.

30. The immunomodulatory protein of any of embodiments 1-5, 7-12, 14-17, 19-29, wherein the variant CD80 extracellular domain polypeptide further comprises an amino acid substitution at position 68, optionally wherein the amino acid substitution is V68M, V68L, V68N, V68T, or V68S.

31. The immunomodulatory protein of any of embodiments 1-5, 7-12, 14-17, 19-30, wherein the variant CD80 extracellular domain polypeptide comprises the amino acid substitutions V11Y/T28Y/M47L/V68M.

32. The immunomodulatory protein of any of embodiments 1-5, 7-12, 14-17, 19-30, wherein the variant CD80 extracellular domain polypeptide comprises the amino acid substitutions V11Y/T28Y/M47L/V68L.

33. The immunomodulatory protein of any of embodiments 1-5, 7-12, 14-17, 19-29, wherein the variant CD80 extracellular domain polypeptide comprises the amino acid substitutions E10G/V11W/T28Y/M47L.

34. The immunomodulatory protein of any of embodiments 1-33, wherein the variant CD80 extracellular domain polypeptide further comprises an amino acid substitution at position 7 in the sequence set forth in SEQ ID NO:2 or a portion thereof comprising an IgV domain.

35. The immunomodulatory protein of any of embodiments 1-34, wherein the variant CD80 extracellular domain polypeptide further comprises an amino acid substitution at position 18 in the sequence set forth in SEQ ID NO:2 or a portion thereof comprising an IgV domain.

36. The immunomodulatory protein of embodiment 35, wherein the variant CD80 extracellular domain polypeptide comprises amino acid substitutions E10G/V11W/H18Y/T28Y/M47L, V11Y/H18Y/T28Y/M47L or V11Y/H18Y/T28Y/M47L/A71G.

37. An immunomodulatory protein comprising at least one variant CD80 extracellular domain polypeptide, wherein the variant CD80 extracellular domain polypeptide comprises an amino acid substitution at position 7 in the sequence set forth in SEQ ID NO:163 or a portion thereof comprising the IgV domain, wherein the variant CD80 extracellular domain polypeptide exhibits increased binding to PD-L1 compared to the wild-type CD80 extracellular domain polypeptide set forth in SEQ ID NO:2 or the portion thereof comprising the IgV domain.

38. The immunomodulatory protein of embodiment 34 or embodiment 37, wherein the variant CD80 extracellular domain polypeptide comprises an amino acid substitution at position 7 and the substitution is to a polar uncharged amino acid residue.

39. The immunomodulatory protein of embodiment 38, wherein the polar uncharged amino acid is selected from the group consisting of serine (S), asparagine (N), glutamine (Q), threonine (T).

40. The immunomodulatory protein of any of embodiments 34-39, wherein the variant CD80 extracellular domain polypeptide comprises the amino acid substitution E7Q, E7N or E7S.

41. The immunomodulatory protein of embodiment 34 or embodiment 37, wherein the variant CD80 extracellular domain polypeptide comprises an amino acid substitution at position 7 and the substitution is to a basic amino acid.

42. The immunomodulatory protein of embodiment 41, wherein the basic amino acid is selected from the group consisting of arginine, histidine or lysine.

43. The immunomodulatory protein of any of embodiments 34, 37, 41 and 42, wherein the variant CD80 extracellular domain polypeptide comprises the amino acid substitution E7H or E7K.

44. The immunomodulatory protein of any of embodiments 34-43, wherein the variant CD80 extracellular domain polypeptide comprises the amino acid substitutions E7S/H18I/V20L/A26K/M47L/A71N, E7K/V11W/N63H/A71G/Y87K, E7N/E35D/T101R, E7H/H18L/V20I/T28Y/D46S/A71G, E7N/E35D/F59S, or E7Q/V11Y/R29H/M47L/V68T.

45. The immunomodulatory protein of any of embodiments 1-44, further comprising an amino acid substitution at position 101 in the sequence set forth in SEQ ID NO:2 or a portion thereof comprising an IgV domain.

46. An immunomodulatory protein comprising at least one variant CD80 extracellular domain polypeptide, wherein the variant CD80 extracellular domain polypeptide comprises an amino acid substitution at position 101 in the sequence set forth in SEQ ID NO:2 or a portion thereof comprising an IgV domain, wherein the variant CD80 extracellular domain polypeptide exhibits increased binding to PD-L1 compared to wild-type CD80 set forth in SEQ ID NO:2 or the portion thereof comprising the IgV domain.

47. The immunomodulatory protein of embodiment 45 or embodiment 46, wherein the variant CD80 extracellular domain polypeptide comprises an amino acid substitution at position 101 and the substitution is to a charged amino acid residue.

48. The immunomodulatory protein of embodiment 47, wherein the charged amino acid residue is basic and the amino acid substitution is to a histidine (H), lysine (K) or arginine (R).

49. The immunomodulatory protein of embodiment 48, wherein the variant CD80 extracellular domain polypeptide comprises an amino acid substitution T101K or T101R.

50. The immunomodulatory protein of embodiment 47, wherein the charged amino acid residue is acidic and the amino acid substitution is to aspartate (D), glutamate (E), asparagine (N) or glutamine (Q).

51. The immunomodulatory protein of embodiment 50, wherein the variant CD80 extracellular domain polypeptide comprises an amino acid substitution T101Q.

52. The immunomodulatory protein of any of embodiments 45-51, wherein the variant CD80 extracellular domain polypeptide comprises the amino acid substitutions E7N/E35D/T101R, V11Y/E35D/Y87Q/T101R, E35D/V68T/T101K, K9R/E10A/E35G/V68T/T101K, E10G/E35G/D46E/M47V/V68M/T101K, or K9N/E10G/Y87K/T101Q.

53. The immunomodulatory protein of any of embodiments 45-49 and 52, wherein the variant CD80 extracellular domain polypeptide comprises the amino acid substitutions V11Y/E35D/Y87Q/T101R.

54. The immunomodulatory protein of any of embodiments 1-53, further comprising an additional amino acid substitution at a different position wherein the amino acid substitution is selected from the group consisting of E7S, E7K, E7N, E7H, E7Q, K9N, K9R, K9S, E10G, E10S, E10R, E10A, V11Y, V11F, V11W, H18I, H18Y, H18F, H18V, H18L, H18T, V20L, V20I, V22S, A26K, A26G, A26Q, A26E, A26S, A26T, Q27F, Q27T, T28Y, T28P, T28H, T28R, T28V, R29S, R29H, E35G, E35D, E35A, M42I, M42L, M42G, M42W, M42R, D46E, D46S, D46K, D46V, D46Q, D46N, M47V, M47L, M47R, M47W, E52K, F59S, F59M, F59Y, T62S, T62A, T62E, N63S, N63I, N63H, V68M, V68L, V68N, V68T, V68S, A71G, A71N, A71V, R73D, R73E, R73T, E77G, E81K, L85E, L85Q, Y87R, Y87I, Y87K, Y87Q, Y87N, Y87P, D90G, F92L, T101R, TIO1K, or T101Q, or a conservative amino acid substitution of any of the foregoing.

55. An immunomodulatory protein comprising at least one variant CD80 extracellular domain polypeptide, wherein the variant CD80 extracellular domain polypeptide comprises one or more amino acid substitutions in the sequence set forth in SEQ ID NO:2 or a portion thereof comprising the IgV domain, wherein the one or more amino acid substitutions is selected from E7S, E7K, E7N, E7H, E7Q, K9N, K9R, K9S, E10G, E10S, E10A, V11Y, V11F, V11W, V20I, V22S, Q27F, Q27T, T28P, T28H, T28R, T28V, R29S, E35A, M42L, M42G, M42W, M42R, D46S, D46K, D46Q, M47R, M47W, E52K, F59S, T62S, T62A, N63I, N63H, V68N, V68T, V68S, A71N, A71V, R73D, R73E, R73T, L85Q, Y87R, Y87I, Y87K, Y87P, T101R, TIO1K, and T101Q, wherein the variant CD80 polypeptide exhibits increased binding to PD-L1 compared to wild-type CD80 comprising the sequence set forth in SEQ ID NO:2 or the portion thereof comprising the IgV domain.

56. The immunomodulatory protein of any of embodiments 1-55, wherein the variant CD80 extracellular domain polypeptide comprises 2, 3, 4, 5, 6, 7, 8, 9 or 10 amino acid substitutions compared to SEQ ID NO:2 or the portion thereof comprising the IgV domain.

57. The immunomodulatory protein of any of embodiments 1-56, comprising no more than 4 amino acid substitutions compared to SEQ ID NO:2 or the portion thereof comprising the IgV domain.

58. The immunomodulatory protein of any of embodiments 1-57, comprising 2, 3 or 4 amino acid substitutions compared to SEQ ID NO:2 or the portion thereof comprising the IgV domain.

59. An immunomodulatory polypeptide comprising at least one variant CD80 extracellular domain polypeptide, wherein the variant CD80 extracellular domain polypeptide comprises the sequence set forth in SEQ ID NO:2 or a portion thereof comprising an IgV domain, in which is contained amino acid substitutions selected from V11Y/M47V/F59S/L85E, V11Y/M47V/L85E, V11Y/M42I/M47V/A71G, K9N/E10G/N63S/L85E, E10G/E35G/D46E/M47V/V68M, K9N/E10G/E35G/D46E/M47V/V68M, E7S/H18I/V20L/A26K/M47L/A71N, K9R/E10S/V11Y/M42L/F59M/V68M/L85E, V11Y/T28Y/M47L/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, V11Y/T28Y/L85E/Y87I, H18F/M42G/F59Y/V68N, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E35D/V68L/L85E, E10G/V11W/M47V/L85E, T28Y/M47L, V11Y/T28Y/M47L/V68L/L85E, E7N/E35D/T101R, V11Y/M42W/T62A/L85E, V11Y/M42W/T62A, V11Y/M42W/F59Y/V68N, V11Y/M42W/E52K/T62A/L85E, V11Y/E35D/Y87Q/T101R, H18Y/A26E/R29S/E35D/M47L/V68M/A71G/E77G/D90G, E10G/H18Y, K9N/E10R/H18V/T28Y/A71G, E10G/H18Y/T28Y/M47W/T62S, E7H/H18L/V20I/T28Y/D46S/A71G, K9N/E10A/V11W/H18F/T28H/M47L/T62E/R73D, H18V/V20I/T28Y/E35G/M47V/R73E, E10G/D46K/L85E, E10G/H18T/Q27T/D46E/M47L, E35D/V68T/T101K, V11Y/E35G/M42G/F59S, V11Y/T28R/E35G/M47L/F59S, E7N/E35D/F59S, V11Y/T28R/E35G/M47L/A71G, K9R/E10A/E35G/V68T/T101K, V11Y/V68T, E10G/E35G/D46E/M47V/V68M/T101K, K9R/E10A/E35G/V68L/L85E, V11W/T28Y/D46V/R73E/F92L, K9N/E10G/Y87K/T101Q, V11W/T28Y/D46V/V68T/R73T/Y87N, E10S/V11Y/M42R/A71V, H18F/T28V/M47L/V68S, E10G/A26S/T28Y, E35D/D46Q/L85E, E10S/V68M/Y87P, K9N/V11W/M47L/V68T/R73T/Y87N, V11Y/M47L/V68T, K9R/A26T/T28Y/E35A/M47L/A71G, V11W/T28H/D46Q/V68L/L85E, E7Q/V11Y/R29H/M47L/V68T, V11Y/M47V/A71G/L85E, E10G/Q27F/D46N/A71G/D90G, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

60. The immunomodulatory protein of any one of embodiments 1-59, wherein the variant CD80 extracellular domain polypeptide exhibits at least 85% sequence identity to the sequence set forth in SEQ ID No: 2 or the portion thereof comprising the IgV domain.

61. The immunomodulatory protein of any one of embodiments 1-60, wherein the variant CD80 extracellular domain polypeptide exhibits at least 90% sequence identity to the sequence set forth in SEQ ID No: 2 or the portion thereof comprising the IgV domain.

62. The immunomodulatory protein of any one of embodiments 1-61, wherein the variant CD80 extracellular domain polypeptide exhibits at least 95% sequence identity to the sequence set forth in SEQ ID No: 2 or the portion thereof comprising the IgV domain.

63. The immunomodulatory protein of any one of embodiments 1-62, wherein the variant CD80 extracellular domain polypeptide exhibits at least 97% sequence identity to the sequence set forth in SEQ ID No: 2 or the portion thereof comprising the IgV domain.

64. The immunomodulatory protein of any of embodiments 1-63, wherein the portion of SEQ ID NO:2 comprising the IgV domain comprises amino acids 1-101 of SEQ ID NO:2 and has a length of no more than 110 amino acids.

65. The immunomodulatory protein of any of embodiments 1-64, wherein the portion of SEQ ID NO:2 comprising the IgV domain is set forth in SEQ ID NO:163.

66. The immunomodulatory protein of any of embodiments 1-63, wherein the portion of SEQ ID NO:2 comprising the IgV domain is set forth as amino acids 1-107 of SEQ ID NO:2 (SEQ ID NO:164).

67. The immunomodulatory protein of any one of embodiments 1-66, wherein the variant CD80 extracellular domain polypeptide comprises the sequence of amino acids set forth in any of SEQ ID NOS: 165-244 or a sequence of amino acids that exhibits at least 95% sequence identity to any of SEQ ID NOS: 165-244.

68. The immunomodulatory protein of any one of embodiments 1-67, wherein the variant CD80 extracellular domain polypeptide comprises the sequence of amino acids set forth in any of SEQ ID NOS: 165-244.

69. The immunomodulatory protein of any of embodiments 1-68, wherein the variant CD80 extracellular domain polypeptide is set forth in any one of SEQ ID NOS: 165-244.

70. The immunomodulatory protein of any of embodiments 1-69, wherein the variant CD80 extracellular domain polypeptide is set forth in SEQ ID NO:180.

71. The immunomodulatory protein of any of embodiments 1-69, wherein the variant CD80 extracellular domain polypeptide is set forth in SEQ ID NO:185.

72. The immunomodulatory protein of any of embodiments 1-69, wherein the variant CD80 extracellular domain polypeptide is set forth in SEQ ID NO:197.

73. The immunomodulatory protein of any of embodiments 1-69, wherein the variant CD80 extracellular domain polypeptide is set forth in SEQ ID NO:233.

74. The immunomodulatory protein of any of embodiments 1-69, wherein the variant CD80 extracellular domain polypeptide is set forth in SEQ ID NO:234.

75. The immunomodulatory protein of any of embodiments 1-74, comprising a heterologous moiety that is linked to the at least one variant CD80 polypeptide, optionally via a linker.

76. The immunomodulatory protein of embodiment 75, wherein the heterologous moiety is a half-life extending moiety, a multimerization domain, a targeting moiety that binds to a molecule on the surface of a cell, or a detectable label.

77. The immunomodulatory protein of embodiment 76, wherein the half-life extending moiety comprises a multimerization domain, albumin, an albumin-binding polypeptide, Pro/Ala/Ser (PAS), a C-terminal peptide (CTP) of the beta subunit of human chorionic gonadotropin, polyethylene glycol (PEG), long unstructured hydrophilic sequences of amino acids (XTEN), hydroxyethyl starch (HES), an albumin-binding small molecule, or a combination thereof.

78. The immunomodulatory protein of any of embodiments 1-75, wherein the immunomodulatory protein is a variant CD80-Fc fusion protein comprising the at least one variant polypeptide and an Fc region of an immunoglobulin.

79. The immunomodulatory protein of embodiment 78, wherein the at least one variant CD80 polypeptide is linked to the Fc region via a linker, optionally a peptide linker.

80. The immunomodulatory protein of embodiment 79, wherein the linker comprises a peptide linker and the peptide linker is selected from GGGGS (G4S; SEQ ID NO: 328), GSGGGGS (SEQ ID NO: 325), GGGGSGGGGS (2×GGGGS; SEQ ID NO: 329), GGGGSGGGGSGGGGS (3×GGGGS; SEQ ID NO: 330), GGGGSGGGGSGGGGSGGGGS (4×GGGGS, SEQ ID NO:331), GGGGSGGGGSGGGGSGGGGSGGGGS (5×GGGGS, SEQ ID NO: 332), GGGGSSA (SEQ ID NO: 333), or GSGGGGSGGGGS (SEQ ID NO:335) or combinations thereof.

81. The immunomodulatory protein of any of embodiments 78-80, wherein the immunoglobulin Fc is an IgG1 Fc domain, or is a variant Fc domain that exhibits reduced binding affinity to an Fc receptor and/or reduced effector function, optionally as compared to a wild-type IgG1 Fc domain.

82. The immunomodulatory protein of embodiment 81, wherein the immunoglobulin Fc is a variant IgG1 Fc domain comprising one or more amino acid substitutions selected from L234A, L234V, L235A, L235E, G237A, S267K, R292C, N297G, and V302C, by EU numbering.

83. The immunomodulatory protein of embodiment 82, wherein the immunoglobulin Fc region comprises the amino acid substitutions L234A, L235E an G237A by EU numbering, optionally wherein the Fc region is set forth in any of SEQ ID NOS: 344, 345, 348 or 351.

84. The immunomodulatory protein of embodiment 78-83, wherein the immunoglobulin Fc region comprises the sequence set forth in SEQ ID NO:344.

85. The immunomodulatory protein of any of embodiments 78-80, wherein the immunoglobulin Fc is an IgG4 Fc domain, optionally comprising the amino acid substitution S228P.

86. The immunomodulatory protein of any of embodiments 78-80 and 85, wherein the immunoglobulin Fc region comprises the sequence set forth in SEQ ID NO:326.

87. The immunomodulatory protein of any one of embodiments 78-86, wherein the variant CD80-Fc fusion protein comprises the structure: variant CD80 polypeptide (vCD80)-Linker-Fc region.

88. The immunomodulatory protein of any of embodiments 78-87, wherein the variant CD80-Fc fusion protein comprises the sequence of amino acids set forth in any one of SEQ ID NOS: 245-324 or a sequence that exhibits at least 85%, at least 90%, at least 95% or at least 97% sequence identity to any one of SEQ ID NOS: 245-324.

89. The immunomodulatory protein of any of embodiments 78-86, wherein the variant CD80-Fc fusion protein comprises the structure: (vCD80)-Linker-Fc region-Linker-(vCD80).

90. The immunomodulatory protein of any of embodiments 78-86 and 89, wherein the variant CD80-Fc fusion protein comprises the sequence of amino acids set forth in any one of SEQ ID NOS: 336, 338, 339 or 341, or a sequence that exhibits at least 85%, at least 90%, at least 95% or at least 97% sequence identity to any one of SEQ ID NOS: 336, 338, 339 or 341.

91. The immunomodulatory protein of any of embodiments 78-86, wherein the variant CD80-Fc fusion protein comprises the structure: (vCD80)-Linker-(vCD80)-Linker-Fc region.

92. The immunomodulatory protein of any of embodiments 78-86 and 91, wherein the variant CD80-Fc fusion protein comprises the sequence of amino acids set forth in SEQ ID NO: 340 or 342, or a sequence that exhibits at least 85%, at least 90%, at least 95% or at least 97% sequence identity to SEQ ID NO: 340 or 342.

93. The immunomodulatory protein of any of embodiments 78-92 that is a homodimer comprising two identical copies of the variant CD80-Fc fusion protein.

94. The immunomodulatory protein of any one of embodiments 1-93, wherein the PD-L1 is human PD-L1.

95. The immunomodulatory protein of any one of embodiments 1-94, wherein the binding affinity of the variant CD80 extracellular domain to PD-L1 is increased greater than 1.1-fold compared to wild-type CD80 set forth in SEQ ID NO:2 or the portion comprising the IgV domain.

96. The immunomodulatory protein of embodiment 95, wherein the binding affinity is increased greater than 1.5-fold, greater than 2-fold, greater than 3-fold, greater than 4-fold, greater than 5-fold, greater than 6-fold, greater than 7-fold, greater than 8-fold, greater than 9-fold or greater than 10-fold.

97. The immunomodulatory protein of any one of embodiments 1-96, wherein the binding affinity is determined by Mean Fluorescence Intensity (MFI) as measured by flow cytometry in a cell-based binding assay for a PD-L1-expressing cell.

98. The immunomodulatory protein of any of embodiments 1-97, wherein the immunomodulatory protein blocks binding of PD-L1 to PD-1.

99. The immunomodulatory protein of any of embodiments 1-98, wherein the variant CD80 extracellular polypeptide exhibit an off-rate (Koff) for binding to PD-L1 of less than 50×10⁻³ s⁻¹.

100. The immunomodulatory protein of any of embodiments 1-99, wherein the variant CD80 extracellular polypeptide has a Koff for binding to PD-L1 of at or about or less than 40×10⁻³ s⁻¹, 30×10⁻³ s⁻¹, 20×10⁻³ s⁻¹, 15×10⁻³ s⁻¹, 10×10⁻³ s⁻¹, 5×10⁻³ s⁻¹, or 1×10⁻³ s⁻¹.

101. The immunomodulatory protein of any of embodiments 1-99, wherein the variant CD80 extracellular polypeptide has a Koff for binding to PD-L1 of between 1×10⁻³ s⁻¹ and 50×10⁻³ s⁻¹, 1×10⁻³ s⁻¹ and 30×10⁻³ s⁻¹, 1×10⁻³ s⁻¹ and 20×10⁻³ s⁻¹, 1×10⁻³ s⁻¹ and 15×10⁻³ s⁻¹, 1×10⁻³ s⁻¹ and 10×10⁻³ s⁻¹, 1×10⁻³ s⁻¹ and 5×10⁻³ s⁻¹, 5×10⁻³ s⁻¹ and 50×10⁻³ s⁻¹, 5×10⁻³ s⁻¹ and 30×10⁻³ s⁻¹, 5×10⁻³ s⁻¹ and 20×10⁻³ s⁻¹, 5×10⁻³ s⁻¹ and 15×10⁻³ s⁻¹, 5×10⁻³ s⁻¹ and 10×10⁻³ s⁻¹, 10×10⁻³ s⁻¹ and 50×10⁻³ s⁻¹, 10×10⁻³ s⁻¹ and 30×10⁻³ s⁻¹, 10×10⁻³ s⁻¹ and 20×10⁻³ s⁻¹, 1×10⁻³ s⁻¹ and 15×10⁻³ s⁻¹, 5×10⁻³ s⁻¹ and 50×10⁻³ s⁻¹, 15×10⁻³ s⁻¹ and 30×10⁻³ s⁻¹, 15×10⁻³ s⁻¹ and 20×10⁻³ s⁻¹, 20×10⁻³ s⁻¹ and 50×10⁻³ s⁻¹, 20×10⁻³ s⁻¹ and 30×10⁻³ s⁻¹, or 30×10⁻³ s⁻¹ and 50×10⁻³ s⁻¹.

102. The immunomodulatory protein of any of embodiments 1-101, wherein the variant CD80 polypeptide binds CD28, optionally with a binding affinity is 0.8-fold to 30-fold of the binding affinity of wild-type CD80 set forth in SEQ ID NO:2 or the portion thereof comprising the IgV domain.

103. The immunomodulatory protein of any of embodiments 1-102, wherein the variant CD80 polypeptide exhibits increased binding to CD28 compared to wild-type CD80 set forth in SEQ ID NO:2 or the portion thereof comprising the IgV domain.

104. The immunomodulatory protein of any one of embodiments 1-94, wherein the binding affinity of the variant CD80 extracellular domain to CD80 is increased greater than 1.1-fold compared to wild-type CD80 set forth in SEQ ID NO:2 or the portion comprising the IgV domain.

105. The immunomodulatory protein of embodiment 103 or embodiment 104, wherein the binding affinity is increased greater than 1.5-fold, greater than 2-fold, greater than 3-fold, greater than 4-fold, greater than 5-fold, greater than 6-fold, greater than 7-fold, greater than 8-fold, greater than 9-fold or greater than 10-fold.

106. The immunomodulatory protein of any one of embodiments 102-105, wherein the binding affinity is determined by Mean Fluorescence Intensity (MFI) as measured by flow cytometry in a cell-based binding assay for a CD28-expressing cell.

107. The immunomodulatory protein of any one of embodiments 1-106, wherein the immunomodulatory protein exhibits CD28 agonism, optionally as determined in a T reporter assay.

108. The immunomodulatory protein of embodiment 107, wherein the CD28 agonism is PD-L1 dependent, optionally as determined in a T cell reporter assay in the presence of PD-L1 expressing cells.

109. The immunomodulatory protein of any of embodiments 1-108, wherein the immunomodulatory protein blocks binding of CTLA-4 to its ligand CD80 or CD86.

110. The immunomodulatory protein of any of embodiments 1-109 that is a soluble protein.

111. The immunomodulatory protein of any of embodiments 1-110 that is a purified protein.

112. A nucleic acid molecule(s) encoding the immunomodulatory protein of any of embodiments 1-111.

113. The nucleic acid molecule of embodiment 112, that is a synthetic nucleic acid.

114. The nucleic acid molecule of embodiment 112 or embodiment 113 that is a cDNA.

115. A vector, comprising the nucleic acid molecule of any of embodiments 111-113.

116. The vector of embodiment 115 that is an expression vector.

117. The vector of embodiment 115 or embodiment 116, wherein the vector is a mammalian expression vector or a viral vector.

118. An immune cell comprising the immunomodulatory protein of any of embodiments 1-111.

119. The immune cell of embodiment 118, wherein the immune cell further comprises a T cell receptor (TCR) or a chimeric antigen receptor (CAR).

120. The immune cell of embodiment 118 or embodiment 119, wherein the immune cell is a lymphocyte.

121. The immune cell of embodiment 120, wherein the lymphocyte is a T cell.

122. The immune cell of any of embodiments 118-121, wherein the immunomodulatory protein is a transmembrane protein expressed on the surface of the immune cell.

123. The immune cell of any of embodiments 118-122, wherein the immunomodulatory protein is secretable from the immune cell.

124. A method of producing an immunomodulatory protein comprising introducing the nucleic acid molecule of any of embodiments 111-113 or vector of any of embodiments 115-117 into a host cell under conditions to express the protein in the cell, and isolating or purifying the protein from the cell.

125. A purified immunomodulatory protein produced by the method of embodiment 124.

126. A pharmaceutical composition comprising the immunomodulatory protein of any of embodiments 1-111 or 125.

127. The pharmaceutical composition of embodiment 126, comprising a pharmaceutically acceptable excipient.

128. The pharmaceutical composition of embodiment 126 or embodiment 127, wherein the pharmaceutical composition is sterile.

129. An article of manufacture comprising the pharmaceutical composition of any of embodiments 126-128 in a vial or container.

130. The article of manufacture of embodiment 129, wherein the vial or container is sealed.

131. A kit comprising the pharmaceutical composition of any of embodiments 126-128, and instructions for use.

132. A method of stimulating an immune response in a subject, comprising administering an immunomodulatory protein of any of embodiments 1-111, the immune cell of any of embodiments 118-123, or the pharmaceutical composition of any of embodiments 126-128 to a subject in need thereof.

133. The method of embodiment 132, wherein stimulating the immune response treats a disease or condition in the subject.

134. A method of treating a disease or condition in a subject, the method comprising administering the immunomodulatory protein of any of embodiments 1-111, the immune cell of any of embodiments 118-123, or the pharmaceutical composition of any of embodiments 126-128 to a subject having the disease or condition.

135. The method of embodiment 133 or embodiment 134, wherein the disease or condition is a cancer.

136. The method of any of embodiments 132-135, wherein the subject has a PD-L1-expressing tumor.

137. The method of any of embodiments 132-136, wherein prior to the administering, the method comprises selecting a subject having an PD-L1-expressing tumor.

Also, among provided embodiments are:

1. An immune cell comprising an immunomodulatory protein comprising at least one variant CD80 extracellular domain polypeptide, wherein the variant CD80 extracellular domain polypeptide comprises an amino acid substitution at position 11 in the sequence set forth in SEQ ID NO:2 or a portion thereof comprising an IgV domain, wherein the variant CD80 extracellular domain polypeptide exhibits increased binding to PD-L1 compared to wild-type CD80 set forth in SEQ ID NO:2 or the portion thereof comprising the IgV domain.

2. An immune cell comprising an immunomodulatory protein comprising at least one variant CD80 extracellular domain polypeptide, wherein the variant CD80 extracellular domain polypeptide comprises 2 to 10 amino acid substitutions at positions in the sequence set forth in SEQ ID NO:2 or a portion thereof comprising an IgV domain, wherein at least one substitution is at a position selected from among 9, 10, or 11, wherein the variant CD80 extracellular domain polypeptide exhibits increased binding to PD-L1 compared to wild-type CD80 set forth in SEQ ID NO:2 or the portion thereof comprising the IgV domain.

3. The immune cell of embodiment 1 or embodiment 2, wherein the variant CD80 extracellular domain polypeptide comprises an amino acid substitution at position 11 and the substitution is to an aromatic amino acid residue.

4. The immune cell of embodiment 2, wherein the aromatic amino acid residues are selected from the group consisting of tyrosine (Y), tryptophan (W) or phenylalanine (F), optionally wherein the amino acid substitution is V11Y, V11F, or V11W.

5. The immune cell of any of embodiments 1-4, wherein the variant CD80 extracellular domain polypeptide comprises the amino acid substitution V11Y.

6. The immune cell of any of embodiments 1-5, wherein the variant CD80 extracellular domain polypeptide comprises the amino acid substitutions V11Y/M47V/F59S/L85E, V11Y/M47V/L85E, V11Y/M42I/M47V/A71G, V11Y/T28Y/M47L/L85E, E10S/V11F/T28Y/M47L/T62S, E10G/V11W/V22S/T28P/A71G/E81K/Y87R, V11Y/T28Y/L85E/Y87I, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E10G/V11W/M47V/L85E, V11Y/T28Y/M47L/V68L/L85E, V11Y/M42W/T62A/L85E, V11Y/M42W/T62A, V11Y/M42W/F59Y/V68N, V11Y/M42W/E52K/T62A/L85E, V11Y/E35D/Y87Q/T101R, K9N/E10A/V11W/H18F/T28H/M47L/T62E/R73D, V11Y/E35G/M42G/F59S, V11Y/T28R/E35G/M47L/F59S, V11Y/T28R/E35G/M47L/A71G, V11Y/V68T, V11W/T28Y/D46V/R73E/F92L, V11W/T28Y/D46V/V68T/R73T/Y87N, E10S/V11Y/M42R/A71V, K9N/V11W/M47L/V68T/R73T/Y87N, V11Y/M47L/V68T, V11W/T28H/D46Q/V68L/L85E, E7Q/V11Y/R29H/M47L/V68T, V11Y/M47V/A71G/L85E, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

7. The immune cell of embodiment 2, wherein the variant CD80 extracellular domain polypeptide comprises an amino acid substitution at position 9 and is substitution to a polar uncharged amino acid residue.

8. The immune cell of embodiment 7, wherein the polar uncharged amino acid is selected from the group consisting of serine (S), asparagine (N), glutamine (Q), threonine (T).

9. The immune cell of any of embodiments 2, 7 and 8, wherein the variant CD80 extracellular domain polypeptide comprises the amino acid substitution K9S or K9N.

10. The immune cell of embodiment 2, wherein the variant CD80 extracellular domain polypeptide comprises an amino acid substitution at position 9 and the substitution is to another basic amino acid.

11. The immune cell of embodiment 10, wherein the other basic amino acid is selected from the group consisting of arginine or histidine.

12. The immune cell of any of embodiments 2, 10 and 11, wherein the variant CD80 extracellular domain polypeptide comprises the amino acid substitution is K9R.

13. The immune cell of any of embodiments 2 and 7-12, wherein the variant CD80 extracellular domain polypeptide comprises the amino acid substitutions K9N/E10G/N63S/L85E, K9N/E10G/E35G/D46E/M47V/V68M, K9R/E10S/V11Y/M42L/F59M/V68M/L85E, K9S/E10R/V11Y/M47L/A71G, K9N/E10R/H18V/T28Y/A71G, K9N/E10A/V11W/H18F/T28H/M47L/T62E/R73D, K9R/E10A/E35G/V68T/T101K, K9R/E10A/E35G/V68L/L85E, K9N/E10G/Y87K/T101Q, K9N/V11W/M47L/V68T/R73T/Y87N, K9R/A26T/T28Y/E35A/M47L/A71G.

14. The immune cell of embodiment 2, wherein the variant CD80 extracellular domain comprises an amino acid substitution at position 10 and the substitution is to a nonpolar amino acid.

15. The immune cell of embodiment 14, wherein the nonpolar amino acid is glycine, alanine or valine.

16. The immune cell of embodiment 2, 14 or 15, wherein the variant CD80 extracellular domain comprises the amino acid substitution E10G or E10A

17. The immune cell of embodiment 2, wherein the variant CD80 extracellular domain comprises an amino acid substitution at position 10 and the amino acid substitution is selected from the group consisting of E10G, E10S, E10R, and E10A.

18. The immune cell of any of embodiments 2 and 14-17, wherein the variant CD80 extracellular domain polypeptide comprises the amino acid substitutions K9N/E10G/N63S/L85E, E10G/E35G/D46E/M47V/V68M, K9N/E10G/E35G/D46E/M47V/V68M, K9R/E10S/V11Y/M42L/F59M/V68M/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, E10G/V11W/T28Y/M47L, K9S/E10R/V11Y/M47L/A71G, E10G/V1IW/E35G/M47L/A71G/Y87R, E10G/V11W/M47V/L85E, E10G/H18Y, K9N/E10R/H18V/T28Y/A71G, E10G/H18Y/T28Y/M47W/T62S, K9N/E10A/V11W/H18F/T28H/M47L/T62E/R73D, E10G/D46K/L85E, E10G/H18T/Q27T/D46E/M47L, K9R/E10A/E35G/V68T/T101K, E10G/E35G/D46E/M47V/V68M/T101K, K9R/E10A/E35G/V68L/L85E, K9N/E10G/Y87K/T101Q, E10S/V11Y/M42R/A71V, E10G/A26S/T28Y, E10S/V68M/Y87P, E10G/Q27F/D46N/A71G/D90G, or E10S/V11F/T28Y/M47L.

19. The immune cell of any of embodiments 1-5, 7-12 and 14-17, wherein the variant CD80 extracellular domain polypeptide further comprises an amino acid substitution at position 47 in the sequence set forth in SEQ ID NO:2 or the portion thereof comprising an IgV domain.

20. The immune cell of embodiment 19, wherein the amino acid substitution at position 47 is to another hydrophobic amino acid.

21. The immune cell of embodiment 20, wherein the hydrophobic amino acid is selected from the group consisting of valine, leucine, isoleucine or proline.

22. The immune cell of any of embodiments 19-21, wherein the variant CD80 extracellular domain comprises the amino acid substitution M47L or M47V.

23. The immune cell of any of embodiments 19-22, wherein the variant CD80 extracellular domain polypeptide comprises the amino acid substitutions V11W/M47L, V11F/M47L, V11Y/M47L, V11W/M47V, or V11Y/M47V.

24. The immune cell of any of embodiments 19-23, wherein the variant CD80 extracellular domain polypeptide comprises the amino acid substitutions V11Y/M47V/F59S/L85E, V11Y/M47V/L85E, V11Y/M42I/M47V/A71G, V11Y/T28Y/M47L/L85E, E10S/V11F/T28Y/M47L/T62S, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E10G/V11W/M47V/L85E, V11Y/T28Y/M47L/V68L/L85E, K9N/E10A/V11W/H18F/T28H/M47L/T62E/R73D, V11Y/T28R/E35G/M47L/F59S, V11Y/T28R/E35G/M47L/A71G, K9N/V11W/M47L/V68T/R73T/Y87N, V11Y/M47L/V68T, E7Q/V11Y/R29H/M47L/V68T, V11Y/M47V/A71G/L85E, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

25. The immune cell of any of embodiments 1-5, 7-12, 14-17 and 19-23, wherein the variant CD80 extracellular domain polypeptide further comprises an amino acid substitution at position 28 in the sequence set forth in SEQ ID NO:2 or the portion thereof comprising an IgV domain.

26. The immune cell of any of embodiments 1-5, 7-12, 14-17, 19-23 and 25, wherein the variant CD80 extracellular domain polypeptide comprises an amino acid substitution selected from T28Y, T28P, T28H, T28R, or T28V.

27. The immune cell of embodiment 26, wherein the amino acid substitution is T28Y.

28. The immune cell of any of embodiments 19-23 and 25-27, wherein the variant CD80 extracellular domain polypeptide comprises the amino acid substitutions V11W/T28Y/M47L, V11F/T28Y/M47L, V11Y/T28Y/M47L, V11W/T28Y/M47V, V11F/T28Y/M47V, or V11Y/T28Y/M47V.

29. The immune cell of any of embodiments 1-5, 7-12, 14-17, 19-28, wherein the variant CD80 extracellular domain polypeptide comprises the amino acid substitutions V11Y/T28Y/M47L.

30. The immune cell of any of embodiments 1-5, 7-12, 14-17, 19-29, wherein the variant CD80 extracellular domain polypeptide further comprises an amino acid substitution at position 68, optionally wherein the amino acid substitution is V68M, V68L, V68N, V68T, or V68S.

31. The immune cell of any of embodiments 1-5, 7-12, 14-17, 19-30, wherein the variant CD80 extracellular domain polypeptide comprises the amino acid substitutions V11Y/T28Y/M47L/V68M.

32. The immune cell of any of embodiments 1-5, 7-12, 14-17, 19-30, wherein the variant CD80 extracellular domain polypeptide comprises the amino acid substitutions V11Y/T28Y/M47L/V68L.

33. The immune cell of any of embodiments 1-5, 7-12, 14-17, 19-29, wherein the variant CD80 extracellular domain polypeptide comprises the amino acid substitutions E10G/V11W/T28Y/M47L.

34. The immune cell of any of embodiments 1-33, wherein the variant CD80 extracellular domain polypeptide further comprises an amino acid substitution at position 7 in the sequence set forth in SEQ ID NO:2 or a portion thereof comprising an IgV domain.

35. An immune cell comprising an immunomodulatory protein comprising at least one variant CD80 extracellular domain polypeptide, wherein the variant CD80 extracellular domain polypeptide comprises an amino acid substitution at position 7 in the sequence set forth in SEQ ID NO:163 or a portion thereof comprising the IgV domain, wherein the variant CD80 extracellular domain polypeptide exhibits increased binding to PD-L1 compared to the wild-type CD80 extracellular domain polypeptide set forth in SEQ ID NO:2 or the portion thereof comprising the IgV domain.

36. The immune cell of embodiment 34 or embodiment 35, wherein the variant CD80 extracellular domain polypeptide comprises an amino acid substitution at position 7 and the substitution is to a polar uncharged amino acid residue.

37. The immune cell of embodiment 36, wherein the polar uncharged amino acid is selected from the group consisting of serine (S), asparagine (N), glutamine (Q), threonine (T).

38. The immune cell of any of embodiments 34-37, wherein the variant CD80 extracellular domain polypeptide comprises the amino acid substitution E7Q, E7N or E7S.

39. The immune cell of embodiment 34 or embodiment 35, wherein the variant CD80 extracellular domain polypeptide comprises an amino acid substitution at position 7 and the substitution is to a basic amino acid.

40. The immune cell of embodiment 39, wherein the basic amino acid is selected from the group consisting of arginine, histidine or lysine.

41. The immune cell of any of embodiments 34, 35, 39 and 40, wherein the variant CD80 extracellular domain polypeptide comprises the amino acid substitution E7H or E7K.

42. The immune cell of any of embodiments 34-41, wherein the variant CD80 extracellular domain polypeptide comprises the amino acid substitutions E7S/H18I/V20L/A26K/M47L/A71N, E7K/V11W/N63H/A71G/Y87K, E7N/E35D/T101R, E7H/H18L/V20I/T28Y/D46S/A71G, E7N/E35D/F59S, or E7Q/V11Y/R29H/M47L/V68T.

43. The immune cell of any of embodiments 1-42, further comprising an amino acid substitution at position 101 in the sequence set forth in SEQ ID NO:2 or a portion thereof comprising an IgV domain.

44. An immune cell comprising an immunomodulatory protein comprising at least one variant CD80 extracellular domain polypeptide, wherein the variant CD80 extracellular domain polypeptide comprises an amino acid substitution at position 101 in the sequence set forth in SEQ ID NO:2 or a portion thereof comprising an IgV domain, wherein the variant CD80 extracellular domain polypeptide exhibits increased binding to PD-L1 compared to wild-type CD80 set forth in SEQ ID NO:2 or the portion thereof comprising the IgV domain.

45. The immune cell of embodiment 43 or embodiment 44, wherein the variant CD80 extracellular domain polypeptide comprises an amino acid substitution at position 101 and the substitution is to a charged amino acid residue.

46. The immune cell of embodiment 45, wherein the charged amino acid residue is basic and the amino acid substitution is to a histidine (H), lysine (K) or arginine (R).

47. The immune cell of embodiment 46, wherein the variant CD80 extracellular domain polypeptide comprises an amino acid substitution T101K or T101R.

48. The immune cell of embodiment 45, wherein the charged amino acid residue is acidic and the amino acid substitution is to aspartate (D), glutamate (E), asparagine (N) or glutamine (Q).

49. The immune cell of embodiment 48, wherein the variant CD80 extracellular domain polypeptide comprises an amino acid substitution T101Q.

50. The immune cell of any of embodiments 43-49, wherein the variant CD80 extracellular domain polypeptide comprises the amino acid substitutions E7N/E35D/T101R, V11Y/E35D/Y87Q/T101R, E35D/V68T/T101K, K9R/E10A/E35G/V68T/T101K, E10G/E35G/D46E/M47V/V68M/T101K, or K9N/E10G/Y87K/T101Q.

51. The immune cell of any of embodiments 43-47 and 50, wherein the variant CD80 extracellular domain polypeptide comprises the amino acid substitutions V11Y/E35D/Y87Q/T101R.

52. The immune cell of any of embodiments 1-51, further comprising an additional amino acid substitution at a different position wherein the amino acid substitution is selected from the group consisting of E7S, E7K, E7N, E7H, E7Q, K9N, K9R, K9S, E10G, E10S, E10R, E10A, V11Y, V11F, V11W, H18I, H18Y, H18F, H18V, H18L, H18T, V20L, V20I, V22S, A26K, A26G, A26Q, A26E, A26S, A26T, Q27F, Q27T, T28Y, T28P, T28H, T28R, T28V, R29S, R29H, E35G, E35D, E35A, M42I, M42L, M42G, M42W, M42R, D46E, D46S, D46K, D46V, D46Q, D46N, M47V, M47L, M47R, M47W, E52K, F59S, F59M, F59Y, T62S, T62A, T62E, N63S, N63I, N63H, V68M, V68L, V68N, V68T, V68S, A71G, A71N, A71V, R73D, R73E, R73T, E77G, E81K, L85E, L85Q, Y87R, Y87I, Y87K, Y87Q, Y87N, Y87P, D90G, F92L, T101R, T101K, or T101Q, or a conservative amino acid substitution of any of the foregoing.

53. An immune cell comprising an immunomodulatory protein comprising at least one variant CD80 extracellular domain polypeptide, wherein the variant CD80 extracellular domain polypeptide comprises one or more amino acid substitutions in the sequence set forth in SEQ ID NO:2 or a portion thereof comprising the IgV domain, wherein the one or more amino acid substitutions is selected from E7S, E7K, E7N, E7H, E7Q, K9N, K9R, K9S, E10G, E10S, E10A, V11Y, V11F, V11W, V20I, V22S, Q27F, Q27T, T28P, T28H, T28R, T28V, R29S, E35A, M42L, M42G, M42W, M42R, D46S, D46K, D46Q, M47R, M47W, E52K, F59S, T62S, T62A, N63I, N63H, V68N, V68T, V68S, A71N, A71V, R73D, R73E, R73T, L85Q, Y87R, Y87I, Y87K, Y87P, T101R, TIOlK, and T101Q, wherein the variant CD80 polypeptide exhibits increased binding to PD-L1 compared to wild-type CD80 comprising the sequence set forth in SEQ ID NO:2 or the portion thereof comprising the IgV domain.

54. The immune cell of any of embodiments 1-53, wherein the variant CD80 extracellular domain polypeptide comprises 2, 3, 4, 5, 6, 7, 8, 9 or 10 amino acid substitutions compared to SEQ ID NO:2 or the portion thereof comprising the IgV domain.

55. The immune cell of any of embodiments 1-54, comprising no more than 4 amino acid substitutions compared to SEQ ID NO:2 or the portion thereof comprising the IgV domain.

56. The immune cell of any of embodiments 1-55, comprising 2, 3 or 4 amino acid substitutions compared to SEQ ID NO:2 or the portion thereof comprising the IgV domain.

57. An immune cell comprising an immunomodulatory polypeptide comprising at least one variant CD80 extracellular domain polypeptide, wherein the variant CD80 extracellular domain polypeptide comprises the sequence set forth in SEQ ID NO:2 or a portion thereof comprising an IgV domain, in which is contained amino acid substitutions selected from V11Y/M47V/F59S/L85E, V11Y/M47V/L85E, V11Y/M42I/M47V/A71G, K9N/E10G/N63S/L85E, E10G/E35G/D46E/M47V/V68M, K9N/E10G/E35G/D46E/M47V/V68M, E7S/H18I/V20L/A26K/M47L/A71N, K9R/E10S/V11Y/M42L/F59M/V68M/L85E, V11Y/T28Y/M47L/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, V11Y/T28Y/L85E/Y87I, H18F/M42G/F59Y/V68N, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E35D/V68L/L85E, E10G/V11W/M47V/L85E, T28Y/M47L, V11Y/T28Y/M47L/V68L/L85E, E7N/E35D/T101R, V11Y/M42W/T62A/L85E, V11Y/M42W/T62A, V11Y/M42W/F59Y/V68N, V11Y/M42W/E52K/T62A/L85E, V11Y/E35D/Y87Q/T101R, H18Y/A26E/R29S/E35D/M47L/V68M/A71G/E77G/D90G, E10G/H18Y, K9N/E10R/H18V/T28Y/A71G, E10G/H18Y/T28Y/M47W/T62S, E7H/H18L/V20I/T28Y/D46S/A71G, K9N/E10A/V11W/H18F/T28H/M47L/T62E/R73D, H18V/V20I/T28Y/E35G/M47V/R73E, E10G/D46K/L85E, E10G/H18T/Q27T/D46E/M47L, E35D/V68T/T101K, V11Y/E35G/M42G/F59S, V11Y/T28R/E35G/M47L/F59S, E7N/E35D/F59S, V11Y/T28R/E35G/M47L/A71G, K9R/E10A/E35G/V68T/T101K, V11Y/V68T, E10G/E35G/D46E/M47V/V68M/T101K, K9R/E10A/E35G/V68L/L85E, V11W/T28Y/D46V/R73E/F92L, K9N/E10G/Y87K/T101Q, V11W/T28Y/D46V/V68T/R73T/Y87N, E10S/V11Y/M42R/A71V, H18F/T28V/M47L/V68S, E10G/A26S/T28Y, E35D/D46Q/L85E, E10S/V68M/Y87P, K9N/V11W/M47L/V68T/R73T/Y87N, V11Y/M47L/V68T, K9R/A26T/T28Y/E35A/M47L/A71G, V11W/T28H/D46Q/V68L/L85E, E7Q/V11Y/R29H/M47L/V68T, V11Y/M47V/A71G/L85E, E10G/Q27F/D46N/A71G/D90G, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

58. The immune cell of any one of embodiments 1-57, wherein the variant CD80 extracellular domain polypeptide exhibits at least 85% sequence identity to the sequence set forth in SEQ ID No: 2 or the portion thereof comprising the IgV domain.

59. The immune cell of any one of embodiments 1-58, wherein the variant CD80 extracellular domain polypeptide exhibits at least 90% sequence identity to the sequence set forth in SEQ ID No: 2 or the portion thereof comprising the IgV domain.

60. The immune cell of any one of embodiments 1-59, wherein the variant CD80 extracellular domain polypeptide exhibits at least 95% sequence identity to the sequence set forth in SEQ ID No: 2 or the portion thereof comprising the IgV domain.

61. The immune cell of any one of embodiments 1-60, wherein the variant CD80 extracellular domain polypeptide exhibits at least 97% sequence identity to the sequence set forth in SEQ ID No: 2 or the portion thereof comprising the IgV domain.

62. The immune cell of any of embodiments 1-61, wherein the portion of SEQ ID NO:2 comprising the IgV domain comprises amino acids 1-101 of SEQ ID NO:2 and has a length of no more than 110 amino acids.

63. The immune cell of any of embodiments 1-62, wherein the portion of SEQ ID NO:2 comprising the IgV domain is set forth in SEQ ID NO:163.

64. The immune cell of any of embodiments 1-61, wherein the portion of SEQ ID NO:2 comprising the IgV domain is set forth as amino acids 1-107 of SEQ ID NO:2 (SEQ ID NO:164).

65. The immune cell of any one of embodiments 1-64, wherein the variant CD80 extracellular domain polypeptide comprises the sequence of amino acids set forth in any of SEQ ID NOS: 165-244 or a sequence of amino acids that exhibits at least 95% sequence identity to any of SEQ ID NOS: 165-244.

66. The immune cell of any one of embodiments 1-65, wherein the variant CD80 extracellular domain polypeptide comprises the sequence of amino acids set forth in any of SEQ ID NOS: 165-244.

67. The immune cell of any of embodiments 1-66, wherein the variant CD80 extracellular domain polypeptide is set forth in any one of SEQ ID NOS: 165-244.

68. The immune cell of any of embodiments 1-67, wherein the variant CD80 extracellular domain polypeptide is set forth in SEQ ID NO:180.

69. The immune cell of any of embodiments 1-67, wherein the variant CD80 extracellular domain polypeptide is set forth in SEQ ID NO:185.

70. The immune cell of any of embodiments 1-67, wherein the variant CD80 extracellular domain polypeptide is set forth in SEQ ID NO:197.

71. The immune cell of any of embodiments 1-67, wherein the variant CD80 extracellular domain polypeptide is set forth in SEQ ID NO:233.

72. The immune cell of any of embodiments 1-67, wherein the variant CD80 extracellular domain polypeptide is set forth in SEQ ID NO:234.

73. The immune cell of any of embodiments 1-72, comprising a detectable moiety.

74. The immune cell of any of embodiments 1-72, comprising a multimerization that is linked to the at least one variant CD80 polypeptide, optionally via a linker a multimerization domain, or a detectable label.

75. The immune cell of embodiment 74, wherein the multimerization domain is an Fc region.

76. The immune cell of any of embodiments 1-75, wherein the immunomodulatory protein is a variant CD80-Fc fusion protein comprising the at least one variant polypeptide and an Fc region of an immunoglobulin.

77. The immune cell of embodiment 75 or embodiment 75, wherein the at least one variant CD80 polypeptide is linked to the Fc region via a linker, optionally a peptide linker.

78. The immune cell of embodiment 77, wherein the linker comprises a peptide linker and the peptide linker is selected from GGGGS (G4S; SEQ ID NO: 328), GSGGGGS (SEQ ID NO: 325), GGGGSGGGGS (2×GGGGS; SEQ ID NO: 329), GGGGSGGGGSGGGGS (3×GGGGS; SEQ ID NO: 330), GGGGSGGGGSGGGGSGGGGS (4×GGGGS, SEQ ID NO:331), GGGGSGGGGSGGGGSGGGGSGGGGS (5×GGGGS, SEQ ID NO: 332), GGGGSSA (SEQ ID NO: 333), or GSGGGGSGGGGS (SEQ ID NO:335) or combinations thereof.

79. The immune cell of any of embodiments 76-78, wherein the immunoglobulin Fc is an IgG1 Fc domain, or is a variant Fc domain that exhibits reduced binding affinity to an Fc receptor and/or reduced effector function, optionally as compared to a wild-type IgG1 Fc domain.

80. The immune cell of embodiment 79, wherein the immunoglobulin Fc is a variant IgG1 Fc domain comprising one or more amino acid substitutions selected from L234A, L234V, L235A, L235E, G237A, S267K, R292C, N297G, and V302C, by EU numbering.

81. The immune cell of embodiment 80, wherein the immunoglobulin Fc region comprises the amino acid substitutions L234A, L235E an G237A by EU numbering, optionally wherein the Fc region is set forth in any of SEQ ID NOS: 344, 345, 348 or 351.

82. The immune cell of embodiment 76-81, wherein the immunoglobulin Fc region comprises the sequence set forth in SEQ ID NO:344.

83. The immune cell of any of embodiments 76-78, wherein the immunoglobulin Fc is an IgG4 Fc domain, optionally comprising the amino acid substitution S228P.

84. The immune cell of any of embodiments 76-78 and 83, wherein the immunoglobulin Fc region comprises the sequence set forth in SEQ ID NO:326.

85. The immune cell of any one of embodiments 76-84, wherein the variant CD80-Fc fusion protein comprises the structure: variant CD80 polypeptide (vCD80)-Linker-Fc region.

86. The immune cell of any of embodiments 76-85, wherein the variant CD80-Fc fusion protein comprises the sequence of amino acids set forth in any one of SEQ ID NOS: 245-324 or a sequence that exhibits at least 85%, at least 90%, at least 95% or at least 97% sequence identity to any one of SEQ ID NOS: 245-324.

87. The immune cell of any of embodiments 76-84, wherein the variant CD80-Fc fusion protein comprises the structure: (vCD80)-Linker-Fc region-Linker-(vCD80).

88. The immune cell of any of embodiments 76-84 and 87, wherein the variant CD80-Fc fusion protein comprises the sequence of amino acids set forth in any one of SEQ ID NOS: 336, 338, 339 or 341, or a sequence that exhibits at least 85%, at least 90%, at least 95% or at least 97% sequence identity to any one of SEQ ID NOS: 336, 338, 339 or 341.

89. The immune cell of any of embodiments 76-84, wherein the variant CD80-Fc fusion protein comprises the structure: (vCD80)-Linker-(vCD80)-Linker-Fc region.

90. The immune cell of any of embodiments 76-84 and 89, wherein the variant CD80-Fc fusion protein comprises the sequence of amino acids set forth in SEQ ID NO: 340 or 342, or a sequence that exhibits at least 85%, at least 90%, at least 95% or at least 97% sequence identity to SEQ ID NO: 340 or 342.

91. The immune cell of any of embodiments 76-90 that is a homodimer comprising two identical copies of the variant CD80-Fc fusion protein.

92. The immune cell of any one of embodiments 1-91, wherein the PDL1 is human PDL1.

93. The immune cell of any one of embodiments 1-92, wherein the binding affinity of the variant CD80 extracellular domain to PD-L1 is increased greater than 1.1-fold compared to wild-type CD80 set forth in SEQ ID NO:2 or the portion comprising the IgV domain.

94. The immune cell of embodiment 93, wherein the binding affinity is increased greater than 1.5-fold, greater than 2-fold, greater than 3-fold, greater than 4-fold, greater than 5-fold, greater than 6-fold, greater than 7-fold, greater than 8-fold, greater than 9-fold or greater than 10-fold.

95. The immune cell of any one of embodiments 1-94, wherein the binding affinity is determined by Mean Fluorescence Intensity (MFI) as measured by flow cytometry in a cell-based binding assay for a PD-L1-expressing cell.

96. The immune cell of any of embodiments 1-95, wherein the immunomodulatory protein blocks binding of PD-L1 to PD-1.

97. The immune cell of any of embodiments 1-96, wherein the variant CD80 extracellular polypeptide exhibit a Koff for binding to PD-L1 of less than 50×10⁻³ s⁻¹.

98. The immune cell of any of embodiments 1-97, wherein the variant CD80 extracellular polypeptide has a Koff for binding to PD-L1 of at or about or less than 40×10⁻³ s⁻¹, 30×10⁻³ s⁻¹, 20×10³s⁻¹, 5×10⁻³ s⁻¹, 10×10⁻³ s⁻¹, 5×10⁻³ s⁻¹, or 1×10⁻³ s⁻¹.

99. The immune cell of any of embodiments 1-97, wherein the variant CD80 extracellular polypeptide has a Koff for binding to PD-L1 of between 1×10⁻³ s⁻¹ and 50×10⁻³ s⁻¹, 1×10⁻³ s⁻¹ and 30×10⁻³ s⁻¹, 1×10⁻³ s⁻¹ and 20×10⁻³ s⁻¹, 1×10⁻³ s⁻¹ and 15×10⁻³ s⁻¹, 1×10⁻³ s⁻¹ and 10×10⁻³ s⁻¹, 1×10⁻³ s⁻¹ and 5×10⁻³ s⁻¹, 5×10⁻³ s⁻¹ and 50×10⁻³ s⁻¹, 5×10⁻³ s⁻¹ and 30×10⁻³ s⁻¹, 5×10⁻³ s⁻¹ and 20×10⁻³ s⁻¹, 5×10⁻³ s⁻¹ and 15×10⁻³ s⁻¹, 5×10⁻³ s⁻¹ and 10×10⁻³ s⁻¹, 10×10⁻³ s⁻¹ and 50×10⁻³ s⁻¹, 10×10⁻³ s⁻¹ and 30×10⁻³ s⁻¹, 10×10⁻³ s⁻¹ and 20×10⁻³ s⁻¹, 1×10⁻³ s⁻¹ and 15×10⁻³ s⁻¹, 5×10⁻³ s⁻¹ and 50×10⁻³ s⁻¹, 15×10⁻³ s⁻¹ and 30×10⁻³ s⁻¹, 15×10⁻³ s⁻¹ and 20×10⁻³ s⁻¹, 20×10⁻³ s⁻¹ and 50×10⁻³ s⁻¹, 20×10⁻³ s⁻¹ and 30×10⁻³ s⁻¹, or 30×10⁻³ s⁻¹ and 50×10⁻³ s⁻¹.

100. The immune cell of any of embodiments 1-99, wherein the variant CD80 polypeptide binds CD28, optionally with a binding affinity is 0.8-fold to 30-fold of the binding affinity of wild-type CD80 set forth in SEQ ID NO:2 or the portion thereof comprising the IgV domain.

101. The immune cell of any of embodiments 1-100, wherein the variant CD80 polypeptide exhibits increased binding to CD28 compared to wild-type CD80 set forth in SEQ ID NO:2 or the portion thereof comprising the IgV domain.

102. The immune cell of any one of embodiments 1-92, wherein the binding affinity of the variant CD80 extracellular domain to CD80 is increased greater than 1.1-fold compared to wild-type CD80 set forth in SEQ ID NO:2 or the portion comprising the IgV domain.

103. The immune cell of embodiment 101 or embodiment 102, wherein the binding affinity is increased greater than 1.5-fold, greater than 2-fold, greater than 3-fold, greater than 4-fold, greater than 5-fold, greater than 6-fold, greater than 7-fold, greater than 8-fold, greater than 9-fold or greater than 10-fold.

104. The immune cell of any one of embodiments 100-103, wherein the binding affinity is determined by Mean Fluorescence Intensity (MFI) as measured by flow cytometry in a cell-based binding assay for a CD28-expressing cell.

105. The immune cell of any one of embodiments 1-104, wherein the immunomodulatory protein exhibits CD28 agonism, optionally as determined in a T reporter assay.

106. The immune cell of embodiment 105, wherein the CD28 agonism is PD-L1 dependent, optionally as determined in a T cell reporter assay in the presence of PD-L1 expressing cells.

107. The immune cell of any of embodiments 1-106, wherein the immunomodulatory protein blocks binding of CTLA-4 to its ligand CD80 or CD86.

108. The immune cell of any of embodiments 1-107, wherein the immune cell further comprises a T cell receptor (TCR) or a chimeric antigen receptor (CAR).

109. The immune cell of any of embodiments 1-108, wherein the immune cell is a lymphocyte.

110. The immune cell of embodiment 109, wherein the lymphocyte is a T cell.

111. The immune cell of any of embodiments 1-73 and 92-110, wherein the immunomodulatory protein is a transmembrane protein expressed on the surface of the immune cell.

112. The immunomodulatory protein of any of embodiments 1-110 that is a soluble protein.

113. The immune cell of any of embodiments 1-110 and 112, wherein the immunomodulatory protein is secretable from the immune cell.

114. A pharmaceutical composition comprising the immune cell of any of embodiments 1-113.

115. The pharmaceutical composition of embodiment 114, comprising a pharmaceutically acceptable excipient.

116. The pharmaceutical composition of embodiment 114 or embodiment 115, wherein the pharmaceutical composition is sterile.

117. An article of manufacture comprising the pharmaceutical composition of any of embodiments 114-116 in a vial or container.

118. The article of manufacture of embodiment 117, wherein the vial or container is sealed.

119. A kit comprising the pharmaceutical composition of any of embodiments 114-116, and instructions for use.

120. A method of stimulating an immune response in a subject, comprising administering the immune cell of any of embodiments 1-113 or the pharmaceutical composition of any of embodiments 114-116 to a subject in need thereof.

121. The method of embodiment 120, wherein stimulating the immune response treats a disease or condition in the subject.

122. A method of treating a disease or condition in a subject, the method comprising administering the immune cell of any of embodiments 1-113 or the pharmaceutical composition of any of embodiments 114-116 to a subject having the disease or condition.

123. The method of embodiment 121 or embodiment 122, wherein the disease or condition is a cancer.

### VIII. Examples

The following examples are included for illustrative purposes only and are not intended to limit the scope of the invention.

### Example 1: Crystal Structure of a Variant CD80 and PD-L1

This example describes the determination of the crystal structure of the interaction of PD-L1 with an exemplary variant CD80 composed only of a variant CD80 IgV (vIgD), in which the variant was engineered to have improved binding affinity for PD-L1 as compared to the extracellular domain of wild-type (WT) CD80.

Monomeric His-tagged proteins derived from the CD80 vIgD and the extracellular domain (ECD) of PD-L1 (residues 1-220 of the mature protein), were co-crystallized and used to determine the structure of the CD80 vIgD/PD-L1 complex. Monomeric CD80 vIgD domain and PD-L1 ECD domain were prepared as carboxyl terminal His tagged recombinant proteins expressed in Expi293F™ cells, purified by affinity chromatography on IMAC columns (HisTrap Excel, GE Health Care) and buffer exchanged into 10 mM acetate, 9% sucrose, pH 5.0. The CD80 vIgD/PD-L1 complex was prepared by mixing CD80 vIgD His and PD-L1 ECD His proteins at a 1.2:1 molar ratio and the CD80 vIgD/PD-L1 complex was isolated by SEC fractionation. The complex was concentrated, flash frozen in liquid nitrogen and stored at −80° C. until ready for crystallization.

Crystals of the CD80 vIgD in complex with PD-L1 were grown using the vapor diffusion method. The complex crystals were grown at 6.7 mg ml⁻¹ from reservoir solution (0.03 M sodium nitrate, 0.03 M sodium phosphate dibasic, 0.03 M ammonium sulfate, 0.1 M sodium HEPES/MOPS (acid), pH 7.5, 20% v/v glycerol, 10% w/v PEG 4000). Crystals were frozen directly from the crystallization drop.

Datasets were collected at 100 K at station 103, Diamond Light Source, Didcot, England (λ=0.9763 Å) equipped with a Pilatus3 6M detector. Data were processed using XDS (Kabsch et al. Acta Crystallog D Biol Crystallogr, 2010, 66:125-32) and merged using Aimless (Evans et al. Nat Immunol, 2005:6:271-9). A data set for CD80 vIgD in complex with PD-L1 was collected to 3.15 Å. Crystals belonged to space group P2₁2₁2₁ and had cell dimensions: a=59.9, b=122.2, c=152.7 Å; α, β, and γ=90°.

Utilizing PDB structures 1DR9 (PDB: 1DR9 Ikemizu et. al., Immunity, 2000 12:51-60) and 5JDR (PDB: 5JDR, Zhang et al., Cell Discov, 2017, 3:17004) for molecular replacement, the binding surface and contact residues within 4 Å between the CD80 vIgD and the PD-L1 ECD were determined by using CONTACT of the CCP4 software package ((M. D. Winn et al. Acta. Crystalogr D Biol Crystallogr, 2011, 67: 235-242).

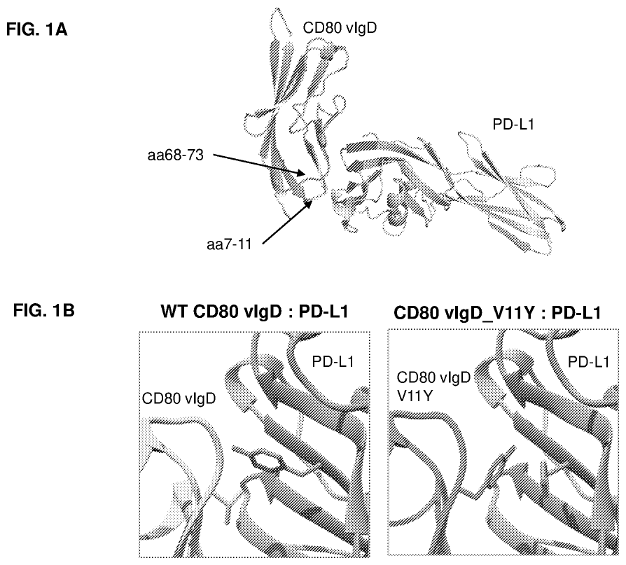

Interaction residues are summarized in Table E1. As shown in FIG. 1A, residues involved in the interaction between CD80 and PD-L1 within 4 Å of each other included CD80 residues Lys9, Glu10, and Val11, among others as indicated.

Based on the modeling, it was predicted that an amino acid substitution of V11 to an aromatic residue (e.g. tyrosine, V11Y), could Π stack Tyr-PD-L1 residue and fill interfacial cavity to improve hydrophobic packing compared to WT CD80 vIgD without an amino acid substitution at V11 (FIG. 1B)

TABLE E1
Residues in the CD80 vIgD/PD-L1 ECD
structure with atoms within 4Å

| | Interaction residues |
|---|---|
| CD80 | Lys9, Glu10, Val11, Lys54, Asn55, Arg56, Thr57, Ile58, |
| (chain A) | Asp60, Met68, Leu70, Gly71, Arg73 |
| PD-L1 | Ile36, Tyr38, Gln48, Val50, His51, Glu53, Arg95, Ile98, |
| (chain B) | Ser99, Gly102, Ala103, Asp104, Tyr105 |
| CD80 | Lys9, Glu10, Val11, Lys54, Asn55, Arg56, Thr 57, Ile58, |
| (chain C) | Asp60, Leu70, Gly71, Arg73 |
| PD-L1 | Ile36, Tyr38, Gln48, Val50, His51, Glu53, Met97, Ile98, |
| (chain D) | Ser99, Gly102, Ala103, Asp104, Tyr105 |

### Example 2: Engineering of CD80 Via Directed Evolution

This Example describes the generation of mutant DNA constructs of human CD80 IgV domains for translation and expression on the surface of yeast as yeast display libraries, introduction of DNA libraries into yeast, and selection of yeast cells expressing affinity-modified variants of CD80.

Constructs were generated based on a wildtype human CD80 sequence set forth in SEQ ID NO: 164 containing the immunoglobulin-like-V-type (IgV) domain as follows:

VIHVTKEVKEVATLSCGHNVSVEELAQTRIYWQKEKKMVLTMMSG
DMNIWPEYKNRTIFDITNNLSIVILALRPSDEGTYECVVLKYEKD
AFKREHLAEVTLSVKAD

A rationale design of vIgD CD80 DNA libraries were constructed to identify variants of the CD80 IgV that have improved binding to PD-L1. Positions for mutagenesis included: 7, 9, 10, 11, 18, 20, 22, 26, 27, 28, 29, 35, 46, 42, 46, 47, 52, 59, 62, 63, 68, 71, 73, 77, 81, 85, 87, 90, 92, and 101. Selection of CD80 residues to be targeted for mutagenesis employed two independent design strategies to analyze the CD80 vIgD-PD-L1 crystal structure, a consensus and saturating mutagenesis design.

The consensus design strategy targeted residues that are most dissimilar from superfamily consensus sequence. NCBI BLASTP (https://blast.ncbi.nlm.nih.gov/Blast.cgi?PAGE=Proteins) was queried with CD80 WT ECD (SEQ ID NO:2). The output list was filtered to retain 500 sequences with highest homology (30-100%). To reduce duplication bias, sequences with >90% identity to any other individual sequence were removed. The remaining sequences were aligned and a frequency of amino acid use at each position in the consensus was calculated. CD80 WT IgV (SEQ ID NO:164) was compared to the consensus and a dissimilarity score was calculated for each amino acid. The 20 positions having the highest dissimilarity scores were considered for mutagenesis after cross referencing with a list of residues selected from the saturating mutagenesis protocol discussed below. The rationale for this approach is that the most dissimilar residues may be evolutionarily unfavorable and the molecule's stability may be improved by targeting these.

A complimentary design strategy using insilico saturating mutagenesis employed the PMUT SCAN application within the Rosetta software suite (University of Washington) to analyze the CD80-PD-L1 crystal structure. Prior to running the PMUT scan, Pymol was used to prepare a single chain structure in which one chain each from the CD80 vIgD and PD-L1 was removed. Additionally the IgC1 domain of PD-L1 was removed leaving a single domain of CD80 vIgD complexed with PD-L1 IgV. Separately, IgC domain was removed from the CD80 WT structure (1Dr9) and the remaining IgV structure was overlaid on and aligned to the CD80 vIgD portion of CD80 vIgD-PD-L1 structure. The CD80 vIgD domain was removed leaving a model composed of CD80WT IgV complexed with PD-L1 IgV. This model was conditioned using FAST RELAX in Rosetta for 4-5 iterations. In each run the top 5 models were used as inputs for scoring the next round of Fast Relax. This process was repeated until convergence was achieved as judged by a lack of significant change in Rosetta energy score. The 5 relaxed models with lowest Rosetta energy scores were used and run through an interphase design protocol within Rosetta. Outputs from the interphase design were then run in PMUT SCAN. This protocol carries out saturating mutagenesis of each input model while holding the backbone fixed. Each substitution was scored by Rosetta energy units and the effects of substitutions were evaluated using REU averaged for all 5 input models. The average value at each residue was used to construct a position specific heat map to identify positions that show high potential for mutability as determined by the number of times beneficial mutations are scored. The top 20% of such residues were cross referenced with the consensus design output.

After obtaining a list of residues to target with mutation a DNA library was built. A list of full length CD80 IgV DNA sequences were compiled where each sequence contained a random combination of 4 substitutions at positions from the list of 30 positions. Of the four amino acid positions targeted per sequence, 2 were targeted using NNK mixed codon, and 2 were targeted using NNX where X corresponds to the WT CD80 sequence at this base position. NNK mixed base codon encodes all 20 amino acids and a stop codon while NNX encodes wild type and up to 15 additional amino acids. A total of about 2000 sequences were generated, each of which possessed a random shuffling of 4 codons derived from the list of 30 targeted positions. The variant CD80 sequences were amplified, agarose gel purified and separately mixed with 2 digested yeast expression vectors (P1959 or P5075) and electroporated into yeast.

For the P5075 vector library, amplified DNA was cloned into the modified yeast expression vector P5075 which places the CD80 IgV N-terminal to the yeast surface anchoring domain AGA2 with an in-frame HA fusion tag C-between CD80 IgV and the AGA2 anchoring domain. Expression in this vector is driven off of the inducible gal-1 promoter. The mutated (variant) CD80 library DNA was inserted into electroporation-competent, EBY100 yeast cells (ATCC) using standard methods.

For the P1959 vector library, amplified DNA was cloned to place the CD80 IgV N-terminal to the yeast surface anchoring domain AGA2 with an in-frame HA fusion tag C-between CD80 IgV and the AGA2 anchoring domain. Expression in this vector is driven off of the constitutively expressed GAP promoter. The mutated (variant) CD80 library DNA was inserted into electroporation-competent, modified strain Y1007. This strain was created stepwise by first introducing AGA1 under the control of GAP promoter and Tryp selection. Next a gene conferring G418 resistance, an aminoglycoside 3′-phosphotransferase (APT 3 II), was knocked into the URA3 locus using standard methods.

Cells were sub-cultured and grown in minimal media supplemented with appropriate auxotrophic amino acid supplements to minimize the fraction of untransformed cells and to allow for segregation of plasmid from cells that may contain two or more library variants. Cells from the second saturated culture were resuspended in fresh medium and frozen and stored at −80° C. (frozen library stock). Cells from the library were thawed from individual library stocks and grown overnight. The next day the P5075 vector library cells were resuspending in galactose containing induction media (SCDG-Leu media) and grown overnight at 30° C. to induce expression of library proteins on the yeast cell surface. The P1959 vector library was grown in minimal selection media at 30° C.

For selection of hits, constitutive and induced libraries underwent a series of 3 to 5 serial rounds of FACs selection and outgrowth. Selection reagents utilized were hPD-L1 ECD fused to an immunoglobulin Fc domain (PD-L1-Fc) yielding a dimeric PDL1 molecule, hPD-L1 ECD fused to a single chain dimer of immunoglobulin Fc domain (PD-L1-scFc) yielding a monomeric PD-L1 molecule, hCD28 ECD fused an immunoglobulin Fc domain (CD28-Fc) yielding a dimeric CD28 molecule to reduce non-binders and enrich for CD80 variants with the ability to bind PD-L1 while maintaining CD28 binding affinity. To select for variants with slower PD-L1 off rate we utilized both monomeric PD-L1 (PD-L1-scFc1.1), monomeric secondary detection reagent goat anti human FAB (AffiniPure Fab Fragment Goat Anti-Human IgG, Fcγ fragment specific, R-Phycoerythrin, 109-117-008. Jackson Immuno Research, West Grove, PA) and overnight 4° C. wash conditions.

Amino acid substitutions in selected variant CD80 IgVs that were identified by selection are set forth in Table E2.A.

A series of consensus variants also were generated based on hits selected from the rational design screen. The consensus variants contained 3-4 mutations chosen from amino acid substitutions at positions 10, 11, 26, 28, 35, 46, 47, 62, 68, 71, 85, 87 and 101. All consensus variants contained a mutation at position 11 to an aromatic amino acid (e.g. V11W, V11F or V11Y) and contained a mutation at position 47 to a hydrophobic amino acid (e.g. a hydrophobic aliphatic amino acid with branched side changes, e.g. M47V or M47L). Amino acid substitutions in consensus variant CD80 IgVs are set forth in Table E2.B.

For further screening for functional activity, selected CD80 IgV hit variants were further formatted as Fc fusion proteins with the exemplary Fc domain, human IgG4 Fc sequence containing an S228P mutation, numbering according to EU numbering system (IgG4 Fc set forth in SEQ ID NO: 326; S228P corresponds to SIOP by numbering of SEQ ID NO:326). After sequence analysis and identification of clones of interest, plasmid DNA was prepared using the MidiPlus kit (Qiagen). The DNA encoded generated affinity-modified (variant) CD80 Fc fusion proteins as follows: variant CD80 domain followed by a linker of 7 amino acids (GSGGGGS; SEQ ID NO: 325), followed by a human IgG4 Fc sequence (SEQ ID NO:326). Sequences of generated vIgD-Fc are set forth in Table E2.A and Table E2.B.

(SEQ ID NO: 326)
ESKYGPPCPPCPAPEFLGGPSVFLFPPKPKDTLMISRTPEVTCVV
VDVSQEDPEVQFNWYVDGVEVHNAKTKPREEQFNSTYRVVSVLT
VLHQDWLNGKEYKCKVSNKGLPSSIEKTISKAKGQPREPQVYTLP
PSQEEMTKNQVSLTCLVKGFYPSDIAVEWESNGQPENNYKTTPPV
LDSDGSFFLYSRLTVDKSRWQEGNVFSCSVMHEALHNHYTQKSLS
LSLG

Recombinant variant CD80 IgV-Fc fusion proteins were produced from suspension-adapted human embryonic kidney (HEK) 293 cells using the Expi293 expression system (Invitrogen, USA). Supernatant was harvested and the Fc Protein was captured on Mab SelectSure. (GE Healthcare cat. no. 17543801). Protein was eluted from the column using 50mMV Acetate pH3.6. The MabSelect Sure eluate was pooled and the pH was adjusted to above pH5.0. This material was buffer exchanged into 50 mM Acetate pH 5.0. The protein purity was assessed by analytic SEC. Material was vialed and stored at −80.

In Table E2.A and E2.B, the exemplary amino acid substitutions are indicated in the second column and are designated by amino acid position number corresponding to numbering of the respective reference unmodified (wildtype) IgV sequence (e.g. set forth in SEQ ID NO:164 or SEQ ID NO:2). The amino acid position is indicated in the middle, with the corresponding unmodified (e.g., wild-type) amino acid listed before the number and the identified variant amino acid substitution listed after the number. The last four columns set forth the SEQ ID NO identifier for the nucleotide (nt) or amino acid (aa) of the variant IgV or the IgV-Fc fusion molecule for each variant molecule.

TABLE E2.A
Exemplary variant CD80 (Rational Design)

| | | vIgD | | vIgD-Fc | |
|---|---|---|---|---|---|
| | | SEQ ID NO | | SEQ ID NO | |
| Name | Amino Acid Substitution(s) | Nt | aa | nt | aa |
| WT_IgV | Wild-type CD80 IgV | | 164 | | |
| WT_ECD | Wild-type CD80 ECD | | 2 | | 327 |
| CD80_165 | V11Y, M47V, F59S, L85E | 3 | 165 | 83 | 245 |
| CD80_166 | V11Y, M47V, L85E | 4 | 166 | 84 | 246 |
| CD80_167 | V11Y, M42I, M47V, A71G | 5 | 167 | 85 | 247 |
| CD80_168 | K9N, E10G, N63S, L85E | 6 | 168 | 86 | 248 |
| CD80_169 | E10G, E35G, D46E, M47V, V68M | 7 | 169 | 87 | 249 |
| CD80_170 | K9N, E10G, E35G, D46E, M47V, V68M | 8 | 170 | 88 | 250 |
| CD80_171 | E7S, H18I, V20L, A26K, M47L, A71N | 9 | 171 | 89 | 251 |
| CD80_172 | K9R, E10S, V11Y, M42L, F59M, | 10 | 172 | 90 | 252 |
| | V68M, L85E | | | | |
| CD80_173 | V11Y, T28Y, M47L, L85E | 11 | 173 | 91 | 253 |
| CD80_174 | E10R, M47R, N63I | 12 | 174 | 92 | 254 |
| CD80_175 | E10S, V11F, T28Y, M47L, T62S | 13 | 175 | 93 | 255 |
| CD80_176 | E10R, H18Y, A26G, E35D, V68L, L85E | 14 | 176 | 94 | 256 |
| CD80_177 | E10G, V11W, V22S, T28P, A71G, | 15 | 177 | 95 | 257 |
| | E81K, Y87R | | | | |
| CD80_178 | V11Y, T28Y, L85E, Y87I | 16 | 178 | 96 | 258 |
| CD80_179 | H18F, M42G, F59Y, V68N | 17 | 179 | 97 | 259 |
| CD80_180 | E10G, V11W, T28Y, M47L | 18 | 180 | 98 | 260 |
| CD80_181 | K9S, E10R, V11Y, M47L, A71G | 19 | 181 | 99 | 261 |
| CD80_182 | E10G, V11W, E35G, M47L, A71G, | 20 | 182 | 100 | 262 |
| | Y87R | | | | |
| CD80_183 | V11Y, T28Y, M47L, A71G, Y87R | 21 | 183 | 101 | 263 |
| CD80_184 | V11Y, A26Q, M47L, A71G, Y87R | 22 | 184 | 102 | 264 |
| CD80_185 | V11Y, T28Y, M47L | 23 | 185 | 103 | 265 |
| CD80_186 | E7K, V11W, N63H, A71G, Y87K | 24 | 186 | 104 | 266 |
| CD80_187 | V11Y, H18Y, E35G, L85Q | 25 | 187 | 105 | 267 |
| CD80_188 | E35D, V68L, L85E | 26 | 188 | 106 | 268 |
| CD80_189 | E10G, V11W, M47V, L85E | 27 | 189 | 107 | 269 |
| CD80_190 | T28Y, M47L | 28 | 190 | 108 | 270 |
| CD80_191 | V11Y, T28Y, M47L, V68L, L85E | 29 | 191 | 109 | 271 |
| CD80_192 | E7N, E35D, T101R | 30 | 192 | 110 | 272 |
| CD80_193 | V11Y, M42W, T62A, L85E | 31 | 193 | 111 | 273 |
| CD80_194 | V11Y, M42W, T62A | 32 | 194 | 112 | 274 |
| CD80_195 | V11Y, M42W, F59Y, V68N | 33 | 195 | 113 | 275 |
| CD80_196 | V11Y, M42W, E52K, T62A, L85E | 34 | 196 | 114 | 276 |
| CD80_197 | V11Y, E35D, Y87Q, T101R | 35 | 197 | 115 | 277 |
| CD80_198 | H18Y, A26E, R29S, E35D, M47L, | 36 | 198 | 116 | 278 |
| | V68M, A71G, E77G, D90G | | | | |
| CD80_199 | E10G, H18Y | 37 | 199 | 117 | 279 |
| CD80_200 | K9N, E10R, H18V, T28Y, A71G | 38 | 200 | 118 | 280 |
| CD80_201 | E10G, H18Y, T28Y, M47W, T62S | 39 | 201 | 119 | 281 |
| CD80_202 | E7H, H18L, V20I, T28Y, D46S, A71G | 40 | 202 | 120 | 282 |
| CD80_203 | K9N, E10A, V11W, H18F, T28H, | 41 | 203 | 121 | 283 |
| | M47L, T62E, R73D | | | | |
| CD80_204 | H18V, V20I, T28Y, E35G, M47V, R73E | 42 | 204 | 122 | 284 |
| CD80_205 | E10G, D46K, L85E | 43 | 205 | 123 | 285 |
| CD80_206 | E10G, H18T, Q27T, D46E, M47L | 44 | 206 | 124 | 286 |
| CD80_207 | E35D, V68T, T101K | 45 | 207 | 125 | 287 |
| CD80_208 | V11Y, E35G, M42G, F59S | 46 | 208 | 126 | 288 |
| CD80_209 | V11Y, T28R, E35G, M47L, F59S | 47 | 209 | 127 | 289 |
| CD80_210 | E7N, E35D, F59S | 48 | 210 | 128 | 290 |
| CD80_211 | V11Y, T28R, E35G, M47L, A71G | 49 | 211 | 129 | 291 |
| CD80_212 | K9R, E10A, E35G, V68T, T101K | 50 | 212 | 130 | 292 |
| CD80_213 | V11Y, V68T | 51 | 213 | 131 | 293 |
| CD80_214 | E10G, E35G, D46E, M47V, V68M, | 52 | 214 | 132 | 294 |
| | T101K | | | | |
| CD80_215 | K9R, E10A, E35G, V68L, L85E | 53 | 215 | 133 | 295 |
| CD80_216 | V11W, T28Y, D46V, R73E, F92L | 54 | 216 | 134 | 296 |
| CD80_217 | K9N, E10G, Y87K, T101Q | 55 | 217 | 135 | 297 |
| CD80-218 | V11W, T28Y, D46V, V68T, R73T, | 56 | 218 | 136 | 298 |
| | Y87N | | | | |
| CD80-219 | E10S, V11Y, M42R, A71V | 57 | 219 | 137 | 299 |
| CD80-220 | H18F, T28V, M47L, V68S | 58 | 220 | 138 | 300 |
| CD80_221 | E10G, A26S, T28Y | 59 | 221 | 139 | 301 |
| CD80_222 | E35D, D46Q, L85E | 60 | 222 | 140 | 302 |
| CD80_223 | E10S, V68M, Y87P | 61 | 223 | 141 | 303 |
| CD80_224 | K9N, V11W, M47L, V68T, R73T, Y87N | 62 | 224 | 142 | 304 |
| CD80_225 | V11Y, M47L, V68T | 63 | 225 | 143 | 305 |
| CD80_226 | K9R, A26T, T28Y, E35A, M47L, A71G | 64 | 226 | 144 | 306 |
| CD80_227 | V11W, T28H, D46Q, V68L, L85E | 65 | 227 | 145 | 307 |
| CD80_228 | E7Q, V11Y, R29H, M47L, V68T | 66 | 228 | 146 | 308 |
| CD80_229 | V11Y, M47V, A71G, L85E | 67 | 229 | 147 | 309 |
| CD80_230 | E10G, Q27F, D46N, A71G, D90G | 68 | 230 | 148 | 310 |

TABLE E2.B
Exemplary variant CD80 (Consensus)

| | | vIgD | | vIgD-Fc | |
|---|---|---|---|---|---|
| | | SEQ ID NO | | SEQ ID NO | |
| Name | Mutation(s) | nt | aa | nt | aa |
| WT_IgV | Wild-type CD80 IgV | | 164 | | |
| WT_ECD | Wild-type CD80 ECD | | 2 | | 327 |
| CD80_231 | V11W, T28Y, M47L | 69 | 231 | 149 | 311 |
| CD80_232 | V11F, T28Y, M47L | 70 | 232 | 150 | 312 |
| CD80_233 | V11Y, T28Y, M47L, V68M | 71 | 233 | 151 | 313 |
| CD80_234 | V11Y, T28Y, M47L, V68L | 72 | 234 | 152 | 314 |
| CD80_235 | V11Y, M47L, A71G | 73 | 235 | 153 | 315 |
| CD80_236 | V11Y, T28Y, M47L, A71G | 74 | 236 | 154 | 316 |
| CD80_237 | V11Y, M47V, V68M | 75 | 237 | 155 | 317 |
| CD80_238 | V11Y, D46E, M47V, V68M | 76 | 238 | 156 | 318 |
| CD80_239 | V11Y, T28Y, D46E, M47V | 77 | 239 | 157 | 319 |
| CD80_240 | V11Y, T28Y, D46E, M47V, | 78 | 240 | 158 | 320 |
| | V68M | | | | |
| CD80_241 | E10S, V11F, T28Y, M47L | 79 | 241 | 159 | 321 |
| CD80_242 | V11F, T28Y, M47L, T62S | 80 | 242 | 160 | 322 |
| CD80_243 | V11Y, M47L, A71G, Y87R | 81 | 243 | 161 | 323 |
| CD80_244 | V11Y, T28Y, M47L, Y87R | 82 | 244 | 162 | 324 |

### Example 3: Assessment of Binding of Affinity-Matured IgSF Domain-Containing Molecules

This Example describes Fc-fusion binding studies to cell-expressed counter structures to show specificity and affinity of CD80 domain variant immunomodulatory proteins for the cognate binding partners CD28, CTLA-4, and PD-L1.

Binding affinity to CD28 was determined using primary human T cells which endogenously express CD28. Binding to PD-L1 was determined using SCC152 cells transduced with human PD-L1. Binding to CTLA-4 was measured using Chinese hamster ovary (CHO) cells stably transduced to express human CTLA-4.

For staining and analysis by flow cytometry, 75,000 human T cells, SCC152/PD-L1 cells, or CHO/CTLA-4 cells were plated in 96-well round-bottom plates. Cells were spun down and resuspended in staining buffer (PBS (phosphate buffered saline), 1% BSA (bovine serum albumin), 1 mM EDTA, and 0.1% sodium azide) for 20 minutes to block non-specific binding. Afterwards, cells were centrifuged, stain buffer aspirated, and then resuspended in 50 μL of a 1:5 serial dilution of each CD80-Fc candidate variant starting at 100 nM ending at 0.8 nM. Primary staining was performed on ice for 60 minutes, before washing cells in 200 L staining buffer three times. PE-conjugated anti-human Fc (Jackson ImmunoResearch, USA) was diluted 1:250 in 50 μL staining buffer and added to cells and incubated another 30 minutes at 4° C. Cells were washed two times in 150 μL stain buffer and then fixed in 2% formaldehyde/PBS for 5 minutes at room temperature. Samples were analyzed CytoFLEX LX flow cytometer (Beckman Coulter, USA). Median Fluorescence Intensity (MFI) was calculated for each cell type using the CytExpert software from Beckman Coulter.

Results for exemplary CD80 variants are shown in Table E3 (binding to PD-L1), Table E4 (binding to CD28) and Table E5 (binding to CTLA-4). As a control, binding of the unmodified (wildytpe) CD80 ECD sequence fused to the Ig4 Fc sequence also was assessed (set forth in SEQ ID NO:327). Also shown is the binding activity as measured by the Mean Fluorescence Intensity (MFI) value for the binding of each variant CD80 Fc-fusion molecule to cells selected to express the indicated cognate counter structure ligand (i.e., CTLA-4, PD-L1, or CD28) and the fold change of the MFI of the variant CD80 IgV-Fc, compared to the binding of the corresponding unmodified CD80 ECD-Fc fusion molecule not containing the amino acid substitution(s), to the same cell-expressed counter structure ligand.

As shown, the selections resulted in the identification of a number of CD80 IgV domain variants that were affinity-modified to exhibit increased binding for the PD-L1 counter structure. In addition, the results indicate that a number of variants were selected that exhibit improved binding to CD28.

TABLE E3
PD-L1 Binding

| | | | | | | | @20 nM |
|---|---|---|---|---|---|---|---|
| | Variant CD80 IgV | | | | | | Fold |
| | | SEQ ID | Anti-Human Fc (MFI) | | | | change vs |
| Name | mutations | NO | 100 nM | 20 nM | 4 nM | 0.8 nM | WT CD80 |
| | Rational Design Variants | | | | | | |
| CD80_165 | V11Y, M47V, | 165 | 3634 | 3488 | 1957 | 1014 | 5.1 |
| | F59S, L85E | | | | | | |
| CD80_166 | V11Y, M47V, | 166 | 633 | 627 | 629 | 635 | 0.9 |
| | L85E | | | | | | |
| CD80_167 | V11Y, M42I, | 167 | 1145 | 736 | 665 | 643 | 1.1 |
| | M47V, A71G | | | | | | |
| CD80_168 | K9N, E10G, N63S, | 168 | 829 | 754 | 711 | 667 | 1.1 |
| | L85E | | | | | | |
| CD80_169 | E10G, E35G, | 169 | 1780 | 1400 | 1182 | 960 | 2.1 |
| | D46E, M47V, | | | | | | |
| | V68M | | | | | | |
| CD80_170 | K9N, E10G, E35G, | 170 | 1218 | 1069 | 976 | 843 | 1.6 |
| | D46E, M47V, | | | | | | |
| | V68M | | | | | | |
| CD80_171 | E7S, H18I, V20L, | 171 | 738 | 692 | 653 | 627 | 1.0 |
| | A26K, M47L, | | | | | | |
| | A71N | | | | | | |
| CD80_172 | K9R, E10S, V11Y, | 172 | 1692 | 1092 | 803 | 677 | 1.6 |
| | M42L, F59M, | | | | | | |
| | V68M, L85E | | | | | | |
| CD80_173 | V11Y, T28Y, | 173 | 5933 | 3853 | 1665 | 952 | 5.6 |
| | M47L, L85E | | | | | | |
| CD80_175 | E10S, V11F, T28Y, | 175 | 6774 | 6846 | 5702 | 2828 | 10.0 |
| | M47L, T62S | | | | | | |
| CD80_176 | E10R, H18Y, | 176 | 3316 | 2966 | 2494 | 1570 | 4.3 |
| | A26G, E35D, | | | | | | |
| | V68L, L85E | | | | | | |
| CD80_177 | E10G, V11W, | 177 | 2432 | 1215 | 873 | 727 | 1.8 |
| | V22S, T28P, A71G, | | | | | | |
| | E81K, Y87R | | | | | | |
| CD80_178 | V11Y, T28Y, | 178 | 3247 | 1650 | 1024 | 780 | 2.4 |
| | L85E, Y87I | | | | | | |
| CD80_179 | H18F, M42G, | 179 | 3230 | 1663 | 999 | 786 | 2.4 |
| | F59Y, V68N | | | | | | |
| CD80_180 | E10G, V11W, | 180 | 6569 | 4196 | 1833 | 1003 | 6.2 |
| | T28Y, M47L | | | | | | |
| CD80_181 | K9S, E10R, V11Y, | 181 | 5110 | 2209 | 1086 | 811 | 3.2 |
| | M47L, A71G | | | | | | |
| CD80_182 | E10G, V11W, | 182 | 5532 | 2737 | 1225 | 854 | 4.0 |
| | E35G, M47L, | | | | | | |
| | A71G, Y87R | | | | | | |
| CD80_183 | V11Y, T28Y, | 183 | 10759 | 7902 | 3265 | 1325 | 11.6 |
| | M47L, A71G, | | | | | | |
| | Y87R | | | | | | |
| CD80_184 | V11Y, A26Q, | 184 | 11479 | 9398 | 3991 | 1586 | 13.8 |
| | M47L, A71G, | | | | | | |
| | Y87R | | | | | | |
| CD80_185 | V11Y, T28Y, | 185 | 6262 | 4633 | 2163 | 1142 | 6.8 |
| | M47L | | | | | | |
| CD80_186 | E7K, V11W, | 186 | 1179 | 900 | 766 | 762 | 1.3 |
| | N63H, A71G, | | | | | | |
| | Y87K | | | | | | |
| CD80_187 | V11Y, H18Y, | 187 | 3357 | 1875 | 1073 | 836 | 2.7 |
| | E35G, L85Q | | | | | | |
| CD80_188 | E35D, V68L, L85E | 188 | 3345 | 2797 | 1968 | 1235 | 4.1 |
| CD80_189 | E10G, V11W, | 189 | 1953 | 1165 | 854 | 806 | 1.7 |
| | M47V, L85E | | | | | | |
| CD80_190 | T28Y, M47L | 190 | 659 | 561 | 597 | 589 | 0.8 |
| CD80_191 | V11Y, T28Y, | 191 | 9619 | 8684 | 5249 | 1944 | 12.7 |
| | M47L, V68L, L85E | | | | | | |
| CD80_192 | E7N, E35D, T101R | 192 | 1124 | 818 | 661 | 607 | 1.2 |
| CD80_193 | V11Y, M42W, | 193 | 3148 | 1774 | 981 | 710 | 2.6 |
| | T62A, L85E | | | | | | |
| CD80_194 | V11Y, M42W, | 194 | 2921 | 1481 | 859 | 694 | 2.2 |
| | T62A | | | | | | |
| CD80_196 | V11Y, M42W, | 196 | 2890 | 1688 | 932 | 680 | 2.5 |
| | E52K, T62A, L85E | | | | | | |
| CD80_197 | V11Y, E35D, | 197 | 6099 | 3221 | 1272 | 799 | 4.7 |
| | Y87Q, T101R | | | | | | |
| CD80_198 | H18Y, A26E, | 198 | 1239 | 1052 | 843 | 691 | 1.5 |
| | R29S, E35D, | | | | | | |
| | M47L, V68M, | | | | | | |
| | A71G, E77G, | | | | | | |
| | D90G | | | | | | |
| CD80_199 | E10G, H18Y | 199 | 811 | 802 | 711 | 65 | 1.2 |
| CD80_200 | K9N, E10R, H18V, | 200 | 2298 | 2175 | 1821 | 1285 | 3.2 |
| | T28Y, A71G | | | | | | |
| CD80_201 | E10G, H18Y, | 201 | 1446 | 1337 | 1099 | 932 | 2.0 |
| | T28Y, M47W, | | | | | | |
| | T62S | | | | | | |
| CD80_202 | E7H, H18L, V20I, | 202 | 1141 | 951 | 789 | 700 | 1.4 |
| | T28Y, D46S, A71G | | | | | | |
| CD80_203 | K9N, E10A, | 203 | 5032 | 4168 | 1908 | 1048 | 6.1 |
| | V11W, H18F, | | | | | | |
| | T28H, M47L, | | | | | | |
| | T62E, R73D | | | | | | |
| CD80_204 | H18V, V20I, T28Y, | 204 | 1298 | 831 | 665 | 662 | 1.2 |
| | E35G, M47V, | | | | | | |
| | R73E | | | | | | |
| CD80_205 | E10G, D46K, L85E | 205 | 817 | 678 | 649 | 685 | 1.0 |
| CD80_206 | E10G, H18T, | 206 | 936 | 860 | 806 | 672 | 1.3 |
| | Q27T, D46E, | | | | | | |
| | M47L | | | | | | |
| CD80_207 | E35D, V68T, | 207 | 793 | 652 | 623 | 657 | 1.0 |
| | T101K | | | | | | |
| CD80_208 | V11Y, E35G, | 208 | 714 | 653 | 652 | 688 | 1.0 |
| | M42G, F59S | | | | | | |
| CD80_209 | V11Y, T28R, | 209 | 746 | 634 | 644 | 666 | 0.9 |
| | E35G, M47L, F59S | | | | | | |
| CD80_210 | E7N, E35D, F59S | 210 | 640 | 630 | 630 | 688 | 0.9 |
| CD80_211 | V11Y, T28R, | 211 | 7022 | 3827 | 1546 | 922 | 5.6 |
| | E35G, M47L, | | | | | | |
| | A71G | | | | | | |
| CD80_212 | K9R, E10A, E35G, | 212 | 1507 | 948 | 734 | 698 | 1.4 |
| | V68T, T101K | | | | | | |
| CD80_213 | V11Y, V68T | 213 | 1177 | 1024 | 839 | 792 | 1.5 |
| CD80_214 | E10G, E35G, | 214 | 2029 | 1729 | 1498 | 1074 | 2.5 |
| | D46E, M47V, | | | | | | |
| | V68M, T101K | | | | | | |
| CD80_215 | K9R, E10A, E35G, | 215 | 2419 | 1669 | 1004 | 757 | 2.4 |
| | V68L, L85E | | | | | | |
| CD80_216 | V11W, T28Y, | 216 | 975 | 786 | 668 | 636 | 1.2 |
| | D46V, R73E, F92L | | | | | | |
| CD80_217 | K9N, E10G, Y87K, | 217 | 834 | 759 | 706 | 640 | 1.1 |
| | T101Q | | | | | | |
| CD80-218 | V11W, T28Y, | 218 | 797 | 740 | 702 | 596 | 1.1 |
| | D46V, V68T, | | | | | | |
| | R73T, Y87N | | | | | | |
| CD80-219 | E10S, V11Y, | 219 | 830 | 730 | 639 | 615 | 1.1 |
| | M42R, A71V | | | | | | |
| CD80-220 | H18F, T28V, | 220 | 698 | 622 | 621 | 635 | 0.9 |
| | M47L, V68S | | | | | | |
| CD80_221 | E10G, A26S, T28Y | 221 | 917 | 809 | 767 | 686 | 1.2 |
| CD80_222 | E35D, D46Q, L85E | 222 | 753 | 683 | 653 | 647 | 1.0 |
| CD80_223 | E10S, V68M, Y87P | 223 | 796 | 675 | 667 | 669 | 1.0 |
| CD80_224 | K9N, V11W, | 224 | 1061 | 955 | 856 | 764 | 1.4 |
| | M47L, V68T, | | | | | | |
| | R73T, Y87N | | | | | | |
| CD80_225 | V11Y, M47L, | 225 | 2301 | 1922 | 1283 | 931 | 2.8 |
| | V68T | | | | | | |
| CD80_226 | K9R, A26T, T28Y, | 226 | 1367 | 1177 | 1126 | 949 | 1.7 |
| | E35A, M47L, | | | | | | |
| | A71G | | | | | | |
| CD80_227 | V11W, T28H, | 227 | 1197 | 943 | 756 | 720 | 1.4 |
| | D46Q, V68L, L85E | | | | | | |
| CD80_228 | E7Q, V11Y, R29H, | 228 | 2081 | 1921 | 1445 | 998 | 2.8 |
| | M47L, V68T | | | | | | |
| CD80_229 | V11Y, M47V, | 229 | 4265 | 2217 | 1160 | 771 | 3.3 |
| | A71G, L85E | | | | | | |
| CD80_230 | E10G, Q27F, | 230 | 2112 | 1620 | 1113 | 833 | 2.4 |
| | D46N, A71G, | | | | | | |
| | D90G | | | | | | |

Consensus Variants

| CD80_231 | V11W, T28Y, | 231 | 1043 | 853 | 692 | 683 | 1.3 |
|---|---|---|---|---|---|---|---|
| | M47L | | | | | | |
| CD80_232 | V11F, T28Y, M47L | 232 | 1821 | 1569 | 1096 | 816 | 2.3 |
| CD80_233 | V11Y, T28Y, | 233 | 7174 | 6908 | 6516 | 3649 | 10.1 |
| | M47L, V68M | | | | | | |
| CD80_234 | V11Y, T28Y, | 234 | 9480 | 9551 | 6444 | 2463 | 14.0 |
| | M47L, V68L | | | | | | |
| CD80_235 | V11Y, M47L, | 235 | 8938 | 5236 | 1986 | 995 | 7.7 |
| | A71G | | | | | | |
| CD80_236 | V11Y, T28Y, | 236 | 10428 | 8998 | 4239 | 1675 | 13.2 |
| | M47L, A71G | | | | | | |
| CD80_238 | V11Y, D46E, | 238 | 5401 | 3602 | 1660 | 957 | 5.3 |
| | M47V, V68M | | | | | | |
| CD80_239 | V11Y, T28Y, | 239 | 4381 | 2191 | 1224 | 863 | 3.2 |
| | D46E, M47V | | | | | | |
| CD80_240 | V11Y, T28Y, | 240 | 9702 | 8755 | 7570 | 4224 | 12.8 |
| | D46E, M47V, | | | | | | |
| | V68M | | | | | | |
| CD80_241 | E10S, V11F, T28Y, | 241 | 6485 | 5146 | 3206 | 1505 | 7.5 |
| | M47L | | | | | | |
| CD80_242 | V11F, T28Y, | 242 | 2501 | 1766 | 1495 | 963 | 2.6 |
| | M47L, T62S | | | | | | |
| CD80_243 | V11Y, M47L, | 243 | 9476 | 5102 | 1854 | 960 | 7.5 |
| | A71G, Y87R | | | | | | |
| CD80_244 | V11Y, T28Y, | 244 | 6817 | 3502 | 1368 | 819 | 5.1 |
| | M47L, Y87R | | | | | | |
| | Controls | | | | | | |
| WT CD80 | | 327 | 637 | 652 | 652 | 644 | 1.0 |
| ECD Fc IgG4 | | | | | | | |
| IgG4 Fc | | 326 | 694 | 652 | 636 | 650 | 1.0 |
| Control | | | | | | | |

TABLE E4
CD28 Binding

| | | | | | | | @20 nM |
|---|---|---|---|---|---|---|---|
| | Variant CD80 IgV | | | | | | Fold |
| | | SEQ ID | Anti-Human Fc (MFI) | | | | change vs |
| Name | mutations | NO | 100 nM | 20 nM | 4 nM | 0.8 nM | WT CD80 |
| | Rational Design Variants | | | | | | |
| CD80_165 | V11Y, M47V, | 165 | 191 | 196 | 195 | 189 | 1.1 |
| | F59S, L85E | | | | | | |
| CD80_166 | V11Y, M47V, | 166 | 205 | 199 | 192 | 179 | 1.1 |
| | L85E | | | | | | |
| CD80_167 | V11Y, M42I, | 167 | 216 | 207 | 192 | 183 | 1.2 |
| | M47V, A71G | | | | | | |
| CD80_168 | K9N, E10G, N63S, | 168 | 206 | 187 | 187 | 181 | 1.0 |
| | L85E | | | | | | |
| CD80_169 | E10G, E35G, | 169 | 1045 | 756 | 455 | 271 | 4.2 |
| | D46E, M47V, | | | | | | |
| | V68M | | | | | | |
| CD80_170 | K9N, E10G, E35G, | 170 | 888 | 696 | 455 | 269 | 3.9 |
| | D46E, M47V, | | | | | | |
| | V68M | | | | | | |
| CD80_171 | E7S, H18I, V20L, | 171 | 1872 | 1108 | 526 | 280 | 6.2 |
| | A26K, M47L, | | | | | | |
| | A71N | | | | | | |
| CD80_172 | K9R, E10S, V11Y, | 172 | 1201 | 722 | 372 | 223 | 4.0 |
| | M42L, F59M, | | | | | | |
| | V68M, L85E | | | | | | |
| CD80_173 | V11Y, T28Y, | 173 | 436 | 269 | 202 | 186 | 1.5 |
| | M47L, L85E | | | | | | |
| CD80_175 | E10S, V11F, T28Y, | 175 | 1190 | 796 | 470 | 276 | 4.4 |
| | M47L, T62S | | | | | | |
| CD80_176 | E10R, H18Y, | 176 | 463 | 272 | 201 | 183 | 1.5 |
| | A26G, E35D, | | | | | | |
| | V68L, L85E | | | | | | |
| CD80_177 | E10G, V11W, | 177 | 255 | 201 | 170 | 208 | 1.1 |
| | V22S, T28P, A71G, | | | | | | |
| | E81K, Y87R | | | | | | |
| CD80_178 | V11Y, T28Y, | 178 | 209 | 202 | 193 | 186 | 1.1 |
| | L85E, Y87I | | | | | | |
| CD80_179 | H18F, M42G, | 179 | 246 | 212 | 194 | 192 | 1.2 |
| | F59Y, V68N | | | | | | |
| CD80_180 | E10G, V11W, | 180 | 1474 | 633 | 319 | 216 | 3.5 |
| | T28Y, M47L | | | | | | |
| CD80_181 | K9S, E10R, V11Y, | 181 | 313 | 265 | 212 | 200 | 1.5 |
| | M47L, A71G | | | | | | |
| CD80_182 | E10G, V11W, | 182 | 5467 | 2854 | 1031 | 436 | 15.9 |
| | E35G, M47L, | | | | | | |
| | A71G, Y87R | | | | | | |
| CD80_183 | V11Y, T28Y, | 183 | 3182 | 2350 | 1233 | 571 | 13.1 |
| | M47L, A71G, | | | | | | |
| | Y87R | | | | | | |
| CD80_184 | V11Y, A26Q, | 184 | 2779 | 2211 | 1188 | 556 | 12.3 |
| | M47L, A71G, | | | | | | |
| | Y87R | | | | | | |
| CD80_185 | V11Y, T28Y, | 185 | 1425 | 680 | 335 | 220 | 3.8 |
| | M47L | | | | | | |
| CD80_186 | E7K, V11W, | 186 | 446 | 298 | 220 | 183 | 1.7 |
| | N63H, A71G, | | | | | | |
| | Y87K | | | | | | |
| CD80_187 | V11Y, H18Y, | 187 | 205 | 188 | 175 | 174 | 1.0 |
| | E35G, L85Q | | | | | | |
| CD80_188 | E35D, V68L, L85E | 188 | 285 | 224 | 191 | 201 | 1.2 |
| CD80_189 | E10G, V11W, | 189 | 192 | 175 | 201 | 197 | 1.0 |
| | M47V, L85E | | | | | | |
| CD80_190 | T28Y, M47L | 190 | 1566 | 1032 | 600 | 359 | 5.7 |
| CD80_191 | V11Y, T28Y, | 191 | 543 | 401 | 302 | 268 | 2.2 |
| | M47L, V68L, L85E | | | | | | |
| CD80_192 | E7N, E35D, T101R | 192 | 640 | 423 | 295 | 277 | 2.3 |
| CD80_193 | V11Y, M42W, | 193 | 276 | 267 | 249 | 236 | 1.5 |
| | T62A, L85E | | | | | | |
| CD80_194 | V11Y, M42W, | 194 | 298 | 262 | 246 | 245 | 1.5 |
| | T62A | | | | | | |
| CD80_196 | V11Y, M42W, | 196 | 301 | 274 | 247 | 245 | 1.5 |
| | E52K, T62A, L85E | | | | | | |
| CD80_197 | V11Y, E35D, | 197 | 1547 | 699 | 293 | 218 | 3.9 |
| | Y87Q, T101R | | | | | | |
| CD80_198 | H18Y, A26E, | 198 | 205 | 195 | 197 | 186 | 1.1 |
| | R29S, E35D, | | | | | | |
| | M47L, V68M, | | | | | | |
| | A71G, E77G, | | | | | | |
| | D90G | | | | | | |
| CD80_199 | E10G, H18Y | 199 | 738 | 493 | 310 | 222 | 2.7 |
| CD80_200 | K9N, E10R, H18V, | 200 | 497 | 372 | 268 | 198 | 2.1 |
| | T28Y, A71G | | | | | | |
| CD80_201 | E10G, H18Y, | 201 | 285 | 254 | 201 | 199 | 1.4 |
| | T28Y, M47W, | | | | | | |
| | T62S | | | | | | |
| CD80_202 | E7H, H18L, V20I, | 202 | 810 | 387 | 252 | 226 | 2.1 |
| | T28Y, D46S, A71G | | | | | | |
| CD80_203 | K9N, E10A, | 203 | 993 | 550 | 341 | 269 | 3.1 |
| | V11W, H18F, | | | | | | |
| | T28H, M47L, | | | | | | |
| | T62E, R73D | | | | | | |
| CD80_204 | H18V, V201, T28Y, | 20 | 2114 | 971 | 457 | 289 | 5.4 |
| | E35G, M47V, | | | | | | |
| | R73E | | | | | | |
| CD80_205 | E10G, D46K, L85E | 205 | 431 | 299 | 265 | 233 | 1.7 |
| CD80_206 | E10G, H18T, | 206 | 1582 | 1207 | 565 | 342 | 6.7 |
| | Q27T, D46E, | | | | | | |
| | M47L | | | | | | |
| CD80_207 | E35D, V68T, | 207 | 1428 | 1105 | 661 | 352 | 6.1 |
| | T101K | | | | | | |
| CD80_208 | V11Y, E35G, | 208 | 708 | 387 | 283 | 235 | 2.1 |
| | M42G, F59S | | | | | | |
| CD80_209 | V11Y, T28R, | 209 | 357 | 231 | 193 | 188 | 1.3 |
| (H5890) | E35G, M47L, F59S | | | | | | |
| CD80_210 | E7N, E35D, F59S | 210 | 258 | 203 | 184 | 182 | 1.1 |
| CD80_211 | V11Y, T28R, | 211 | 430 | 309 | 229 | 188 | 1.7 |
| | E35G, M47L, | | | | | | |
| | A71G | | | | | | |
| CD80_212 | K9R, E10A, E35G, | 212 | 799 | 639 | 380 | 244 | 3.5 |
| | V68T, T101K | | | | | | |
| CD80_213 | V11Y, V68T | 213 | 851 | 630 | 381 | 219 | 3.5 |
| CD80_214 | E10G, E35G, | 214 | 999 | 722 | 480 | 375 | 4.0 |
| | D46E, M47V, | | | | | | |
| | V68M, T101K | | | | | | |
| CD80_215 | K9R, E10A, E35G, | 215 | 719 | 413 | 249 | 212 | 2.3 |
| | V68L, L85E | | | | | | |
| CD80_216 | V11W, T28Y, | 216 | 1585 | 771 | 355 | 247 | 4.3 |
| | D46V, R73E, F92L | | | | | | |
| CD80_217 | K9N, E10G, Y87K, | 217 | 4862 | 4551 | 2677 | 1036 | 25.3 |
| | T101Q | | | | | | |
| CD80-218 | V11W, T28Y, | 218 | 3455 | 2235 | 919 | 384 | 12.4 |
| | D46V, V68T, | | | | | | |
| | R73T, Y87N | | | | | | |
| CD80-219 | E10S, V11Y, | 219 | 3193 | 1750 | 578 | 280 | 9.7 |
| | M42R, A71V | | | | | | |
| CD80-220 | H18F, T28V, | 220 | 3443 | 2922 | 1830 | 687 | 16.2 |
| | M47L, V68S | | | | | | |
| CD80_221 | E10G, A26S, T28Y | 221 | 1332 | 1015 | 544 | 285 | 5.6 |
| CD80_222 | E35D, D46Q, L85E | 222 | 636 | 336 | 213 | 189 | 1.9 |
| CD80_223 | E10S, V68M, Y87P | 223 | 318 | 223 | 195 | 190 | 1.2 |
| CD80_224 | K9N, V11W, | 224 | 3355 | 2683 | 1651 | 610 | 14.9 |
| | M47L, V68T, | | | | | | |
| | R73T, Y87N | | | | | | |
| CD80_225 | V11Y, M47L, | 225 | 1279 | 807 | 422 | 236 | 4.5 |
| | V68T | | | | | | |
| CD80_226 | K9R, A26T, T28Y, | 226 | 628 | 411 | 284 | 207 | 2.3 |
| | E35A, M47L, | | | | | | |
| | A71G | | | | | | |
| CD80_227 | V11W, T28H, | 227 | 283 | 224 | 209 | 193 | 1.2 |
| | D46Q, V68L, L85E | | | | | | |
| CD80_228 | E7Q, V11Y, R29H, | 228 | 227 | 203 | 199 | 195 | 1.1 |
| | M47L, V68T | | | | | | |
| CD80_229 | V11Y, M47V, | 229 | 218 | 203 | 196 | 198 | 1.1 |
| | A71G, L85E | | | | | | |
| CD80_230 | E10G, Q27F, | 230 | 2200 | 1681 | 991 | 458 | 9.3 |
| | D46N, A71G, | | | | | | |
| | D90G | | | | | | |
| | Consensus Variants | | | | | | |
| CD80_231 | V11W, T28Y, | 231 | 2757 | 978 | 397 | 232 | 5.4 |
| | M47L | | | | | | |
| CD80_232 | V11F, T28Y, M47L | 232 | 1639 | 910 | 410 | 234 | 5.1 |
| CD80_233 | V11Y, T28Y, | 233 | 2763 | 2290 | 1346 | 540 | 12.7 |
| | M47L, V68M | | | | | | |
| CD80_234 | V11Y, T28Y, | 234 | 1161 | 812 | 453 | 261 | 4.5 |
| | M47L, V68L | | | | | | |
| CD80_235 | V11Y, M47L, | 235 | 546 | 330 | 237 | 192 | 1.8 |
| | A71G | | | | | | |
| CD80_236 | V11Y, T28Y, | 236 | 1246 | 680 | 376 | 247 | 3.8 |
| | M47L, A71G | | | | | | |
| CD80_238 | V11Y, D46E, | 238 | 1148 | 547 | 290 | 208 | 3.0 |
| | M47V, V68M | | | | | | |
| CD80_239 | V11Y, T28Y, | 239 | 3111 | 1185 | 479 | 264 | 6.6 |
| | D46E, M47V | | | | | | |
| CD80_240 | V11Y, T28Y, | 240 | 2765 | 2138 | 1234 | 542 | 11.9 |
| | D46E, M47V, | | | | | | |
| | V68M | | | | | | |
| CD80_241 | E10S, V11F, T28Y, | 241 | 1565 | 965 | 486 | 267 | 5.4 |
| | M47L | | | | | | |
| CD80_242 | V11F, T28Y, | 242 | 980 | 757 | 403 | 255 | 4.2 |
| | M47L, T62S | | | | | | |
| CD80_243 | V11Y, M47L, | 243 | 3028 | 1989 | 873 | 401 | 11.1 |
| | A71G, Y87R | | | | | | |
| CD80_244 | V11Y, T28Y, | 244 | 4193 | 2598 | 1422 | 525 | 14.4 |
| | M47L, Y87R | | | | | | |
| | Controls | | | | | | |
| WT CD80 | | 327 | 184 | 180 | 168 | 178 | 1.0 |
| ECD Fc IgG4 | | | | | | | |
| IgG4 Fc | | 326 | 172 | 170 | 167 | 189 | 0.9 |
| Control | | | | | | | |

TABLE E5
CTLA-4 Binding

| | | | | | | | @20 nM |
|---|---|---|---|---|---|---|---|
| | Variant CD80 IgV | | | | | | Fold |
| | | SEQ | Anti-Human Fc (MFI) | | | | change vs |
| Name | mutations | ID NO | 100 nM | 20 nM | 4 nM | 0.8 nM | WT CD80 |
| | Rational Design Variants | | | | | | |
| CD80_165 | V11Y, M47V, | 165 | 87696 | 12517 | 10761 | 3680 | 0.02 |
| | F59S, L85E | | | | | | |
| CD80_166 | V11Y, M47V, | 166 | 310 | 333 | 312 | 305 | 0.00 |
| | L85E | | | | | | |
| CD80_167 | V11Y, M42I, | 167 | 35278 | 1340 | 2476 | 892 | 0.00 |
| | M47V, A71G | | | | | | |
| CD80_168 | K9N, E10G, N63S, | 168 | 3158 | 830 | 631 | 415 | 0.00 |
| | L85E | | | | | | |
| CD80_169 | E10G, E35G, | 169 | 621000 | 591000 | 563000 | 284255 | 0.71 |
| | D46E, M47V, | | | | | | |
| | V68M | | | | | | |
| CD80_170 | K9N, E10G, E35G, | 170 | 597000 | 555000 | 505000 | 251647 | 0.67 |
| | D46E, M47V, | | | | | | |
| | V68M | | | | | | |
| CD80_171 | E7S, H18I, V20L, | 171 | 559000 | 565000 | 557000 | 374326 | 0.68 |
| | A26K, M47L, | | | | | | |
| | A71N | | | | | | |
| CD80_172 | K9R, E10S, V11Y, | 172 | 567000 | 573000 | 491000 | 256064 | 0.69 |
| | M42L, F59M, | | | | | | |
| | V68M, L85E | | | | | | |
| CD80_173 | V11Y, T28Y, | 173 | 521000 | 505000 | 387606 | 208550 | 0.61 |
| | M47L, L85E | | | | | | |
| CD80_175 | E10S, V11F, | 175 | 321110 | 542000 | 529000 | 339521 | 0.65 |
| | T28Y, M47L, T62S | | | | | | |
| CD80_176 | E10R, H18Y, | 176 | 403000 | 213688 | 75097 | 23710 | 0.26 |
| | A26G, E35D, | | | | | | |
| | V68L, L85E | | | | | | |
| CD80_177 | E10G, V11W, | 177 | 126955 | 36868 | 11111 | 2961 | 0.04 |
| | V22S, T28P, | | | | | | |
| | A71G, E81K, | | | | | | |
| | Y87R | | | | | | |
| CD80_178 | V11Y, T28Y, | 178 | 10761 | 3028 | 956 | 495 | 0.00 |
| | L85E, Y87I | | | | | | |
| CD80_179 | H18F, M42G, | 179 | 25267 | 6680 | 1623 | 660 | 0.01 |
| | F59Y, V68N | | | | | | |
| CD80_180 | E10G, V11W, | 180 | 534000 | 512000 | 452000 | 310116 | 0.62 |
| | T28Y, M47L | | | | | | |
| CD80_181 | K9S, E10R, V11Y, | 181 | 547000 | 365303 | 136066 | 45682 | 0.44 |
| | M47L, A71G | | | | | | |
| CD80_182 | E10G, V11W, | 182 | 497000 | 481000 | 435000 | 319993 | 0.58 |
| | E35G, M47L, | | | | | | |
| | A71G, Y87R | | | | | | |
| CD80_183 | V11Y, T28Y, | 183 | 587000 | 538000 | 498000 | 362765 | 0.65 |
| | M47L, A71G, | | | | | | |
| | Y87R | | | | | | |
| CD80_184 | V11Y, A26Q, | 184 | 595000 | 585000 | 523000 | 329038 | 0.71 |
| | M47L, A71G, | | | | | | |
| | Y87R | | | | | | |
| CD80_185 | V11Y, T28Y, | 185 | 547000 | 555000 | 521000 | 343089 | 0.67 |
| | M47L | | | | | | |
| CD80_186 | E7K, V11W, | 186 | 634000 | 645000 | 575000 | 332496 | 0.78 |
| | N63H, A71G, | | | | | | |
| | Y87K | | | | | | |
| CD80_187 | V11Y, H18Y, | 187 | 614000 | 616000 | 571000 | 242196 | 0.74 |
| | E35G, L85Q | | | | | | |
| CD80_188 | E35D, V68L, L85E | 188 | 525000 | 490000 | 351562 | 195221 | 0.59 |
| CD80_189 | E10G, V11W, | 189 | 130976 | 46786 | 13098 | 3628 | 0.06 |
| | M47V, L85E | | | | | | |
| CD80_190 | T28Y, M47L | 190 | 538000 | 544000 | 468000 | 299500 | 0.66 |
| CD80_191 | V11Y, T28Y, | 191 | 254288 | 59042 | 27479 | 7797 | 0.07 |
| | M47L, V68L, | | | | | | |
| | L85E | | | | | | |
| CD80_192 | E7N, E35D, T101R | 192 | 577000 | 591000 | 503000 | 291270 | 0.71 |
| CD80_193 | V11Y, M42W, | 193 | 398570 | 148903 | 43404 | 13618 | 0.18 |
| | T62A, L85E | | | | | | |
| CD80_194 | V11Y, M42W, | 194 | 559000 | 509000 | 250772 | 103867 | 0.61 |
| | T62A | | | | | | |
| CD80_196 | V11Y, M42W, | 196 | 32416 | 9748 | 2476 | 921 | 0.01 |
| | E52K, T62A, L85E | | | | | | |
| CD80_197 | V11Y, E35D, | 197 | 518000 | 559000 | 481000 | 286242 | 0.67 |
| | Y87Q, T101R | | | | | | |
| CD80_198 | H18Y, A26E, | 198 | 201425 | 75356 | 22931 | 6303 | 0.09 |
| | R29S, E35D, | | | | | | |
| | M47L, V68M, | | | | | | |
| | A71G, E77G, | | | | | | |
| | D90G | | | | | | |
| CD80_199 | E10G, H18Y | 199 | 591000 | 604000 | 534000 | 293306 | 0.73 |
| CD80_200 | K9N, E10R, H18V, | 200 | 507000 | 234729 | 478000 | 306892 | 0.28 |
| | T28Y, A71G | | | | | | |
| CD80_201 | E10G, H18Y, | 201 | 423000 | 345489 | 282282 | 159050 | 0.42 |
| | T28Y, M47W, | | | | | | |
| | T62S | | | | | | |
| CD80_202 | E7H, H18L, V20I, | 202 | 488000 | 458000 | 419000 | 263295 | 0.55 |
| | T28Y, D46S, | | | | | | |
| | A71G | | | | | | |
| CD80_203 | K9N, E10A, | 203 | 426000 | 316666 | 148387 | 58638 | 0.38 |
| | V11W, H18F, | | | | | | |
| | T28H, M47L, | | | | | | |
| | T62E, R73D | | | | | | |
| CD80_204 | H18V, V20I, | 204 | 503000 | 497000 | 436000 | 270731 | 0.60 |
| | T28Y, E35G, | | | | | | |
| | M47V, R73E | | | | | | |
| CD80_205 | E10G, D46K, L85E | 205 | 471000 | 256064 | 101034 | 27758 | 0.31 |
| CD80_206 | E10G, H18T, | 206 | 587000 | 361503 | 511000 | 294329 | 0.44 |
| | Q27T, D46E, | | | | | | |
| | M47L | | | | | | |
| CD80_207 | E35D, V68T, | 207 | 514000 | 478000 | 450000 | 279349 | 0.58 |
| | T101K | | | | | | |
| CD80_208 | V11Y, E35G, | 208 | 453000 | 218954 | 79352 | 27479 | 0.26 |
| | M42G, F59S | | | | | | |
| CD80_209 | V11Y, T28R, | 209 | 268852 | 96263 | 28040 | 7994 | 0.12 |
| | E35G, M47L, F59S | | | | | | |
| CD80_210 | E7N, E35D, F59S | 210 | 187899 | 58437 | 13797 | 4683 | 0.07 |
| CD80_211 | V11Y, T28R, | 211 | 414000 | 417000 | 393050 | 272623 | 0.50 |
| | E35G, M47L, | | | | | | |
| | A71G | | | | | | |
| CD80_212 | K9R, E10A, E35G, | 212 | 403000 | 376945 | 331339 | 220482 | 0.45 |
| | V68T, T101K | | | | | | |
| CD80_213 | V11Y, V68T | 213 | 523000 | 531000 | 455000 | 222795 | 0.64 |
| CD80_214 | E10G, E35G, | 214 | 357742 | 551000 | 399962 | 179602 | 0.66 |
| | D46E, M47V, | | | | | | |
| | V68M, T101K | | | | | | |
| CD80_215 | K9R, E10A, E35G, | 215 | 452000 | 237192 | 77195 | 20888 | 0.29 |
| | V68L, L85E | | | | | | |
| CD80_216 | V11W, T28Y, | 216 | 520000 | 502000 | 406000 | 212207 | 0.61 |
| | D46V, R73E, F92L | | | | | | |
| CD80_217 | K9N, E10G, Y87K, | 217 | 694000 | 689000 | 546000 | 249033 | 0.83 |
| | T101Q | | | | | | |
| CD80-218 | V11W, T28Y, | 218 | 573000 | 547000 | 365303 | 149940 | 0.66 |
| | D46V, V68T, | | | | | | |
| | R73T, Y87N | | | | | | |
| CD80-219 | E10S, V11Y, | 219 | 567000 | 471000 | 225916 | 73309 | 0.57 |
| | M42R, A71V | | | | | | |
| CD80-220 | H18F, T28V, | 220 | 591000 | 542000 | 468000 | 232292 | 0.65 |
| | M47L, V68S | | | | | | |
| CD80_221 | E10G, A26S, T28Y | 221 | 581000 | 563000 | 463000 | 250772 | 0.68 |
| CD80_222 | E35D, D46Q, L85E | 222 | 497000 | 216682 | 61734 | 16846 | 0.26 |
| CD80_223 | E10S, V68M, | 223 | 149940 | 44452 | 10422 | 2443 | 0.05 |
| | Y87P | | | | | | |
| CD80_224 | K9N, V11W, | 224 | 520000 | 503000 | 55130 | 245590 | 0.61 |
| | M47L, V68T, | | | | | | |
| | R73T, Y87N | | | | | | |
| CD80_225 | V11Y, M47L, | 225 | 493000 | 476000 | 390318 | 234729 | 0.57 |
| | V68T | | | | | | |
| CD80_226 | K9R, A26T, T28Y, | 226 | 534000 | 555000 | 490000 | 335990 | 0.67 |
| | E35A, M47L, | | | | | | |
| | A71G | | | | | | |
| CD80_227 | V11W, T28H, | 227 | 452000 | 184023 | 59652 | 18173 | 0.22 |
| | D46Q, V68L, L85E | | | | | | |
| CD80_228 | E7Q, V11Y, R29H, | 228 | 156314 | 80453 | 41103 | 20612 | 0.10 |
| | M47L, V68T | | | | | | |
| CD80_229 | V11Y, M47V, | 229 | 82134 | 29994 | 8645 | 2228 | 0.04 |
| | A71G, L85E | | | | | | |
| CD80_230 | E10G, Q27F, | 230 | 701000 | 670000 | 597000 | 356498 | 0.81 |
| | D46N, A71G, | | | | | | |
| | D90G | | | | | | |
| | Consensus Variants | | | | | | |
| CD80_231 | V11W, T28Y, | 231 | 549000 | 525000 | 520000 | 370432 | 0.63 |
| | M47L | | | | | | |
| CD80_232 | V11F, T28Y, | 232 | 575000 | 563000 | 521000 | 358991 | 0.68 |
| | M47L | | | | | | |
| CD80_233 | V11Y, T28Y, | 233 | 606000 | 593000 | 559000 | 349120 | 0.72 |
| | M47L, V68M | | | | | | |
| CD80_234 | V11Y, T28Y, | 234 | 567000 | 571000 | 546000 | 288243 | 0.69 |
| | M47L, V68L | | | | | | |
| CD80_235 | V11Y, M47L, | 235 | 636000 | 619000 | 497000 | 246446 | 0.75 |
| | A71G | | | | | | |
| CD80_236 | V11Y, T28Y, | 236 | 491000 | 507000 | 483000 | 267918 | 0.61 |
| | M47L, A71G | | | | | | |
| CD80_238 | V11Y, D46E, | 238 | 498000 | 337163 | 123486 | 44909 | 0.41 |
| | M47V, V68M | | | | | | |
| CD80_239 | V11Y, T28Y, | 239 | 555000 | 565000 | 516000 | 345489 | 0.68 |
| | D46E, M47V | | | | | | |
| CD80_240 | V11Y, T28Y, | 240 | 589000 | 625000 | 531000 | 321110 | 0.75 |
| | D46E, M47V, | | | | | | |
| | V68M | | | | | | |
| CD80_241 | E10S, V11F, | 241 | 491000 | 505000 | 473000 | 253404 | 0.61 |
| | T28Y, M47L | | | | | | |
| CD80_242 | V11F, T28Y, | 242 | 507000 | 520000 | 498000 | 307963 | 0.63 |
| | M47L, T62S | | | | | | |
| CD80_243 | V11Y, M47L, | 243 | 575000 | 555000 | 326753 | 334821 | 0.67 |
| | A71G, Y87R | | | | | | |
| CD80_244 | V11Y, T28Y, | 244 | 527000 | 497000 | 507000 | 305825 | 0.60 |
| | M47L, Y87R | | | | | | |
| | Controls | | | | | | |
| WT CD80 | | 327 | 856000 | 829000 | 525000 | 216682 | 1.00 |
| ECD Fc IgG4 | | | | | | | |
| IgG4 Fc | | 326 | 303 | 305 | 312 | 303 | 0.00 |
| Control | | | | | | | |

### Example 4: Assessment of Bioactivity of Affinity-Matured CD80 IgV Domain-Containing Molecules Using a Jurkat/IL2 Reporter Assay

This Example describes a Jurkat/IL2 reporter assay to assess bioactivity of CD80 domain variant immunomodulatory proteins for PD-L1-dependent CD28 costimulation.

Jurkat/IL2-reporter cells (Promega, USA), were counted and resuspended at 1×10⁶ cells/mL in assay buffer (RPMI 1640+5% fetal bovine serum (FBS)). Exemplary variant CD80 IgV-Fc fusion molecules, wildtype CD80 ECD-Fc control molecules, or negative control Fc alone were diluted to a concentration of 500 nM in assay buffer. A four-point, 1:5 serial dilution of each exemplary variant or control protein was made in assay buffer. Modified K562 cells expressing PD-L1 and membrane-bound anti-CD3 single-chain Fc from OKT3 were counted and brought to a concentration of 1.1×10⁶ cells/mL in assay buffer. K562 cells were plated at 25 μL/well into U-bottom 96-well polypropylene plates. 25 μL of each exemplary variant or control protein was then added to the K562 cells and plate was incubated at room temp for 30 minutes. The plate was then washed twice with 150 μL assay media and 110 μL of Jurkat effector cells added to each well to resuspend the K562 cells. 100 μL/well of the resuspended cells (Jurkat+K562 cells) was then transferred to a 96-well flat-bottom opaque white assay plate. The assay plate was briefly spun down (10 seconds at 1200 RPM) and placed in a 37° C., 5% CO₂ incubator for 5 hours.

After the 5-hour incubation, the plate was removed and equilibrated to room temperature for 15 minutes. 100 μL/well of Bio-Glo (Promega) was added to the assay plate, which was then placed on an orbital shaker for 10 minutes. Luminescence was measured with a 250 millisecond per well integration time using a BioTek Cytation 3 luminometer. An average relative luminescence value was determined for each variant CD80 IgV-Fc and a fold increase in IL-2 reporter signal was calculated for each variant compared to wildtype CD80 ECD-Fc protein. The results are provided in Table E6 below.

As shown in Table E6, co-culturing most of the exemplary variant CD80 IgV-Fc molecules with K562/OKT3/PD-L1 and Jurkat cells expressing an IL-2-luciferase reporter, resulted in increased CD28 costimulation (i.e. PD-L1 dependent CD28 costimulation) compared to WT CD80 ECD-Fc or Fc-only negative control.

TABLE E6
K562/OKT3/PD-L1+ Jurkat/IL-2 Reporter Cell Assay

| | Variant CD80 IgV | | | | | | Fold |
|---|---|---|---|---|---|---|---|
| | | SEQ | Rel. Luminescence Units (RLU) | | | | Increase over |
| Name | mutations | ID NO | 250 nM | 50 nM | 10 nM | 2 nM | WT at 50 nM |

Rational Design

| | | | | | | | |
|---|---|---|---|---|---|---|---|
| CD80_165 | V11Y, M47V, F59S, | 165 | 3461 | 3296 | 3125 | 2857 | 1.35 |
| | L85E | | | | | | |
| CD80_166 | V11Y, M47V, L85E | 166 | 2668 | 2508 | 2462 | 2619 | 1.03 |
| CD80_167 | V11Y, M42I, M47V, | 167 | 2868 | 2622 | 2673 | 2326 | 1.07 |
| | A71G | | | | | | |
| CD80_168 | K9N, E10G, N63S, | 168 | 2524 | 2649 | 2665 | 2264 | 1.09 |
| | L85E | | | | | | |
| CD80_169 | E10G, E35G, D46E, | 169 | 13097 | 8167 | 5404 | 3887 | 3.35 |
| | M47V, V68M | | | | | | |
| CD80_170 | K9N, E10G, E35G, | 170 | 10351 | 6178 | 3900 | 2985 | 2.53 |
| | D46E, M47V, | | | | | | |
| | V68M | | | | | | |
| CD80_171 | E7S, H18I, V20L, | 171 | 3285 | 2413 | 2367 | 2102 | 0.99 |
| | A26K, M47L, A71N | | | | | | |
| CD80_172 | K9R, E10S, V11Y, | 172 | 4894 | 3266 | 2513 | 2251 | 1.34 |
| | M42L, F59M, | | | | | | |
| | V68M, L85E | | | | | | |
| CD80_173 | V11Y, T28Y, M47L, | 173 | 6948 | 4231 | 2665 | 2486 | 1.73 |
| | L85E | | | | | | |
| CD80_175 | E10S, V11F, T28Y, | 175 | 16019 | 12850 | 10525 | 7506 | 5.27 |
| | M47L, T62S | | | | | | |
| CD80_176 | E10R, H18Y, A26G, | 176 | 4840 | 3228 | 2938 | 2722 | 1.32 |
| | E35D, V68L, L85E | | | | | | |
| CD80_177 | E10G, V11W, V22S, | 177 | 7300 | 3453 | 2941 | 2841 | 1.42 |
| | T28P, A71G, E81K, | | | | | | |
| | Y87R | | | | | | |
| CD80_178 | V11Y, T28Y, L85E, | 178 | 2762 | 2998 | 3004 | 3480 | 1.23 |
| | Y87I | | | | | | |
| CD80_179 | H18F, M42G, F59Y, | 179 | 3250 | 2662 | 2616 | 2952 | 1.09 |
| | V68N | | | | | | |
| CD80_180 | E10G, V11W, | 180 | 8804 | 6560 | 3459 | 3096 | 2.69 |
| | T28Y, M47L | | | | | | |
| CD80_181 | K9S, E10R, V11Y, | 181 | 8015 | 4006 | 2540 | 2427 | 1.64 |
| | M47L, A71G | | | | | | |
| CD80_182 | E10G, V11W, | 182 | 8262 | 4989 | 2716 | 2394 | 2.04 |
| | E35G, M47L, A71G, | | | | | | |
| | Y87R | | | | | | |
| CD80_183 | V11Y, T28Y, M47L, | 183 | 10937 | 8647 | 5016 | 2849 | 3.54 |
| | A71G, Y87R | | | | | | |
| CD80_184 | V11Y, A26Q, | 184 | 11248 | 9435 | 5867 | 2966 | 3.87 |
| | M47L, A71G, Y87R | | | | | | |
| CD80_185 | V11Y, T28Y, M47L | 185 | 8891 | 6612 | 3808 | 2773 | 2.71 |
| CD80_186 | E7K, V11W, N63H, | 186 | 3266 | 2500 | 2275 | 2819 | 1.02 |
| | A71G, Y87K | | | | | | |
| CD80_187 | V11Y, H18Y, E35G, | 187 | 5463 | 3832 | 2716 | 2757 | 1.57 |
| | L85Q | | | | | | |
| CD80_188 | E35D, V68L, L85E | 188 | 4550 | 3147 | 2892 | 3163 | 1.29 |
| CD80_189 | E10G, V11W, | 189 | 3098 | 3250 | 2882 | 2765 | 1.33 |
| | M47V, L85E | | | | | | |
| CD80_190 | T28Y, M47L | 190 | 3320 | 2852 | 2573 | 2500 | 1.17 |
| CD80_191 | V11Y, T28Y, M47L, | 191 | 2654 | 2410 | 2535 | 2565 | 0.99 |
| | V68L, L85E | | | | | | |
| CD80_192 | E7N, E35D, T101R | 192 | 2795 | 2611 | 2408 | 2432 | 1.07 |
| CD80_193 | V11Y, M42W, | 193 | 2492 | 2305 | 2424 | 2215 | 0.94 |
| | T62A, L85E | | | | | | |
| CD80_194 | V11Y, M42W, | 194 | 3323 | 2616 | 2131 | 2183 | 1.07 |
| | T62A | | | | | | |
| CD80_196 | V11Y, M42W, | 196 | 2475 | 2269 | 2283 | 2286 | 0.93 |
| | E52K, T62A, L85E | | | | | | |
| CD80_197 | V11Y, E35D, Y87Q, | 197 | 10264 | 5571 | 2773 | 2318 | 2.28 |
| | T101R | | | | | | |
| CD80_198 | H18Y, A26E, R29S, | 198 | 5417 | 3396 | 2435 | 2459 | 1.39 |
| | E35D, M47L, | | | | | | |
| | V68M, A71G, | | | | | | |
| | E77G, D90G | | | | | | |
| CD80_199 | E10G, H18Y | 199 | 6051 | 3421 | 2616 | 2478 | 1.40 |
| CD80_200 | K9N, E10R, H18V, | 200 | 9625 | 6303 | 4561 | 3310 | 2.58 |
| | T28Y, A71G | | | | | | |
| CD80_201 | E10G, H18Y, T28Y, | 201 | 10370 | 6864 | 4201 | 3545 | 2.81 |
| | M47W, T62S | | | | | | |
| CD80_202 | E7H, H18L, V20I, | 202 | 4680 | 3572 | 3074 | 3383 | 1.46 |
| | T28Y, D46S, A71G | | | | | | |
| CD80_203 | K9N, E10A, V11W, | 203 | 11937 | 8828 | 6276 | 3843 | 3.62 |
| | H18F, T28H, M47L, | | | | | | |
| | T62E, R73D | | | | | | |
| CD80_204 | H18V, V20I, T28Y, | 204 | 4006 | 2795 | 2830 | 2735 | 1.15 |
| | E35G, M47V, R73E | | | | | | |
| CD80_205 | E10G, D46K, L85E | 205 | 2502 | 2307 | 2589 | 2700 | 0.95 |
| CD80_206 | E10G, H18T, Q27T, | 206 | 5626 | 3196 | 2706 | 2825 | 1.31 |
| | D46E, M47L | | | | | | |
| CD80_207 | E35D, V68T, T101K | 207 | 2554 | 2167 | 2272 | 2329 | 0.89 |
| CD80_208 | V11Y, E35G, | 208 | 2232 | 2158 | 2210 | 2454 | 0.88 |
| | M42G, F59S | | | | | | |
| CD80_209 | V11Y, T28R, E35G, | 209 | 2297 | 2180 | 2196 | 2511 | 0.89 |
| | M47L, F59S | | | | | | |
| CD80_210 | E7N, E35D, F59S | 210 | 2302 | 2340 | 2329 | 2581 | 0.96 |
| CD80_211 | V11Y, T28R, E35G, | 211 | 11153 | 6661 | 3247 | 2741 | 2.73 |
| | M47L, A71G | | | | | | |
| CD80_212 | K9R, E10A, E35G, | 212 | 6531 | 3131 | 2632 | 2697 | 1.28 |
| | V68T, T101K | | | | | | |
| CD80_213 | V11Y, V68T | 213 | 9254 | 5959 | 4109 | 3125 | 2.44 |
| CD80_214 | E10G, E35G, D46E, | 214 | 13902 | 9221 | 6696 | 5092 | 3.78 |
| | M47V, V68M, | | | | | | |
| | T101K | | | | | | |
| CD80_215 | K9R, E10A, E35G, | 215 | 6555 | 5366 | 3640 | 2692 | 2.20 |
| | V68L, L85E | | | | | | |
| CD80_216 | V11W, T28Y, | 216 | 3778 | 2543 | 2353 | 2416 | 1.04 |
| | D46V, R73E, F92L | | | | | | |
| CD80_217 | K9N, E10G, Y87K, | 217 | 8327 | 4260 | 2662 | 2362 | 1.75 |
| | T101Q | | | | | | |
| CD80-218 | V11W, T28Y, | 218 | 3775 | 2654 | 2223 | 2242 | 1.09 |
| | D46V, V68T, R73T, | | | | | | |
| | Y87N | | | | | | |
| CD80-219 | E10S, V11Y, M42R, | 219 | 3881 | 2581 | 2440 | 2191 | 1.06 |
| | A71V | | | | | | |
| CD80-220 | H18F, T28V, M47L, | 220 | 3288 | 2470 | 2324 | 2364 | 1.01 |
| | V68S | | | | | | |
| CD80_221 | E10G, A26S, T28Y | 221 | 8709 | 3933 | 2947 | 2538 | 1.61 |
| CD80_222 | E35D, D46Q, L85E | 222 | 3732 | 2557 | 2592 | 2649 | 1.05 |
| CD80_223 | E10S, V68M, Y87P | 223 | 2982 | 2627 | 2746 | 2362 | 1.08 |
| CD80_224 | K9N, V11W, M47L, | 224 | 13780 | 8202 | 4174 | 3109 | 3.36 |
| | V68T, R73T, Y87N | | | | | | |
| CD80_225 | V11Y, M47L, V68T | 225 | 7655 | 5878 | 4450 | 3488 | 2.41 |
| CD80_226 | K9R, A26T, T28Y, | 226 | 5452 | 3152 | 3096 | 3025 | 1.29 |
| | E35A, M47L, A71G | | | | | | |
| CD80_227 | V11W, T28H, | 227 | 2524 | 2424 | 2467 | 3001 | 0.99 |
| | D46Q, V68L, L85E | | | | | | |
| CD80_228 | E7Q, V11Y, R29H, | 228 | 4385 | 3339 | 3215 | 2876 | 1.37 |
| | M47L, V68T | | | | | | |
| CD80_229 | V11Y, M47V, | 229 | 2635 | 2643 | 2375 | 2497 | 1.08 |
| | A71G, L85E | | | | | | |
| CD80_230 | E10G, Q27F, D46N, | 230 | 7777 | 5184 | 3236 | 2454 | 2.12 |
| | A71G, D90G | | | | | | |

Consensus Variants

| | | | | | | | |
|---|---|---|---|---|---|---|---|
| CD80_231 | V11W, T28Y, M47L | 23 | 3258 | 2733 | 2454 | 2454 | 1.12 |
| CD80_232 | V11F, T28Y, M47L | 232 | 5962 | 4160 | 3096 | 2706 | 1.70 |
| CD80_233 | V11Y, T28Y, M47L, | 233 | 12912 | 11573 | 10110 | 9609 | 4.74 |
| | V68M | | | | | | |
| CD80_234 | V11Y, T28Y, M47L, | 234 | 13642 | 12286 | 10403 | 6238 | 5.04 |
| | V68L | | | | | | |
| CD80_235 | V11Y, M47L, A71G | 235 | 12639 | 8536 | 4014 | 3375 | 3.50 |
| CD80_236 | V11Y, T28Y, M47L, | 236 | 16550 | 13360 | 8254 | 3794 | 5.48 |
| | A71G | | | | | | |
| CD80_238 | V11Y, D46E, | 238 | 13099 | 9820 | 4225 | 2841 | 4.02 |
| | M47V, V68M | | | | | | |
| CD80_239 | V11Y, T28Y, D46E, | 239 | 8552 | 4144 | 2353 | 2083 | 1.70 |
| | M47V | | | | | | |
| CD80_240 | V11Y, T28Y, D46E, | 240 | 12381 | 11026 | 8446 | 6539 | 4.52 |
| | M47V, V68M | | | | | | |
| CD80_241 | E10S, V11F, T28Y, | 241 | 10538 | 7644 | 4965 | 3334 | 3.13 |
| | M47L | | | | | | |
| CD80_242 | V11F, T28Y, M47L, | 242 | 8847 | 5818 | 4228 | 3112 | 2.38 |
| | T62S | | | | | | |
| CD80_243 | V11Y, M47L, | 243 | 10731 | 7587 | 3515 | 2329 | 3.11 |
| | A71G, Y87R | | | | | | |
| CD80_244 | V11Y, T28Y, M47L, | 244 | 11473 | 7538 | 2998 | 2324 | 3.09 |
| | Y87R | | | | | | |

Controls

| | | | | | | |
|---|---|---|---|---|---|---|
| WT CD80 | 327 | 2639 | 2440 | 2659.5 | 2828.5 | 1.00 |
| ECD-Fc IgG4 | | | | | | |
| IgG4 Fc | 326 | 2193.5 | 2164 | 2560.5 | 2375 | 0.89 |
| Control | | | | | | |

### Example 5: Generation of Multivalent Variant CD80 IgSF Domain Fusion Proteins and Binding Assessment

This Example describes generation of variant CD80 IgSF domain fusion proteins containing four affinity modified IgV domains from identified variant CD80 polypeptides. Specifically, two units of exemplary variants CD80 IgV with E10G/V11W/T28Y/M47L (SEQ ID NO: 180), V11Y/T28Y/M47L (SEQ ID NO: 185) or V11Y/E35D/Y87Q/T101R (SEQ ID NO: 197) were linked together and fused to an Fc in various configurations that, following dimerization of the Fc chains, result in the generation of tetravalent molecules.

### A. Generation of Multivalent Proteins

Multivalent variant CD80 IgSF domain fusion proteins were generated in various configurations as follows. In the generated multivalent proteins, the variant CD80 IgV variants were variously linked to the N- or C-terminus of a human IgG4 Fc region (SEQ ID NO:326) or an effectorless human IgG1 (containing mutations L234A/L235E/G237A by EU numbering; SEQ ID NO:362) via a peptide linker.

For each multivalent protein, the encoding nucleic acid molecule was designed to produce proteins in various configurations with sequences in the order shown in Table E7:

TABLE E7
Multivalent Constructs

| | CD80 vIgD | | SEQ ID |
|---|---|---|---|
| Name | (mutations) | Structure- | NO |
| CD80-Fc- | CD80_180 | CD80 vIgD (SEQ ID NO: 180)- | 336 |
| CD80_336 | E10G/V11W/T28Y/M47L | GSGGGGS (SEQ ID NO: 325)- | |
| | | IgG4 Fc (SEQ ID NO: 326)-3x | |
| | | GGGGS (SEQ ID NO: 330)-CD80 | |
| | | vIgD (SEQ ID NO: 180) | |
| CD80- | CD80_180 | CD80 vIgD (SEQ ID NO: 180)-3x | 337 |
| CD80- | E10G/V11W/T28Y/M47L | GGGGS (SEQ ID NO: 330)-CD80 | |
| Fc_337 | CD80_415 E10G/V11W/ | vIgD (SEQ ID NO: 415)- | |
| | /T28Y/M47L | GSGGGGS (SEQ ID NO: 325)- | |
| | | IgG4 Fc (SEQ ID NO: 326) | |
| CD80-Fc- | CD80_180 | CD80 vIgD (SEQ ID NO: 180)- | 338 |
| CD80_338 | E10G/V11W/T28Y/M47L | GSGGGGS (SEQ ID NO: 325)- | |
| | | IgG1 Fc (SEQ ID NO:362)-3x | |
| | | GGGGS (SEQ ID NO: 330)-CD80 | |
| | | vIgD (SEQ ID NO: 185) | |
| CD80-Fc- | CD80_185 | CD80 vIgD (SEQ ID NO: 185)- | 339 |
| CD80_339 | V11Y/T28Y/M47L | GSGGGGS (SEQ ID NO: 325)- | |
| | | IgG4 Fc (SEQ ID NO: 326)-3x | |
| | | GGGGS (SEQ ID NO: 330)-CD80 | |
| | | vIgD (SEQ ID NO: 185) | |
| CD80- | CD80_185 | CD80 vIgD (SEQ ID NO: 185)-3x | 340 |
| CD80- | V11Y/T28Y/M47L | GGGGS (SEQ ID NO: 330)-CD80 | |
| Fc_340 | CD80_416 V11Y/ | vIgD (SEQ ID NO: 416)- | |
| | /T28Y/M47L | GSGGGGS (SEQ ID NO: 325)- | |
| | | IgG4 Fc (SEQ ID NO: 326) | |
| CD80-Fc- | CD80_197 | CD80 vIgD (SEQ ID NO: 197)- | 341 |
| CD80_341 | V11Y/E35D/ | GSGGGGS (SEQ ID NO: 325)- | |
| | Y87Q/T101R | IgG4 Fc (SEQ ID NO: 326)-3x | |
| | | GGGGS (SEQ ID NO: 330)-CD80 | |
| | | vIgD (SEQ ID NO: 197) | |
| CD80- | CD80_197 | CD80 vIgD (SEQ ID NO: 197)-3x | 342 |
| CD80- | V11Y/E35D/ | GGGGS (SEQ ID NO: 330)-CD80 | |
| Fc_342 | Y87Q/T101R | vIgD (SEQ ID NO: 197)- | |
| | | GSGGGGS (SEQ ID NO: 325)- | |
| | | IgG4 Fc (SEQ ID NO: 326) | |
| CD80- | CD80_236 | CD80 vIgD (SEQ ID NO: 236)-3x | 414 |
| CD80- | V11Y/T28Y/M47L/A71G | GGGGS (SEQ ID NO: 330)-CD80 | |
| Fc_414 | CD80_417 | vIgD (SEQ ID NO: 417)- | |
| | V11Y/ | GSGGGGS (SEQ ID NO: 325)- | |
| | /T28Y/M47L/A71G | IgG4 Fc (SEQ ID NO: 326) | |

### B. Binding Assessment

Binding assays were carried out to assess the specificity and affinity of multivalent proteins to cell-expressed CTLA-4, CD28, and PD-L1 counter structures. The multivalent variant CD80 IgSF domain fusion proteins were tested for binding, substantially as described in Example 3.

Results for exemplary CD80 variants are shown in Table E8 (binding to PD-L1), Table E9 (binding to CD28) and Table E10 (binding to CTLA-4). As a control, binding of the unmodified (wildytpe) CD80 ECD sequence fused to the Ig4 Fc sequence also was assessed (set forth in SEQ ID NO:327). Also shown is the binding activity as measured by the Median Fluorescence Intensity (MFI) value for the binding of each variant CD80 Fc-fusion molecule to cells selected to express the indicated cognate counter structure ligand (i.e., CTLA-4, PD-L1, or CD28) and the fold change of the MFI of the variant CD80 IgV-Fc, compared to the binding of the corresponding unmodified CD80 ECD-Fc fusion molecule not containing the amino acid substitution(s), to the same cell-expressed counter structure ligand.

As shown, the multivalent variant CD80 IgSF domain fusion proteins exhibited increased binding for one or more of the counter structures.

TABLE E8
PD-L1 Binding

| | | | | | | @20 nM |
|---|---|---|---|---|---|---|
| Multivalent CD80 IgV | | Anti-Human Fc (MFI) | | | | Fold change |
| name | SEQ ID NO | 100 nM | 20 nM | 4 nM | 0.8 nM | vs WT CD80 |
| CD80-Fc- | 336 | 8448 | 7003 | 5746 | 3804 | 10.3 |
| CD80_336 | | | | | | |
| CD80-CD80- | 337 | 8659 | 8724 | 7864 | 5337 | 12.8 |
| Fc_337 | | | | | | |
| CD80-Fc- | 338 | 12456 | 11048 | 9298 | 6250 | 16.2 |
| CD80_338 | | | | | | |
| CD80-Fc- | 339 | 8087 | 7119 | 6184 | 4181 | 10.4 |
| CD80_339 | | | | | | |
| CD80-CD80- | 340 | 9019 | 8597 | 7794 | 5102 | 12.6 |
| Fc_340 | | | | | | |
| CD80-Fc- | 341 | 7814 | 7102 | 6412 | 4747 | 10.4 |
| CD80_341 | | | | | | |
| CD80-CD80- | 342 | 7631 | 7502 | 5456 | 2201 | 11.0 |
| Fc_342 | | | | | | |

Controls

| | | | | | | |
|---|---|---|---|---|---|---|
| WT CD80 ECD- | 327 | 637 | 652 | 652 | 644 | 1.0 |
| Fc IgG4 | | | | | | |
| IgG4 Fc Control | 326 | 694 | 652 | 636 | 650 | 1.0 |

TABLE E9
CD28 Binding

| | | | | | | @20 nM |
|---|---|---|---|---|---|---|
| Multivalent CD80 IgV | | Anti-Human Fc (MFI) | | | | Fold change |
| name | SEQ ID NO | 100 nM | 20 nM | 4 nM | 0.8 nM | vs WT CD80 |
| CD80-Fc- | 336 | 1472 | 844 | 496 | 329 | 4.7 |
| CD80_336 | | | | | | |
| CD80-CD80- | 337 | 3869 | 3420 | 3082 | 1529 | 19.0 |
| Fc_337 | | | | | | |
| CD80-Fc- | 338 | 2328 | 1350 | 926 | 552 | 7.5 |
| CD80_338 | | | | | | |
| CD80-Fc- | 339 | 1384 | 1023 | 793 | 520 | 5.7 |
| CD80_339 | | | | | | |
| CD80-CD80- | 340 | 3637 | 3605 | 3075 | 1660 | 20.0 |
| Fc_340 | | | | | | |
| CD80-Fc- | 341 | 1741 | 1410 | 1069 | 676 | 7.8 |
| CD80_341 | | | | | | |
| CD80-CD80- | 342 | 3692 | 2968 | 1648 | 611 | 16.5 |
| Fc_342 | | | | | | |

Controls

| | | | | | | |
|---|---|---|---|---|---|---|
| WT CD80 ECD- | 327 | 184 | 180 | 168 | 178 | 1.0 |
| Fc IgG4 | | | | | | |
| IgG4 Fc Control | 326 | 172 | 170 | 167 | 189 | 0.9 |

TABLE E10
CTLA-4 Binding

| | | | | | | @20 nM |
|---|---|---|---|---|---|---|
| | | | | | | Fold change |
| Variant CD80 IgV | | Anti-Human Fc (MFI) | | | | vs WT |
| name | SEQ ID NO | 100 nM | 20 nM | 4 nM | 0.8 nM | CD80 |
| CD80-Fc- | 336 | 284255 | 292286 | 283267 | 186598 | 0.35 |
| CD80_336 | | | | | | |
| CD80-CD80- | 337 | 93317 | 332496 | 204956 | 209276 | 0.40 |
| Fc_337 | | | | | | |
| CD80-Fc- | 338 | 511000 | 470000 | 439000 | 366578 | 0.57 |
| CD80_338 | | | | | | |
| CD80-Fc- | 339 | 319993 | 322231 | 304761 | 210005 | 0.39 |
| CD80_339 | | | | | | |
| CD80-CD80- | 340 | 352789 | 317772 | 317772 | 235547 | 0.38 |
| Fc_340 | | | | | | |
| CD80-Fc- | 341 | 282282 | 297421 | 280323 | 206386 | 0.36 |
| CD80_341 | | | | | | |
| CD80-CD80- | 342 | 357742 | 352789 | 216682 | 162962 | 0.43 |
| Fc_342 | | | | | | |

Controls

| | | | | | | |
|---|---|---|---|---|---|---|
| WT CD80 ECD- | 327 | 856000 | 829000 | 525000 | 216682 | 1.00 |
| Fc IgG4 | | | | | | |
| IgG4 Fc Control | 326 | 303 | 305 | 312 | 303 | 0.00 |

### C. PD-L1-Dependent CD28 Costimulation Bioactivity in a Jurkat/IL-2 Reporter Assay

A Jurkat/ML2 reporter assay to assess bioactivity of multivalent CD8 domain variant immunomodulatory proteins for PD-L1-dependent CD28 costimulation. The multivalent variant CD80 IgSF domain fusion proteins were tested for bioactivity, substantially as described in Example 4.

As shown in Table E11, co-culturing the exemplary multivalent variant CD8 IgV-Fc molecules with K562/OKT3/PD-L1 and Jurkat cells expressing an IL-2-luciferase reporter, resulted in increased CD28 costimulation (i.e. PD-L1 dependent CD28 costimulation) compared to WT CD80 ECD-Fc or Fc-only negative control.

TABLE E11
K562/OKT3/PD-L1+ Jurkat/IL-2 Reporter Cell Assay

| | | | | | | Fold Increase |
|---|---|---|---|---|---|---|
| Multivalent Variant CD80 IgV | | Rel. Luminescence Units (RLU) | | | | over WT at |
| Name | SEQ ID NO | 250 nM | 50 nM | 10 nM | 2 nM | 50 nM |
| CD80-Fc- | 336 | 12067 | 13173 | 12441 | 10560 | 5.40 |
| CD80_336 | | | | | | |
| CD80-CD80- | 337 | 11826 | 12628 | 12769 | 11644 | 5.18 |
| Fc_337 | | | | | | |
| CD80-Fc- | 338 | 13300 | 12254 | 12753 | 10904 | 5.02 |
| CD80_338 | | | | | | |
| CD80-Fc- | 339 | 13455 | 13541 | 13506 | 11926 | 5.55 |
| CD80_339 | | | | | | |
| CD80-CD80- | 340 | 12940 | 13666 | 13216 | 12921 | 5.60 |
| Fc_340 | | | | | | |
| CD80-Fc- | 341 | 15214 | 15656 | 15968 | 13129 | 6.42 |
| CD80_341 | | | | | | |
| CD80-CD80- | 342 | 16141 | 15100 | 14607 | 11771 | 6.19 |
| Fc_342 | | | | | | |

Controls

| | | | | | | |
|---|---|---|---|---|---|---|
| WT CD80 ECD- | 327 | 2639 | 2440 | 2659.5 | 2828.5 | 1.00 |
| Fc IgG4 | | | | | | |
| IgG4 Fc Control | 326 | 2193.5 | 2164 | 2560.5 | 2375 | 0.89 |

### Example 6: PD-L1 Binding Off-Rate Assessment of Variant CD80 Immunomodulatory Proteins

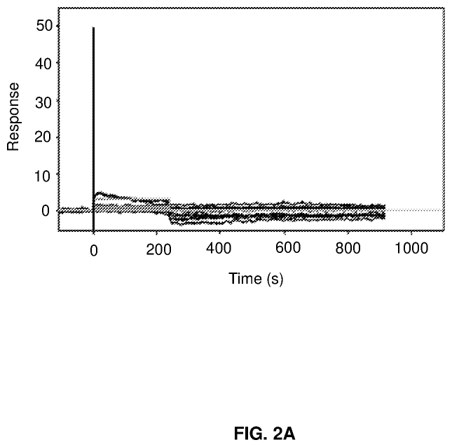

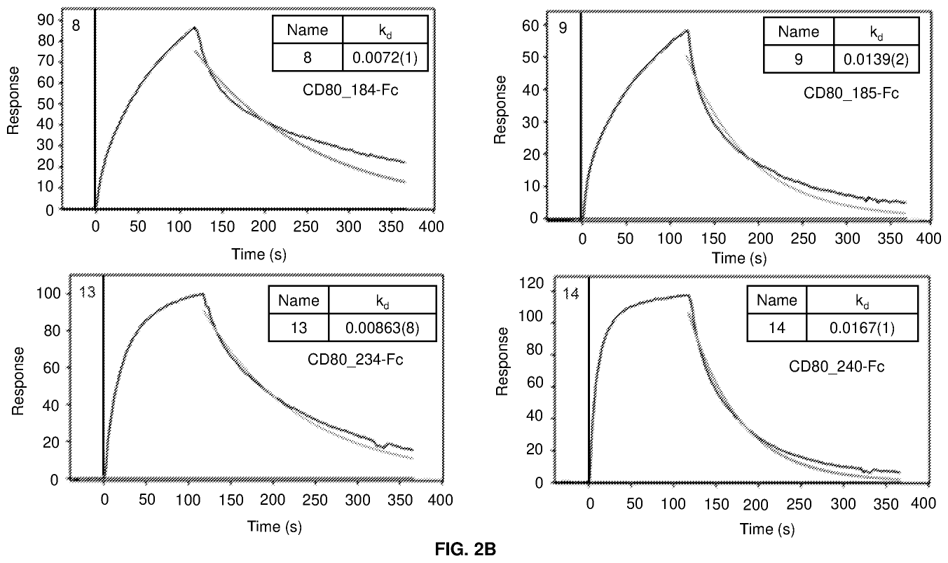

A binding study was carried out to determine the binding affinity of wild-type CD80 ECD-Fc to PD-L1 by surface plasmon resonance (SPR). Kinetic rate coefficients were recovered from binding analysis experiment performed with a Biacore 3000 biosensor. Three-fold concentration series of 1500 nM to 18.5 nM PD-L1 were run in triplicate against captured wildtype CD83 ECD-Fc surfaces. The data were collected using a 240 s association phase and a 600 s dissociation phase. The graphs are the sensorgrams and the association and dissociation phase data were globally fit to a 1:1 binding model to determine the association rate coefficient (ka), dissociation rate coefficient (kd), and the Rₘₐₓ value. The interactions of wild-type CD80 ECD-Fc to PD-L1 were weak and thus estimated and expressed as a minimal Kd using the theoretical Rmax as a fixed value when fitting the data. An exemplary SPR sensorgram ofwild-type CD8 ECD-Fc binding to PD-L1 is shown in FIG. 2A.

Similar analysis was carried out on exemplary tested vIgD CD81-Fc variants generated as described in Example 2, except that the data were collected with a 120 s association phase and a 240 s dissociation phase. Exemplary SPR sensorgrams are shown in FIG. 2B.

Results indicate that the tested variant CD80 immunomodulatory Fc fusion proteins exhibited a PD-L1 off-rate (k_{d}) of less than 20×10⁻³ s⁻¹. Table E12 sets forth binding results of exemplary variant CD80-Fc fusion proteins for binding PD-L1.

TABLE E12
CD80 vIgD-Fc binding to PD-L1

| | | vIgD | | | |
|---|---|---|---|---|---|
| | | SEQ ID | Fc SEQ | k_{d} | Complex |
| N# | Mutations | NO | ID NO | 1 × 10 (s⁻¹) | half-life (s) |
| CD80_175-Fc | E10S, V11F, T28Y, | 175 | 255 | 0.018 | 38.5 |
| | M47L, T62S | | | | |
| CD80_180-Fc | E10G, V11W, | 180 | 260 | 0.014 | 50.6 |
| | T28Y, M47L | | | | |
| CD80_181-Fc | K9S, E10R, V11Y, | 181 | 261 | 0.007 | 100.0 |
| | M47L, A71G | | | | |
| CD80_183-Fc | V11Y, T28Y, | 183 | 263 | 0.007 | 93.5 |
| | M47L, A71G, | | | | |
| | Y87R | | | | |
| CD80_184-Fc | V11Y, A26Q, | 184 | 264 | 0.007 | 96.9 |
| | M47L, A71G, | | | | |
| | Y87R | | | | |
| CD80_185-Fc | V11Y, T28Y, M47L | 185 | 265 | 0.014 | 50.0 |
| CD80_197-Fc | V11Y, E35D, | 197 | 277 | 0.014 | 50.4 |
| | Y87Q, T101R | | | | |
| CD80_171-Fc | E7S, H18I, V20L, | 171 | 251 | 0.009 | 73.6 |
| | A26K, M47L, | | | | |
| | A71N | | | | |
| CD80_233-Fc | V11Y, T28Y, | 233 | 313 | 0.016 | 42.2 |
| | M47L, V68M | | | | |
| CD80_234-Fc | V11Y, T28Y, | 234 | 314 | 0.009 | 80.4 |
| | M47L, V68L | | | | |
| CD80_240-Fc | V11Y, T28Y, | 240 | 320 | 0.017 | 41.4 |
| | D46E, M47V, | | | | |
| | V68M | | | | |
| CD80_241-Fc | E10S, V11F, T28Y, | 241 | 321 | 0.018 | 37.8 |
| | M47L | | | | |

Binding of CD28, and PD-L1 (analytes) to variant CD80-Fc fusion proteins was further assessed by SPR, including assessment of additional variants. Analyte concentration series were prepared using 3-fold dilution steps in running buffer. The concentration ranges were 500 nM to 2.06 nM for CD28, and 1500 nM to 6.17 nM for PD-L1. SPR analysis was conducted as described above. The association phases for all analyte concentrations were monitored for 240 s while the dissociation phases were collected for 600 s, at a flow rate of 30 uL/min. For the PD-L1 data, the 240 s association phase data and the 600 s dissociation phase data were globally fit to the 1:1 binding model, to determine the determine the association rate coefficient (ka), dissociation rate coefficient (kd), and the Rmax value. For the CD28 data, the 240 s association phase data and the first 120 s of the 600 s dissociation phase data were globally fit to the 1:1 binding model, to determine the ka, kd, and Rmax values. The Kd is the ratio of the dissociation rate to the association rate.

Table E13 sets for binding data of CD28 and PD-L1 to exemplary assessed variant CD80-Fc fusion proteins. The results show varying dissociation rates (kd) for CD80 variants with CD80_236-Fc demonstrating the slowest dissociation for CD28 and tightest binding. CD80_236-Fc also exhibited among the fastest association rates (L1 association rates (ka) for PD-L1, while the ka of other CD80 variants for PD-L1 was slower. The dissociation rate constant or off-rate (k_{d}) of CD80 variants for PD-L1 also was relatively slow.

TABLE E13
CD28 and PD-L1 Binding

| | | | kₐ 1 × 10⁵ | k_{d} 1 × 10⁻³ | Rₘₐₓ | K_{d} |
|---|---|---|---|---|---|---|
| Name | Mutations | Analyte | (M⁻¹s⁻¹) | (s⁻¹) | (RU) | (nM) |
| CD80_180-Fc | E10G, | rhu CD28 | 0.641 | 6.16 | 23.1 | 96 |
| | V11W, | rhu PD-L1 | 0.082 | 5.63 | 46.8 | 680 |
| | T28Y, | | | | | |
| | M47L | | | | | |
| CD80_185-Fc | V11Y, | rhu CD28 | 0.644 | 2.05 | 43.6 | 31.7 |
| | T28Y, | rhu PD-L1 | 0.484 | 3.55 | 51.8 | 73.5 |
| | M47L | | | | | |
| CD80_233-Fc | V11Y, | rhu CD28 | 0.688 | 6.01 | 33.5 | 87.4 |
| | T28Y, | rhu PD-L1 | 0.143 | 3.80 | 35.2 | 265 |
| | M47L, | | | | | |
| | V68M | | | | | |
| CD80_234-Fc | V11Y, | rhu CD28 | 0.620 | 3.94 | 43.6 | 63.6 |
| | T28Y, | rhu PD-L1 | 0.203 | 2.80 | 56.4 | 138 |
| | M47L, | | | | | |
| | V68L | | | | | |
| CD80_236-Fc | V11Y, | rhu CD28 | 0.724 | 0.320 | 50.9 | 4.42 |
| | T28Y, | rhu PD-L1 | 1.66 | 5.81 | 52.3 | 35.1 |
| | M47L, | | | | | |
| | A71G | | | | | |

### Example 7: Assessment of Cell-Surface PD-L1 Binding and Functional Blockade of PD-1/PD-L1 Interaction of Variant CD80 Fusion Proteins

PD-L1 binding and PD-1 blocking studies of variant CD80 Fc-fusion were performed using cell lines expressing a range of predetermined PD-L1 receptor densities to show affinity and avidity effects of the CD80 domain variant immunomodulatory proteins.

The number of PD-L1 molecules expressed on the surface of four cell lines, was determined using Quantum™ Simply Cellular® (QSC) microspheres following the manufacturer's instructions, and PD-L1 expression from highest to lowest was determined as follows: K562/OKT3/PD-L1 (260K PD-L1 copies)>HCC-827 (65K PD-L1 copies)>SCC 152/PD-L1 (38K PD-L1 copies)>A704 (7K PD-L1 copies)). Avidity is more likely to play a role in apparent binding on cell lines with higher PD-L1 densities, such as K562/OKT3/PD-L1. At lower receptor densities, avidity effects are less impactful and monomeric interactions result in a more accurate assessment of affinity.

Certain variant CD80-Fc fusion protein dimers and multivalent tandem formats were assessed for binding and bioactivity, as follows: CD80_233-Fc (SEQ ID NO: 313; V11Y, T28Y, M47L, V68M); CD80_234-Fc (SEQ ID NO: 314; V11Y, T28Y, M47L, V68L); CD80_236-Fc (SEQ ID NO: 316; V11Y, T28Y, M47L, A71G); CD80-CD80-Fc_414 (SEQ ID NO: 414; V11Y, T28Y, M47L, A71G); CD80-CD80-Fc_337 (SEQ ID NO: 337; E10G, V11W, T28Y, M47L), CD80-CD80-Fc_340 (SEQ ID NO: 340; V11Y, T28Y, M47L). As a control, binding and bioactivity also were compared to WT CD80 (ECD)-Fc (SEQ ID NO:327), as well as the corresponding Fc fusion protein containing the IgV domain of CD80 instead of the full ECD (WT CD80 (IgV)-Fc; WT CD80 IgV set forth in SEQ ID NO:164).

### A. Cell-Based PD-L1 Binding Assessment

Staining analysis by flow cytometry, was conducted in a similar manner for all four cell lines tested. 50,000-100,000 cells were plated in 96-well round-bottom plates. Cells were spun down and resuspended in staining buffer (PBS (phosphate buffered saline), 1% BSA (bovine serum albumin), 1 mM EDTA, and 0.1% sodium azide) for 20 minutes to block non-specific binding. Afterwards, cells were centrifuged, stain buffer aspirated, and then resuspended in 50 L of a 1:3 serial dilution of each CD80-Fc candidate variant starting at 1000 nM ending at 6 pM. Primary staining was performed on ice for 60 minutes, before washing cells in 200 L staining buffer three times. APC-conjugated anti-human Fc (BioLegend Inc. USA, Clone HP6017) was diluted 1:250 in 50 L staining buffer and added to cells and incubated another 60 minutes on ice. Cells were washed two times in 150 L stain buffer and then fixed in 2% formaldehyde/PBS for 5 minutes at room temperature. Samples were analyzed CytoFLEX LX flow cytometer (Beckman Coulter, USA). Median Fluorescence Intensity (MFI) was calculated for each cell type using the CytExpert software from Beckman Coulter.

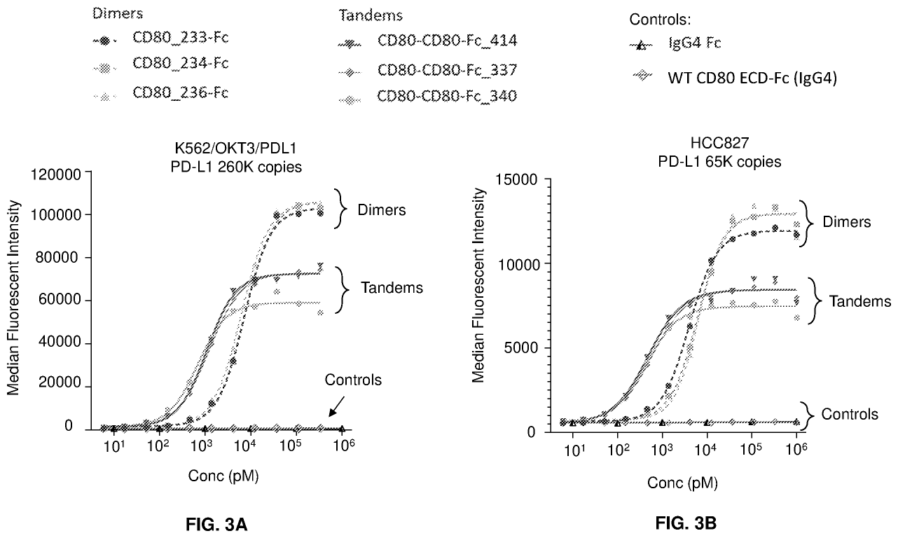

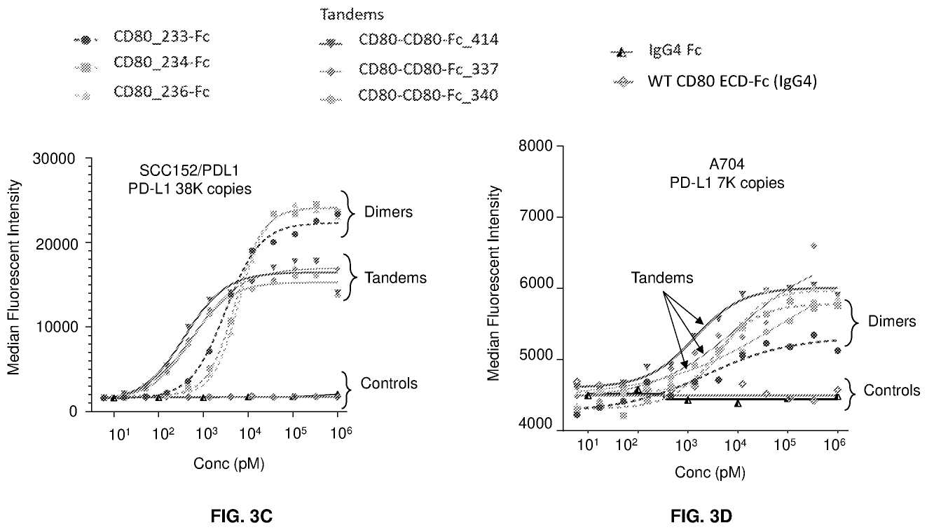

FIGS. 3A-3D depict binding of variants to the different cell lines with a range of PD-L1 receptor densities. All variant CD80 molecules exhibited substantially increased binding to the cell lines compared to WT CD80-Fc fusion protein controls. Differences in binding were observed between dimer and tandem formats, which is consistent with valency differences between dimers and tandems since a tandem format can bind four PD-L1 while a dimer format can bind two PD-L1.

### B. Functional Assessment of PD-1/PD-L1 Blockade

K562/OKT3/PD-L1 and SCC152/PD-L1 low/OKT3 cell lines were resuspended at 2.4×10⁶ cells/mL in assay buffer (AssayComplete Cell Plating 0 reagent, Eurofins DiscoverX Products, LLC, USA). HCC827/OKT3 cell line was resuspended at 3×10⁶ cells/mL in culture medium (RPMI+10% fetal bovine serum (FBS)). To prevent any PD-1/PD-L2 interaction, all cells were incubated for 20 minutes at room temperature (RT) with 40 nM of an αPD-L2 blocking antibody (BioLegend, USA). All cells were then washed with 3× excess culture medium volume and centrifuged, then resuspended at 2.4×10⁶ cells/mL in assay buffer. Each target cell line was added to a round bottom polypropylene 96-well plate, 25 μL/well.

Exemplary variant CD80 IgV-Fc fusion molecules described above, αPD-L1 antibody (Atezolizumab), negative control Fc alone, or WT CD80-Fc fusions were diluted to a concentration of 600 nM in assay buffer. A ten-point, 1:4.5 serial dilution of each exemplary variant or control protein was made in assay buffer to test with K562/OKT3/PD-L1 cells. A ten-point 1:3.5 serial dilution of each exemplary variant or control protein was made in assay buffer to test with HCC827/OKT3 or SCC152/PD L1 low/OKT3 cells. 25 μL of each exemplary variant or control protein was then added to the target cells and plates were incubated at RT for 30 minutes, shaking at 80 rpm. Jurkat/PD-1/SHP2 reporter cells (Eurofins DiscoverX Products, LLC, USA), were counted and resuspended at 8×10⁵ cells/mL in assay buffer. Jurkat/PD-1/SHP2 cells were added to assay plates, 50 μL/well. The assay plate was briefly spun down (10 seconds at 1200 RPM) and placed in a 37° C., 5% CO₂ incubator for 2 hours.

After the 2-hour incubation, the plate was removed from the incubator. 90 μL from each well was transferred from 96-well polypropylene assay plate to a 96-well flat-bottom opaque white assay plate. Detection Reagent 1 (Eurofins DiscoverX Products, LLC, USA) was added, 9 μL/well, and plates were shaken for 60 seconds at 350 RPM. Plates were then incubated for 15 minutes at RT, protected from light. Detection Reagent 2 (Eurofins DiscoverX Products, LLC, USA) was added, 36 μL/well and plates were incubated for 60 minutes at RT, protected from light. After incubation, luminescence was measured with a X millisecond per well integration time using a Molecular Devices iD5 luminometer. A relative luminescence value was determined for each variant CD80 IgV Fc and control protein, and blockade of SHP2 recruitment, and therefore PD-1-PD-L1 interaction, is demonstrated by reduced RLU values.

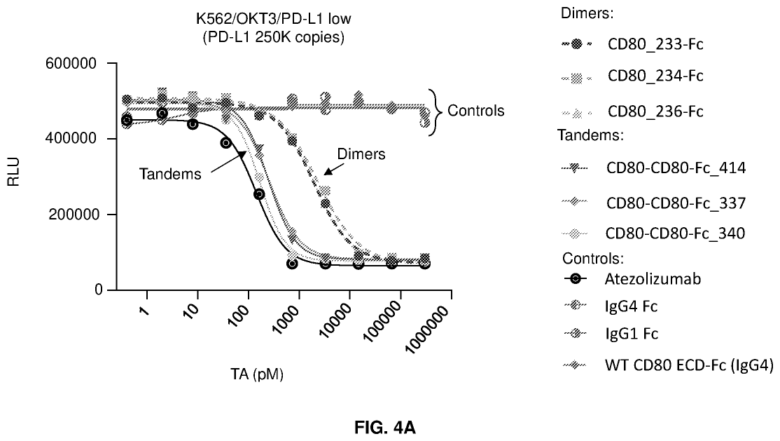

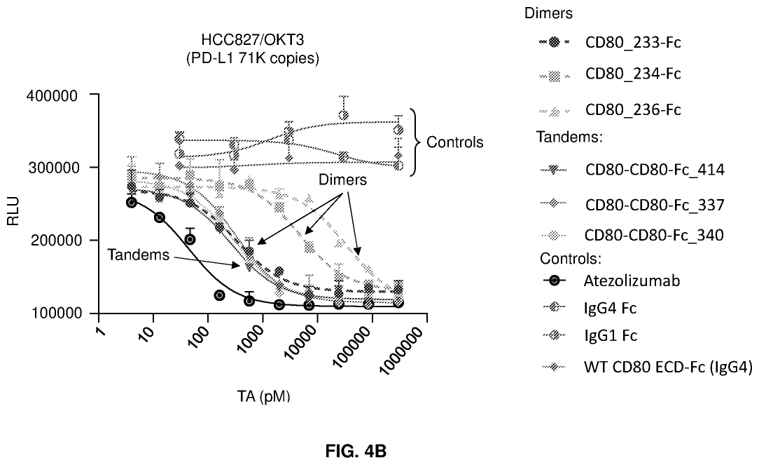

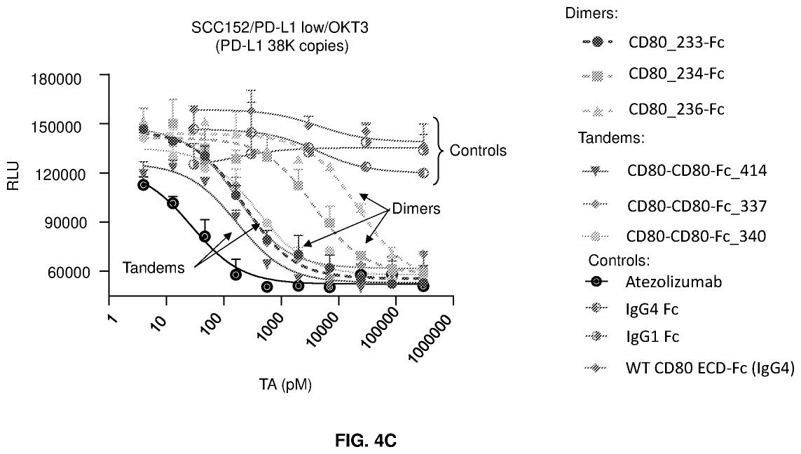

As shown in FIG. 4A-4C, co-culturing the exemplary variant CD80 IgV-Fc molecules with the PD-L1-expressing target cells and Jurkat cells expressing an IL-2-luciferase reporter, resulted in increased T cell stimulation compared to WT CD80 ECD-Fc or Fc-only negative control. The tandem variant CD80 IgV-Fc (SEQ ID NOS: 414, 337 and 340) show the greatest potency for PD-1:PD-L1 blockade.

### Example 8: Assessment of PD-L1-Dependent CD28 Costimulation of Primary T Cells with Affinity-Matured CD80 IgSF Domain-Containing Molecules

This Example describes a primary T cell activation assay to assess the bioactivity of CD80 domain variant immunomodulatory proteins for PD-L1-dependent CD28 costimulation using artificial antigen presenting cells (aAPCs) expressing a range of PD-L1 densities.

The aAPCs used in these studies include the four cell lines described in Example 7 shown to exhibit varying PD-L1 expression levels and engineered to express membrane-bound anti-CD3 (OKT3) single-chain Fc (K562/OKT3/PD-L1, HCC-827/OKT3, SCC152/OKT3/PD-L1, and A704/OKT3). When combined with cell lines expressing lower PD-L1 densities, exemplary variant CD80 IgV-Fc fusion molecules with higher affinity should induce more CD28 costimulation than the lower affinity variants.

Variant CD80-Fc fusion protein dimers and multivalent tandem formats were assessed as follows: CD80_233-Fc (SEQ ID NO: 313; V11Y, T28Y, M47L, V68M); CD80_234-Fc (SEQ ID NO: 314; V11Y, T28Y, M47L, V68L); CD80-CD80-Fc_337 (SEQ ID NO: 337; E10G, V11W, T28Y, M47L), CD80-CD80-Fc_340 (SEQ ID NO: 340; V11Y, T28Y, M47L).

Primary human T cells were counted and resuspended at 1×10⁶ cells/mL in assay buffer (X-VIVO15 (Lonza Inc., USA)+10% fetal bovine serum (FBS)). Exemplary variant CD80 IgV-Fc fusion molecules, wildtype CD80 ECD-Fc control molecules, or negative control Fc alone were 8-point, serially diluted in assay buffer with concentrations ranging from 100 nM to 1.5 pM. The four aAPC lines expressing PD-L1 and membrane-bound anti-CD3 single-chain Fc were counted and brought to a concentration of 0.8×10⁵ cells/mL in assay buffer. The aAPCs were plated at 50 μL/well into U-bottom 96-well polypropylene plates. 50 μL of each exemplary variant or control protein (at 4× final concentration) was then added to the aAPCs. 100,000 primary human T cells were then added to each well and the plate was then placed in a 37° C., 5% CO₂ incubator for 24 hours.

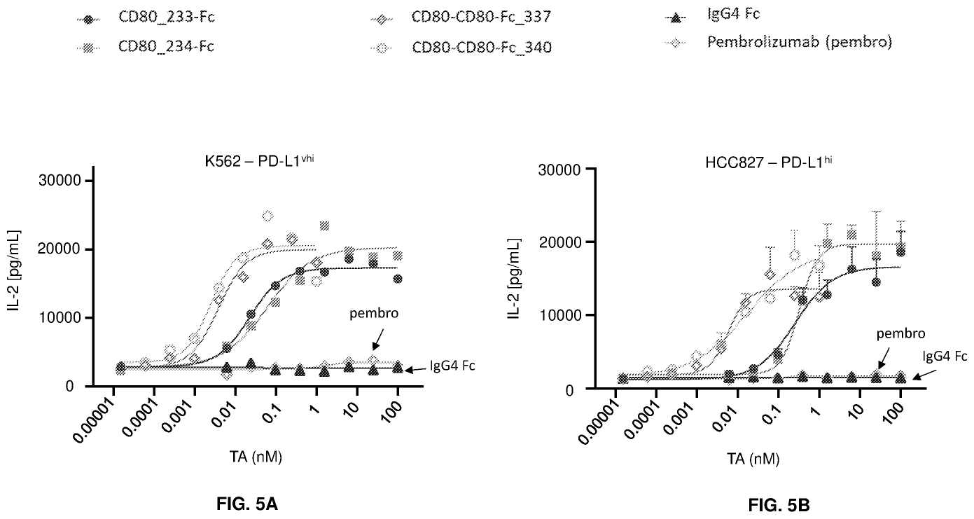

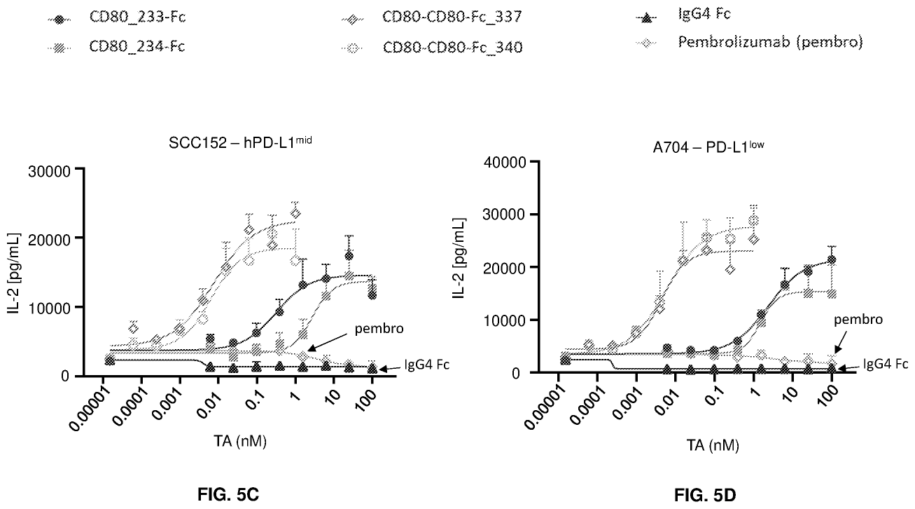

After the 24-hour incubation, the plate was removed from the incubator and 50 μL of culture supernatant was collected from each well. Secreted IL-2 was then measured using a bead-based IL-2 detection kit (Millipore, USA) following the manufacturer's instruction. An average IL-2 concentration was determined for each variant CD80 IgV-Fc and a fold increase in IL-2 was calculated for each variant compared to wildtype CD80 ECD-Fc protein. The results are provided in FIG. 5A-5D.

As shown in FIG. 5A-5D, co-culturing the exemplary variant CD80 IgV-Fc molecules with T cells and PD-L1 expressing aAPCs, resulted in increased IL-2 production (i.e. PD-L1-dependent CD28 costimulation) compared to WT CD80 ECD-Fc or Fc-only negative control.

### Example 9: Assessment of Efficacy of Variant CD80-Fc Fusion Proteins in a Human PD-L1(Low) Tumor Model

Variant CD80-Fc fusion proteins, including dimer and tandem formats, were tested for effects on tumor growth in the low human PD-L1 (hPD-L1) MC38 mouse tumor model. MC38 cells were transduced with hPD-L1 to achieve low level of hPD-L1 expression as determined by flow cytometry. On Day 0 of the model, 100 μL (1.5×10⁶) of MC38-hPD-L1 low cells were injected by subcutaneous (SC) injection in the right lower/mid flank of each mouse.

Mice were randomized intro groups (n=8) based up on tumor volume on study day 7. Tumors were measured every 3-4 days starting on day 7. Beginning on day 14 mice were also weighed on the day of tumor measurement. Treatments were administered via retro-orbital vein injection (ROI) injection to the groups listed in Table E14 on study days 8 and 11.

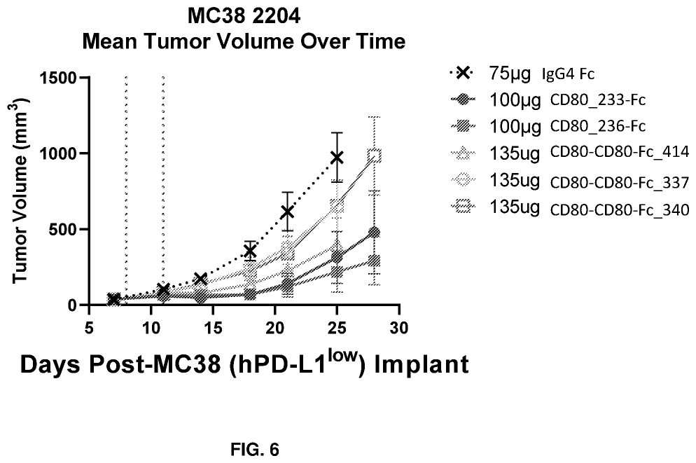

Tumor volumes are shown in FIG. 6 over time. As shown, all tested molecules showed efficacy in reducing tumor volume in this model.

TABLE E14
treatment groups

| | | | | Amount | |
|---|---|---|---|---|---|
| | | | | TA/mouse | |
| Group # | Test Article (TA) | Format | SEQ ID NO | (mg) | n |
| 1 | Fc Control | control | 326 | 0.75 | 8 |
| 2 | CD80_233-Fc | Dimer | 313 | 0.100 | 8 |
| 3 | CD80_236-Fc | Dimer | 316 | 0.100 | 8 |
| 4 | CD80-CD80-Fc_414 | Tandem | 414 | 0.135 | 8 |
| 5 | CD80-CD80-Fc_337 | Tandem | 337 | 0.135 | 8 |
| 6 | CD80-CD80-Fc_340 | Tandem | 340 | 0.135 | 8 |

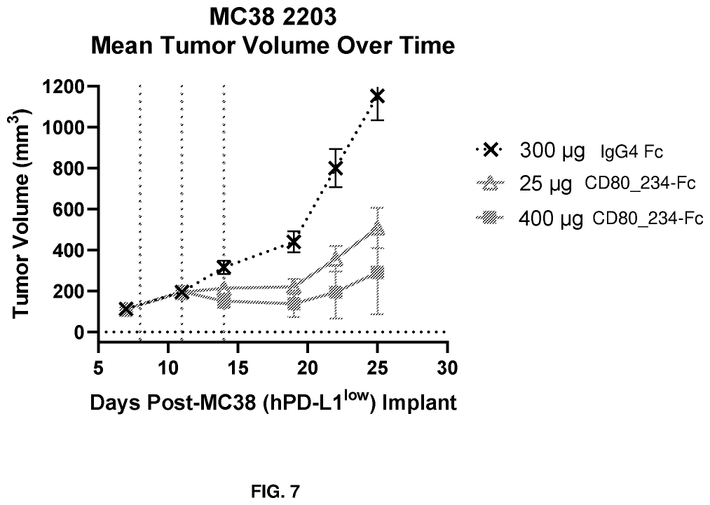

In a similar study, low hPD-L1 MC38 tumor bearing mice generated as described above were administered CD80_234-Fc (SEQ ID NO: 314; V11Y, T28Y, M47L, V68L) at a dose of 25 μg or 400 μg via ROI injection. Tumor volume was monitored over time. Anti-tumor activity of the variant CD80-Fc fusion protein was observed at both administered doses in this model (FIG. 7).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCE TABLE

| SEQ | | |
|---|---|---|
| ID NO | SEQUENCE | ANNOTATION |
| 1 | VIHVTKEVKEVATLSCGHNVSVEELAQTRIYWQKEKKMVLTMMSGDMNIW | mature |
| | PEYKNRTIFDITNNLSIVILALRPSDEGTYECVVLKYEKDAFKREHLAEV | |
| | TLSVKADFPTPSISDFEIPTSNIRRIICSTSGGFPEPHLSWLENGEELNA | |
| | INTTVSQDPETELYAVSSKLDFNMTTNHSFMCLIKYGHLRVNQTFNWNTT | |
| | KQEHFPDNLLPSWAITLISVNGIFVICCLTYCFAPRCRERRRNERLRRES | |
| | VRPV | |
| 2 | VIHVTKEVKEVATLSCGHNVSVEELAQTRIYWQKEKKMVLTMMSGDMNIW | WT ECD |
| | PEYKNRTIFDITNNLSIVILALRPSDEGTYECVVLKYEKDAFKREHLAEV | |
| | TLSVKADFPTPSISDFEIPTSNIRRIICSTSGGFPEPHLSWLENGEELNA | |
| | INTTVSQDPETELYAVSSKLDFNMTTNHSFMCLIKYGHLRVNQTFNWNTT | |
| | KQEHFPDN | |
| 3 | GTTATCCACGTGACCAAGGAAGTGAAAGAATATGCAACGCTGTCCTGTGG | CD80_165 |
| | TCACAATGTTTCTGTTGAAGAGCTGGCACAAACTCGCATCTACTGGCAAA | |
| | AGGAGAAGAAAATGGTGCTGACTATGATGTCTGGGGACGTGAATATATGG | |
| | CCCGAGTACAAGAACCGGACCATCAGTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 4 | GTTATCCACGTGACCAAGGAAGTGAAAGAATATGCAACGCTGTCCTGTGG | CD80_166 |
| | TCACAATGTTTCTGTTGAAGAGCTGGCACAAACTCGCATCTACTGGCAAA | |
| | AGGAGAAGAAAATGGTGCTGACTATGATGTCTGGGGACGTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 5 | GTTATCCACGTGACCAAGGAAGTGAAAGAATATGCAACGCTGTCCTGTGG | CD80_167 |
| | TCACAATGTTTCTGTTGAAGAGCTGGCACAAACTCGCATCTACTGGCAAA | |
| | AGGAGAAGAAAATGGTGCTGACTATTATGTCTGGGGACGTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 6 | GTTATCCACGTGACCAAGGAAGTGAATGGAGTGGCAACGCTGTCCTGTGG | CD80_168 |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTTCTAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 7 | GTTATCCACGTGACCAAGGAAGTGAAAGGAGTGGCAACGCTGTCCTGTGG | CD80_169 |
| | TCACAATGTTTCTGTTGAAGAGCTGGCACAAACTCGCATCTACTGGCAAA | |
| | AGGGGAAGAAAATGGTGCTGACTATGATGTCTGGGGAGGTTAATATATGG | |
| | CCCGAGTACAAGAACCGGACCATCTTTGATATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 8 | GTTATCCACGTGACCAAGGAAGTGAATGGAGTGGCAACGCTGTCCTGTGG | CD80_170 |
| | TCACAATGTTTCTGTTGAAGAGCTGGCACAAACTCGCATCTACTGGCAAA | |
| | AGGGGAAGAAAATGGTGCTGACTATGATGTCTGGGGAGGTTAATATATGG | |
| | CCCGAGTACAAGAACCGGACCATCTTTGATATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 9 | GTTATCCACGTGACCAAGTCTGTGAAAGAAGTGGCAACGCTGTCCTGTGG | CD80_171 |
| | TATCAATTTGTCTGTTGAAGAGCTGAAGCAAACTCGCATCTACTGGCAAA | |
| | AGGAGAAGAAAATGGTGCTGACTATGATGTCTGGGGACCTGAATATATGG | |
| | CCCGAGTACAAGAACCGGACCATTTTTGATATCACTAATAACCTCTCCAT | |
| | TGTGATCCTGAATCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 10 | GTTATCCACGTGACCAAGGAAGTGAGATCTTATGCAACGCTGTCCTGTGG | CD80_172 |
| | TCACAATGTTTCTGTTGAAGAGCTGGCACAAACTCGCATCTACTGGCAAA | |
| | AGGAGAAGAAAATGGTGCTGACTCTGATGTCIGGGGACATGAATATATGG | |
| | CCCGAGTACAAGAACCGGACCATCATGGATATCACTAATAACCTCTCCAT | |
| | TATGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 11 | GTTATCCACGTGACCAAGGAAGTGAAAGAATATGCAACGCTGTCCTGTGG | CD80_173 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 12 | GTTATCCACGTGACCAAGGAAGTGAAACGGGTGGCAACGCTGTCCTGTGG | CD80_174 |
| | TCACAATGTTTCTGTTGAAGAGCTGGCACAAACTCGCATCTACTGGCAAA | |
| | AGGAGAAGAAAATGGTGCTGACTATGATGTCTGGGGACAGGAATATATGG | |
| | CCCGAGTACAAGAACCGGACCATCTTTGATATCACTATTAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 13 | GTTATCCACGTGACCAAGGAAGTGAAATCATTTGCAACGCTGTCCTGTGG | CD80_175 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCTCTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 14 | GTTATCCACGTGACCAAGGAAGTGAAACGGGTGGCAACGCTGTCCTGTGG | CD80_176 |
| | TTATAATGTTTCTGTTGAAGAGCTGGGACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 15 | GTTATCCACGTGACCAAGGAAGTGAAAGGGTGGGCAACGCTGTCCTGTGG | CD80_177 |
| | TCACAATGTTTCTTCTGAAGAGCTGGCACAACCTCGCATCTACTGGCAAA | |
| | AGGAGAAAAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACAAGTGTGTTG | |
| | TTCTGAAGAGGGAAAAAGACGCTTTCAAGCGGGAACACCTGGCTGAAGTG | |
| | ACGTTATCAGTCAAAGCTGAC | |
| 16 | GTTATCCACGTGACCAAGGAAGTGAAAGAATATGCAACGCTGTCCTGTGG | CD80_178 |
| | TCACAATGTTTCTGTTGAAGAGCTGGCACAATATCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGATTGAAAAAGACGCTTTCAAGCGGGAACACCTGGCTGAAGTG | |
| | ACGTTATCAGTCAAAGCTGAC | |
| 17 | GTTATCCACGTGACCAAGGAAGTGAAAGAAGTGGCAACGCTGTCCTGTGG | CD80_179 |
| | TTTCAATGTTTCTGTTGAAGAGCTGGCACAAACTCGCATCTACTGGCAAA | |
| | AGGAGAAGAAAATGGTGCTGACTGGGATGTCTGGGGACATGAATATATGG | |
| | CCCGAGTACAAGAACCGGACCATCTATGATATCACTAATAACCTCTCCAT | |
| | TAATATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 18 | GTTATCCACGTGACCAAGGAAGTGAAAGGGTGGGCAACGCTGTCCTGTGG | CD80_180 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 19 | GTTATCCACGTGACCAAGGAAGTGAGTCGATATGCAACGCTGTCCTGTGG | CD80_181 |
| | TCACAATGTTTCTGTTGAAGAGCTGGCACAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 20 | GTTATCCACGTGACCAAGGAAGTGAAAGGGTGGGCAACGCTGTCCTGTGG | CD80_182 |
| | TCACAATGTTTCTGTTGAAGAGCTGGCACAAACTCGCATCTACTGGCAAA | |
| | AGGGGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGAGGGAAAAAGACGCTTTCAAGCGGGAACACCTGGCTGAAGTG | |
| | ACGTTATCAGTCAAAGCTGAC | |
| 21 | GTTATCCACGTGACCAAGGAAGTGAAAGAATATGCAACGCTGTCCTGTGG | CD80_183 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGAGGGAAAAAGACGCTTTCAAGCGGGAACACCTGGCTGAAGTG | |
| | ACGTTATCAGTCAAAGCTGAC | |
| 22 | GTTATCCACGTGACCAAGGAAGTGAAAGAATATGCAACGCTGTCCTGTGG | CD80_184 |
| | TCACAATGTTTCTGTTGAAGAGCTGCAGCAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGAGGGAAAAAGACGCTTTCAAGCGGGAACACCTGGCTGAAGTG | |
| | ACGTTATCAGTCAAAGCTGAC | |
| 23 | GTTATCCACGTGACCAAGGAAGTGAAAGAATATGCAACGCTGTCCTGTGG | CD80_185 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 24 | GTTATCCACGTGACCAAGAAAGTGAAAGAATGGGCAACGCTGTCCTGTGG | CD80_186 |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTCATAACCTCTCCAT | |
| | TGTGATCCTGGGTCTGCGCCCATCTGACGAGGGCACATACGAGTGTGTTG | |
| | TTCTGAAGAAGGAAAAAGACGCTTTCAAGCGGGAACACCTGGCTGAAGTG | |
| | ACGTTATCAGTCAAAGCTGAC | |
| 25 | GTTATCCACGTGACCAAGGAAGTGAAAGAATATGCAACGCTGTCCTGTGG | CD80_187 |
| | TTATAATGTTTCTGTTGAAGAGCTGGCACAAACTCGCATCTACTGGCAAA | |
| | AGGGGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCAGAAGTATGAAAAAGACGCTTTCAAGCGGGAACACCTGGCTGAAGTG | |
| | ACGTTATCAGTCAAAGCTGAC | |
| 26 | GTTATCCACGTGACCAAGGAAGTGAAAGAAGTGGCAACGCTGTCCTGTGG | CD80_188 |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 27 | GTTATCCACGTGACCAAGGAAGTGAAAGGGTGGGCAACGCTGTCCTGTGG | CD80_189 |
| | TCACAATGTTTCTGTTGAAGAGCTGGCACAAACTCGCATCTACTGGCAAA | |
| | AGGAGAAGAAAATGGTGCTGACTATGATGTCTGGGGACGTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 28 | GTTATCCACGTGACCAAGGAAGTGAAAGAAGTGGCAACGCTGTCCTGTGG | CD80_190 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 29 | GTTATCCACGTGACCAAGGAAGTGAAAGAATATGCAACGCTGTCCTGTGG | CD80_191 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 30 | GTTATCCACGTGACCAAGAATGTGAAAGAAGTGGCAACGCTGTCCTGTGG | CD80_192 |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | CGTTTATCAGTCAAAGCTGAC | |
| 31 | GTTATCCACGTGACCAAGGAAGTGAAAGAATATGCAACGCTGTCCTGTGG | CD80_193 |
| | TCACAATGTTTCTGTTGAAGAGCTGGCACAAACTCGCATCTACTGGCAAA | |
| | AGGAGAAGAAAATGGTGCTGACTTGGATGTCTGGGGACATGAATATATGG | |
| | CCCGAGTACAAGAACCGGACCATCTTTGATATCGCTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 32 | GTTATCCACGTGACCAAGGAAGTGAAAGAATATGCAACGCTGTCCTGTGG | CD80_194 |
| | TCACAATGTTTCTGTTGAAGAGCTGGCACAAACTCGCATCTACTGGCAAA | |
| | AGGAGAAGAAAATGGTGCTGACTTGGATGTCTGGGGACATGAATATATGG | |
| | CCCGAGTACAAGAACCGGACCATCTTTGATATCGCTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 33 | GTTATCCACGTGACCAAGGAAGTGAAAGAATATGCAACGCTGTCCTGTGG | CD80_195 |
| | TCACAATGTTTCTGTTGAAGAGCTGGCACAAACTCGCATCTACTGGCAAA | |
| | AGGAGAAGAAAATGGTGCTGACTTGGATGTCTGGGGACATGAATATATGG | |
| | CCCGAGTACAAGAACCGGACCATCTATGATATCACTAATAACCTCTCCAT | |
| | TAATATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 34 | GTTATCCACGTGACCAAGGAAGTGAAAGAATATGCAACGCTGTCCTGTGG | CD80_196 |
| | TCACAATGTTTCTGTTGAAGAGCTGGCACAAACTCGCATCTACTGGCAAA | |
| | AGGAGAAGAAAATGGTGCTGACTTGGATGTCTGGGGACATGAATATATGG | |
| | CCCAAGTACAAGAACCGGACCATCTTTGATATCGCTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 35 | GTTATCCACGTGACCAAGGAAGTGAAAGAATATGCAACGCTGTCCTGTGG | CD80_197 |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGCAGGAAAAAGACGCTTTCAAGCGGGAACACCTGGCTGAAGTG | |
| | AGGTTATCAGTCAAAGCTGAC | |
| 36 | GTTATCCACGTGACCAAGGAAGTGAAAGAAGTGGCAACGCTGTCGTGTGG | CD80_198 |
| | TTACAATGTTTCTGTTGAAGAGCTGGAACAAACTAGCATCTACTGGCAAA | |
| | AGGATAAGAAAATGGTGCTGACTATGATGTCTGGGGACTTGAATATATGG | |
| | CCCGAGTACAAGAACCGGACCATCTTTGATATCACTAATAACCTCTCCAT | |
| | TATGATCCTGGGTCTGCGCCCATCTGACGGGGGCACATACGAGTGTGTTG | |
| | TTCTGAAGTATGAAAAAGGCGCTTTCAAGCGGGAACACCTGGCTGAAGTG | |
| | ACGTTATCAGTCAAAGCTGAC | |
| 37 | GTTATCCACGTGACCAAGGAAGTGAAAGGGGGGCAACGCTGTCCTGTGG | CD80_199 |
| | TTACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 38 | GTTATCCACGTGACCAAGGAAGTGAATAGAGTGGCAACGCTGTCCTGTGG | CD80_200 |
| | TGTGAATGTTTCTGTTGAAGAGCTGGCACAATATCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGGGCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 39 | GTTATCCACGTGACCAAGGAAGTGAAAGGGGTGGCAACGCTGTCCTGTGG | CD80_201 |
| | TTACAATGTTTCTGTTGAAGAGCTGGCACAATATCGCATCTACTGGCAAA | |
| | AGGAGAAGAAAATGGTGCTGACTATGATGTCTGGGGACTGGAATATATGG | |
| | CCCGAGTACAAGAACCGGACCATCTTTGATATCTCTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 40 | GTTATCCACGTGACCAAGCATGTGAAAGAAGTGGCAACGCTGTCCTGTGG | CD80_202 |
| | TCTCAATATTTCTGTTGAAGAGCTGGCACAATATCGCATCTACTGGCAAA | |
| | AGGAGAAGAAAATGGTGCTGACTATGATGTCTGGGTCCATGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 41 | GTTATCCACGTGACCAAGGAAGTGAATGCATGGGCAACGCTGTCCTGTGG | CD80_203 |
| | TTTCAATGTTTCTGTTGAAGAGCTGGCACAACATCGCATCTACTGGCAAA | |
| | AGGAGAAGAAAATGGTGCTGACTATGATGTCTGGGGACTTGAATATATGG | |
| | CCCGAGTACAAGAACCGGACCATCTTTGATATCGAGAATAACCTCTCCAT | |
| | TGTGATCCTGGCTCTGGACCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 42 | GTTATCCACGTGACCAAGGAAGTGAAAGAAGTGGCAACGCTGTCCTGTGG | CD80_204 |
| | TGTTAATATTTCTGTGGAAGAGCTGGCACAATATCGCATCTACTGGCAAA | |
| | AGGGGAAGAAAATGGTGCTGACTATGATGTCTGGGGACGTGAATATATGG | |
| | CCCGAGTACAAGAACCGGACCATCTTTGATATCACTAATAACCTCTCCAT | |
| | TGTGATCCTGGCTCTGGAGCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 43 | GTTATCCACGTGACCAAGGAAGTGAAAGGTGTGGCAACGCTGTCCTGTGG | CD80_205 |
| | TCACAATGTTTCTGTTGAAGAGCTGGCACAAACTCGCATCTACTGGCAAA | |
| | AGGAGAAGAAAATGGTGCTGACTATGATGTCTGGGAAGATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 44 | GTTATCCACGTGACCAAGGAAGTGAAAGGAGTGGCAACGCTGTCCTGTGG | CD80_206 |
| | TACCAATGTTTCTGTTGAAGAGCTGGCAACAACTCGCATCTACTGGCAAA | |
| | AGGAGAAGAAAATGGTGCTGACTATGATGTCTGGGGAGTTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 45 | GTTATCCACGTGACCAAGGAAGTGAAAGAAGTGGCAACGCTGTCCTGTGG | CD80_207 |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TACTATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | AAGTTATCAGTCAAAGCTGAC | |
| 46 | GTTATCCACGTGACCAAGGAAGTGAAAGAATATGCAACGCTGTCCTGTGG | CD80_208 |
| | TCACAATGTTTCTGTTGAAGAGCTGGCACAAACTCGCATCTACTGGCAAA | |
| | AGGGGAAGAAAATGGTGCTGACTGGTATGTCTGGGGACATGAATATATGG | |
| | CCCGAGTACAAGAACCGGACCATCAGTGATATCACTAATAACCTCTCCAT | |
| | TGTGATCCTGGCTCTGCGCCCATCTGACGAGGGCACATACGAGTGTGTTG | |
| | TTTTGAAGTATGAAAAAGACGCTTTCAAGCGGGAACACCTGGCTGAAGTG | |
| | ACGTTATCAGTCAAAGCTGAC | |
| 47 | GTTATCCACGTGACCAAGGAAGTGAAAGAATATGCAACGCTGTCCTGTGG | CD80_209 |
| | TCACAATGTTTCTGTTGAAGAGCTGGCACAACGTCGCATCTACTGGCAAA | |
| | AGGGGAAGAAAATGGTGCTGACTATGATGTCTGGGGACTTGAATATATGG | |
| | CCCGAGTACAAGAACCGGACCATCAGTGATATCACTAATAACCTCTCCAT | |
| | TGTGATCCTGGCTCTGCGCCCATCTGACGAGGGCACATACGAGTGTGTTG | |
| | TTTTGAAGTATGAAAAAGACGCTTTCAAGCGGGAACACCTGGCTGAAGTG | |
| | ACGTTATCAGTCAAAGCTGAC | |
| 48 | GTTATCCACGTGACCAAGAATGTGAAAGAAGTGGCAACGCTGTCCTGTGG | CD80_210 |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCAGTGATATCACTAATAACCTCTCCAT | |
| | TGTGATCCTGGCTCTGCGCCCATCTGACGAGGGCACATACGAGTGTGTTG | |
| | TTTTGAAGTATGAAAAAGACGCTTTCAAGCGGGAACACCTGGCTGAAGTG | |
| | ACGTTATCAGTCAAAGCTGAC | |
| 49 | GTTATCCACGTGACCAAGGAAGTGAAAGAATATGCAACGCTGTCCTGTGG | CD80_211 |
| | TCACAATGTTTCTGTTGAAGAGCTGGCACAACGTCGCATCTACTGGCAAA | |
| | AGGGGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 50 | GTTATCCACGTGACCAAGGAAGTGCGAGCAGTGGCAACGCTGTCCTGTGG | CD80_212 |
| | TCACAATGTTTCTGTTGAAGAGCTGGCACAAACTCGCATCTACTGGCAAA | |
| | AGGGTAAGAAAATGGTGCTGACTATGATGTCTGGGGACATGAATATATGG | |
| | CCCGAGTACAAGAACCGGACCATCTTTGATATCACTAATAACCTCTCCAT | |
| | TACTATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | AAGTTATCAGTCAAAGCTGAC | |
| 51 | GTTATCCACGTGACCAAGGAAGTGAAAGAATATGCAACGCTGTCCTGTGG | CD80_213 |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TACGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 52 | GTTATCCACGTGACCAAGGAAGTGAAAGGAGTGGCAACGCTGTCCTGTGG | CD80_214 |
| | TCACAATGTTTCTGTTGAAGAGCTGGCACAAACTCGCATCTACTGGCAAA | |
| | AGGGGAAGAAAATGGTGCTGACTATGATGTCTGGGGAGGTTAATATATGG | |
| | CCCGAGTACAAGAACCGGACCATCTTTGATATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | AAGTTATCAGTCAAAGCTGAC | |
| 53 | GTTATCCACGTGACCAAGGAAGTGCGAGCAGTGGCAACGCTGTCCTGTGG | CD80_215 |
| | TCACAATGTTTCTGTTGAAGAGCTGGCACAAACTCGCATCTACTGGCAAA | |
| | AGGGTAAGAAAATGGTGCTGACTATGATGTCTGGGGACATGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 54 | GTTATCCACGTGACCAAGGAAGTGAAAGAATGGGCAACGCTGTCCTGTGG | CD80_216 |
| | TCACAATGTTTCTGTTGAAGAGCTGGCACAATATCGCATCTACTGGCAAA | |
| | AGGAGAAGAAAATGGTGCTGACTATGATGTCTGGGGTTATGAATATATGG | |
| | CCCGAGTACAAGAACCGGACCATCTTTGATATCACTAATAACCTCTCCAT | |
| | TGTGATCCTGGCTCTGGAGCCATCTGACGAGGGCACATACGAGTGTGTTG | |
| | TTCTGAAGTATGAAAAAGACGCTTTAAAGCGGGAACACCTGGCTGAAGTG | |
| | ACGTTATCAGTCAAAGCTGAC | |
| 55 | GTTATCCACGTGACCAAGGAAGTGAATGGAGTGGCAACGCTGTCCTGTGG | CD80_217 |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGAAGGAAAAAGACGCTTTCAAGCGGGAACACCTGGCTGAAGTG | |
| | CAGTTATCAGTCAAAGCTGAC | |
| 56 | GTTATCCACGTGACCAAGGAAGTGAAAGAATGGGCAACGCTGTCCTGTGG | CD80_218 |
| | TCACAATGTTTCTGTTGAAGAGCTGGCACAATATCGCATCTACTGGCAAA | |
| | AGGAGAAGAAAATGGTGCTGACTATGATGTCTGGGGTTATGAATATATGG | |
| | CCCGAGTACAAGAACCGGACCATCTTTGATATCACTAATAACCTCTCCAT | |
| | TACTATCCTGGCTCTGACCCCATCTGACGAGGGCACATACGAGTGTGTTG | |
| | TTCTGAAGAATGAAAAAGACGCTTTCAAGCGGGAACACCTGGCTGAAGTG | |
| | ACGTTATCAGTCAAAGCTGAC | |
| 57 | GTTATCCACGTGACCAAGGAAGTGAAATCATATGCAACGCTGTCCTGTGG | CD80_219 |
| | TCACAATGTTTCTGTTGAAGAGCTGGCACAAACTCGCATCTACTGGCAAA | |
| | AGGAGAAGAAAATGGTGCTGACTCGGATGTCTGGGGACATGAATATATGG | |
| | CCCGAGTACAAGAACCGGACCATCTTTGATATCACTAATAACCTCTCCAT | |
| | TGTGATCCTGGTGCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 58 | GTTATCCACGTGACCAAGGAAGTGAAAGAAGTGGCAACGCTGTCCTGTGG | CD80_220 |
| | TTTTAATGTTTCTGTTGAAGAGCTGGCACAAGTTCGCATCTACTGGCAAA | |
| | AGGAGAAGAAAATGGTGCTGACTATGATGTCTGGGGACCTTAATATATGG | |
| | CCCGAGTACAAGAACCGGACCATCTTTGATATCACTAATAACCTCTCCAT | |
| | TTCGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 59 | GTTATCCACGTGACCAAGGAAGTGAAAGGGGTGGCAACGCTGTCCTGTGG | CD80_221 |
| | TCACAATGTTTCTGTTGAAGAGCTGTCACAATATCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 60 | GTTATCCACGTGACCAAGGAAGTGAAAGAAGTGGCAACGCTGTCCTGTGG | CD80_222 |
| | TCACAATGTTTCTGTTGAAGAGCTGGCACAAACTCGCATCTACTGGCAAA | |
| | AGGATAAGAAAATGGTGCTGACTATGATGTCTGGGCAGATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 61 | GTTATCCACGTGACCAAGGAAGTGAAATCGGTGGCAACGCTGTCCTGTGG | CD80_223 |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TATGATCCTGGCTCTGCGCCCATCTGACGAGGGCACATACGAGTGTGTTG | |
| | TTCTGAAGCCTGAAAAAGACGCTTTCAAGCGGGAACACCTGGCTGAAGTG | |
| | ACGTTATCAGTCAAAGCTGAC | |
| 62 | GTTATCCACGTGACCAAGGAAGTGAATGAATGGGCAACGCTGTCCTGTGG | CD80_224 |
| | TCACAATGTTTCTGTTGAAGAGCTGGCACAAACTCGCATCTACTGGCAAA | |
| | AGGAGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TACTATCCTGGCTCTGACCCCATCTGACGAGGGCACATACGAGTGTGTTG | |
| | TTCTGAAGAATGAAAAAGACGCTTTCAAGCGGGAACACCTGGCTGAAGTG | |
| | ACGTTATCAGTCAAAGCTGAC | |
| 63 | GTTATCCACGTGACCAAGGAAGTGAAAGAATATGCAACGCTGTCCTGTGG | CD80_225 |
| | TCACAATGTTTCTGTTGAAGAGCTGGCACAAACTCGCATCTACTGGCAAA | |
| | AGGAGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TACTATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 64 | GTTATCCACGTGACCAAGGAAGTGAGAGAAGTGGCAACGCTGTCCTGTGG | CD80_226 |
| | TCACAATGTTTCTGTTGAAGAGCTGACGCAATATCGCATCTACTGGCAAA | |
| | AGGCGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 65 | GTTATCCACGTGACCAAGGAAGTGAAAGAATGGGCAACGCTGTCCTGTGG | CD80_227 |
| | TCACAATGTTTCTGTTGAAGAGCTGGCACAACATCGCATCTACTGGCAAA | |
| | AGGAGAAGAAAATGGTGCTGACTATGATGTCTGGGCAGATGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 66 | GTTATCCACGTGACCAAGCAAGTGAAAGAATATGCAACGCTGTCCTGTGG | CD80_228 |
| | TCACAATGTTTCTGTTGAAGAGCTGGCACAAACTCACATCTACTGGCAAA | |
| | AGGAGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TACTATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 67 | GTTATCCACGTGACCAAGGAAGTGAAAGAATATGCAACGCTGTCCTGTGG | CD80_229 |
| | TCACAATGTTTCTGTTGAAGAGCTGGCACAAACTCGCATCTACTGGCAAA | |
| | AGGAGAAGAAAATGGTGCTGACTATGATGTCTGGGGACGTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGAC | |
| 68 | GTTATCCACGTGACCAAGGAAGTGAAAGGAGTGGCAACGCTGTCCTGTGG | CD80_230 |
| | TCACAATGTTTCTGTTGAAGAGCTGGCATTTACTCGCATCTACTGGCAAA | |
| | AGGAGAAGAAAATGGTGCTGACTATGATGTCTGGGAACATGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTTTGAAGTATGAAAAAGGTGCTTTCAAGCGGGAACACCTGGCTGAAGTG | |
| | ACGTTATCAGTCAAAGCTGAC | |
| 69 | GTTATCCACGTGACCAAGGAAGTGAAAGAATGGGCAACGCTGTCCTGTGG | CD80_231 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 70 | GTTATCCACGTGACCAAGGAAGTGAAAGAATTTGCAACGCTGTCCTGTGG | CD80_232 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 71 | GTTATCCACGTGACCAAGGAAGTGAAAGAATATGCAACGCTGTCCTGTGG | CD80_233 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 72 | GTTATCCACGTGACCAAGGAAGTGAAAGAATATGCAACGCTGTCCTGTGG | CD80_234 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TCTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 73 | GTTATCCACGTGACCAAGGAAGTGAAAGAATATGCAACGCTGTCCTGTGG | CD80_235 |
| | TCACAATGTTTCTGTTGAAGAGCTGGCACAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 74 | GTTATCCACGTGACCAAGGAAGTGAAAGAATATGCAACGCTGTCCTGTGG | CD80_236 |
| | TCACAATGTTTCTGTTGAAGAGCTGGCACAATACCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 75 | GTTATCCACGTGACCAAGGAAGTGAAAGAATATGCAACGCTGTCCTGTGG | CD80_237 |
| | TCACAATGTTTCTGTTGAAGAGCTGGCACAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 76 | GTTATCCACGTGACCAAGGAAGTGAAAGAATATGCAACGCTGTCCTGTGG | CD80_238 |
| | TCACAATGTTTCTGTTGAAGAGCTGGCACAAACTCGCATCTACTGGCAAA | |
| | AGGAGAAGAAAATGGTGCTGACTATGATGTCTGGGGAGGTGAATATATGG | |
| | CCCGAGTACAAGAACCGGACCATCTTTGACATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 77 | GTTATCCACGTGACCAAGGAAGTGAAAGAATATGCAACGCTGTCCTGTGG | CD80_239 |
| | TCACAATGTTTCTGTTGAAGAGCTGGCACAATACCGCATCTACTGGCAAA | |
| | AGGAGAAGAAAATGGTGCTGACTATGATGTCTGGGGAGGTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 78 | GTTATCCACGTGACCAAGGAAGTGAAAGAATATGCAACGCTGTCCTGTGG | CD80_240 |
| | TCACAATGTTTCTGTTGAAGAGCTGGCACAATACCGCATCTACTGGCAAA | |
| | AGGAGAAGAAAATGGTGCTGACTATGATGTCTGGGGAGGTGAATATATGG | |
| | CCCGAGTACAAGAACCGGACCATCTTTGACATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 79 | GTTATCCACGTGACCAAGGAAGTGAAATCCTTTGCAACGCTGTCCTGTGG | CD80_241 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 80 | GTTATCCACGTGACCAAGGAAGTGAAAGAATTTGCAACGCTGTCCTGTGG | CD80_242 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCTCCAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 81 | GTTATCCACGTGACCAAGGAAGTGAAAGAATATGCAACGCTGTCCTGTGG | CD80_243 |
| | TCACAATGTTTCTGTTGAAGAGCTGGCACAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGGTCTGCGCCCATCTGACGAGGGCACATACGAGTGTGTTG | |
| | TTCTGAAGAGAGAAAAAGACGCTTTCAAGCGGGAACACCTGGCTGAAGTG | |
| | ACGTTATCAGTCAAAGCTGAC | |
| 82 | GTTATCCACGTGACCAAGGAAGTGAAAGAATATGCAACGCTGTCCTGTGG | CD80_244 |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGAGAGAAAAAGACGCTTTCAAGCGGGAACACCTGGCTGAAGTG | |
| | ACGTTATCAGTCAAAGCTGAC | |
| 83 | GTTATCCACGTGACCAAGGAAGTGAAAGAATATGCAACGCTGTCCTGTGG | CD80_165-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACGTGAATATATGG | |
| | CCCGAGTACAAGAACCGGACCATCAGTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 84 | GTTATCCACGTGACCAAGGAAGTGAAAGAATATGCAACGCTGTCCTGTGG | CD80_166-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACGTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 85 | GTTATCCACGTGACCAAGGAAGTGAAAGAATATGCAACGCTGTCCTGTGG | CD80_167-Fc |
| | TCACAATGTTTCTGTTGAAGAGCTGGCACAAACTCGCATCTACTGGCAAA | |
| | AGGAGAAGAAAATGGTGCTGACTATTATGTCTGGGGACGTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 86 | GTTATCCACGTGACCAAGGAAGTGAATGGAGTGGCAACGCTGTCCTGTGG | CD80_168-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTTCTAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 87 | GTTATCCACGTGACCAAGGAAGTGAAAGGAGTGGCAACGCTGTCCTGTGG | CD80_169-Fc |
| | TCACAATGTTTCTGTTGAAGAGCTGGCACAAACTCGCATCTACTGGCAAA | |
| | AGGGGAAGAAAATGGTGCTGACTATGATGTCTGGGGAGGTTAATATATGG | |
| | CCCGAGTACAAGAACCGGACCATCTTTGATATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 88 | GTTATCCACGTGACCAAGGAAGTGAATGGAGTGGCAACGCTGTCCTGTGG | CD80_170-Fc |
| | TCACAATGTTTCTGTTGAAGAGCTGGCACAAACTCGCATCTACTGGCAAA | |
| | AGGGGAAGAAAATGGTGCTGACTATGATGTCTGGGGAGGTTAATATATGG | |
| | CCCGAGTACAAGAACCGGACCATCTTTGATATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 89 | GTTATCCACGTGACCAAGTCTGTGAAAGAAGTGGCAACGCTGTCCTGTGG | CD80_171-Fc |
| | TATCAATTTGTCTGTTGAAGAGCTGAAGCAAACTCGCATCTACTGGCAAA | |
| | AGGAGAAGAAAATGGTGCTGACTATGATGTCTGGGGACCTGAATATATGG | |
| | CCCGAGTACAAGAACCGGACCATTTTTGATATCACTAATAACCTCTCCAT | |
| | TGTGATCCTGAATCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 90 | GTTATCCACGTGACCAAGGAAGTGAGATCTTATGCAACGCTGTCCTGTGG | CD80_172-Fc |
| | TCACAATGTTTCTGTTGAAGAGCTGGCACAAACTCGCATCTACTGGCAAA | |
| | AGGAGAAGAAAATGGTGCTGACTCTGATGTCTGGGGACATGAATATATGG | |
| | CCCGAGTACAAGAACCGGACCATCATGGATATCACTAATAACCTCTCCAT | |
| | TATGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 91 | GTTATCCACGTGACCAAGGAAGTGAAAGAATATGCAACGCTGTCCTGTGG | CD80_173-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 92 | GTTATCCACGTGACCAAGGAAGTGAAACGGGTGGCAACGCTGTCCTGTGG | CD80_174-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACAGGAATATATGG | |
| | CCCGAGTACAAGAACCGGACCATCTTTGATATCACTATTAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 93 | GTTATCCACGTGACCAAGGAAGTGAAATCATTTGCAACGCTGTCCTGTGG | CD80_175-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCTCTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 94 | GTTATCCACGTGACCAAGGAAGTGAAACGGGTGGCAACGCTGTCCTGTGG | CD80_176-Fc |
| | TTATAATGTTTCTGTTGAAGAGCTGGGACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 95 | GTTATCCACGTGACCAAGGAAGTGAAAGGGTGGGCAACGCTGTCCTGTGG | CD80_177-Fc |
| | TCACAATGTTTCTTCTGAAGAGCTGGCACAACCTCGCATCTACTGGCAAA | |
| | AGGAGAAAAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACAAGTGTGTTG | |
| | TTCTGAAGAGGGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 96 | GTTATCCACGTGACCAAGGAAGTGAAAGAATATGCAACGCTGTCCTGTGG | CD80_178-Fc |
| | TCACAATGTTTCTGTTGAAGAGCTGGCACAATATCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGATTGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 97 | GTTATCCACGTGACCAAGGAAGTGAAAGAAGTGGCAACGCTGTCCTGTGG | CD80_179-Fc |
| | TTTCAATGTTTCTGTTGAAGAGCTGGCACAAACTCGCATCTACTGGCAAA | |
| | AGGAGAAGAAAATGGTGCTGACTGGGATGTCTGGGGACATGAATATATGG | |
| | CCCGAGTACAAGAACCGGACCATCTATGATATCACTAATAACCTCTCCAT | |
| | TAATATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 98 | GTTATCCACGTGACCAAGGAAGTGAAAGGGTGGGCAACGCTGTCCTGTGG | CD80_180-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGIGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 99 | GTTATCCACGTGACCAAGGAAGTGAGTCGATATGCAACGCTGTCCTGTGG | CD80_181-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 100 | GTTATCCACGTGACCAAGGAAGTGAAAGGGTGGGCAACGCTGTCCTGTGG | CD80_182-Fc |
| | TCACAATGTTTCTGTTGAAGAGCTGGCACAAACTCGCATCTACTGGCAAA | |
| | AGGGGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGAGGGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 101 | GTTATCCACGTGACCAAGGAAGTGAAAGAATATGCAACGCTGTCCTGTGG | CD80_183-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGAGGGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 102 | GTTATCCACGTGACCAAGGAAGTGAAAGAATATGCAACGCTGTCCTGTGG | CD80_184-Fc |
| | TCACAATGTTTCTGTTGAAGAGCTGCAGCAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGAGGGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 103 | GTTATCCACGTGACCAAGGAAGTGAAAGAATATGCAACGCTGTCCTGTGG | CD80_185-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 104 | GTTATCCACGTGACCAAGAAAGTGAAAGAATGGGCAACGCTGTCCTGTGG | CD80_186-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTCATAACCTCTCCAT | |
| | TGTGATCCTGGGTCTGCGCCCATCTGACGAGGGCACATACGAGTGTGTTG | |
| | TTCTGAAGAAGGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 105 | GTTATCCACGTGACCAAGGAAGTGAAAGAATATGCAACGCTGTCCTGTGG | CD80_187-Fc |
| | TTATAATGTTTCTGTTGAAGAGCTGGCACAAACTCGCATCTACTGGCAAA | |
| | AGGGGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 106 | GTTATCCACGTGACCAAGGAAGTGAAAGAAGTGGCAACGCTGTCCTGTGG | CD80_188-Fc |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 107 | GTTATCCACGTGACCAAGGAAGTGAAAGGGGGGCAACGCTGTCCTGTGG | CD80_189-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACGTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 108 | GTTATCCACGTGACCAAGGAAGTGAAAGAAGTGGCAACGCTGTCCTGTGG | CD80_190-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 109 | GTTATCCACGTGACCAAGGAAGTGAAAGAATATGCAACGCTGTCCTGTGG | CD80_191-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 110 | GTTATCCACGTGACCAAGAATGTGAAAGAAGTGGCAACGCTGTCCTGTGG | CD80_192-Fc |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | CGTTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 111 | GTTATCCACGTGACCAAGGAAGTGAAAGAATATGCAACGCTGTCCTGTGG | CD80_193-Fc |
| | TCACAATGTTTCTGTTGAAGAGCTGGCACAAACTCGCATCTACTGGCAAA | |
| | AGGAGAAGAAAATGGTGCTGACTTGGATGTCTGGGGACATGAATATATGG | |
| | CCCGAGTACAAGAACCGGACCATCTTTGATATCGCTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 112 | GTTATCCACGTGACCAAGGAAGTGAAAGAATATGCAACGCTGTCCTGTGG | CD80_194-Fc |
| | TCACAATGTTTCTGTTGAAGAGCTGGCACAAACTCGCATCTACTGGCAAA | |
| | AGGAGAAGAAAATGGTGCTGACTTGGATGTCTGGGGACATGAATATATGG | |
| | CCCGAGTACAAGAACCGGACCATCTTTGATATCGCTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 113 | GTTATCCACGTGACCAAGGAAGTGAAAGAATATGCAACGCTGTCCTGTGG | CD80_195-Fc |
| | TCACAATGTTTCTGTTGAAGAGCTGGCACAAACTCGCATCTACTGGCAAA | |
| | AGGAGAAGAAAATGGTGCTGACTTGGATGTCTGGGGACATGAATATATGG | |
| | CCCGAGTACAAGAACCGGACCATCTATGATATCACTAATAACCTCTCCAT | |
| | TAATATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGITCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 114 | GTTATCCACGTGACCAAGGAAGTGAAAGAATATGCAACGCTGTCCTGTGG | CD80_196-Fc |
| | TCACAATGTTTCTGTTGAAGAGCTGGCACAAACTCGCATCTACTGGCAAA | |
| | AGGAGAAGAAAATGGTGCTGACTTGGATGTCTGGGGACATGAATATATGG | |
| | CCCAAGTACAAGAACCGGACCATCTTTGATATCGCTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 115 | GTTATCCACGTGACCAAGGAAGTGAAAGAATATGCAACGCTGTCCTGTGG | CD80_197-Fc |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGCAGGAAAAAGACGCTTTCAAGCGGGAACACCTGGCTGAAGTG | |
| | AGGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 116 | GTTATCCACGTGACCAAGGAAGTGAAAGAAGTGGCAACGCTGTCGTGTGG | CD80_198-Fc |
| | TTACAATGTTTCTGTTGAAGAGCTGGAACAAACTAGCATCTACTGGCAAA | |
| | AGGATAAGAAAATGGTGCTGACTATGATGTCTGGGGACTTGAATATATGG | |
| | CCCGAGTACAAGAACCGGACCATCTTTGATATCACTAATAACCTCTCCAT | |
| | TATGATCCTGGGTCTGCGCCCATCTGACGGGGGCACATACGAGTGTGTTG | |
| | TTCTGAAGTATGAAAAAGGCGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 117 | GTTATCCACGTGACCAAGGAAGTGAAAGGGGGGCAACGCTGTCCTGTGG | CD80_199-Fc |
| | TTACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 118 | GTTATCCACGTGACCAAGGAAGTGAATAGAGTGGCAACGCTGTCCTGTGG | CD80_200-Fc |
| | TGTGAATGTTTCTGTTGAAGAGCTGGCACAATATCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGGGCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 119 | GTTATCCACGTGACCAAGGAAGTGAAAGGGGTGGCAACGCTGTCCTGTGG | CD80_201-Fc |
| | TTACAATGTTTCTGTTGAAGAGCTGGCACAATATCGCATCTACTGGCAAA | |
| | AGGAGAAGAAAATGGTGCTGACTATGATGTCTGGGGACTGGAATATATGG | |
| | CCCGAGTACAAGAACCGGACCATCTTTGATATCTCTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 120 | GTTATCCACGTGACCAAGCATGTGAAAGAAGTGGCAACGCTGTCCTGTGG | CD80_202-Fc |
| | TCTCAATATTTCTGTTGAAGAGCTGGCACAATATCGCATCTACTGGCAAA | |
| | AGGAGAAGAAAATGGTGCTGACTATGATGTCTGGGTCCATGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 121 | GTTATCCACGTGACCAAGGAAGTGAATGCATGGGCAACGCTGTCCTGTGG | CD80_203-Fc |
| | TTTCAATGTTTCTGTTGAAGAGCTGGCACAACATCGCATCTACTGGCAAA | |
| | AGGAGAAGAAAATGGTGCTGACTATGATGTCTGGGGACTTGAATATATGG | |
| | CCCGAGTACAAGAACCGGACCATCTTTGATATCGAGAATAACCTCTCCAT | |
| | TGTGATCCTGGCTCTGGACCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 122 | GTTATCCACGTGACCAAGGAAGTGAAAGAAGTGGCAACGCTGTCCTGTGG | CD80_204-Fc |
| | TGTTAATATTTCTGTGGAAGAGCTGGCACAATATCGCATCTACTGGCAAA | |
| | AGGGGAAGAAAATGGTGCTGACTATGATGTCTGGGGACGTGAATATATGG | |
| | CCCGAGTACAAGAACCGGACCATCTTTGATATCACTAATAACCTCTCCAT | |
| | TGTGATCCTGGCTCTGGAGCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 123 | GTTATCCACGTGACCAAGGAAGTGAAAGGTGTGGCAACGCTGTCCTGTGG | CD80_205-Fc |
| | TCACAATGTTTCTGTTGAAGAGCTGGCACAAACTCGCATCTACTGGCAAA | |
| | AGGAGAAGAAAATGGTGCTGACTATGATGTCTGGGAAGATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 124 | GTTATCCACGTGACCAAGGAAGTGAAAGGAGTGGCAACGCTGTCCTGTGG | CD80_206-Fc |
| | TACCAATGTTTCTGTTGAAGAGCTGGCAACAACTCGCATCTACTGGCAAA | |
| | AGGAGAAGAAAATGGTGCTGACTATGATGTCTGGGGAGTTGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 125 | GTTATCCACGTGACCAAGGAAGTGAAAGAAGTGGCAACGCTGTCCTGTGG | CD80_207-Fc |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCTTTGATATCACTAATAACCTCTCCAT | |
| | TACTATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | AAGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 126 | GTTATCCACGTGACCAAGGAAGTGAAAGAATATGCAACGCTGTCCTGTGG | CD80_208-Fc |
| | TCACAATGTTTCTGTTGAAGAGCTGGCACAAACTCGCATCTACTGGCAAA | |
| | AGGGGAAGAAAATGGTGCTGACTGGTATGTCTGGGGACATGAATATATGG | |
| | CCCGAGTACAAGAACCGGACCATCAGTGATATCACTAATAACCTCTCCAT | |
| | TGTGATCCTGGCTCTGCGCCCATCTGACGAGGGCACATACGAGTGTGTTG | |
| | TTTTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 127 | GTTATCCACGTGACCAAGGAAGTGAAAGAATATGCAACGCTGTCCTGTGG | CD80_209-Fc |
| | TCACAATGTTTCTGTTGAAGAGCTGGCACAACGTCGCATCTACTGGCAAA | |
| | AGGGGAAGAAAATGGTGCTGACTATGATGTCTGGGGACTTGAATATATGG | |
| | CCCGAGTACAAGAACCGGACCATCAGTGATATCACTAATAACCTCTCCAT | |
| | TGTGATCCTGGCTCTGCGCCCATCTGACGAGGGCACATACGAGTGTGTTG | |
| | TTTTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 128 | GTTATCCACGTGACCAAGAATGTGAAAGAAGTGGCAACGCTGTCCTGTGG | CD80_210-Fc |
| | TCACAATGTTTCTGTTGAAGAGCTGGCACAAACTCGCATCTACTGGCAAA | |
| | AGGATAAGAAAATGGTGCTGACTATGATGTCTGGGGACATGAATATATGG | |
| | CCCGAGTACAAGAACCGGACCATCAGTGATATCACTAATAACCTCTCCAT | |
| | TGTGATCCTGGCTCTGCGCCCATCTGACGAGGGCACATACGAGTGTGTTG | |
| | TTTTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 129 | GTTATCCACGTGACCAAGGAAGTGAAAGAATATGCAACGCTGTCCTGTGG | CD80_211-Fc |
| | TCACAATGTTTCTGTTGAAGAGCTGGCACAACGTCGCATCTACTGGCAAA | |
| | AGGGGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 130 | GTTATCCACGTGACCAAGGAAGTGCGAGCAGTGGCAACGCTGTCCTGTGG | CD80_212-Fc |
| | TCACAATGTTTCTGTTGAAGAGCTGGCACAAACTCGCATCTACTGGCAAA | |
| | AGGGTAAGAAAATGGTGCTGACTATGATGTCTGGGGACATGAATATATGG | |
| | CCCGAGTACAAGAACCGGACCATCTTTGATATCACTAATAACCTCTCCAT | |
| | TACTATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | AAGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 131 | GTTATCCACGTGACCAAGGAAGTGAAAGAATATGCAACGCTGTCCTGTGG | CD80_213-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TACGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 132 | GTTATCCACGTGACCAAGGAAGTGAAAGGAGTGGCAACGCTGTCCTGTGG | CD80_214-Fc |
| | TCACAATGTTTCTGTTGAAGAGCTGGCACAAACTCGCATCTACTGGCAAA | |
| | AGGGGAAGAAAATGGTGCTGACTATGATGTCTGGGGAGGTTAATATATGG | |
| | CCCGAGTACAAGAACCGGACCATCTTTGATATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | AAGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 133 | GTTATCCACGTGACCAAGGAAGTGCGAGCAGTGGCAACGCTGTCCTGTGG | CD80_215-Fc |
| | TCACAATGTTTCTGTTGAAGAGCTGGCACAAACTCGCATCTACTGGCAAA | |
| | AGGGTAAGAAAATGGTGCTGACTATGATGTCTGGGGACATGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 134 | GTTATCCACGTGACCAAGGAAGTGAAAGAATGGGCAACGCTGTCCTGTGG | CD80_216-Fc |
| | TCACAATGTTTCTGTTGAAGAGCTGGCACAATATCGCATCTACTGGCAAA | |
| | AGGAGAAGAAAATGGTGCTGACTATGATGTCTGGGGTTATGAATATATGG | |
| | CCCGAGTACAAGAACCGGACCATCTTTGATATCACTAATAACCTCTCCAT | |
| | TGTGATCCTGGCTCTGGAGCCATCTGACGAGGGCACATACGAGTGTGTTG | |
| | TTCTGAAGTATGAAAAAGACGCTTTAAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 135 | GTTATCCACGTGACCAAGGAAGTGAATGGAGTGGCAACGCTGTCCTGTGG | CD80_217-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGAAGGAAAAAGACGCTTTCAAGCGGGAACACCTGGCTGAAGTG | |
| | CAGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 136 | GTTATCCACGTGACCAAGGAAGTGAAAGAATGGGCAACGCTGTCCTGTGG | CD80_218-Fc |
| | TCACAATGTTTCTGTTGAAGAGCTGGCACAATATCGCATCTACTGGCAAA | |
| | AGGAGAAGAAAATGGTGCTGACTATGATGTCTGGGGTTATGAATATATGG | |
| | CCCGAGTACAAGAACCGGACCATCTTTGATATCACTAATAACCTCTCCAT | |
| | TACTATCCTGGCTCTGACCCCATCTGACGAGGGCACATACGAGTGTGTTG | |
| | TTCTGAAGAATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 137 | GTTATCCACGTGACCAAGGAAGTGAAATCATATGCAACGCTGTCCTGTGG | CD80_219-Fc |
| | TCACAATGTTTCTGTTGAAGAGCTGGCACAAACTCGCATCTACTGGCAAA | |
| | AGGAGAAGAAAATGGTGCTGACTCGGATGTCTGGGGACATGAATATATGG | |
| | CCCGAGTACAAGAACCGGACCATCTTTGATATCACTAATAACCTCTCCAT | |
| | TGTGATCCTGGTGCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 138 | GTTATCCACGTGACCAAGGAAGTGAAAGAAGTGGCAACGCTGTCCTGTGG | CD80_220-Fc |
| | TTTTAATGTTTCTGTTGAAGAGCTGGCACAAGTTCGCATCTACTGGCAAA | |
| | AGGAGAAGAAAATGGTGCTGACTATGATGTCTGGGGACCTTAATATATGG | |
| | CCCGAGTACAAGAACCGGACCATCTTTGATATCACTAATAACCTCTCCAT | |
| | TTCGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 139 | GTTATCCACGTGACCAAGGAAGTGAAAGGGGTGGCAACGCTGTCCTGTGG | CD80_221-Fc |
| | TCACAATGTTTCTGTTGAAGAGCTGTCACAATATCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 140 | GTTATCCACGTGACCAAGGAAGTGAAAGAAGTGGCAACGCTGTCCTGTGG | CD80_222-Fc |
| | TCACAATGTTTCTGTTGAAGAGCTGGCACAAACTCGCATCTACTGGCAAA | |
| | AGGATAAGAAAATGGTGCTGACTATGATGTCTGGGCAGATGAATATATGG | |
| | CCCGAGTACAAGAACCGGACCATCTTTGATATCACTAATAACCTCTCCAT | |
| | TGTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 141 | GTTATCCACGTGACCAAGGAAGTGAAATCGGTGGCAACGCTGTCCTGTGG | CD80_223-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACATGAATATATGG | |
| | CCCGAGTACAAGAACCGGACCATCTTTGATATCACTAATAACCTCTCCAT | |
| | TATGATCCTGGCTCTGCGCCCATCTGACGAGGGCACATACGAGTGTGTTG | |
| | TTCTGAAGCCTGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 142 | GTTATCCACGTGACCAAGGAAGTGAATGAATGGGCAACGCTGTCCTGTGG | CD80_224-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TACTATCCTGGCTCTGACCCCATCTGACGAGGGCACATACGAGTGTGTTG | |
| | TTCTGAAGAATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 143 | GTTATCCACGTGACCAAGGAAGTGAAAGAATATGCAACGCTGTCCTGTGG | CD80_225-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TACTATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 144 | GTTATCCACGTGACCAAGGAAGTGAGAGAAGTGGCAACGCTGTCCTGTGG | CD80_226-Fc |
| | TCACAATGTTTCTGTTGAAGAGCTGACGCAATATCGCATCTACTGGCAAA | |
| | AGGCGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 145 | GTTATCCACGTGACCAAGGAAGTGAAAGAATGGGCAACGCTGTCCTGTGG | CD80_227-Fc |
| | TCACAATGTTTCTGTTGAAGAGCTGGCACAACATCGCATCTACTGGCAAA | |
| | AGGAGAAGAAAATGGTGCTGACTATGATGTCTGGGCAGATGAATATATGG | |
| | CCCGAGTACAAGAACCGGACCATCTTTGATATCACTAATAACCTCTCCAT | |
| | TTTGATCCTGGCTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 146 | GTTATCCACGTGACCAAGCAAGTGAAAGAATATGCAACGCTGTCCTGTGG | CD80_228-Fc |
| | TCACAATGTTTCTGTTGAAGAGCTGGCACAAACTCACATCTACTGGCAAA | |
| | AGGAGAAGAAAATGGTGCTGACTATGATGTCTGGGGACTTGAATATATGG | |
| | CCCGAGTACAAGAACCGGACCATCTTTGATATCACTAATAACCTCTCCAT | |
| | TACTATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 147 | GTTATCCACGTGACCAAGGAAGTGAAAGAATATGCAACGCTGTCCTGTGG | CD80_229-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACGTGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTGAGAAGTATGAAAAAGACGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 148 | GTTATCCACGTGACCAAGGAAGTGAAAGGAGTGGCAACGCTGTCCTGTGG | CD80_230-Fc |
| | TCACAATGTTTCTGTTGAAGAGCTGGCATTTACTCGCATCTACTGGCAAA | |
| | AGGAGAAGAAAATGGTGCTGACTATGATGTCTGGGAACATGAATATATGG | |
| | CCCGAGTACAAGAACCGGACCATCTTTGATATCACTAATAACCTCTCCAT | |
| | TGTGATCCTGGGTCTGCGCCCATCTGACGAGGGCACATACGAGTGTGTTG | |
| | TTTTGAAGTATGAAAAAGGTGCTTTCAAGCGGGAACACCTGGCTGAAGTG | |
| | ACGTTATCAGTCAAAGCTGACGGATCCGGTGGAGGAGGGTCAGAGTCCAA | |
| | ATATGGTCCCCCATGCCCACCATGCCCAGCACCTGAGTTCCTGGGGGGAC | |
| | CATCAGTCTTCCTGTTCCCCCCAAAACCCAAGGACACTCTCATGATCTCC | |
| | CGGACCCCTGAGGTCACGTGCGTGGTGGTGGACGTGAGCCAGGAAGACCC | |
| | CGAGGTCCAGTTCAACTGGTACGTGGATGGCGTGGAGGTGCATAATGCCA | |
| | AGACAAAGCCGCGGGAGGAGCAGTTCAACAGCACGTACCGTGTGGTCAGC | |
| | GTCCTCACCGTCCTGCACCAGGACTGGCTGAACGGCAAGGAGTACAAGTG | |
| | CAAGGTCTCCAACAAAGGCCTCCCGTCCTCCATCGAGAAAACCATCTCCA | |
| | AAGCCAAAGGGCAGCCCCGAGAGCCACAGGTGTACACCCTGCCCCCATCC | |
| | CAGGAGGAGATGACCAAGAACCAGGTCAGCCTGACCTGCCTGGTCAAAGG | |
| | CTTCTACCCCAGCGACATCGCCGTGGAGTGGGAGAGCAATGGGCAGCCGG | |
| | AGAACAACTACAAGACCACGCCTCCCGTGCTGGACTCCGACGGCTCCTTC | |
| | TTCCTCTACAGCAGGCTAACCGTGGACAAGAGCAGGTGGCAGGAGGGGAA | |
| | TGTCTTCTCATGCTCCGTGATGCATGAGGCTCTGCACAACCACTACACAC | |
| | AGAAGAGCCTCTCCCTGTCTCTGGGT | |
| 149 | GTTATCCACGTGACCAAGGAAGTGAAAGAATGGGCAACGCTGTCCTGTGG | CD80_231-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 150 | GTTATCCACGTGACCAAGGAAGTGAAAGAATTTGCAACGCTGTCCTGTGG | CD80_232-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 151 | GTTATCCACGTGACCAAGGAAGTGAAAGAATATGCAACGCTGTCCTGTGG | CD80_233-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 152 | GTTATCCACGTGACCAAGGAAGTGAAAGAATATGCAACGCTGTCCTGTGG | CD80_234-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TCTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 153 | GTTATCCACGTGACCAAGGAAGTGAAAGAATATGCAACGCTGTCCTGTGG | CD80_235-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 154 | GTTATCCACGTGACCAAGGAAGTGAAAGAATATGCAACGCTGTCCTGTGG | CD80_236-Fc |
| | TCACAATGTTTCTGTTGAAGAGCTGGCACAATACCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGGTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 155 | GTTATCCACGTGACCAAGGAAGTGAAAGAATATGCAACGCTGTCCTGTGG | CD80_237-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 156 | GTTATCCACGTGACCAAGGAAGTGAAAGAATATGCAACGCTGTCCTGTGG | CD80_238-Fc |
| | TCACAATGTTTCTGTTGAAGAGCTGGCACAAACTCGCATCTACTGGCAAA | |
| | AGGAGAAGAAAATGGTGCTGACTATGATGTCTGGGGAGGTGAATATATGG | |
| | CCCGAGTACAAGAACCGGACCATCTTTGACATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 157 | GTTATCCACGTGACCAAGGAAGTGAAAGAATATGCAACGCTGTCCTGTGG | CD80_239-Fc |
| | TCACAATGTTTCTGTTGAAGAGCTGGCACAATACCGCATCTACTGGCAAA | |
| | AGGAGAAGAAAATGGTGCTGACTATGATGTCTGGGGAGGTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 158 | GTTATCCACGTGACCAAGGAAGTGAAAGAATATGCAACGCTGTCCTGTGG | CD80_240-Fc |
| | TCACAATGTTTCTGTTGAAGAGCTGGCACAATACCGCATCTACTGGCAAA | |
| | AGGAGAAGAAAATGGTGCTGACTATGATGTCTGGGGAGGTGAATATATGG | |
| | CCCGAGTACAAGAACCGGACCATCTTTGACATCACTAATAACCTCTCCAT | |
| | TATGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 159 | GTTATCCACGTGACCAAGGAAGTGAAATCCTTTGCAACGCTGTCCTGTGG | CD80_241-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 160 | GTTATCCACGTGACCAAGGAAGTGAAAGAATTTGCAACGCTGTCCTGTGG | CD80_242-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCTCCAATAACCTCTCCAT | |
| | TGTGATCCTGGCTCTGCGCCCATCTGACGAGGGCACATACGAGTGTGTTG | |
| | TTCTGAAGTATGAAAAAGACGCTTTCAAGCGGGAACACCTGGCTGAAGTG | |
| | ACGTTATCAGTCAAAGCTGAC | |
| 161 | GTTATCCACGTGACCAAGGAAGTGAAAGAATATGCAACGCTGTCCTGTGG | CD80_243-Fc |
| | TCACAATGTTTCTGTTGAAGAGCTGGCACAAACTCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGGTCTGCGCCCATCTGACGAGGGCACATACGAGTGTGTTG | |
| | TTCTGAAGAGAGAAAAAGACGCTTTCAAGCGGGAACACCTGGCTGAAGTG | |
| | ACGTTATCAGTCAAAGCTGAC | |
| 162 | GTTATCCACGTGACCAAGGAAGTGAAAGAATATGCAACGCTGTCCTGTGG | CD80_244-Fc |
| | TCACAATGTTTCTGTTGAAGAGCTGGCACAATATCGCATCTACTGGCAAA | |
| | AGGAGAAGAAAATGGTGCTGACTATGATGTCTGGGGACCTGAATATATGG | |
| | CCCGAGTACAAGAACCGGACCATCTTTGACATCACTAATAACCTCTCCAT | |
| | TGTGATCCTGGCTCTGCGCCCATCTGACGAGGGCACATACGAGTGTGTTG | |
| | TTCTGAAGAGAGAAAAAGACGCTTTCAAGCGGGAACACCTGGCTGAAGTG | |
| | ACGTTATCAGTCAAAGCTGAC | |
| 163 | VIHVTKEVKEVATLSCGHNVSVEELAQTRIYWQKEKKMVLTMMSGDMNIW | CD80 WT |
| | PEYKNRTIFDIINNLSIVILALRPSDEGTYECVVLKYEKDAFKREHLAEV | |
| | T | |
| 164 | VIHVTKEVKEVATLSCGHNVSVEELAQTRIYWQKEKKMVLTMMSGDMNIW | WT IgV |
| | PEYKNRTIFDIINNLSIVILALRPSDEGTYECVVLKYEKDAFKREHLAEV | |
| | TLSVKAD | |
| 165 | VIHVTKEVKEYATLSCGHNVSVEELAQTRIYWQKEKKMVLTMMSGDVNIW | CD80_165 |
| | PEYKNRTISDITNNLSIVILALRPSDEGTYECVVEKYEKDAFKREHLAEV | |
| | TLSVKAD | |
| 166 | VIHVTKEVKEYATLSCGHNVSVEELAQTRIYWQKEKKMVLTMMSGDVNIW | CD80_166 |
| | PEYKNRTIFDITNNLSIVILALRPSDEGTYECVVEKYEKDAFKREHLAEV | |
| | TLSVKAD | |
| 167 | VIHVTKEVKEYATLSCGHNVSVEELAQTRIYWQKEKKMVLTIMSGDVNIW | CD80_167 |
| | PEYKNRTIFDITNNLSIVILGLRPSDEGTYECVVLKYEKDAFKREHLAEV | |
| | TLSVKAD | |
| 168 | VIHVTKEVNGVATLSCGHNVSVEELAQTRIYWQKEKKMVLTMMSGDMNIW | CD80_168 |
| | PEYKNRTIFDITSNLSIVILALRPSDEGTYECVVEKYEKDAFKREHLAEV | |
| | TLSVKAD | |
| 169 | VIHVTKEVKGVATLSCGHNVSVEELAQTRIYWQKGKKMVLTMMSGEVNIW | CD80_169 |
| | PEYKNRTIFDITNNLSIMILALRPSDEGTYECVVLKYEKDAFKREHLAEV | |
| | TLSVKAD | |
| 170 | VIHVTKEVNGVATLSCGHNVSVEELAQTRIYWQKGKKMVLTMMSGEVNIW | CD80_170 |
| | PEYKNRTIFDITNNLSIMILALRPSDEGTYECVVLKYEKDAFKREHLAEV | |
| | TLSVKAD | |
| 171 | VIHVTKSVKEVATLSCGINLSVEELKQTRIYWQKEKKMVLTMMSGDLNIW | CD80_171 |
| | PEYKNRTIFDITNNLSIVILNLRPSDEGTYECVVLKYEKDAFKREHLAEV | |
| | TLSVKAD | |
| 172 | VIHVTKEVRSYATLSCGHNVSVEELAQTRIYWQKEKKMVLTLMSGDMNIW | CD80_172 |
| | PEYKNRTIMDITNNLSIMILALRPSDEGTYECVVEKYEKDAFKREHLAEV | |
| | TLSVKAD | |
| 173 | VIHVTKEVKEYATLSCGHNVSVEELAQYRIYWQKEKKMVLTMMSGDLNIW | CD80_173 |
| | PEYKNRTIFDIINNLSIVILALRPSDEGTYECVVXKYEKDAFKREHLAEV | |
| | TLSVKAD | |
| 174 | VIHVTKEVKRVATLSCGHNVSVEELAQTRIYWQKEKKMVLTMMSGDRNIW | CD80_174 |
| | PEYKNRTIFDITINLSIVILALRPSDEGTYECVVLKYEKDAFKREHLAEV | |
| | TLSVKAD | |
| 175 | VIHVTKEVKSFATLSCGHNVSVEELAQYRIYWQKEKKMVLTMMSGDLNIW | CD80_175 |
| | PEYKNRTIFDISNNLSIVILALRPSDEGTYECVVLKYEKDAFKREHLAEV | |
| | TLSVKAD | |
| 176 | VIHVTKEVKRVATLSCGYNVSVEELGQTRIYWQKDKKMVLTMMSGDMNIW | CD80_176 |
| | PEYKNRTIFDITNNLSILILALRPSDEGTYECVVEKYEKDAFKREHLAEV | |
| | TLSVKAD | |
| 177 | VIHVTKEVKGWATLSCGHNVSSEELAQPRIYWQKEKKMVLTMMSGDMNIW | CD80_177 |
| | PEYKNRTIFDIINNLSIVILGLRPSDEGTYKCVVLKREKDAFKREHLAEV | |
| | TLSVKAD | |
| 178 | VIHVTKEVKEYATLSCGHNVSVEELAQYRIYWQKEKKMVLTMMSGDMNIW | CD80_178 |
| | PEYKNRTIFDIINNLSIVILALRPSDEGTYECVVEKIEKDAFKREHLAEV | |
| | TLSVKAD | |
| 179 | VIHVTKEVKEVATLSCGFNVSVEELAQTRIYWQKEKKMVLIGMSGDMNIW | CD80_179 |
| | PEYKNRTIYDITNNLSINILALRPSDEGTYECVVLKYEKDAFKREHLAEV | |
| | TLSVKAD | |
| 180 | VIHVTKEVKGWATLSCGHNVSVEELAQYRIYWQKEKKMVLTMMSGDLNIW | CD80_180 |
| | PEYKNRTIFDIINNLSIVILALRPSDEGTYECVVLKYEKDAFKREHLAEV | |
| | TLSVKAD | |
| 181 | VIHVTKEVSRYATLSCGHNVSVEELAQTRIYWQKEKKMVLTMMSGDLNIW | CD80_181 |
| | PEYKNRTIFDITNNLSIVILGLRPSDEGTYECVVLKYEKDAFKREHLAEV | |
| | TLSVKAD | |
| 182 | VIHVTKEVKGWATLSCGHNVSVEELAQTRIYWQKGKKMVLTMMSGDLNIW | CD80_182 |
| | PEYKNRTIFDITNNLSIVILGLRPSDEGTYECVVLKREKDAFKREHLAEV | |
| | TLSVKAD | |
| 183 | VIHVTKEVKEYATLSCGHNVSVEELAQYRIYWQKEKKMVLTMMSGDLNIW | CD80_183 |
| | PEYKNRTIFDIINNLSIVILGLRPSDEGTYECVVLKREKDAFKREHLAEV | |
| | TLSVKAD | |
| 184 | VIHVTKEVKEYATLSCGHNVSVEELQQTRIYWQKEKKMVLTMMSGDLNIW | CD80_184 |
| | PEYKNRTIFDITNNLSIVILGLRPSDEGTYECVVLKREKDAFKREHLAEV | |
| | TLSVKAD | |
| 185 | VIHVTKEVKEYATLSCGHNVSVEELAQYRIYWQKEKKMVLTMMSGDLNIW | CD80_185 |
| | PEYKNRTIFDITNNLSIVILALRPSDEGTYECVVLKYEKDAFKREHLAEV | |
| | TLSVKAD | |
| 186 | VIHVTKKVKEWATLSCGHNVSVEELAQTRIYWQKEKKMVLTMMSGDMNIW | CD80_186 |
| | PEYKNRTIFDITHNLSIVILGLRPSDEGTYECVVLKKEKDAFKREHLAEV | |
| | TLSVKAD | |
| 187 | VIHVTKEVKEYATLSCGYNVSVEELAQTRIYWQKGKKMVLTMMSGDMNIW | CD80_187 |
| | PEYKNRTIFDIINNLSIVILALRPSDEGTYECVVQKYEKDAFKREHLAEV | |
| | TLSVKAD | |
| 188 | VIHVTKEVKEVATLSCGHNVSVEELAQTRIYWQKDKKMVLTMMSGDMNIW | CD80_188 |
| | PEYKNRTIFDIINNLSILILALRPSDEGTYECVVEKYEKDAFKREHLAEV | |
| | TLSVKAD | |
| 189 | VIHVTKEVKGWATLSCGHNVSVEELAQTRIYWQKEKKMVLTMMSGDVNIW | CD80_189 |
| | PEYKNRTIFDITNNLSIVILALRPSDEGTYECVVEKYEKDAFKREHLAEV | |
| | TLSVKAD | |
| 190 | VIHVTKEVKEVATLSCGHNVSVEELAQYRIYWQKEKKMVLTMMSGDLNIW | CD80_190 |
| | PEYKNRTIFDITNNLSIVILALRPSDEGTYECVVLKYEKDAFKREHLAEV | |
| | TLSVKAD | |
| 191 | VIHVTKEVKEYATLSCGHNVSVEELAQYRIYWQKEKKMVLTMMSGDLNIW | CD80_191 |
| | PEYKNRTIFDITNNLSILILALRPSDEGTYECVVEKYEKDAFKREHLAEV | |
| | TLSVKAD | |
| 192 | VIHVTKNVKEVATLSCGHNVSVEELAQTRIYWQKDKKMVLTMMSGDMNIW | CD80_192 |
| | PEYKNRTIFDITNNLSIVILALRPSDEGTYECVVLKYEKDAFKREHLAEV | |
| | RLSVKAD | |
| 193 | VIHVTKEVKEYATLSCGHNVSVEELAQTRIYWQKEKKMVLTWMSGDMNIW | CD80_193 |
| | PEYKNRTIFDIANNLSIVILALRPSDEGTYECVVEKYEKDAFKREHLAEV | |
| | TLSVKAD | |
| 194 | VIHVTKEVKEYATLSCGHNVSVEELAQTRIYWQKEKKMVLTWMSGDMNIW | CD80_194 |
| | PEYKNRTIFDIANNLSIVILALRPSDEGTYECVVLKYEKDAFKREHLAEV | |
| | TLSVKAD | |
| 195 | VIHVTKEVKEYATLSCGHNVSVEELAQTRIYWQKEKKMVLTWMSGDMNIW | CD80_195 |
| | PEYKNRTIYDITNNLSINILALRPSDEGTYECVVLKYEKDAFKREHLAEV | |
| | TLSVKAD | |
| 196 | VIHVTKEVKEYATLSCGHNVSVEELAQTRIYWQKEKKMVLTWMSGDMNIW | CD80_196 |
| | PKYKNRTIFDIANNLSIVILALRPSDEGTYECVVEKYEKDAFKREHLAEV | |
| | TLSVKAD | |
| 197 | VIHVTKEVKEYATLSCGHNVSVEELAQTRIYWQKDKKMVLIMMSGDMNIW | CD80_197 |
| | PEYKNRTIFDITNNLSIVILALRPSDEGTYECVVLKQEKDAFKREHLAEV | |
| | RLSVKAD | |
| 198 | VIHVTKEVKEVATLSCGYNVSVEELEQTSIYWQKDKKMVLTMMSGDLNIW | CD80_198 |
| | PEYKNRTIFDITNNLSIMILGLRPSDGGTYECVVLKYEKGAFKREHLAEV | |
| | TLSVKAD | |
| 199 | VIHVTKEVKGVATLSCGYNVSVEELAQTRIYWQKEKKMVLTMMSGDMNIW | CD80_199 |
| | PEYKNRTIFDITNNLSIVILALRPSDEGTYECVVLKYEKDAFKREHLAEV | |
| | TLSVKAD | |
| 200 | VIHVTKEVNRVATLSCGVNVSVEELAQYRIYWQKEKKMVLTMMSGDMNIW | CD80_200 |
| | PEYKNRTIFDITNNLSIVILGLRPSDEGTYECVVLKYEKDAFKREHLAEV | |
| | TLSVKAD | |
| 201 | VIHVTKEVKGVATLSCGYNVSVEELAQYRIYWQKEKKMVLTMMSGDWNIW | CD80_201 |
| | PEYKNRTIFDISNNLSIVILALRPSDEGTYECVVLKYEKDAFKREHLAEV | |
| | TLSVKAD | |
| 202 | VIHVTKHVKEVATLSCGLNISVEELAQYRIYWQKEKKMVLTMMSGSMNIW | CD80_202 |
| | PEYKNRTIFDIINNLSIVILGLRPSDEGTYECVVLKYEKDAFKREHLAEV | |
| | TLSVKAD | |
| 203 | VIHVTKEVNAWATLSCGFNVSVEELAQHRIYWQKEKKMVLTMMSGDLNIW | CD80_203 |
| | PEYKNRTIFDIENNLSIVILALDPSDEGTYECVVLKYEKDAFKREHLAEV | |
| | TLSVKAD | |
| 204 | VIHVTKEVKEVATLSCGVNISVEELAQYRIYWQKGKKMVLTMMSGDVNIW | CD80_204 |
| | PEYKNRTIFDITNNLSIVILALEPSDEGTYECVVLKYEKDAFKREHLAEV | |
| | TLSVKAD | |
| 205 | VIHVTKEVKGVATLSCGHNVSVEELAQTRIYWQKEKKMVLTMMSGKMNIW | CD80_205 |
| | PEYKNRTIFDITNNLSIVILALRPSDEGTYECVVEKYEKDAFKREHLAEV | |
| | TLSVKAD | |
| 206 | VIHVTKEVKGVATLSCGTNVSVEELATTRIYWQKEKKMVLTMMSGELNIW | CD80_206 |
| | PEYKNRTIFDIINNLSIVILALRPSDEGTYECVVLKYEKDAFKREHLAEV | |
| | TLSVKAD | |
| 207 | VIHVTKEVKEVATLSCGHNVSVEELAQTRIYWQKDKKMVLTMMSGDMNIW | CD80_207 |
| | PEYKNRTIFDITNNLSITILALRPSDEGTYECVVLKYEKDAFKREHLAEV | |
| | KLSVKAD | |
| 208 | VIHVTKEVKEYATLSCGHNVSVEELAQTRIYWQKGKKMVLIGMSGDMNIW | CD80_208 |
| | PEYKNRTISDITNNLSIVILALRPSDEGTYECVVLKYEKDAFKREHLAEV | |
| | TLSVKAD | |
| 209 | VIHVTKEVKEYATLSCGHNVSVEELAQRRIYWQKGKKMVLTMMSGDLNIW | CD80_209 |
| | PEYKNRTISDITNNLSIVILALRPSDEGTYECVVLKYEKDAFKREHLAEV | |
| | TLSVKAD | |
| 210 | VIHVTKNVKEVATLSCGHNVSVEELAQTRIYWQKDKKMVLTMMSGDMNIW | CD80_210 |
| | PEYKNRTISDITNNLSIVILALRPSDEGTYECVVLKYEKDAFKREHLAEV | |
| | TLSVKAD | |
| 211 | VIHVTKEVKEYATLSCGHNVSVEELAQRRIYWQKGKKMVLTMMSGDLNIW | CD80_211 |
| | PEYKNRTIFDIINNLSIVILGLRPSDEGTYECVVLKYEKDAFKREHLAEV | |
| | TLSVKAD | |
| 212 | VIHVTKEVRAVATLSCGHNVSVEELAQTRIYWQKGKKMVLTMMSGDMNIW | CD80_212 |
| | PEYKNRTIFDITNNLSITILALRPSDEGTYECVVLKYEKDAFKREHLAEV | |
| | KLSVKAD | |
| 213 | VIHVTKEVKEYATLSCGHNVSVEELAQTRIYWQKEKKMVLTMMSGDMNIW | CD80_213 |
| | PEYKNRTIFDIINNLSITILALRPSDEGTYECVVLKYEKDAFKREHLAEV | |
| | TLSVKAD | |
| 214 | VIHVTKEVKGVATLSCGHNVSVEELAQTRIYWQKGKKMVLTMMSGEVNIW | CD80_214 |
| | PEYKNRTIFDIINNLSIMILALRPSDEGTYECVVLKYEKDAFKREHLAEV | |
| | KLSVKAD | |
| 215 | VIHVTKEVRAVATLSCGHNVSVEELAQTRIYWQKGKKMVLTMMSGDMNIW | CD80_215 |
| | PEYKNRTIFDITNNLSILILALRPSDEGTYECVVEKYEKDAFKREHLAEV | |
| | TLSVKAD | |
| 216 | VIHVTKEVKEWATLSCGHNVSVEELAQYRIYWQKEKKMVLTMMSGVMNIW | CD80_216 |
| | PEYKNRTIFDITNNLSIVILALEPSDEGTYECVVLKYEKDALKREHLAEV | |
| | TLSVKAD | |
| 217 | VIHVTKEVNGVATLSCGHNVSVEELAQTRIYWQKEKKMVLTMMSGDMNIW | CD80_217 |
| | PEYKNRTIFDIINNLSIVILALRPSDEGTYECVVLKKEKDAFKREHLAEV | |
| | QLSVKAD | |
| 218 | VIHVTKEVKEWATLSCGHNVSVEELAQYRIYWQKEKKMVLTMMSGVMNIW | CD80_218 |
| | PEYKNRTIFDITNNLSITILALTPSDEGTYECVVLKNEKDAFKREHLAEV | |
| | TLSVKAD | |
| 219 | VIHVTKEVKSYATLSCGHNVSVEELAQTRIYWQKEKKMVLTRMSGDMNIW | CD80_219 |
| | PEYKNRTIFDITNNLSIVILVLRPSDEGTYECVVLKYEKDAFKREHLAEV | |
| | TLSVKAD | |
| 220 | VIHVTKEVKEVATLSCGFNVSVEELAQVRIYWQKEKKMVLTMMSGDLNIW | CD80_220 |
| | PEYKNRTIFDITNNLSISILALRPSDEGTYECVVLKYEKDAFKREHLAEV | |
| | TLSVKAD | |
| 221 | VIHVTKEVKGVATLSCGHNVSVEELSQYRIYWQKEKKMVLTMMSGDMNIW | CD80_221 |
| | PEYKNRTIFDITNNLSIVILALRPSDEGTYECVVLKYEKDAFKREHLAEV | |
| | TLSVKAD | |
| 222 | VIHVTKEVKEVATLSCGHNVSVEELAQTRIYWQKDKKMVLTMMSGQMNIW | CD80 222 |
| | PEYKNRTIFDITNNLSIVILALRPSDEGTYECVVEKYEKDAFKREHLAEV | |
| | TLSVKAD | |
| 223 | VIHVTKEVKSVATLSCGHNVSVEELAQTRIYWQKEKKMVLTMMSGDMNIW | CD80_223 |
| | PEYKNRTIFDIINNLSIMILALRPSDEGTYECVVLKPEKDAFKREHLAEV | |
| | TLSVKAD | |
| 224 | VIHVTKEVNEWATLSCGHNVSVEELAQTRIYWQKEKKMVLTMMSGDLNIW | CD80_224 |
| | PEYKNRTIFDITNNLSITILALTPSDEGTYECVVLKNEKDAFKREHLAEV | |
| | TLSVKAD | |
| 225 | VIHVTKEVKEYATLSCGHNVSVEELAQTRIYWQKEKKMVLTMMSGDLNIW | CD80_225 |
| | PEYKNRTIFDITNNLSITILALRPSDEGTYECVVLKYEKDAFKREHLAEV | |
| | TLSVKAD | |
| 226 | VIHVTKEVREVATLSCGHNVSVEELTQYRIYWQKAKKMVLTMMSGDLNIW | CD80_226 |
| | PEYKNRTIFDIINNLSIVILGLRPSDEGTYECVVLKYEKDAFKREHLAEV | |
| | TLSVKAD | |
| 227 | VIHVTKEVKEWATLSCGHNVSVEELAQHRIYWQKEKKMVLTMMSGQMNIW | CD80_227 |
| | PEYKNRTIFDIINNLSILILALRPSDEGTYECVVEKYEKDAFKREHLAEV | |
| | TLSVKAD | |
| 228 | VIHVTKQVKEYATLSCGHNVSVEELAQTHIYWQKEKKMVLTMMSGDLNIW | CD80_228 |
| | PEYKNRTIFDITNNLSITILALRPSDEGTYECVVLKYEKDAFKREHLAEV | |
| | TLSVKAD | |
| 229 | VIHVTKEVKEYATLSCGHNVSVEELAQTRIYWQKEKKMVLTMMSGDVNIW | CD80_229 |
| | PEYKNRTIFDIINNLSIVILGLRPSDEGTYECVVEKYEKDAFKREHLAEV | |
| | TLSVKAD | |
| 230 | VIHVTKEVKGVATLSCGHNVSVEELAFTRIYWQKEKKMVLTMMSGNMNIW | CD80_230 |
| | PEYKNRTIFDITNNLSIVILGLRPSDEGTYECVVLKYEKGAFKREHLAEV | |
| | TLSVKAD | |
| 231 | VIHVTKEVKEWATLSCGHNVSVEELAQYRIYWQKEKKMVLTMMSGDLNIW | CD80_231 |
| | PEYKNRTIFDIINNLSIVILALRPSDEGTYECVVLKYEKDAFKREHLAEV | |
| | TLSVKAD | |
| 232 | VIHVTKEVKEFATLSCGHNVSVEELAQYRIYWQKEKKMVLTMMSGDLNIW | CD80_232 |
| | PEYKNRTIFDITNNLSIVILALRPSDEGTYECVVLKYEKDAFKREHLAEV | |
| | TLSVKAD | |
| 233 | VIHVTKEVKEYATLSCGHNVSVEELAQYRIYWQKEKKMVLTMMSGDLNIW | CD80_233 |
| | PEYKNRTIFDITNNLSIMILALRPSDEGTYECVVLKYEKDAFKREHLAEV | |
| | TLSVKAD | |
| 234 | VIHVTKEVKEYATLSCGHNVSVEELAQYRIYWQKEKKMVLTMMSGDLNIW | CD80_234 |
| | PEYKNRTIFDIINNLSILILALRPSDEGTYECVVLKYEKDAFKREHLAEV | |
| | TLSVKAD | |
| 235 | VIHVTKEVKEYATLSCGHNVSVEELAQTRIYWQKEKKMVLTMMSGDLNIW | CD80_235 |
| | PEYKNRTIFDIINNLSIVILGLRPSDEGTYECVVLKYEKDAFKREHLAEV | |
| | TLSVKAD | |
| 236 | VIHVTKEVKEYATLSCGHNVSVEELAQYRIYWQKEKKMVLTMMSGDLNIW | CD80_236 |
| | PEYKNRTIFDITNNLSIVILGLRPSDEGTYECVVLKYEKDAFKREHLAEV | |
| | TLSVKAD | |
| 237 | VIHVTKEVKEYATLSCGHNVSVEELAQTRIYWQKEKKMVLTMMSGDLNIW | CD80_237 |
| | PEYKNRTIFDITNNLSIMILALRPSDEGTYECVVLKYEKDAFKREHLAEV | |
| | TLSVKAD | |
| 238 | VIHVTKEVKEYATLSCGHNVSVEELAQTRIYWQKEKKMVLTMMSGEVNIW | CD80_238 |
| | PEYKNRTIFDITNNLSIMILALRPSDEGTYECVVLKYEKDAFKREHLAEV | |
| | TLSVKAD | |
| 239 | VIHVTKEVKEYATLSCGHNVSVEELAQYRIYWQKEKKMVLTMMSGEVNIW | CD80_239 |
| | PEYKNRTIFDITNNLSIVILALRPSDEGTYECVVLKYEKDAFKREHLAEV | |
| | TLSVKAD | |
| 240 | VIHVTKEVKEYATLSCGHNVSVEELAQYRIYWQKEKKMVLTMMSGEVNIW | CD80_240 |
| | PEYKNRTIFDITNNLSIMILALRPSDEGTYECVVLKYEKDAFKREHLAEV | |
| | TLSVKAD | |
| 241 | VIHVTKEVKSFATLSCGHNVSVEELAQYRIYWQKEKKMVLTMMSGDLNIW | CD80_241 |
| | PEYKNRTIFDITNNLSIVILALRPSDEGTYECVVLKYEKDAFKREHLAEV | |
| | TLSVKAD | |
| 242 | VIHVTKEVKEFATLSCGHNVSVEELAQYRIYWQKEKKMVLTMMSGDLNIW | CD80_242 |
| | PEYKNRTIFDISNNLSIVILALRPSDEGTYECVVLKYEKDAFKREHLAEV | |
| | TLSVKAD | |
| 243 | VIHVTKEVKEYATLSCGHNVSVEELAQTRIYWQKEKKMVLTMMSGDLNIW | CD80_243 |
| | PEYKNRTIFDITNNLSIVILGLRPSDEGTYECVVLKREKDAFKREHLAEV | |
| | TLSVKAD | |
| 244 | VIHVTKEVKEYATLSCGHNVSVEELAQYRIYWQKEKKMVLTMMSGDLNIW | CD80_244 |
| | PEYKNRTIFDIINNLSIVILALRPSDEGTYECVVLKREKDAFKREHLAEV | |
| | TLSVKAD | |
| 245 | VIHVTKEVKEYATLSCGHNVSVEELAQTRIYWQKEKKMVLTMMSGDVNIW | CD80_165-Fc |
| | PEYKNRTISDIINNLSIVILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 246 | VIHVTKEVKEYATLSCGHNVSVEELAQTRIYWQKEKKMVLTMMSGDVNIW | CD80_166-Fc |
| | PEYKNRTIFDIINNLSIVILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 247 | VIHVTKEVKEYATLSCGHNVSVEELAQTRIYWQKEKKMVLTIMSGDVNIW | CD80_167-Fc |
| | PEYKNRTIFDIINNLSIVILGLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 248 | VIHVTKEVNGVATLSCGHNVSVEELAQTRIYWQKEKKMVLTMMSGDMNIW | CD80_168-Fc |
| | PEYKNRTIFDITSNLSIVILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 249 | VIHVTKEVKGVATLSCGHNVSVEELAQTRIYWQKGKKMVLTMMSGEVNIW | CD80_169-Fc |
| | PEYKNRTIFDITNNLSIMILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 250 | VIHVTKEVNGVATLSCGHNVSVEELAQTRIYWQKGKKMVLTMMSGEVNIW | CD80_170-Fc |
| | PEYKNRTIFDIINNLSIMILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 251 | VIHVTKSVKEVATLSCGINLSVEELKQTRIYWQKEKKMVLTMMSGDLNIW | CD80_171-Fc |
| | PEYKNRTIFDITNNLSIVILNLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 252 | VIHVTKEVRSYATLSCGHNVSVEELAQTRIYWQKEKKMVLTLMSGDMNIW | CD80_172-Fc |
| | PEYKNRTIMDITNNLSIMILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 253 | VIHVTKEVKEYATLSCGHNVSVEELAQYRIYWQKEKKMVLTMMSGDLNIW | CD80_173-Fc |
| | PEYKNRTIFDIINNLSIVILALRPSDEGTYECVVXKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 254 | VIHVTKEVKRVATLSCGHNVSVEELAQTRIYWQKEKKMVLTMMSGDRNIW | CD80_174-Fc |
| | PEYKNRTIFDITINLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 255 | VIHVTKEVKSFATLSCGHNVSVEELAQYRIYWQKEKKMVLTMMSGDLNIW | CD80_175-Fc |
| | PEYKNRTIFDISNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 256 | VIHVTKEVKRVATLSCGYNVSVEELGQTRIYWQKDKKMVLTMMSGDMNIW | CD80_176-Fc |
| | PEYKNRTIFDITNNLSILILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 257 | VIHVTKEVKGWATLSCGHNVSSEELAQPRIYWQKEKKMVLTMMSGDMNIW | CD80_177-Fc |
| | PEYKNRTIFDITNNLSIVILGLRPSDEGTYKCVVLKREKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 258 | VIHVTKEVKEYATLSCGHNVSVEELAQYRIYWQKEKKMVLTMMSGDMNIW | CD80_178-Fc |
| | PEYKNRTIFDIINNLSIVILALRPSDEGTYECVVEKIEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 259 | VIHVTKEVKEVATLSCGFNVSVEELAQTRIYWQKEKKMVLIGMSGDMNIW | CD80_179-Fc |
| | PEYKNRTIYDIINNLSINILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 260 | VIHVTKEVKGWATLSCGHNVSVEELAQYRIYWQKEKKMVLTMMSGDLNIW | CD80_180-Fc |
| | PEYKNRTIFDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 261 | VIHVTKEVSRYATLSCGHNVSVEELAQTRIYWQKEKKMVLTMMSGDLNIW | CD80_181-Fc |
| | PEYKNRTIFDIINNLSIVILGLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 262 | VIHVTKEVKGWATLSCGHNVSVEELAQTRIYWQKGKKMVLTMMSGDLNIW | CD80_182-Fc |
| | PEYKNRTIFDITNNLSIVILGLRPSDEGTYECVVLKREKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 263 | VIHVTKEVKEYATLSCGHNVSVEELAQYRIYWQKEKKMVLTMMSGDLNIW | CD80_183-Fc |
| | PEYKNRTIFDIINNLSIVILGLRPSDEGTYECVVLKREKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 264 | VIHVTKEVKEYATLSCGHNVSVEELQQTRIYWQKEKKMVLTMMSGDLNIW | CD80_184-Fc |
| | PEYKNRTIFDIINNLSIVILGLRPSDEGTYECVVLKREKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 265 | VIHVTKEVKEYATLSCGHNVSVEELAQYRIYWQKEKKMVLTMMSGDLNIW | CD80_185-Fc |
| | PEYKNRTIFDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 266 | VIHVTKKVKEWATLSCGHNVSVEELAQTRIYWQKEKKMVLTMMSGDMNIW | CD80_186-Fc |
| | PEYKNRTIFDITHNLSIVILGLRPSDEGTYECVVLKKEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 267 | VIHVTKEVKEYATLSCGYNVSVEELAQTRIYWQKGKKMVLTMMSGDMNIW | CD80_187-Fc |
| | PEYKNRTIFDITNNLSIVILALRPSDEGTYECVVQKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 268 | VIHVTKEVKEVATLSCGHNVSVEELAQTRIYWQKDKKMVLTMMSGDMNIW | CD80_188-Fc |
| | PEYKNRTIFDITNNLSILILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 269 | VIHVTKEVKGWATLSCGHNVSVEELAQTRIYWQKEKKMVLTMMSGDVNIW | CD80_189-Fc |
| | PEYKNRTIFDITNNLSIVILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 270 | VIHVTKEVKEVATLSCGHNVSVEELAQYRIYWQKEKKMVLTMMSGDLNIW | CD80_190-Fc |
| | PEYKNRTIFDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 271 | VIHVTKEVKEYATLSCGHNVSVEELAQYRIYWQKEKKMVLTMMSGDLNIW | CD80_191-Fc |
| | PEYKNRTIFDITNNLSILILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 272 | VIHVTKNVKEVATLSCGHNVSVEELAQTRIYWQKDKKMVLTMMSGDMNIW | CD80_192-Fc |
| | PEYKNRTIFDITNNLSIVILALRPSDEGTYECVVLKYEKDAFKREHLAEV | |
| | RLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 273 | VIHVTKEVKEYATLSCGHNVSVEELAQTRIYWQKEKKMVLTWMSGDMNIW | CD80_193-Fc |
| | PEYKNRTIFDIANNLSIVILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 274 | VIHVTKEVKEYATLSCGHNVSVEELAQTRIYWQKEKKMVLTWMSGDMNIW | CD80_194-Fc |
| | PEYKNRTIFDIANNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 275 | VIHVTKEVKEYATLSCGHNVSVEELAQTRIYWQKEKKMVLTWMSGDMNIW | CD80_195-Fc |
| | PEYKNRTIYDITNNLSINILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 276 | VIHVTKEVKEYATLSCGHNVSVEELAQTRIYWQKEKKMVLTWMSGDMNIW | CD80_196-Fc |
| | PKYKNRTIFDIANNLSIVILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 277 | VIHVTKEVKEYATLSCGHNVSVEELAQTRIYWQKDKKMVLTMMSGDMNIW | CD80_197-Fc |
| | PEYKNRTIFDITNNLSIVILALRPSDEGTYECVVLKQEKDAFKREHLAEV | |
| | RLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 278 | VIHVTKEVKEVATLSCGYNVSVEELEQTSIYWQKDKKMVLTMMSGDLNIW | CD80_198-Fc |
| | PEYKNRTIFDIINNLSIMILGLRPSDGGTYECVVLKYEKGAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 279 | VIHVTKEVKGVATLSCGYNVSVEELAQTRIYWQKEKKMVLTMMSGDMNIW | CD80_199-Fc |
| | PEYKNRTIFDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 280 | VIHVTKEVNRVATLSCGVNVSVEELAQYRIYWQKEKKMVLTMMSGDMNIW | CD80_200-Fc |
| | PEYKNRTIFDITNNLSIVILGLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 281 | VIHVTKEVKGVATLSCGYNVSVEELAQYRIYWQKEKKMVLTMMSGDWNIW | CD80_201-Fc |
| | PEYKNRTIFDISNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 282 | VIHVTKHVKEVATLSCGLNISVEELAQYRIYWQKEKKMVLTMMSGSMNIW | CD80_202-Fc |
| | PEYKNRTIFDITNNLSIVILGLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 283 | VIHVTKEVNAWATLSCGFNVSVEELAQHRIYWQKEKKMVLTMMSGDLNIW | CD80_203-Fc |
| | PEYKNRTIFDIENNLSIVILALDPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 284 | VIHVTKEVKEVATLSCGVNISVEELAQYRIYWQKGKKMVLTMMSGDVNIW | CD80_204-Fc |
| | PEYKNRTIFDITNNLSIVILALEPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 285 | VIHVTKEVKGVATLSCGHNVSVEELAQTRIYWQKEKKMVLTMMSGKMNIW | CD80_205-Fc |
| | PEYKNRTIFDITNNLSIVILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 286 | VIHVTKEVKGVATLSCGTNVSVEELATTRIYWQKEKKMVLTMMSGELNIW | CD80_206-Fc |
| | PEYKNRTIFDIINNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 287 | VIHVTKEVKEVATLSCGHNVSVEELAQTRIYWQKDKKMVLTMMSGDMNIW | CD80_207-Fc |
| | PEYKNRTIFDIINNLSITILALRPSDEGTYECVVLKYEKDAFKREHLAEV | |
| | KLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 288 | VIHVTKEVKEYATLSCGHNVSVEELAQTRIYWQKGKKMVLTGMSGDMNIW | CD80_208-Fc |
| | PEYKNRTISDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 289 | VIHVTKEVKEYATLSCGHNVSVEELAQRRIYWQKGKKMVLTMMSGDLNIW | CD80_209-Fc |
| | PEYKNRTISDIINNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 290 | VIHVTKNVKEVATLSCGHNVSVEELAQTRIYWQKDKKMVLTMMSGDMNIW | CD80_210-Fc |
| | PEYKNRTISDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 291 | VIHVTKEVKEYATLSCGHNVSVEELAQRRIYWQKGKKMVLTMMSGDLNIW | CD80_211-Fc |
| | PEYKNRTIFDITNNLSIVILGLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 292 | VIHVTKEVRAVATLSCGHNVSVEELAQTRIYWQKGKKMVLTMMSGDMNIW | CD80_212-Fc |
| | PEYKNRTIFDIINNLSITILALRPSDEGTYECVVLKYEKDAFKREHLAEV | |
| | KLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 293 | VIHVTKEVKEYATLSCGHNVSVEELAQTRIYWQKEKKMVLTMMSGDMNIW | CD80_213-Fc |
| | PEYKNRTIFDITNNLSITILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 294 | VIHVTKEVKGVATLSCGHNVSVEELAQTRIYWQKGKKMVLTMMSGEVNIW | CD80_214-Fc |
| | PEYKNRTIFDITNNLSIMILALRPSDEGTYECVVLKYEKDAFKREHLAEV | |
| | KLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 295 | VIHVTKEVRAVATLSCGHNVSVEELAQTRIYWQKGKKMVLTMMSGDMNIW | CD80_215-Fc |
| | PEYKNRTIFDITNNLSILILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 296 | VIHVTKEVKEWATLSCGHNVSVEELAQYRIYWQKEKKMVLTMMSGVMNIW | CD80_216-Fc |
| | PEYKNRTIFDIINNLSIVILALEPSDEGTYECVVLKYEKDALKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 297 | VIHVTKEVNGVATLSCGHNVSVEELAQTRIYWQKEKKMVLTMMSGDMNIW | CD80_217-Fc |
| | PEYKNRTIFDITNNLSIVILALRPSDEGTYECVVLKKEKDAFKREHLAEV | |
| | QLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 298 | VIHVTKEVKEWATLSCGHNVSVEELAQYRIYWQKEKKMVLTMMSGVMNIW | CD80_218-Fc |
| | PEYKNRTIFDITNNLSITILALTPSDEGTYECVVLKNEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 299 | VIHVTKEVKSYATLSCGHNVSVEELAQTRIYWQKEKKMVLTRMSGDMNIW | CD80_219-Fc |
| | PEYKNRTIFDITNNLSIVILVLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 300 | VIHVTKEVKEVATLSCGFNVSVEELAQVRIYWQKEKKMVLTMMSGDLNIW | CD80_220-Fc |
| | PEYKNRTIFDITNNLSISILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 301 | VIHVTKEVKGVATLSCGHNVSVEELSQYRIYWQKEKKMVLTMMSGDMNIW | CD80_221-Fc |
| | PEYKNRTIFDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 302 | VIHVTKEVKEVATLSCGHNVSVEELAQTRIYWQKDKKMVLTMMSGQMNIW | CD80_222-Fc |
| | PEYKNRTIFDITNNLSIVILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 303 | VIHVTKEVKSVATLSCGHNVSVEELAQTRIYWQKEKKMVLTMMSGDMNIW | CD80_223-Fc |
| | PEYKNRTIFDITNNLSIMILALRPSDEGTYECVVLKPEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 304 | VIHVTKEVNEWATLSCGHNVSVEELAQTRIYWQKEKKMVLTMMSGDLNIW | CD80_224-Fc |
| | PEYKNRTIFDIINNLSITILALTPSDEGTYECVVLKNEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 305 | VIHVTKEVKEYATLSCGHNVSVEELAQTRIYWQKEKKMVLTMMSGDLNIW | CD80_225-Fc |
| | PEYKNRTIFDITNNLSITILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 306 | VIHVTKEVREVATLSCGHNVSVEELTQYRIYWQKAKKMVLTMMSGDLNIW | CD80_226-Fc |
| | PEYKNRTIFDIINNLSIVILGLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 307 | VIHVTKEVKEWATLSCGHNVSVEELAQHRIYWQKEKKMVLTMMSGQMNIW | CD80_227-Fc |
| | PEYKNRTIFDIINNLSILILALRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 308 | VIHVTKQVKEYATLSCGHNVSVEELAQTHIYWQKEKKMVLTMMSGDLNIW | CD80_228-Fc |
| | PEYKNRTIFDITNNLSITILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 309 | VIHVTKEVKEYATLSCGHNVSVEELAQTRIYWQKEKKMVLTMMSGDVNIW | CD80_229-Fc |
| | PEYKNRTIFDITNNLSIVILGLRPSDEGTYECVVEKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 310 | VIHVTKEVKGVATLSCGHNVSVEELAFTRIYWQKEKKMVLTMMSGNMNIW | CD80_230-Fc |
| | PEYKNRTIFDIINNLSIVILGLRPSDEGTYECVVLKYEKGAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 311 | VIHVTKEVKEWATLSCGHNVSVEELAQYRIYWQKEKKMVLTMMSGDLNIW | CD80_231-Fc |
| | PEYKNRTIFDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 312 | VIHVTKEVKEFATLSCGHNVSVEELAQYRIYWQKEKKMVLTMMSGDLNIW | CD80_232-Fc |
| | PEYKNRTIFDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 313 | VIHVTKEVKEYATLSCGHNVSVEELAQYRIYWQKEKKMVLTMMSGDLNIW | CD80_233-Fc |
| | PEYKNRTIFDITNNLSIMILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 314 | VIHVTKEVKEYATLSCGHNVSVEELAQYRIYWQKEKKMVLTMMSGDLNIW | CD80_234-Fc |
| | PEYKNRTIFDIINNLSILILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 315 | VIHVTKEVKEYATLSCGHNVSVEELAQTRIYWQKEKKMVLTMMSGDLNIW | CD80_235-Fc |
| | PEYKNRTIFDITNNLSIVILGLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 316 | VIHVTKEVKEYATLSCGHNVSVEELAQYRIYWQKEKKMVLTMMSGDLNIW | CD80_236-Fc |
| | PEYKNRTIFDITNNLSIVILGLRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 317 | VIHVTKEVKEYATLSCGHNVSVEELAQTRIYWQKEKKMVLTMMSGDLNIW | CD80_237-Fc |
| | PEYKNRTIFDIINNLSIMILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 318 | VIHVTKEVKEYATLSCGHNVSVEELAQTRIYWQKEKKMVLTMMSGEVNIW | CD80_238-Fc |
| | PEYKNRTIFDITNNLSIMILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 319 | VIHVTKEVKEYATLSCGHNVSVEELAQYRIYWQKEKKMVLTMMSGEVNIW | CD80_239-Fc |
| | PEYKNRTIFDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTIPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 320 | VIHVTKEVKEYATLSCGHNVSVEELAQYRIYWQKEKKMVLTMMSGEVNIW | CD80_240-Fc |
| | PEYKNRTIFDITNNLSIMILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 321 | VIHVTKEVKSFATLSCGHNVSVEELAQYRIYWQKEKKMVLTMMSGDLNIW | CD80_241-Fc |
| | PEYKNRTIFDITNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 322 | VIHVTKEVKEFATLSCGHNVSVEELAQYRIYWQKEKKMVLTMMSGDLNIW | CD80_242-Fc |
| | PEYKNRTIFDISNNLSIVILALRPSDEGTYECVVLKYEKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 323 | VIHVTKEVKEYATLSCGHNVSVEELAQTRIYWQKEKKMVLTMMSGDLNIW | CD80_243-Fc |
| | PEYKNRTIFDITNNLSIVILGLRPSDEGTYECVVLKREKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 324 | VIHVTKEVKEYATLSCGHNVSVEELAQYRIYWQKEKKMVLTMMSGDLNIW | CD80_244-Fc |
| | PEYKNRTIFDIINNLSIVILALRPSDEGTYECVVLKREKDAFKREHLAEV | |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLG | |
| 325 | GSGGGGS | GSG4S AA |
| | | Sequence |
| 326 | ESKYGPPCPPCPAPEFLGGPSVFLFPPKPKDTLMISRTPEVTCVVVDVSQ | IgG4 Fc |
| | EDPEVQFNWYVDGVEVHNAKTKPREEQFNSTYRVVSVLTVLHQDWLNGKE | |
| | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLG | |
| 327 | VIHVTKEVKEVATLSCGHNVSVEELAQTRIYWQKEKKMVLTMMSGDMNIW | WT CD80 ECD- |
| | PEYKNRTIFDIINNLSIVILALRPSDEGTYECVVLKYEKDAFKREHLAEV | Fc |
| | TLSVKADFPTPSISDFEIPTSNIRRIICSTSGGFPEPHLSWLENGEELNA | |
| | INTTVSQDPETELYAVSSKLDFNMTTNHSFMCLIKYGHLRVNQTFNWNTT | |
| | KQEHFPDNGSGGGGSESKYGPPCPPCPAPEFLGGPSVFLFPPKPKDTLMI | |
| | SRTPEVTCVVVDVSQEDPEVQFNWYVDGVEVHNAKTKPREEQFNSTYRVV | |
| | SVLTVLHQDWLNGKEYKCKVSNKGLPSSIEKTISKAKGQPREPQVYTLPP | |
| | SQEEMTKNQVSLTCLVKGFYPSDIAVEWESNGQPENNYKTTPPVLDSDGS | |
| | FFLYSRLTVDKSRWQEGNVFSCSVMHEALHNHYTQKSLSLSLG | |
| 328 | GGGGS | linker |
| 329 | GGGGSGGGGS | linker |
| 330 | GGGGSGGGGSGGGGS | linker |
| 331 | GGGGSGGGGSGGGGSGGGGS | linker |
| 332 | GGGGSGGGGSGGGGSGGGGSGGGGS | linker |
| 333 | GGGGSSA | linker |
| 334 | GGGGSGGGGSAAA | linker |
| 335 | GSGGGGSGGGGS | linker |
| 336 | VIHVTKEVKGWATLSCGHNVSVEELAQYRIYWQKEKKMVLTMMSGDLNIW | CD80-Fc- |
| | PEYKNRTIFDITNNLSIVILALRPSDEGTYECVVLKYEKDAFKREHLAEV | CD80_336 |
| | TLSVKADGSGGGGSESKYGPPCPPCPAPEFLGGPSVFLFPPKPKDTLMIS | |
| | RTPEVTCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLGGGGGSGGG | |
| | GSGGGGSVIHVTKEVKGWATLSCGYNVSVEELAQYRIYWQKEKKMVLTMM | |
| | SGDLNIWPEYKNRTIFDIINNLSIVILALRPSDEGTYECVVLKYEKDAFK | |
| | REHLAEVTLSVKAD | |
| 337 | VIHVTKEVKGWATLSCGHNVSVEELAQYRIYWQKEKKMVLTMMSGDLNIW | CD80-CD80- |
| | PEYKNRTIFDITNNLSIVILALRPSDEGTYECVVLKYEKDAFKREHLAEV | Fc_337 |
| | TLSVKADGGGGSGGGGSGGGGSVIHVTKEVKGWATLSCGYNVSVEELAQY | |
| | RIYWQKEKKMVLTMMSGDLNIWPEYKNRTIFDITNNLSIVILALRPSDEG | |
| | TYECVVLKYEKDAFKREHLAEVTLSVKADGSGGGGSESKYGPPCPPCPAP | |
| | EFLGGPSVFLFPPKPKDTLMISRTPEVTCVVVDVSQEDPEVQFNWYVDGV | |
| | EVHNAKTKPREEQFNSTYRVVSVLTVLHQDWLNGKEYKCKVSNKGLPSSI | |
| | EKTISKAKGQPREPQVYTLPPSQEEMTKNQVSLTCLVKGFYPSDIAVEWE | |
| | SNGQPENNYKTTPPVLDSDGSFFLYSRLTVDKSRWQEGNVFSCSVMHEAL | |
| | HNHYTQKSLSLSLG | |
| 338 | VIHVTKEVKGWATLSCGHNVSVEELAQYRIYWQKEKKMVLTMMSGDLNIW | CD80-Fc- |
| | PEYKNRTIFDITNNLSIVILALRPSDEGTYECVVLKYEKDAFKREHLAEV | CD80_338 |
| | TLSVKADGSGGGGSDKTHTCPPCPAPEAEGAPSVFLFPPKPKDTLMISRT | |
| | PEVTCVVVDVSHEDPEVKFNWYVDGVEVHNAKTKPREEQYNSTYRVVSVL | |
| | TVLHQDWLNGKEYKCKVSNKALPAPIEKTISKAKGQPREPQVYTLPPSRD | |
| | ELTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGGGGGSGGGGS | |
| | GGGGSVIHVTKEVKGWATLSCGYNVSVEELAQYRIYWQKEKKMVLTMMSG | |
| | DLNIWPEYKNRTIFDIINNLSIVILALRPSDEGTYECVVLKYEKDAFKRE | |
| | HLAEVTLSVKAD | |
| 339 | VIHVTKEVKEYATLSCGHNVSVEELAQYRIYWQKEKKMVLTMMSGDLNIW | CD80-Fc- |
| | PEYKNRTIFDITNNLSIVILALRPSDEGTYECVVLKYEKDAFKREHLAEV | CD80_339 |
| | TLSVKADGSGGGGSPCPPCPAPEFLGGPSVFLFPPKPKDTLMISRTPEVT | |
| | CVVVDVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRL | |
| | TVDKSRWQEGNVFSCSVMHEALHNHYTQKSLSLSLGGGGGSGGGGGGGG | |
| | SVIHVTKEVKEYATLSCGYNVSVEELAQYRIYWQKEKKMVLTMMSGDLNI | |
| | WPEYKNRTIFDIINNLSIVILALRPSDEGTYECVVLKYEKDAFKREHLAE | |
| | VTLSVKAD | |
| 340 | VIHVTKEVKEYATLSCGHNVSVEELAQYRIYWQKEKKMVLTMMSGDLNIW | CD80-CD80- |
| | PEYKNRTIFDIINNLSIVILALRPSDEGTYECVVLKYEKDAFKREHLAEV | Fc_340 |
| | TLSVKADGGGGSGGGGSGGGGSVIHVTKEVKEYATLSCGYNVSVEELAQY | |
| | RIYWQKEKKMVLTMMSGDLNIWPEYKNRTIFDITNNLSIVILALRPSDEG | |
| | TYECVVLKYEKDAFKREHLAEVTLSVKADGSGGGGSESKYGPPCPPCPAP | |
| | EFLGGPSVFLFPPKPKDTLMISRTPEVTCVVVDVSQEDPEVQFNWYVDGV | |
| | EVHNAKTKPREEQFNSTYRVVSVLTVLHQDWLNGKEYKCKVSNKGLPSSI | |
| | EKTISKAKGQPREPQVYTLPPSQEEMTKNQVSLTCLVKGFYPSDIAVEWE | |
| | SNGQPENNYKTTPPVLDSDGSFFLYSRLTVDKSRWQEGNVFSCSVMHEAL | |
| | HNHYTQKSLSLSLG | |
| 341 | VIHVTKEVKEYATLSCGHNVSVEELAQTRIYWQKDKKMVLTMMSGDMNIW | CD80-Fc- |
| | PEYKNRTIFDITNNLSIVILALRPSDEGTYECVVLKQEKDAFKREHLAEV | CD80_341 |
| | RLSVKADGSGGGGSPCPPCPAPEFLGGPSVFLFPPKPKDTLMISRTPEVT | |
| | CVVVDVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRL | |
| | TVDKSRWQEGNVFSCSVMHEALHNHYTQKSLSLSLGGGGGSGGGGSGGGG | |
| | SVIHVTKEVKEYATLSCGYNVSVEELAQTRIYWQKDKKMVLTMMSGDMNI | |
| | WPEYKNRTIFDITNNLSIVILALRPSDEGTYECVVLKQEKDAFKREHLAE | |
| | VRLSVKAD | |
| 342 | VIHVTKEVKEYATLSCGHNVSVEELAQTRIYWQKDKKMVLIMMSGDMNIW | CD80-CD80- |
| | PEYKNRTIFDIINNLSIVILALRPSDEGTYECVVLKQEKDAFKREHLAEV | Fc_342 |
| | RLSVKADGGGGSGGGGSGGGGSVIHVTKEVKEYATLSCGYNVSVEELAQT | |
| | RIYWQKDKKMVLTMMSGDMNIWPEYKNRTIFDITNNLSIVILALRPSDEG | |
| | TYECVVLKQEKDAFKREHLAEVRLSVKADGSGGGGSESKYGPPCPPCPAP | |
| | EFLGGPSVFLFPPKPKDTLMISRTPEVTCVVVDVSQEDPEVQFNWYVDGV | |
| | EVHNAKTKPREEQFNSTYRVVSVLTVLHQDWLNGKEYKCKVSNKGLPSSI | |
| | EKTISKAKGQPREPQVYTLPPSQEEMTKNQVSLTCLVKGFYPSDIAVEWE | |
| | SNGQPENNYKTTPPVLDSDGSFFLYSRLTVDKSRWQEGNVFSCSVMHEAL | |
| | HNHYTQKSLSLSLG | |
| 343 | epkscdkthtcppcpapellggpsvflfppkpkdtlmisrtpevtcvvvd | IgG1 Fc |
| | vshedpevkfnwyvdgvevhnaktkpreeqynstyrvvsvltvlhqdwln | |
| | gkeykckvsnkalpapiektiskakgqprepqvytlppsreemtknqvsl | |
| | tclvkgfypsdiavewesngqpennykttppvldsdgsfflyskltvdks | |
| | rwqqgnvfscsvmhealhnhytqkslslspgk | |
| 344 | EPKSSDKTHTCPPCPAPEAEGAPSVFLFPPKPKDTLMISRTPEVTCVVVD | Fc |
| | VSHEDPEVKFNWYVDGVEVHNAKTKPREEQYNSTYRVVSVLTVLHQDWLN | C220S/L234A/ |
| | GKEYKCKVSNKALPAPIEKTISKAKGQPREPQVYTLPPSRDELTKNQVSL | L235E/G237A/ |
| | TCLVKGFYPSDIAVEWESNGQPENNYKTTPPVLDSDGSFFLYSKLTVDKS | K447del |
| | RWQQGNVFSCSVMHEALHNHYTQKSLSLSPG | |
| 345 | EPKSSDKTHTCPPCPAPEAEGAPSVFLFPPKPKDTLMISRTPEVTCVVVD | Fc |
| | VSHEDPEVKFNWYVDGVEVHNAKTKPREEQYNSTYRVVSVLTVLHQDWLN | C220S/L234A/ |
| | GKEYKCKVSNKALPAPIEKTISKAKGQPREPQVYTLPPSRDELTKNQVSL | L235E/G237A/ |
| | TCLVKGFYPSDIAVEWESNGQPENNYKTTPPVLDSDGSFFLYSKLTVDKS | E356D/M358L |
| | RWQQGNVFSCSVMHEALHNHYTQKSLSLSPGK | |
| 346 | EPKSSDKTHTCPPCPAPELLGGPSVFLFPPKPKDTLMISRTPEVTCVVVD | Fc (E356D |
| | VSHEDPEVKFNWYVDGVEVHNAKTKPREEQYNSTYRVVSVLTVLHQDWLN | and M358L) |
| | GKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKS | |
| | RWQQGNVFSCSVMHEALHNHYTQKSLSLSPG | |
| 347 | EPKSSDKTHTCPPCPAPELLGGPSVFLFPPKPKDTLMISRTPEVTCVVVD | Fc (C5S |
| | VSHEDPEVKFNWYVDGVEVHNAKTKPCEEQYGSTYRCVSVLTVLHQDWLN | (C220S), |
| | GKEYKCKVSNKALPAPIEKTISKAKGQPREPQVYTLPPSREEMTKNQVSL | R77C, |
| | TCLVKGFYPSDIAVEWESNGQPENNYKTTPPVLDSDGSFFLYSKLTVDKS | (R292C), |
| | RWQQGNVFSCSVMHEALHNHYTQKSLSLSPGK | N82G |
| | | (N297G), |
| | | V87C |
| | | (V302C) ) |
| 348 | EPKSSDKTHTCPPCPAPEAEGAPSVFLFPPKPKDTLMISRTPEVTCVVVD | Fc with |
| | VSHEDPEVKFNWYVDGVEVHNAKTKPREEQYNSTYRVVSVLTVLHQDWLN | C220S/L234A/ |
| | GKEYKCKVSNKALPAPIEKTISKAKGQPREPQVYTLPPSREEMTKNQVSL | L235E/G237A |
| | TCLVKGFYPSDIAVEWESNGQPENNYKTTPPVLDSDGSFFLYSKLTVDKS | |
| | RWQQGNVFSCSVMHEALHNHYTQKSLSLSPGK | |
| 349 | EPKSSDKTHTCPPCPAPPVAGPSVFLFPPKPKDTLMISRTPEVTCVVVDV | Fc with |
| | KHEDPEVKFNWYVDGVEVHNAKTKPREEQYNSTYRVVSVLTVLHQDWLNG | C220S/E233P/ |
| | KEYKCKVSNKALPAPIEKTISKAKGQPREPQVYTLPPSREEMTKNQVSLT | L234V/L235A/ |
| | CLVKGFYPSDIAVEWESNGQPENNYKTTPPVLDSDGSFFLYSKLTVDKSR | G236del/S267 |
| | WQQGNVFSCSVMHEALHNHYTQKSLSLSPGK | K |
| 350 | EPKSSDKTHTCPPCPAPELLGGPSVFLFPPKPKDTLMISRTPEVTCVVVD | Fc (C5S |
| | VSHEDPEVKFNWYVDGVEVHNAKTKPCEEQYGSTYRCVSVLTVLHQDWLN | (C220S), |
| | GKEYKCKVSNKALPAPIEKTISKAKGQPREPQVYTLPPSREEMTKNQVSL | R77C, |
| | TCLVKGFYPSDIAVEWESNGQPENNYKTTPPVLDSDGSFFLYSKLTVDKS | (R292C), |
| | RWQQGNVFSCSVMHEALHNHYTQKSLSLSPG | N82G |
| | | (N297G), |
| | | V87C |
| | | (V302C), |
| | | L232del |
| | | (K447del) ) |
| 351 | EPKSSDKTHTCPPCPAPEAEGAPSVFLFPPKPKDTLMISRTPEVTCVVVD | Fc with |
| | VSHEDPEVKFNWYVDGVEVHNAKTKPREEQYNSTYRVVSVLTVLHQDWLN | C220S/L234A/ |
| | GKEYKCKVSNKALPAPIEKTISKAKGQPREPQVYTLPPSREEMTKNQVSL | L235E/G237A/ |
| | TCLVKGFYPSDIAVEWESNGQPENNYKTTPPVLDSDGSFFLYSKLTVDKS | K447del |
| | RWQQGNVFSCSVMHEALHNHYTQKSLSLSPG | |
| 352 | EPKSSDKTHTCPPCPAPPVAGPSVFLFPPKPKDTLMISRTPEVTCVVVDV | Fc with |
| | KHEDPEVKFNWYVDGVEVHNAKTKPREEQYNSTYRVVSVLTVLHQDWLNG | C220S/E233P/ |
| | KEYKCKVSNKALPAPIEKTISKAKGQPREPQVYTLPPSREEMTKNQVSLT | L234V/L235A/ |
| | CLVKGFYPSDIAVEWESNGQPENNYKTTPPVLDSDGSFFLYSKLTVDKSR | G236del/S267 |
| | WQQGNVFSCSVMHEALHNHYTQKSLSLSPG | K/K447del |
| 353 | TKVDKTVERKCCVECPPCPAPPVAGPSVFLFPPKPKDTLMISRTPEVTCV | IgG2 Fc |
| | VVDVSHEDPEVQFNWYVDGVEVHNAKTKPREEQFNSTFRVVSVLTVVHQD | |
| | WLNGKEYKCKVSNKGLPAPIEKTISKTKGQPREPQVYTLPPSREEMTKNQ | |
| | VSLTCLVKGFYPSDIAVEWESNGQPENNYKTTPPMLDSDGSFFLYSKLTV | |
| | DKSRWQQGNVFSCSVMHEALHNHYTQKSLSLSPGK | |
| 354 | ESKYGPPCPSCPAPEFLGGPSVFLFPPKPKDTLMISRTPEVTCVVVDVSQ | IgG4 Fc |
| | EDPEVQFNWYVDGVEVHNAKTKPREEQFNSTYRVVSVLTVLHQDWLNGKE | |
| | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGK | |
| 355 | ESKYGPPCPPCPAPEFLGGPSVFLFPPKPKDTLMISRTPEVTCVVVDVSQ | IgG4 Fc |
| | EDPEVQFNWYVDGVEVHNAKTKPREEQFNSTYRVVSVLTVLHQDWLNGKE | S228P |
| | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGK | |
| 356 | DKPHTCPLCPAPELLGGPSVFLFPPKPKDTLMISRTPEVTCVVVDVSHED | Fc |
| | PEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVK | |
| | GFYPSDIAVEWESNGQPENNYKATPPVLDSDGSFFLYSKLTVDKSRWQQG | |
| | NVFSCSVMHEALHNHYTQKSLSLSPGK | |
| 357 | DPKSCDKPHTCPLCPAPELLGGPSVFLFPPKPKDTLMISRTPEVTCVVVD | Fc |
| | VSHEDPEVKFNWYVDGVEVHNAKTKPREEQYNSTYRVVSVLTVLHQDWLN | |
| | GKEYKCKVSNKALPAPIEKTISKAKGQPREPQVYTLPPSRDELTKNQVSL | |
| | TCLVKGFYPSDIAVEWESNGQPENNYKATPPVLDSDGSFFLYSKLTVDKS | |
| | RWQQGNVFSCSVMHEALHNHYTQKSLSLSPGK | |
| 358 | DKTHTCPPCPAPEAEGAPSVFLFPPKPKDTLMISRTPEVTCVVVDVSHED | Fc |
| | PEVKFNWYVDGVEVHNAKTKPREEQYNSTYRVVSVLTVLHQDWLNGKEYK | |
| | CKVSNKALPSSIEKTISKAKGQPREPQVYTLPPSRDELTKNQVSLTCLVK | |
| | GFYPSDIAVEWESNGQPENNYKTTPPVLDSDGSFFLYSKLTVDKSRWQQG | |
| | NVFSCSVMHEALHNHYTQKSLSLSPGK | |
| 359 | EPKSSDKTHTCPPCPAPEAEGAPSVFLFPPKPKDTLMISRTPEVTCVVVD | Fc |
| | VSHEDPEVKFNWYVDGVEVHNAKTKPREEQYNSTYRVVSVLTVLHQDWLN | |
| | GKEYKCKVSNKALPSSIEKTISKAKGQPREPQVYTLPPSRDELTKNQVSL | |
| | TCLVKGFYPSDIAVEWESNGQPENNYKTTPPVLDSDGSFFLYSKLTVDKS | |
| | RWQQGNVFSCSVMHEALHNHYTQKSLSLSPGK | |
| 360 | EPKSSDKTHTCPPCPAPEAEGAPSVFLFPPKPKDTLMISRTPEVTCVVVD | single chain |
| | VSHEDPEVKFNWYVDGVEVHNAKTKPREEQYNSTYRVVSVLTVLHQDWLN | Fc with |
| | GKEYKCKVSNKALPAPIEKTISKAKGQPREPQVYTLPPSRDELTKNQVSL | effectorless |
| | TCLVKGFYPSDIAVEWESNGQPENNYKTTPPVLDSDGSFFLYSKLTVDKS | mutation |
| | RWQQGNVFSCSVMHEALHNHYTQKSLSLSPGGGGGSGGGGSGGGGSGGGG | |
| | SGGGGSGGGGSGGGGSGGGGSGGGGSGGGGSGGGGSGGGGSEPKSSDKTH | |
| | TCPPCPAPEAEGAPSVFLFPPKPKDTLMISRTPEVTCVVVDVSHEDPEVK | |
| | FNWYVDGVEVHNAKTKPREEQYNSTYRVVSVLTVLHQDWLNGKEYKCKVS | |
| | NKALPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVES | |
| | CSVMHEALHNHYTQKSLSLSPG | |
| 361 | VECPPCPAPPVAGPSVFLFPPKPKDTLMISRTPEVTCVVVDVSHEDPEVQ | IgG2 Fc |
| | FNWYVDGVEVHNAKTKPREEQFNSTFRVVSVLTVVHQDWLNGKEYKCKVS | |
| | NKGLPAPIEKTISKTKGQPREPQVYTLPPSREEMTKNQVSLTCLVKGFYP | |
| | SDISVEWESNGQPENNYKTTPPMLDSDGSFFLYSKLTVDKSRWQQGNVFS | |
| | CSVMHEALHNHYTQKSLSLSPG | |
| 362 | DKTHTCPPCPAPEAEGAPSVELFPPKPKDTLMISRTPEVTCVVVDVSHED | IgG1 Fc |
| | PEVKFNWYVDGVEVHNAKTKPREEQYNSTYRVVSVLTVLHQDWLNGKEYK | effectorless |
| | CKVSNKALPAPIEKTISKAKGQPREPQVYTLPPSRDELTKNQVSLTCLVK | (L234A/L235E |
| | GFYPSDIAVEWESNGQPENNYKTTPPVLDSDGSFFLYSKLTVDKSRWQQG | /G237A) and |
| | NVFSCSVMHEALHNHYTQKSLSLSPG | K447del |
| 363 | EPKSC | hinge |
| 364 | EPKSS | Hinge |
| 365 | DIQMTQTTSSLSASLGDRVTISCRASQDISKYLNWYQQKPDGTVKLLIYH | anti-CD19 |
| | TSRLHSGVPS | ScFv |
| 366 | RFSGSGSGTDYSLTISNLEQEDIATYFCQQGNTLPYTFGGGTKLEITGST | CD8-derived |
| | SGSGKPGSGE | hinge and |
| | | transmembrane |
| | | domain |
| 367 | GSTKGEVKLQESGPGLVAPSQSLSVTCTVSGVSLPDYGVSWIRQPPRKGL | CD8-derived |
| | EWLGVIWGSE | hinge and |
| | | transmembrane |
| | | domain |
| 368 | TTYYNSALKSRLTIIKDNSKSQVFLKMNSLQTDDTAIYYCAKHYYYGGSY | CD8 hinge |
| | AMDYWGQGTS | and |
| | | transmembrane |
| | | domain |
| 369 | VTVSS | CD3zeta |
| | | intracellular |
| | | signaling |
| | | domain |
| 370 | KPTTTPAPRPPTPAPTIASQPLSLRPEASRPAAGGAVHTRGLDFASDIYI | 4-1BB- |
| | WAPLAGTCGVLLLSLVITLYC | derived |
| | | costimulatory |
| | | domain |
| 371 | AKPTTTPAPRPPTPAPTIASQPLSLRPEACRPAAGGAVHTRGLDFACDIY | CD28-derived |
| | IWAPLAGTCG | costimulatory |
| | | domain |
| 372 | VLLLSLVIT | CD28-derived |
| | | costimulatory |
| | | domain 2 |
| 373 | KPTTTPAPRPPTPAPTIASQPLSLRPEACRPAAGGAVHTRGLDFACDIYI | CD28-derived |
| | WAPLAGTCGVLLLSLVIT | costimulatory |
| | | domain 3 |
| 374 | RVKFSRSADAPAYQQGQNQLYNELNLGRREEYDVLDKRRGRDPEMGGKPR | T2A protein |
| | RKNPQEGLYNELQKDKMAEAYSEIGMKGERRRGKGHDGLYQGLSTATKDT | |
| | YDALHMQALPPR | |
| 375 | KRGRKKLLYIFKQPFMRPVQTTQEEDGCSCRFPEEEEGGCEL | T2A |
| 376 | SKRSRLLHSDYMNMTPRRPGPTRKHYQPYAPPRDFAAYRS | T2A |
| 377 | RSKRSRLLHSDYMNMTPRRPGPTRKHYQPYAPPRDFAAYRS | Protein |
| 378 | FWVRSKRSRLLHSDYMNMTPRRPGPTRKHYQPYAPPRDFAAYRS | T2A |
| 379 | SGEGRGSLLTCGDVEENPGP | Nucleotide |
| 380 | EGRGSLLTCGDVEENPGP | P2A protein |
| 381 | GSGEGRGSLLTCGDVEENPGP | Blue |
| | | fluorescent |
| | | protein |
| 382 | Ggcagtggcgagggcagaggaagtctgctaacatgcggtgacgtcgagga | Blue |
| | gaatcctggccca | fluorescent |
| | | protein |
| 383 | GSGATNFSLLKQAGDVEENPGP | eGFP |
| 384 | SELIKENMHMKLYMEGTVDNHHFKCTSEGEGKPYEGTQTMRIKVVEGGPL | Green |
| | PFAFDILATS | fluorescent |
| | | protein |
| 385 | FLYGSKTFINHTQGIPDFFKQSFPEGFTWERVTTYEDGGVLTATQDTSLQ | eGFP |
| | DGCLIYNVKI | |
| 386 | RGVNFTSNGPVMQKKTLGWEAFTETLYPADGGLEGRNDMALKLVGGSHLI | antiCD19x28z |
| | ANIKTTYRSK | chimeric |
| | | antigen |
| | | receptor: |
| | | protein |
| 387 | KPAKNLKMPGVYYVDYRLERIKEANNETYVEQHEVAVARYCDLPSKLGHK | 2nd Gen CAR |
| | LN | (without |
| | | signal |
| | | sequence or |
| | | T2A or BFP) |
| 388 | SRSELIKENMHMKLYMEGTVDNHHFKCTSEGEGKPYEGTQTMRIKVVEGG | 2nd Gen CAR |
| | PLPFAFDILATSFLYGSKTFINHTQGIPDFFKQSFPEGFTWERVTTYEDG | with T2A |
| | GVLTATQDTSLQDGCLIYNVKIRGVNFTSNGPVMQKKTLWEAFTETLYPA | |
| | DGGLEGRNDMALKLVGGSHLIANIKTTYRSKKPAKNLKMPGVYYVDYRLE | |
| | RIKEANNETYVEQHEVAARYCDLPSKLGHKLN | |
| 389 | SKGEELFTGVVPILVELDGDVNGHKFSVSGEGEGDATYGKLTLKFICTTG | 2nd Gen CAR |
| | KLPVPWPTL | with CD28- |
| | | derived |
| | | costimulatory |
| | | domain |
| | | (without |
| | | signal |
| | | sequence or |
| | | T2A or BFP) |
| 390 | VTTLTYGVQCFSRYPDHMKQHDFFKSAMPEGYVQERTIFFKDDGNYKTRA | antiCD19 |
| | EVKFEGDTLV | chimeric |
| | | antigen |
| | | receptor |
| | | CAR-2 |
| 391 | NRIELKGIDFKEDGNILGHKLEYNYNSHNVYIMADKQKNGIKVNFKIRHN | antiCD19 |
| | IEDGSVQLAD | chimeric |
| | | antigen |
| | | receptor |
| | | CAR-2 |
| 392 | HYQQNTPIGDGPVLLPDNHYLSTQSALSKDPNEKRDHMVLLEFVTAAGIT | anti-CD19 |
| | HGMDELYK | chimeric |
| | | antigen |
| | | receptor |
| | | CAR-2 |
| 393 | SKGEELFTGVVPILVELDGDVNGHKFSVSGEGEGDATYGKLTLKFICTTG | antiCD19 |
| | KLPVPWPTL | chimeric |
| | | antigen |
| | | receptor |
| | | CAR-1 |
| 394 | VTTFSYGVQCFSRYPDHMKQHDFFKSAMPEGYVQERTIFFKDDGNYKTRA | antiCD19 |
| | EVKFEGDTLV | chimeric |
| | | antigen |
| | | receptor |
| | | CAR-1 |
| 395 | NRIELKGIDFKEDGNILGHKLEYNYNSHNVYIMADKQKNGIKVNFKIRHN | Anti-CD19 |
| | IEDGSVQLAD | scFv |
| 396 | MKWVTFISLLFLFSSAYS | HSA signal |
| | | peptide |
| 397 | MDMRAPAGIFGFLLVLFPGYRS | Ig kappa |
| | | light chain |
| 398 | MTRLTVLALLAGLLASSRA | human |
| | | azurocidin |
| | | preprotein |
| | | signal |
| | | sequence |
| 399 | MELGLSWIFLLAILKGVQC | IgG heavy |
| | | chain signal |
| | | peptide |
| 400 | MELGLRWVFLVAILEGVQC | IgG heavy |
| | | chain signal |
| | | peptide |
| 401 | MKHLWFFLLLVAAPRWVLS | IgG heavy |
| | | chain signal |
| | | peptide |
| 402 | MDWTWRILFLVAAATGAHS | IgG heavy |
| | | chain signal |
| | | peptide |
| 403 | MDWTWRFLFVVAAATGVQS | IgG heavy |
| | | chain signal |
| | | peptide |
| 404 | MEFGLSWLFLVAILKGVQC | IgG heavy |
| | | chain signal |
| | | peptide |
| 405 | MEFGLSWVFLVALFRGVQC | IgG heavy |
| | | chain signal |
| | | peptide |
| 406 | MDLLHKNMKHLWFFLLLVAAPRWVLS | IgG heavy |
| | | chain signal |
| | | peptide |
| 407 | MDMRVPAQLLGLLLLWLSGARC | IgG Kappa |
| | | light chain |
| | | signal |
| | | sequences: |
| 408 | MKYLLPTAAAGLLLLAAQPAMA | IgG Kappa |
| | | light chain |
| | | signal |
| | | sequences: |
| 409 | MGVKVLFALICIAVAEA | Gaussia |
| | | luciferase |
| 410 | MKWVTFISLLFLFSSAYS | Human |
| | | albumin |
| 411 | MAFLWLLSCWALLGTTFG | Human |
| | | chymotrypsinogen |
| 412 | MQLLSCIALILALV | Human |
| | | interleukin- |
| | | 2 |
| 413 | MNLLLILTFVAAAVA | Human |
| | | trypsinogen- |
| | | 2 |
| 414 | VIHVTKEVKEYATLSCGHNVSVEELAQYRIYWQKEKKMVLTMMSGDLNIW | CD80-CD80- |
| | PEYKNRTIFDITNNLSIVILGLRPSDEGTYECVVLKYEKDAFKREHLAEV | Fc_414 |
| | TLSVKADGGGGGSGGGGSGGGGSVIHVTKEVKEYATLSCGYNVSVEELAQ | |
| | YRIYWQKEKKMVLTMMSGDLNIWPEYKNRTIFDIINNLSIVILGLRPSDE | |
| | GTYECVVLKYEKDAFKREHLAEVTLSVKADGSGGGGSESKYGPPCPPCPA | |
| | PEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSS | |
| | IEKTISKAKGQPREPQVYTLPPSQEEMTKNQVSLTCLVKGFYPSDIAVEW | |
| | ESNGQPENNYKTTPPVLDSDGSFFLYSRLTVDKSRWQEGNVFSCSVMHEA | |
| | LHNHYTQKSLSLSLG | |
| 415 | VIHVTKEVKGWATLSCGYNVSVEELAQYRIYWQKEKKMVLTMMSGDLNIW | CD80_415 |
| | PEYKNRTIFDIINNLSIVILALRPSDEGTYECVVLKYEKDAFKREHLAEV | |
| | TLSVKAD | |
| 416 | VIHVTKEVKEYATLSCGYNVSVEELAQYRIYWQKEKKMVLTMMSGDLNIW | CD80_416 |
| | PEYKNRTIFDIINNLSIVILALRPSDEGTYECVVLKYEKDAFKREHLAEV | |
| | TLSVKAD | |
| 417 | VIHVTKEVKEYATLSCGYNVSVEELAQYRIYWQKEKKMVLTMMSGDLNIW | CD80_417 |
| | PEYKNRTIFDITNNLSIVILGLRPSDEGTYECVVLKYEKDAFKREHLAEV | |
| | TLSVKAD | |

1. An immunomodulatory protein comprising at least one variant CD80 extracellular domain polypeptide, wherein the variant CD80 extracellular domain polypeptide comprises an amino acid substitution at position 11 in the sequence set forth in SEQ ID NO:2 or a portion thereof comprising an IgV domain, wherein the variant CD80 extracellular domain polypeptide exhibits increased binding to PD-L1 compared to wild-type CD80 set forth in SEQ ID NO:2 or the portion thereof comprising the IgV domain.

2. An immunomodulatory protein comprising at least one variant CD80 extracellular domain polypeptide, wherein the variant CD80 extracellular domain polypeptide comprises 2 to 10 amino acid substitutions at positions in the sequence set forth in SEQ ID NO:2 or a portion thereof comprising an IgV domain, wherein at least one substitution is at a position selected from among 9, 10, or 11, wherein the variant CD80 extracellular domain polypeptide exhibits increased binding to PD-L1 compared to wild-type CD80 set forth in SEQ ID NO:2 or the portion thereof comprising the IgV domain.

3. The immunomodulatory protein of claim 1 or claim 2, wherein the variant CD80 extracellular domain polypeptide comprises an amino acid substitution at position 11 and the substitution is to an aromatic amino acid residue.

4. The immunomodulatory protein of claim 2, wherein the aromatic amino acid residues are selected from the group consisting of tyrosine (Y), tryptophan (W) or phenylalanine (F), optionally wherein the amino acid substitution is V11Y, V11F, or V11W.

5. The immunomodulatory protein of any of claims 1-4, wherein the variant CD80 extracellular domain polypeptide comprises the amino acid substitution V11Y.

6. The immunomodulatory protein of any of claims 1-5, wherein the variant CD80 extracellular domain polypeptide comprises the amino acid substitutions V11Y/M47V/F59S/L85E, V11Y/M47V/L85E, V11Y/M42I/M47V/A71G, V11Y/T28Y/M47L/L85E, E10S/V11F/T28Y/M47L/T62S, E10G/V11W/V22S/T28P/A71G/E81K/Y87R, V11Y/T28Y/L85E/Y87I, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E10G/V11W/M47V/L85E, V11Y/T28Y/M47L/V68L/L85E, V11Y/M42W/T62A/L85E, V11Y/M42W/T62A, V11Y/M42W/F59Y/V68N, V11Y/M42W/E52K/T62A/L85E, V11Y/E35D/Y87Q/T101R, K9N/E10A/V11W/H18F/T28H/M47L/T62E/R73D, V11Y/E35G/M42G/F59S, V11Y/T28R/E35G/M47L/F59S, V11Y/T28R/E35G/M47L/A71G, V11Y/V68T, V11W/T28Y/D46V/R73E/F92L, V11W/T28Y/D46V/V68T/R73T/Y87N, E10S/V11Y/M42R/A71V, K9N/V11W/M47L/V68T/R73T/Y87N, V11Y/M47L/V68T, V11W/T28H/D46Q/V68L/L85E, E7Q/V11Y/R29H/M47L/V68T, V11Y/M47V/A71G/L85E, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

7. The immunomodulatory protein of claim 2, wherein the variant CD80 extracellular domain polypeptide comprises an amino acid substitution at position 9 and is substitution to a polar uncharged amino acid residue.

8. The immunomodulatory protein of claim 7, wherein the polar uncharged amino acid is selected from the group consisting of serine (S), asparagine (N), glutamine (Q), threonine (T).

9. The immunomodulatory protein of any of claims 2, 7 and 8, wherein the variant CD80 extracellular domain polypeptide comprises the amino acid substitution K9S or K9N.

10. The immunomodulatory protein of claim 2, wherein the variant CD80 extracellular domain polypeptide comprises an amino acid substitution at position 9 and the substitution is to another basic amino acid.

11. The immunomodulatory protein of claim 10, wherein the other basic amino acid is selected from the group consisting of arginine or histidine.

12. The immunomodulatory protein of any of claims 2, 10 and 11, wherein the variant CD80 extracellular domain polypeptide comprises the amino acid substitution is K9R.

13. The immunomodulatory protein of any of claims 2 and 7-12, wherein the variant CD80 extracellular domain polypeptide comprises the amino acid substitutions K9N/E10G/N63S/L85E, K9N/E10G/E35G/D46E/M47V/V68M, K9R/E10S/V11Y/M42L/F59M/V68M/L85E, K9S/E10R/V11Y/M47L/A71G, K9N/E10R/H18V/T28Y/A71G, K9N/E10A/V11W/H18F/T28H/M47L/T62E/R73D, K9R/E10A/E35G/V68T/T101K, K9R/E10A/E35G/V68L/L85E, K9N/E10G/Y87K/T101Q, K9N/V11W/M47L/V68T/R73T/Y87N, K9R/A26T/T28Y/E35A/M47L/A71G.

14. The immunomodulatory protein of claim 2, wherein the variant CD80 extracellular domain comprises an amino acid substitution at position 10 and the substitution is to a nonpolar amino acid.

15. The immunomodulatory protein of claim 14, wherein the nonpolar amino acid is glycine, alanine or valine.

16. The immunomodulatory protein of claim 2, 14 or 15, wherein the variant CD80 extracellular domain comprises the amino acid substitution E10G or E10A

17. The immunomodulatory protein of claim 2, wherein the variant CD80 extracellular domain comprises an amino acid substitution at position 10 and the amino acid substitution is selected from the group consisting of E10G, E10S, E10R, and E10A.

18. The immunomodulatory protein of any of claims 2 and 14-17, wherein the variant CD80 extracellular domain polypeptide comprises the amino acid substitutions K9N/E10G/N63S/L85E, E10G/E35G/D46E/M47V/V68M, K9N/E10G/E35G/D46E/M47V/V68M, K9R/E10S/V11Y/M42L/F59M/V68M/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, E10G/V11W/T28Y/M47L, K9S/E10R/V11Y/M47L/A71G, E10G/V11W/E35G/M47L/A71G/Y87R, E10G/V11W/M47V/L85E, E10G/H18Y, K9N/E10R/H18V/T28Y/A71G, E10G/H18Y/T28Y/M47W/T62S, K9N/E10A/V11W/H18F/T28H/M47L/T62E/R73D, E10G/D46K/L85E, E10G/H18T/Q27T/D46E/M47L, K9R/E10A/E35G/V68T/T101K, E10G/E35G/D46E/M47V/V68M/T101K, K9R/E10A/E35G/V68L/L85E, K9N/E10G/Y87K/T101Q, E10S/V11Y/M42R/A71V, E10G/A26S/T28Y, E10S/V68M/Y87P, E10G/Q27F/D46N/A71G/D90G, or E10S/V11F/T28Y/M47L.

19. The immunomodulatory protein of any of claims 1-5, 7-12 and 14-17, wherein the variant CD80 extracellular domain polypeptide further comprises an amino acid substitution at position 47 in the sequence set forth in SEQ ID NO:2 or the portion thereof comprising an IgV domain.

20. The immunomodulatory protein of claim 19, wherein the amino acid substitution at position 47 is to another hydrophobic amino acid.

21. The immunomodulatory protein of claim 20, wherein the hydrophobic amino acid is selected from the group consisting of valine, leucine, isoleucine or proline.

22. The immunomodulatory protein of any of claims 19-21, wherein the variant CD80 extracellular domain comprises the amino acid substitution M47L or M47V.

23. The immunomodulatory protein of any of claims 19-22, wherein the variant CD80 extracellular domain polypeptide comprises the amino acid substitutions V11W/M47L, V11F/M47L, V11Y/M47L, V11W/M47V, or V11Y/M47V.

24. The immunomodulatory protein of any of claims 19-23, wherein the variant CD80 extracellular domain polypeptide comprises the amino acid substitutions V11Y/M47V/F59S/L85E, V11Y/M47V/L85E, V11Y/M42I/M47V/A71G, V11Y/T28Y/M47L/L85E, E10S/V11F/T28Y/M47L/T62S, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E10G/V11W/M47V/L85E, V11Y/T28Y/M47L/V68L/L85E, K9N/E10A/V11W/H18F/T28H/M47L/T62E/R73D, V11Y/T28R/E35G/M47L/F59S, V11Y/T28R/E35G/M47L/A71G, K9N/V11W/M47L/V68T/R73T/Y87N, V11Y/M47L/V68T, E7Q/V11Y/R29H/M47L/V68T, V11Y/M47V/A71G/L85E, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

25. The immunomodulatory protein of any of claims 1-5, 7-12, 14-17 and 19-23, wherein the variant CD80 extracellular domain polypeptide further comprises an amino acid substitution at position 28 in the sequence set forth in SEQ ID NO:2 or the portion thereof comprising an IgV domain.

26. The immunomodulatory protein of any of claims 1-5, 7-12, 14-17, 19-23 and 25, wherein the variant CD80 extracellular domain polypeptide comprises an amino acid substitution selected from T28Y, T28P, T28H, T28R, or T28V.

27. The immunomodulatory protein of claim 26, wherein the amino acid substitution is T28Y.

28. The immunomodulatory protein of any of claims 19-23 and 25-27, wherein the variant CD80 extracellular domain polypeptide comprises the amino acid substitutions V11W/T28Y/M47L, V11F/T28Y/M47L, V11Y/T28Y/M47L, V11W/T28Y/M47V, V11F/T28Y/M47V, or V11Y/T28Y/M47V.

29. The immunomodulatory protein of any of claims 1-5, 7-12, 14-17, 19-28, wherein the variant CD80 extracellular domain polypeptide comprises the amino acid substitutions V11Y/T28Y/M47L.

30. The immunomodulatory protein of any of claims 1-5, 7-12, 14-17, 19-29, wherein the variant CD80 extracellular domain polypeptide further comprises an amino acid substitution at position 68, optionally wherein the amino acid substitution is V68M, V68L, V68N, V68T, or V68S.

31. The immunomodulatory protein of any of claims 1-5, 7-12, 14-17, 19-30, wherein the variant CD80 extracellular domain polypeptide comprises the amino acid substitutions V11Y/T28Y/M47L/V68M.

32. The immunomodulatory protein of any of claims 1-5, 7-12, 14-17, 19-30, wherein the variant CD80 extracellular domain polypeptide comprises the amino acid substitutions V11Y/T28Y/M47L/V68L.

33. The immunomodulatory protein of any of claims 1-5, 7-12, 14-17, 19-29, wherein the variant CD80 extracellular domain polypeptide comprises the amino acid substitutions E10G/V11W/T28Y/M47L.

34. The immunomodulatory protein of any of claims 1-33, wherein the variant CD80 extracellular domain polypeptide further comprises an amino acid substitution at position 7 in the sequence set forth in SEQ ID NO:2 or a portion thereof comprising an IgV domain.

35. The immunomodulatory protein of any of claims 1-34, wherein the variant CD80 extracellular domain polypeptide further comprises an amino acid substitution at position 18 in the sequence set forth in SEQ ID NO:2 or a portion thereof comprising an IgV domain.

36. The immunomodulatory protein of claim 35, wherein the variant CD80 extracellular domain polypeptide comprises amino acid substitutions E10G/V11W/H18Y/T28Y/M47L, V11Y/H18Y/T28Y/M47L or V11Y/H18Y/T28Y/M47L/A71G.

37. An immunomodulatory protein comprising at least one variant CD80 extracellular domain polypeptide, wherein the variant CD80 extracellular domain polypeptide comprises an amino acid substitution at position 7 in the sequence set forth in SEQ ID NO:163 or a portion thereof comprising the IgV domain, wherein the variant CD80 extracellular domain polypeptide exhibits increased binding to PD-L1 compared to the wild-type CD80 extracellular domain polypeptide set forth in SEQ ID NO:2 or the portion thereof comprising the IgV domain.

38. The immunomodulatory protein of claim 34 or claim 37, wherein the variant CD80 extracellular domain polypeptide comprises an amino acid substitution at position 7 and the substitution is to a polar uncharged amino acid residue.

39. The immunomodulatory protein of claim 38, wherein the polar uncharged amino acid is selected from the group consisting of serine (S), asparagine (N), glutamine (Q), threonine (T).

40. The immunomodulatory protein of any of claims 34-39, wherein the variant CD80 extracellular domain polypeptide comprises the amino acid substitution E7Q, E7N or E7S.

41. The immunomodulatory protein of claim 34 or claim 37, wherein the variant CD80 extracellular domain polypeptide comprises an amino acid substitution at position 7 and the substitution is to a basic amino acid.

42. The immunomodulatory protein of claim 41, wherein the basic amino acid is selected from the group consisting of arginine, histidine or lysine.

43. The immunomodulatory protein of any of claims 34, 37, 41 and 42, wherein the variant CD80 extracellular domain polypeptide comprises the amino acid substitution E7H or E7K.

44. The immunomodulatory protein of any of claims 34-43, wherein the variant CD80 extracellular domain polypeptide comprises the amino acid substitutions E7S/H18I/V20L/A26K/M47L/A71N, E7K/V11W/N63H/A71G/Y87K, E7N/E35D/T101R, E7H/H18L/V20I/T28Y/D46S/A71G, E7N/E35D/F59S, or E7Q/V11Y/R29H/M47L/V68T.

45. The immunomodulatory protein of any of claims 1-44, further comprising an amino acid substitution at position 101 in the sequence set forth in SEQ ID NO:2 or a portion thereof comprising an IgV domain.

46. An immunomodulatory protein comprising at least one variant CD80 extracellular domain polypeptide, wherein the variant CD80 extracellular domain polypeptide comprises an amino acid substitution at position 101 in the sequence set forth in SEQ ID NO:2 or a portion thereof comprising an IgV domain, wherein the variant CD80 extracellular domain polypeptide exhibits increased binding to PD-L1 compared to wild-type CD80 set forth in SEQ ID NO:2 or the portion thereof comprising the IgV domain.

47. The immunomodulatory protein of claim 45 or claim 46, wherein the variant CD80 extracellular domain polypeptide comprises an amino acid substitution at position 101 and the substitution is to a charged amino acid residue.

48. The immunomodulatory protein of claim 47, wherein the charged amino acid residue is basic and the amino acid substitution is to a histidine (H), lysine (K) or arginine (R).

49. The immunomodulatory protein of claim 48, wherein the variant CD80 extracellular domain polypeptide comprises an amino acid substitution T101K or T101R.

50. The immunomodulatory protein of claim 47, wherein the charged amino acid residue is acidic and the amino acid substitution is to aspartate (D), glutamate (E), asparagine (N) or glutamine (Q).

51. The immunomodulatory protein of claim 50, wherein the variant CD80 extracellular domain polypeptide comprises an amino acid substitution T101Q.

52. The immunomodulatory protein of any of claims 45-51, wherein the variant CD80 extracellular domain polypeptide comprises the amino acid substitutions E7N/E35D/T101R, V11Y/E35D/Y87Q/T101R, E35D/V68T/T101K, K9R/E10A/E35G/V68T/T101K, E10G/E35G/D46E/M47V/V68M/T101K, or K9N/E10G/Y87K/T101Q.

53. The immunomodulatory protein of any of claims 45-49 and 52, wherein the variant CD80 extracellular domain polypeptide comprises the amino acid substitutions V11Y/E35D/Y87Q/T101R.

54. The immunomodulatory protein of any of claims 1-53, further comprising an additional amino acid substitution at a different position wherein the amino acid substitution is selected from the group consisting of E7S, E7K, E7N, E7H, E7Q, K9N, K9R, K9S, E10G, E10S, E10R, E10A, V11Y, V11F, V11W, H18I, H18Y, H18F, H18V, H18L, H18T, V20L, V20I, V22S, A26K, A26G, A26Q, A26E, A26S, A26T, Q27F, Q27T, T28Y, T28P, T28H, T28R, T28V, R29S, R29H, E35G, E35D, E35A, M42I, M42L, M42G, M42W, M42R, D46E, D46S, D46K, D46V, D46Q, D46N, M47V, M47L, M47R, M47W, E52K, F59S, F59M, F59Y, T62S, T62A, T62E, N63S, N63I, N63H, V68M, V68L, V68N, V68T, V68S, A71G, A71N, A71V, R73D, R73E, R73T, E77G, E81K, L85E, L85Q, Y87R, Y87I, Y87K, Y87Q, Y87N, Y87P, D90G, F92L, T101R, T101K, or T101Q, or a conservative amino acid substitution of any of the foregoing.

55. An immunomodulatory protein comprising at least one variant CD80 extracellular domain polypeptide, wherein the variant CD80 extracellular domain polypeptide comprises one or more amino acid substitutions in the sequence set forth in SEQ ID NO:2 or a portion thereof comprising the IgV domain, wherein the one or more amino acid substitutions is selected from E7S, E7K, E7N, E7H, E7Q, K9N, K9R, K9S, E10G, E10S, E10A, V11Y, V11F, V11W, V20I, V22S, Q27F, Q27T, T28P, T28H, T28R, T28V, R29S, E35A, M42L, M42G, M42W, M42R, D46S, D46K, D46Q, M47R, M47W, E52K, F59S, T62S, T62A, N63I, N63H, V68N, V68T, V68S, A71N, A71V, R73D, R73E, R73T, L85Q, Y87R, Y87I, Y87K, Y87P, T101R, T101K, and T101Q, wherein the variant CD80 polypeptide exhibits increased binding to PD-L1 compared to wild-type CD80 comprising the sequence set forth in SEQ ID NO:2 or the portion thereof comprising the IgV domain.

56. The immunomodulatory protein of any of claims 1-55, wherein the variant CD80 extracellular domain polypeptide comprises 2, 3, 4, 5, 6, 7, 8, 9 or 10 amino acid substitutions compared to SEQ ID NO:2 or the portion thereof comprising the IgV domain.

57. The immunomodulatory protein of any of claims 1-56, comprising no more than 4 amino acid substitutions compared to SEQ ID NO:2 or the portion thereof comprising the IgV domain.

58. The immunomodulatory protein of any of claims 1-57, comprising 2, 3 or 4 amino acid substitutions compared to SEQ ID NO:2 or the portion thereof comprising the IgV domain.

59. An immunomodulatory polypeptide comprising at least one variant CD80 extracellular domain polypeptide, wherein the variant CD80 extracellular domain polypeptide comprises the sequence set forth in SEQ ID NO:2 or a portion thereof comprising an IgV domain, in which is contained amino acid substitutions selected from V11Y/M47V/F59S/L85E, V11Y/M47V/L85E, V11Y/M42I/M47V/A71G, K9N/E10G/N63S/L85E, E10G/E35G/D46E/M47V/V68M, K9N/E10G/E35G/D46E/M47V/V68M, E7S/H18I/V20L/A26K/M47L/A71N, K9R/E10S/V11Y/M42L/F59M/V68M/L85E, V11Y/T28Y/M47L/L85E, E10R/M47R/N63I, E10S/V11F/T28Y/M47L/T62S, E10R/H18Y/A26G/E35D/V68L/L85E, E10G/V11W/V22S/T28P/A71G/E81K/Y87R, V11Y/T28Y/L85E/Y87I, H18F/M42G/F59Y/V68N, E10G/V11W/T28Y/M47L, K9S/E10R/V11Y/M47L/A71G, E10G/V11W/E35G/M47L/A71G/Y87R, V11Y/T28Y/M47L/A71G/Y87R, V11Y/A26Q/M47L/A71G/Y87R, V11Y/T28Y/M47L, E7K/V11W/N63H/A71G/Y87K, V11Y/H18Y/E35G/L85Q, E35D/V68L/L85E, E10G/V11W/M47V/L85E, T28Y/M47L, V11Y/T28Y/M47L/V68L/L85E, E7N/E35D/T101R, V11Y/M42W/T62A/L85E, V11Y/M42W/T62A, V11Y/M42W/F59Y/V68N, V11Y/M42W/E52K/T62A/L85E, V11Y/E35D/Y87Q/T101R, H18Y/A26E/R29S/E35D/M47L/V68M/A71G/E77G/D90G, E10G/H18Y, K9N/E10R/H18V/T28Y/A71G, E10G/H18Y/T28Y/M47W/T62S, E7H/H18L/V20I/T28Y/D46S/A71G, K9N/E10A/V11W/H18F/T28H/M47L/T62E/R73D, H18V/V20I/T28Y/E35G/M47V/R73E, E10G/D46K/L85E, E10G/H18T/Q27T/D46E/M47L, E35D/V68T/T101K, V11Y/E35G/M42G/F59S, V11Y/T28R/E35G/M47L/F59S, E7N/E35D/F59S, V11Y/T28R/E35G/M47L/A71G, K9R/E10A/E35G/V68T/T101K, V11Y/V68T, E10G/E35G/D46E/M47V/V68M/T101K, K9R/E10A/E35G/V68L/L85E, V11W/T28Y/D46V/R73E/F92L, K9N/E10G/Y87K/T101Q, V11W/T28Y/D46V/V68T/R73T/Y87N, E10S/V11Y/M42R/A71V, H18F/T28V/M47L/V68S, E10G/A26S/T28Y, E35D/D46Q/L85E, E10S/V68M/Y87P, K9N/V11W/M47L/V68T/R73T/Y87N, V11Y/M47L/V68T, K9R/A26T/T28Y/E35A/M47L/A71G, V11W/T28H/D46Q/V68L/L85E, E7Q/V11Y/R29H/M47L/V68T, V11Y/M47V/A71G/L85E, E10G/Q27F/D46N/A71G/D90G, V11W/T28Y/M47L, V11F/T28Y/M47L, V11Y/T28Y/M47L/V68M, V11Y/T28Y/M47L/V68L, V11Y/M47L/A71G, V11Y/T28Y/M47L/A71G, V11Y/M47V/V68M, V11Y/D46E/M47V/V68M, V11Y/T28Y/D46E/M47V, V11Y/T28Y/D46E/M47V/V68M, E10S/V11F/T28Y/M47L, V11F/T28Y/M47L/T62S, V11Y/M47L/A71G/Y87R, or V11Y/T28Y/M47L/Y87R.

60. The immunomodulatory protein of any one of claims 1-59, wherein the variant CD80 extracellular domain polypeptide exhibits at least 85% sequence identity to the sequence set forth in SEQ ID No: 2 or the portion thereof comprising the IgV domain.

61. The immunomodulatory protein of any one of claims 1-60, wherein the variant CD80 extracellular domain polypeptide exhibits at least 90% sequence identity to the sequence set forth in SEQ ID No: 2 or the portion thereof comprising the IgV domain.

62. The immunomodulatory protein of any one of claims 1-61, wherein the variant CD80 extracellular domain polypeptide exhibits at least 95% sequence identity to the sequence set forth in SEQ ID No: 2 or the portion thereof comprising the IgV domain.

63. The immunomodulatory protein of any one of claims 1-62, wherein the variant CD80 extracellular domain polypeptide exhibits at least 97% sequence identity to the sequence set forth in SEQ ID No: 2 or the portion thereof comprising the IgV domain.

64. The immunomodulatory protein of any of claims 1-63, wherein the portion of SEQ ID NO:2 comprising the IgV domain comprises amino acids 1-101 of SEQ ID NO:2 and has a length of no more than 110 amino acids.

65. The immunomodulatory protein of any of claims 1-64, wherein the portion of SEQ ID NO:2 comprising the IgV domain is set forth in SEQ ID NO:163.

66. The immunomodulatory protein of any of claims 1-63, wherein the portion of SEQ ID NO:2 comprising the IgV domain is set forth as amino acids 1-107 of SEQ ID NO:2 (SEQ ID NO:164).

67. The immunomodulatory protein of any one of claims 1-66, wherein the variant CD80 extracellular domain polypeptide comprises the sequence of amino acids set forth in any of SEQ ID NOS: 165-244 or a sequence of amino acids that exhibits at least 95% sequence identity to any of SEQ ID NOS: 165-244.

68. The immunomodulatory protein of any one of claims 1-67, wherein the variant CD80 extracellular domain polypeptide comprises the sequence of amino acids set forth in any of SEQ ID NOS: 165-244.

69. The immunomodulatory protein of any of claims 1-68, wherein the variant CD80 extracellular domain polypeptide is set forth in any one of SEQ ID NOS: 165-244.

70. The immunomodulatory protein of any of claims 1-69, wherein the variant CD80 extracellular domain polypeptide is set forth in SEQ ID NO:180.

71. The immunomodulatory protein of any of claims 1-69, wherein the variant CD80 extracellular domain polypeptide is set forth in SEQ ID NO:185.

72. The immunomodulatory protein of any of claims 1-69, wherein the variant CD80 extracellular domain polypeptide is set forth in SEQ ID NO:197.

73. The immunomodulatory protein of any of claims 1-69, wherein the variant CD80 extracellular domain polypeptide is set forth in SEQ ID NO:233.

74. The immunomodulatory protein of any of claims 1-69, wherein the variant CD80 extracellular domain polypeptide is set forth in SEQ ID NO:234.

75. The immunomodulatory protein of any of claims 1-74, comprising a heterologous moiety that is linked to the at least one variant CD80 polypeptide, optionally via a linker.

76. The immunomodulatory protein of claim 75, wherein the heterologous moiety is a half-life extending moiety, a multimerization domain, a targeting moiety that binds to a molecule on the surface of a cell, or a detectable label.

77. The immunomodulatory protein of claim 76, wherein the half-life extending moiety comprises a multimerization domain, albumin, an albumin-binding polypeptide, Pro/Ala/Ser (PAS), a C-terminal peptide (CTP) of the beta subunit of human chorionic gonadotropin, polyethylene glycol (PEG), long unstructured hydrophilic sequences of amino acids (XTEN), hydroxyethyl starch (HES), an albumin-binding small molecule, or a combination thereof.

78. The immunomodulatory protein of any of claims 1-75, wherein the immunomodulatory protein is a variant CD80-Fc fusion protein comprising the at least one variant polypeptide and an Fc region of an immunoglobulin.

79. The immunomodulatory protein of claim 78, wherein the at least one variant CD80 polypeptide is linked to the Fc region via a linker, optionally a peptide linker.

80. The immunomodulatory protein of claim 79, wherein the linker comprises a peptide linker and the peptide linker is selected from GGGGS (G4S; SEQ ID NO: 328), GSGGGGS (SEQ ID NO: 325), GGGGSGGGGS (2×GGGGS; SEQ ID NO: 329), GGGGSGGGGSGGGGS (3×GGGGS; SEQ ID NO: 330), GGGGSGGGGSGGGGSGGGGS (4×GGGGS, SEQ ID NO:331), GGGGSGGGGSGGGGSGGGGSGGGGS (5×GGGGS, SEQ ID NO: 332), GGGGSSA (SEQ ID NO: 333), or GSGGGGSGGGGS (SEQ ID NO:335) or combinations thereof.

81. The immunomodulatory protein of any of claims 78-80, wherein the immunoglobulin Fc is an IgG1 Fc domain, or is a variant Fc domain that exhibits reduced binding affinity to an Fc receptor and/or reduced effector function, optionally as compared to a wild-type IgG1 Fc domain.

82. The immunomodulatory protein of claim 81, wherein the immunoglobulin Fc is a variant IgG1 Fc domain comprising one or more amino acid substitutions selected from L234A, L234V, L235A, L235E, G237A, S267K, R292C, N297G, and V302C, by EU numbering.

83. The immunomodulatory protein of claim 82, wherein the immunoglobulin Fc region comprises the amino acid substitutions L234A, L235E an G237A by EU numbering, optionally wherein the Fc region is set forth in any of SEQ ID NOS: 344, 345, 348 or 351.

84. The immunomodulatory protein of claim 78-83, wherein the immunoglobulin Fc region comprises the sequence set forth in SEQ ID NO:344.

85. The immunomodulatory protein of any of claims 78-80, wherein the immunoglobulin Fc is an IgG4 Fc domain, optionally comprising the amino acid substitution S228P.

86. The immunomodulatory protein of any of claims 78-80 and 85, wherein the immunoglobulin Fc region comprises the sequence set forth in SEQ ID NO:326.

87. The immunomodulatory protein of any one of claims 78-86, wherein the variant CD80-Fc fusion protein comprises the structure: variant CD80 polypeptide (vCD80)-Linker-Fc region.

88. The immunomodulatory protein of any of claims 78-87, wherein the variant CD80-Fc fusion protein comprises the sequence of amino acids set forth in any one of SEQ ID NOS: 245-324 or a sequence that exhibits at least 85%, at least 90%, at least 95% or at least 97% sequence identity to any one of SEQ ID NOS: 245-324.

89. The immunomodulatory protein of any of claims 78-86, wherein the variant CD80-Fc fusion protein comprises the structure: (vCD80)-Linker-Fc region-Linker-(vCD80).

90. The immunomodulatory protein of any of claims 78-86 and 89, wherein the variant CD80-Fc fusion protein comprises the sequence of amino acids set forth in any one of SEQ ID NOS: 336, 338, 339 or 341, or a sequence that exhibits at least 85%, at least 90%, at least 95% or at least 97% sequence identity to any one of SEQ ID NOS: 336, 338, 339 or 341.

91. The immunomodulatory protein of any of claims 78-86, wherein the variant CD80-Fc fusion protein comprises the structure: (vCD80)-Linker-(vCD80)-Linker-Fc region.

92. The immunomodulatory protein of any of claims 78-86 and 91, wherein the variant CD80-Fc fusion protein comprises the sequence of amino acids set forth in SEQ ID NO: 340 or 342, or a sequence that exhibits at least 85%, at least 90%, at least 95% or at least 97% sequence identity to SEQ ID NO: 340 or 342.

93. The immunomodulatory protein of any of claims 78-92 that is a homodimer comprising two identical copies of the variant CD80-Fc fusion protein.

94. The immunomodulatory protein of any one of claims 1-93, wherein the PD-L1 is human PD-L1.

95. The immunomodulatory protein of any one of claims 1-94, wherein the binding affinity of the variant CD80 extracellular domain to PD-L1 is increased greater than 1.1-fold compared to wild-type CD80 set forth in SEQ ID NO:2 or the portion comprising the IgV domain.

96. The immunomodulatory protein of claim 95, wherein the binding affinity is increased greater than 1.5-fold, greater than 2-fold, greater than 3-fold, greater than 4-fold, greater than 5-fold, greater than 6-fold, greater than 7-fold, greater than 8-fold, greater than 9-fold or greater than 10-fold.

97. The immunomodulatory protein of any one of claims 1-96, wherein the binding affinity is determined by Mean Fluorescence Intensity (MFI) as measured by flow cytometry in a cell-based binding assay for a PD-L1-expressing cell.

98. The immunomodulatory protein of any of claims 1-97, wherein the immunomodulatory protein blocks binding of PD-L1 to PD-1.

99. The immunomodulatory protein of any of claims 1-98, wherein the variant CD80 extracellular polypeptide exhibit an off-rate (Koff) for binding to PD-L1 of less than 50×10⁻³ s⁻¹.

100. The immunomodulatory protein of any of claims 1-99, wherein the variant CD80 extracellular polypeptide has a Koff for binding to PD-L1 of at or about or less than 40×10⁻³ s⁻¹, 30×10⁻³ s⁻¹, 20×10⁻³ s⁻¹, 5×10⁻³ s⁻¹, 10×10⁻³ s⁻¹, 5×10⁻³ s⁻¹, or 1×10⁻³ s⁻¹.

101. The immunomodulatory protein of any of claims 1-99, wherein the variant CD80 extracellular polypeptide has a Koff for binding to PD-L1 of between 1×10⁻³ s⁻¹ and 50×10⁻³ s⁻¹, 1×10⁻³ s⁻¹ and 30×10⁻³ s⁻¹, 1×10⁻³ s⁻¹ and 20×10⁻³ s⁻¹, 1×10⁻³ s⁻¹ and 15×10⁻³ s⁻¹, 1×10⁻³ s⁻¹ and 10×10⁻³ s⁻¹, 1×10⁻³ s⁻¹ and 5×10⁻³ s⁻¹, 5×10⁻³ s⁻¹ and 50×10⁻³ s⁻¹, 5×10⁻³ s⁻¹ and 30×10⁻³ s⁻¹, 5×10⁻³ s⁻¹ and 20×10⁻³ s⁻¹, 5×10⁻³ s⁻¹ and 15×10⁻³ s⁻¹, 5×10⁻³ s⁻¹ and 10×10⁻³ s⁻¹, 10×10⁻³ s⁻¹ and 50×10⁻³ s⁻¹, 10×10⁻³ s⁻¹ and 30×10⁻³ s⁻¹, 10×10⁻³ s⁻¹ and 20×10⁻³ s⁻¹, 1×10⁻³ s⁻¹ and 15×10⁻³ s⁻¹, 5×10⁻³ s⁻¹ and 50×10⁻³ s⁻¹, 15×10⁻³ s⁻¹ and 30×10⁻³ s⁻¹, 15×10⁻³ s⁻¹ and 20×10⁻³ s⁻¹, 20×10⁻³ s⁻¹ and 50×10⁻³ s⁻¹, 20×10⁻³ s⁻¹ and 30×10⁻³ s⁻¹, or 30×10⁻³ s⁻¹ and 50×10⁻³ s⁻¹.

102. The immunomodulatory protein of any of claims 1-101, wherein the variant CD80 polypeptide binds CD28, optionally with a binding affinity is 0.8-fold to 30-fold of the binding affinity of wild-type CD80 set forth in SEQ ID NO:2 or the portion thereof comprising the IgV domain.

103. The immunomodulatory protein of any of claims 1-102, wherein the variant CD80 polypeptide exhibits increased binding to CD28 compared to wild-type CD80 set forth in SEQ ID NO:2 or the portion thereof comprising the IgV domain.

104. The immunomodulatory protein of any one of claims 1-94, wherein the binding affinity of the variant CD80 extracellular domain to CD80 is increased greater than 1.1-fold compared to wild-type CD80 set forth in SEQ ID NO:2 or the portion comprising the IgV domain.

105. The immunomodulatory protein of claim 103 or claim 104, wherein the binding affinity is increased greater than 1.5-fold, greater than 2-fold, greater than 3-fold, greater than 4-fold, greater than 5-fold, greater than 6-fold, greater than 7-fold, greater than 8-fold, greater than 9-fold or greater than 10-fold.

106. The immunomodulatory protein of any one of claims 102-105, wherein the binding affinity is determined by Mean Fluorescence Intensity (MFI) as measured by flow cytometry in a cell-based binding assay for a CD28-expressing cell.

107. The immunomodulatory protein of any one of claims 1-106, wherein the immunomodulatory protein exhibits CD28 agonism, optionally as determined in a T reporter assay.

108. The immunomodulatory protein of claim 107, wherein the CD28 agonism is PD-L1 dependent, optionally as determined in a T cell reporter assay in the presence of PD-L1 expressing cells.

109. The immunomodulatory protein of any of claims 1-108, wherein the immunomodulatory protein blocks binding of CTLA-4 to its ligand CD80 or CD86.

110. The immunomodulatory protein of any of claims 1-109 that is a soluble protein.

111. The immunomodulatory protein of any of claims 1-110 that is a purified protein.

112. A nucleic acid molecule(s) encoding the immunomodulatory protein of any of claims 1-111.

113. The nucleic acid molecule of claim 112, that is a synthetic nucleic acid.

114. The nucleic acid molecule of claim 112 or claim 113 that is a cDNA.

115. A vector, comprising the nucleic acid molecule of any of claims 111-113.

116. The vector of claim 115 that is an expression vector.

117. The vector of claim 115 or claim 116, wherein the vector is a mammalian expression vector or a viral vector.

118. An immune cell comprising the immunomodulatory protein of any of claims 1-111.

119. The immune cell of claim 118, wherein the immune cell further comprises a T cell receptor (TCR) or a chimeric antigen receptor (CAR).

120. The immune cell of claim 118 or claim 119, wherein the immune cell is a lymphocyte.

121. The immune cell of claim 120, wherein the lymphocyte is a T cell.

122. The immune cell of any of claims 118-121, wherein the immunomodulatory protein is a transmembrane protein expressed on the surface of the immune cell.

123. The immune cell of any of claims 118-122, wherein the immunomodulatory protein is secretable from the immune cell.

124. A method of producing an immunomodulatory protein comprising introducing the nucleic acid molecule of any of claims 111-113 or vector of any of claims 115-117 into a host cell under conditions to express the protein in the cell, and isolating or purifying the protein from the cell.

125. A purified immunomodulatory protein produced by the method of claim 124.

126. A pharmaceutical composition comprising the immunomodulatory protein of any of claims 1-111 or 125.

127. The pharmaceutical composition of claim 126, comprising a pharmaceutically acceptable excipient.

128. The pharmaceutical composition of claim 126 or claim 127, wherein the pharmaceutical composition is sterile.

129. An article of manufacture comprising the pharmaceutical composition of any of claims 126-128 in a vial or container.

130. The article of manufacture of claim 129, wherein the vial or container is sealed.

131. A kit comprising the pharmaceutical composition of any of claims 126-128, and instructions for use.

132. A method of stimulating an immune response in a subject, comprising administering an immunomodulatory protein of any of claims 1-111, the immune cell of any of claims 118-123, or the pharmaceutical composition of any of claims 126-128 to a subject in need thereof.

133. The method of claim 132, wherein stimulating the immune response treats a disease or condition in the subject.

134. A method of treating a disease or condition in a subject, the method comprising administering the immunomodulatory protein of any of claims 1-111, the immune cell of any of claims 118-123, or the pharmaceutical composition of any of claims 126-128 to a subject having the disease or condition.

135. The method of claim 133 or claim 134, wherein the disease or condition is a cancer.

136. The method of any of claims 132-135, wherein the subject has a PD-L1-expressing tumor.

137. The method of any of claims 132-136, wherein prior to the administering, the method comprises selecting a subject having an PD-L1-expressing tumor.
